(19) United States
(12) Patent Application Publication
(10) Pub. No.: US 2007/0254313 A1
(43) Pub. Date: Nov. 1, 2007

(54) Gnal Splice Variant and Uses Thereof

(75) Inventors: James Robert Bostwick, West Chester, PA (US); Thomas Defay, West Chester, PA (US); Stephen Furlong, Wilmington, DE (US); Lee T. Hirata, New Castle, DE (US); Vipa Ravyn, Wilmington, DE (US); Alan Robbins, Wilmington, DE (US); John Corradi, Cheshire, CT (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodetalje (SE)

(21) Appl. No.: 10/595,793

(22) PCT Filed: Nov. 11, 2004
(86) PCT No.: PCT/GB04/04749

§ 371 Date: Mar. 14, 2007

Related U.S. Application Data
(60) Provisional application No. 60/519,190, filed on Nov. 11, 2003.
(60) Provisional application No. 60/607,010, filed on Sep. 3, 2004.

(51) Int. Cl. G01N 33/68; C12N 15/63; C12P 21/02

(52) U.S. Cl. 435/7.8; 435/243; 435/320.1; 435/325; 435/348; 435/70.1; 530/350; 536/23.1

(57) ABSTRACT

The present invention relates to a novel splice form of the GNAL gene product and methods for identifying modulators of G protein coupled receptors.

### CROSS-REFERENCE TO RELATED APPLICATIONS

This application is related to U.S. Provisional Application 60/519,190, filed Nov. 11, 2003, and U.S. Provisional Application 60/607,010, filed Sep. 3, 2004, each of which is incorporated herein by reference in its entirety.

### FIELD OF THE INVENTION

The present invention relates to a novel splice form of the GNAL gene product and methods for identifying modulators of G protein coupled receptors.

### BACKGROUND

Schizophrenia and bipolar affective disorder (BPAD) are complex psychiatric disorders that affect 1-2% of the general population. Data from twin and family studies has suggested that genetics confers a significant proportion of the risk for developing both of these diseases. Available information also suggests that for both disorders the genetic liability derives from multiple genes. Furthermore, some researchers have suggested that these disorders have 1 or more susceptibility genes in common. Identification of these genes and/or factors affecting the expression of these genes will greatly enhance our ability to find common underlying disease processes and aid the identification and validation of new drug targets.

Evidence for specific susceptibility genes for both disorders has been reported, including neuregulin on chromosome 8, LG72 on chromosome 13 and dysbindin on chromosome 6. Identification of each of these genes followed from initial genetic linkage studies that identified a chromosomal region likely to contain a candidate gene followed by positional cloning. Other linkage regions have been reported, but have not yet yielded a confirmed candidate gene. One such chromosomal region is chromosome 18p11, one of the most consistently reproducible regions in linkage studies for BPAD. The first report of genetic linkage for bipolar disorder on 18p11 was by Berrettini et al., 1994, Proc. Natl. Acad. Sci. U.S.A., 91:5918-5921. This observation has been replicated in other studies. In addition to the evidence that this region contains a susceptibility locus for bipolar disorder, there is also strong evidence for a schizophrenia gene in this same region.

One candidate gene in this region is the GNAL (also known as Golf) gene, which codes for an olfactory-specific guanosine triphosphate [GTP]-binding protein α subunit, Gαolf. In the region of the GNAL gene, a (CA) repeat has been described that has multiple alleles. In association studies with schizophrenia kindreds using these GNAL CA repeats it was shown that a 124 bp allele was transmitted to ill offspring 45/58 times (p=0.00009). The implication from these studies is that a susceptibility allele must lie within ˜10 kb of the CA repeat. However, no exonic Golf variants have been found among ill persons from either schizophrenia or bipolar (BP) kindreds. Coding variants of Golf that would represent the susceptibility factor have not yet been identified. An open reading frame related to neurospychotropic disorders has been identified within intron 5 of the human GNAL gene (U.S. Pat. No. 6,414,313). Previous characterization of the human GNAL gene structure has identified 12 exons, and no evidence of alternative splicing or coding region polymorphisms has been reported.

Heterotrimeric G-proteins play a critical role in signal transduction initiated by ligand binding to seven transmembrane G-protein coupled receptors (GPCRs). The intracellular α, β,and γ subunits form a complex that associates with the C-terminal end of GPCRs. There is an exchange of GDP for GTP (GDP-GTP exchange) on the α subunit upon receptor activation, followed by dissociation of the βγ subunits, ultimately leading to generation of second messengers, such as cAMP. Two of the known mammalian α subunits are stimulatory in their ability to increase cAMP levels, Gαs and Gαolf.

Gαolf was originally identified by its enriched localization in rat olfactory tissue, and the similarity of its amino acid sequence to Gαs predicted its ability to stimulate adenylyl cyclase. Subsequent studies revealed that Gαolf is also expressed in other regions of the central nervous system (CNS), most prominently in the striatum, as well as in several peripheral tissues. Mice with a targeted disruption of the first four exons of the gene encoding Gαolf have demonstrated that Gαolf is critical for olfaction, and have provided direct evidence for the exclusive coupling of Gαolf to the dopamine D1 and adenosine A2a receptors in the striatum (Belluscio et al., 1998, Neuron, 20:69-81; Corvol et al., 2001, J. Neurochem., 76:1585-1588).

Human Gαolf is encoded by the GNAL gene on chromosome 18p11.2, in a susceptibility region for both bipolar disorder and schizophrenia. The genomic structure of GNAL includes 12 exons spanning more than 80 kb (Vuoristo et al., 2000, Mol. Psychiatry, 5:495-501).

Genomic imprinting is an epigenetic phenomenon that results in the preferential expression of a gene from one allele. Most human genes are expressed equally from both alleles. However, there are currently ˜75 human genes that are known to be ‘imprinted’, i.e., they carry an imprint of their parental origin. This imprint comes in the form of specific methylation of cytosine nucleotides in certain regions of DNA. Cytosines in CG dinucleotides are methylated in regions known as CpG islands, where the prevalence of this dinucleotide is greater than expected. Imprinted genes are therefore differentially methylated, carrying the imprint of methylation on one allele. CpG islands are usually located in the regulatory region of genes and methylation most often has the effect of silencing expression of the gene. Imprinting is the best understood mechanism that can give rise to parent-of-origin effects, where manifestation of disease is dependent on the transmitting parent.

One particularly striking example of the effects of imprinting on human disease is the GNAS locus, encoding Gαs. GNAS is a complex imprinted locus, with both maternally and paternally expressed transcripts (Hayward et al., 1996, Proc. Natl. Acad. Sci. USA, 93:9821-9826). Originally, the GNAS locus was thought to include 13 exons in total, encoding only Gαs. Several additional exons have since been identified, and the locus is now known to encode at least four alternate transcripts from different promoters and first exons. There are CpG islands found within both the 3′ and 5′ promoter regions. The most 5′ exon encodes an alternate first exon of the G-protein, and splices into exon 2 of the Gαs transcript, encoding an extra large form of Gαs, XLGαs. The CpG island associated with this exon, regulating expression of XLGαs, is methylated only on the maternal allele, leading to paternal expression of the transcript.

Thus, for GNAS, it is the XLGαs gene product that is imprinted. The cannonical Gαs transcript is biallelically expressed in most tissues, but maternally expressed in some. In addition, the NESP55 transcript that encodes a transcript for an acidic chromagranin from the same locus is methylated on the paternal allele and expressed only from the maternal allele. Complex regulation and imprinting of this locus lead to the manifestation of a spectrum of symptoms resulting from GNAS mutations, all dependent on the transmitting parent. A host of endocrine disorders arise from both activating and inactivating mutations of Gαs, as well as from an imprinting defect (for a review, see Weinstein et al., 2001, Endocr. Rev., 22:675-705).

The GTPγS assay is considered by many to be the assay of choice for functionally characterizing GPCRs (Sovago et al., 2001, Brain Res. Brain Res. Rev., 38:149-164.; Harrison & Traynor, 2003, Life Sci., 74:489-508; Milligan, 2003, Trends Pharmacol. Sci., 24:87-90). When an agonist activates a G-protein, GDP is released from the G-alpha subunit, and GTP is bound (GDP-GTP exchange). In the GTPγS assay, a non-hydrolyzable analog of GTP is bound that can be subsequently measured to determine GTPγS accumulation, and hence receptor activation. Any time a receptor is activated, a chain of events is stimulated within the cell. This GTP binding event is one of the earliest events that can be measured in this process, as such it is less sensitive to downstream amplification of the signal, and can give very accurate and functionally meaningful pharmacological parameters, such as potency and efficacy, to characterize the receptor.

Unfortunately, the GTPγS assay is not practical to use for many G-protein coupled receptors. For example, despite highly desirable attributes and widespread use, ligand regulation of [³⁵S]-GTPγS binding is mostly restricted to the analysis of ligands at GPCRs that interact with the subset of pertussis-toxin-sensitive Gi family G proteins (Milligan, 2003, supra).

This restriction has significantly limited the ability to screen compounds or drive structure-activity relationship (SAR) with a GTPγS assay on most Gs coupled GPCRs.

The difficulty in screening Gs coupled proteins is primarily a combination of a low stimulated signal from Gs coupled proteins and a high basal signal from Gi proteins. A couple of approaches have been devised to overcome these difficulties for Gs proteins including immuno-enrichment procedures for Gs and Gq coupled proteins, and expression in insect cell lines (Milligan, 2003, supra). Both approaches have had limited success and significant improvements can still be made. Some groups have been successful with the Sf9 insect cell system, including with Gs coupled proteins (Francken et al., 2001, Receptors Channels, 7:303-318; Nasman et al., 2001, Biochem. Pharmacol., 62:913-922; Houston et al., 2002, J. Neurochem., 80:678-696).

Adrenergic β2 receptor (β2) is the prototypic Gαs-coupled receptor and has been studied extensively for decades. Although methods have been described for measuring agonist-induced cAMP accumulation of adenylyl cyclase activity in a mammalian cell system, determining [³⁵S]-GTPγS binding has not been reported in mammalian systems. Agonist-induced [³⁵S]-GTPγS binding has been demonstrated in Sf9 cells coexpressing Gαs or Golf with the β2 receptor (Liu et al., 2001, J. Neurochem., 78:325-338; Seifert et al., 1998, Eur. J. Biochem., 255:369-382). However, such preparations in Sf9 cells resulted in low signal-to-noise ratios, with detection levels only 30%-50% above the baseline.

### SUMMARY

In one aspect, the present invention is directed to a novel splice variant of the Golf G protein, referred to herein as XLGolf. Accordingly, the invention provides an isolated nucleic acid having the nucleotide sequence encoding XLGolf of SEQ ID NO:1, or variants or fragments thereof The invention also provides a nucleic acid molecule comprising the complement of SEQ ID NO: 1, or variants or fragments thereof. In some embodiments, the present invention provides an expression vector containing the claimed nucleic acid molecule. In yet other embodiments, the expression vector containing the claimed nucleic acid molecule is contained within a cell.

In another aspect, the invention provides a purified polypeptide of XLGolf having the amino acid sequence of SEQ ID NO:2, or variants or fragments thereof.

In another aspect, the invention provides an isolated nucleic acid molecule encoding the polypeptide comprising the amino acid sequence of SEQ ID NO:2, or variants thereof. The invention further provides a nucleic acid molecule comprising the complement of the nucleotide sequence encoding the amino acid sequence of SEQ ID NO:2, or fragments of said nucleotide sequence.

In another aspect, the invention provides a method for producing a polypeptide comprising a) culturing a cell expressing a nucleic acid comprising a nucleotide sequence having at least 85% sequence identity to SEQ ID NO:1; and isolating the polypeptide.

In another aspect, the invention also provides a method for producing a polypeptide comprising: a) culturing a cell expressing a nucleic acid comprising a nucleotide sequence encoding a polypeptide comprising the amino acid sequence of SEQ ID NO:2, or a polypeptide having at least 85% sequence identity to SEQ ID NO:2; and b) isolating the polypeptide.

In another aspect, the invention provides a method for identifying compounds that modulate G protein coupled receptor (GPCR) activity comprising: a) providing a GPCR and a polypeptide comprising the amino acid sequence of SEQ ID NO:2, or a polypeptide having at least 80% sequence identity to SEQ ID NO:2; b) contacting the GPCR with a test compound; and c) determining GPCR activity, wherein a change in GPCR activity in the presence of said compound as compared with GPCR activity in the absence of said compound indicates that said compound modulates GPCR activity. In some embodiments, the GPCR is a Gs coupled GPCR In some embodiments, GPCR is selected from dopamine receptor D1, adenosine A2a receptor, and adrenergic β2 receptor. In some embodiments, the GPCR and the polypeptide are provided as cells expressing the GPCR and the polypeptide, or are provided as membranes prepared from said cells. In some embodiments, the cells are selected from mammalian, prokaryotic and insect cells. In some embodiments, GPCR activity is determined by detecting intracellular phospholipase C (PLC) activity, phospholipase A (PLA) activity, adenylyl cyclase activity, cAMP levels, MAP kinase activity, GDP-GTP exchange, intracellular concentration of calcium in the cell, or opening and closing of ion channels. In some embodiments, GDP-GTP exchange is determined by GTPγS binding or Eu-GTP binding. In some embodiments, the GPCR is also contacted with a ligand.

In another aspect, the invention provides a method for identifying compounds that inhibit G protein coupled receptor (GPCR) activity comprising: a) providing a GPCR, a GPCR ligand, and a polypeptide comprising the amino acid sequence of SEQ ID NO:2, or a polypeptide having at least 80% sequence identity to SEQ ID NO:2; b) contacting the GPCR with a test compound; and c) determining GPCR activity, wherein a decrease in GPCR activity in the presence of said compound as compared with GPCR activity in the absence of said compound indicates that said compound inhibits GPCR activity.

In another aspect, the invention provides a method for identifying G protein coupled receptor (GPCR) positive modulators comprising: a) providing a GPCR, a GPCR ligand, and a polypeptide comprising the amino acid sequence of SEQ ID NO:2, or a polypeptide having at least 80% sequence identity to SEQ ID NO:2; b) contacting the GPCR with a test compound; and c) determining GPCR activity, wherein an increase in GPCR activity in the presence of said compound as compared with GPCR activity in the absence of said compound indicates that said compound is a positive modulator of the GPCR

In another aspect, the invention provides a method for identifying compounds that activate a G protein coupled receptor (GPCR) comprising: a) providing a GPCR and a polypeptide comprising the amino acid sequence of SEQ ID NO:2, or a polypeptide having at least 80% sequence identity to SEQ ID NO:2; b) contacting the GPCR with a test compound; and c) determining GPCR activity, wherein an increase in GPCR activity in the presence of said compound as compared with GPCR activity in the absence of said compound indicates that said compound activates the GPCR.

In another aspect, the invention provides a method for identifying compounds that inhibit baseline G protein coupled receptor (GPCR) activity comprising: a) providing a GPCR and a polypeptide comprising the amino acid sequence of SEQ ID NO:2, or a polypeptide having at least 80% sequence identity to SEQ ID NO:2; b) contacting the GPCR with a test compound; and c) determining GPCR activity, wherein a decrease in GPCR activity in the presence of said compound as compared with GPCR activity in the absence of said compound indicates that said compound inhibits GPCR activity.

In another aspect, the invention provides a method for identifying compounds that modulate G protein coupled receptor (GPCR) activity comprising: a) providing a GPCR and a polypeptide comprising the amino acid sequence of SEQ ID NO:6, or a polypeptide having at least 80% sequence identity to SEQ ID NO:6, b) contacting the GPCR with a test compound, and c) determining GPCR activity, wherein a change in GPCR activity in the presence of said compound as compared with GPCR activity in the absence of said compound indicates that said compound modulates GPCR activity.

### BRIEF DESCRIPTION OF THE DRAWINGS

FIG. 1 shows a schematic of the genomic structure of the human GNAL gene (not drawn to scale). Exons 1 through 12 are the previously identified exons. The newly discovered alternate exon 1 (Alt 1) is shown about 60 kb 5′ of previously identified exon 1. The relative transcriptional start sites of XLG(olf) and G(olf) are indicated. Both first exons are spliced directly to exon 2. The relative positions of the CpG island regions are also indicated.

FIG. 2 shows a portion of the human genomic DNA sequence of chromosome 18p11.2 in the region of the alternate exon 1 of GNAL (SEQ ID NO:7). The upper case letters denote the longest 5′ EST sequence available for this exon. The exon lies within a CpG island, where G+C content is greater than 50% and the expected vs. observed ratio of CG dinucleotides is greater than 0.6. The predicted initiation codon is underlined.

FIG. 3 shows the multiple sequence alignment of the translated alternative transcripts encoded by the human GNAL and human GNAS genes: G(olf), Gαs (Galphas), XLG(olf), and XLGαs (XLGalphas) (SEQ ID NOs:8, 9, 10, and 11, respectively). The verticle line denotes the exon1/exon2 boundary. Fully conserved amino acids are indicated with an asterisk; conservative changes with a period. There is conservation between the N-terminal regions of the originally described proteins in the XL forms only in the βγ subunit binding domain. The alignment was carried out using ClustalW.

FIG. 4 is a bar graph showing [³⁵S]-GTPγS binding following A2a receptor activation by the agonist N-ethylcarboxamidoadenosine (NECA), where A2a was coexpressed with Golf (AF), Golf and beta and gamma G protein subunits (AFBG), XLGolf (AXL), or XLGolf and beta and gamma G protein subunits (AXLBG).

FIG. 5 is a line graph showing [³⁵S]-GTPγS binding plotted against dopamine concentration for dopamine-induced activation of the dopamine receptor D1 (DRD1). Membranes from Sf9 cells, infected for 48 hours with D1 dopamine receptor alone (square), or plus Golf (triangle), or plus XLGolf (circle) (MOI=1, 5 or 3, respectively), were analyzed for [35S] -GTPγS binding. Each datum represents the mean±SE of 3 experiments performed in triplicate.

FIG. 6 is a photo of an ethidium bromide stained gel containing amplification products from T98G glioma DNA. Amplification products in each lane were generated using the following primer pairs, with expected size of PCR products indicated in brackets: lane 1, Golf primers M25 and M26 (218 bp); lane 2, Golf primers U27 and U28 (223 bp); lane 3, Golf primers M37 and M38 (158 bp); lane 4, Golf primers U39 and U40 (162 bp); lane 5, XLGolf primers M1 and M2 (178 bp); lane 6, XLGolf primers U3 and U4 (183 bp); lane 7, XLGolf primers M2 and M5 (186 bp); lane 8, XLGolf primers U4 and U6 (190 bp).

FIG. 7 is a photo of an ethidium bromide stained gel containing amplification products from peripheral blood DNA. Amplification products in each lane were generated using the following primer pairs, with expected size of PCR products indicated in brackets: lane 1, Golf primers M25 and M26 (218 bp); lane 2, Golf primers U27 and U28 (223 bp); lane 3, Golf primers M37 and M38 (158 bp); lane 4, Golf primers U39 and U40 (162 bp); lane 5, XLGolf primers M1 and M2 (178 bp); lane 6, XLGolf primers U3 and U4 (183 bp); lane 7, XLGolf primers M2 and M5 (186 bp); lane 8, XLGolf primers U4 and U6 (190 bp).

FIG. 8 shows the complete genomic sequence of the human GNAL gene (SEQ ID NO:12). The genomic sequence is annotated with exons, CpG islands, and the polymorphisms we have identified.

FIG. 9 shows the amino acid sequence of the human Golf protein (SEQ ID NO:4).

FIG. 10 shows the nucleotide sequence (SEQ ID NO:1) encoding and amino acid sequence (SEQ ID NO:2) for the human XLGolf protein.

FIG. 11 shows N-terminal amino acid alignment of the translations of the long and short transcripts of human GNAL and human GNAS: Golf, Gαs, XLGolf, and XLGαs (SEQ ID NOs:42, 43, 44, and 45, respectively). The sequence in bold is that encoded by the common second exons. Asterisks denote identity and dots denote similarity. The alignment was carried out using ClustalW.

FIG. 12 shows an alignment of the nucleic acid coding sequences for human Golf (SEQ ID NO:3) and human XLGolf (SEQ ID NO:1). Upper case indicates identity.

FIG. 13 shows an alignment of the amino acid sequences for human Golf (SEQ ID NO:4) and human XLGolf (SEQ ID NO:2). Upper case indicates identity.

FIG. 14 is a bar graph showing the results of quantitation of Golf and XLGolf transcript levels in human CNS tissues and liver using real-time PCR The results are presented as the absolute amount of Golf or XLGolf transcript in each tissue divided by the relative level of β2-microglobulin for that tissue. The data represent the average of three cDNA preparations from each RNA sample with each cDNA subjected to Taqman PCR in triplicate.

FIG. 15 is a photo of an immunoblot showing expression of Golf and XLGolf in Sf9 cells. Sf9 cells were infected for 48 hours with D1 dopamine receptor (MOI=1) or with Golf (MOI=5) or XLGolf (MOI=3). Lane 1, cells infected with dopamine D1 receptor and Golf; lane 2, cells infected with dopamine D1 receptor and XLGolf; lane 3, cells infected with dopamine D1 receptor only.

FIG. 16 is a line graph showing the results of saturation binding of [³H]—SCH 23390 to membranes from Sf9 cells infected with dopamine D1 receptor with or without infection of Golf variants. Membranes from Sf9 cells infected for 48 hours with D1 dopamine receptor alone (square) or plus Golf (triangle) or plus XLGolf (circle) (MOI=1, 5 or 3, respectively) were evaluated for saturation binding. Each datum represents the mean±SE of 3 experiments performed in triplicate.

FIG. 17 presents photos of two ethidium bromide stained gels containing amplification products from various regions of the brain as well as the glioma cell line T98G. Methylation-specific PCR was used to detect methylated (M) and unmethylated (U) DNA.

FIG. 18 shows the cDNA sequence, including 3′ and 5′ untranslated regions, for mouse XLGolf (SEQ ID NO:5).

FIG. 19 shows the predicted amino acid sequence of the mouse XLGolf (SEQ ID NO:6).

### DETAILED DESCRIPTION

The present invention is based in part upon our discovery of a transcriptional variant of the GNAL gene, encoding a novel splice variant of the G protein alpha subunit protein Golf, referred to herein as XLGolf. The XLGolf protein has an altered N-terminus, as compared to Golf, and is encoded by a novel GNAL transcript having an alternative first exon spliced to the known exon 2 of GNAL.

We have discovered that Golf and XLGolf display different expression patterns in the central nervous system (CNS). We have further discovered that XLGolf can functionally couple to a variety of GPCRs, including the dopamine receptor D1 and the adenosine A2a receptor. In addition, we have discovered that there are CpG islands in the vicinity of both first exons in the GNAL gene that are differentially methylated; a hallmark of genomic imprinting.

XLGolf is useful in assays to screen for compounds that modulate the activity of G protein coupled receptors. For example, we have found that use of XLGolf in a GTPγS assay carried out in Sf9 cells, significantly increases the GTPγS signal. For example, XLGolf can be used to screen for agonists and/or positive modulators of Gs coupled GPCRs. In a particular example, we have found that the use of XLGolf has provided improved signal strength and improved signal to noise ratio for the Adenosine A2a (A2a), the Dopamine Receptor D1 (referred to herein as DRD1 or D1), and the adrenergic β2 GPCR receptors.

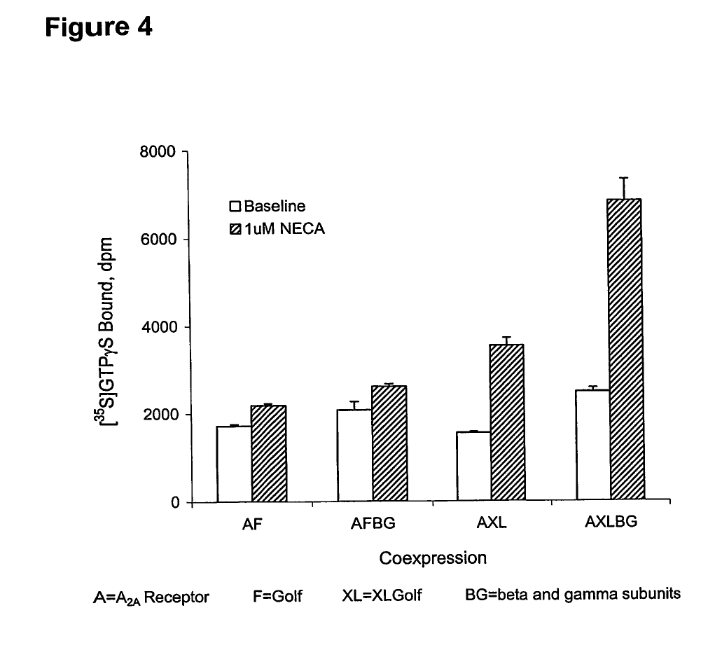

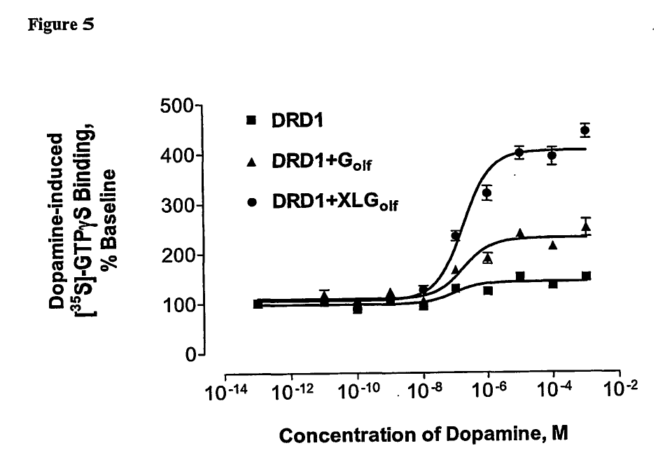

For example, using XLGolf in GTPγS assays of the A2a GPCR receptor produces more than a three-fold induction of GTPγS agonist signal over baseline. The addition of the Beta and Gamma subunits improved the signal still further (see FIG. 4). While induction seen with Golf was approximately 0.2, the use of the XLGolf splice form transformed this assay from one where the signal was barely observable to a highly robust assay, suitable for medium and high-throughput assays. We have seen similar results with the D1 receptor (see FIG. 5).

XLGolf Nucleic Acid Sequence and Polypeptide

The invention encompasses a G protein alpha subunit protein having at least 80%, e.g., 85%, 90%, 95%, 96%, 97%, 98% or 99%, sequence identity to the G protein alpha subunit protein sequence of SEQ ID NO:2. The comparison of sequences and determination of percent sequence identity between two sequences can be accomplished using a mathematical algorithm. For example, the percent identity between two amino acid sequences is determined using the Needleman & Wunsch (1970, J. Mol. Biol., 48:444-453) algorithm which has been incorporated into the GAP program in the GCG software package (available at http://www.gcg.com; see also Devereux et al., 1985, Nucleic Acids Res., 12:216-223), using either a Blosum 62 matrix or a PAM250 matrix, and a gap weight of 16, 14, 12, 10, 8, 6, or 4 and a length weight of 1, 2, 3, 4, 5, or 6. In another example, the percent identity between two nucleotide sequences is determined using the GAP program in the GCG software package (available at http://www.gcg.com; see also Devereux et al., 1985, supra), using a NWSgapdna.CMP matrix and a gap weight of 40, 50, 60, 70, or 80 and a length weight of 1, 2, 3, 4, 5, or 6. In still a further example, percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In a further example, percent identity between two amino acid or nucleotide sequences is determined using the PILEUP program (Devereux et al., 1985, supra).

The invention also encompasses polynucleotides that encode the G protein alpha subunit protein of SEQ ID NO:2, and variants thereof. Accordingly, any nucleic acid sequence which encodes the amino acid sequence of the splice variant can be used to produce recombinant molecules which express the XLGolf. It will be appreciated by those skilled in the art that as a result of the degeneracy of the genetic code, a multitude of nucleotide sequences encoding XLGolf, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of the naturally occurring GNAL gene, and all such variations are to be considered as being specifically disclosed.

The invention also encompasses production of DNA sequences, or fragments thereof, which encode XLGolf and its derivatives, entirely by synthetic chemistry. The polypeptides of the invention can be synthesised chemically. For example, by the Merrifield technique (Merrifield, 1963, J. Amer. Chem. Soc., 85:2149-2154). Numerous automated polypeptide synthesisers, such as Applied Biosystems' 431A Peptide Synthesizer also now exist. After production, the synthetic sequence may be inserted into any of the many available expression vectors and cell systems using reagents that are well known in the art.

Also encompassed by the invention are polynucleotide sequences that are capable of hybridizing to the claimed nucleic acid encoding a G protein alpha subunit protein, and in particular, those shown in SEQ ID NO:1, under various conditions of stringency as taught in Wahl et al., 1987, Methods Enzymol., 152:399-407 and Kimmel, 1987, Methods Enzymol., 152:507-511. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0 SSC at 50° C. to a high stringency of about 0.2×SSC at 50° C. In addition, the temperature in the wash step can be increased from low stringency conditions at room temperature, about 22° C., to high stringency conditions at about 65° C. Moderately stringent conditions are, for example at about 2.0×SSC and about 40° C.

Also included in the invention are G protein alpha subunit polypeptides having at least 80% amino acid sequence identity to the G protein alpha subunit protein of SEQ ID NO:2 and which variants retain the activity of the XLGolf protein. In some embodiments the G protein alpha subunit polypeptide variant is one having at least 85%, 90%, 95%, 96% 97%, 98% or 99% amino acid sequence identity to SEQ ID NO:2.

According to a further aspect of the invention there is provided an isolated polypeptide having at least 95% sequence identity to SEQ ID NO:2.

Also included in the invention are G protein alpha subunit-encoding polynucleotides or nucleic acid molecules having at least 80% sequence identity nucleotide sequence of SEQ ID NO:1. In some embodiments the polynucleotide is one having at least 85%, 90%, 95%, 96% 97%, 98% or 99% sequence identity to SEQ ID NO:1.

According to a further aspect of the invention there is provided an isolated nucleic acid comprising a nucleotide sequence which encodes a G protein alpha subunit protein variant having at least 80% sequence identity to SEQ ID NO:2. In some embodiments, the isolated nucleic acid encodes a G protein alpha subunit protein variant having 85%, 90%, 95%, 96% 97%, 98% or 99% amino acid sequence identity to SEQ ID NO:2

The invention also includes variants of the XLGolf protein which can contain one or more substitutions of amino acid residues which result in a silent change and a functionally equivalent XLGolf protein. Deliberate amino acid substitutions may be made on the basis of similarity in polarity, charge, solubility, hydrophobicity, hydrophilicity, and/or the amphipathic nature of the residues as long as the biological or immunological activity of G protein alpha subunit protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

In order to express a biologically active XLGolf, the nucleotide sequences encoding a XLGolf protein or functional equivalents, may be inserted into an appropriate expression vector, ie., a vector that contains the necessary elements for the transcription and translation of the inserted coding sequence. Methods which are well known to those skilled in the art may be used to construct expression vectors containing sequences encoding the XLGolf protein and appropriate transcriptional and translational control elements. These methods include in vitro recombinant DNA techniques, synthetic techniques, and in vivo genetic recombination. Such techniques are described in Sambrook et al., eds., Molecular Cloning: A Laboratory Manual (3^{rd} ed.) Cold Spring Harbor Laboratory Press, Cold Spring Harbor, N.Y. (2001); Ausubel et al., eds., Current Protocols in Molecular Biology, John Wiley & Sons, New York, N.Y. (2002).

A variety of expression vector/host systems may be utilized to contain and express sequences encoding the XLGolf protein. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV); bacterial expression vectors (e.g., Ti or pBR322 plasmids); or with animal cell systems. The invention is not limited by the host cell employed. When producing the polypeptide by recombinant expression in heterologous host strains, it may be desirable to adopt the codon usage (preference) of the host organism (Murray, 1989, Nucleic Acids Res., 17:477-508).

Control elements or regulatory sequences are those non-translated regions of the vector (enhancers, promoters, 5′ and 3′ untranslated regions) that interact with host cellular proteins to carry out transcription and translation. Such elements may vary in their strength and specificity.

Host cells transformed with nucleotide sequences encoding the XLGolf protein may be cultured under conditions suitable for the expression and recovery of the protein from the cell culture. The protein produced by a transformed cell may be secreted or contained intracellularly depending on the sequence and/or the vector used.

In another embodiment of the invention, natural, modified, or recombinant nucleic acid sequences encoding the XLGolf protein may be ligated to a heterologous sequence to encode a fusion protein. For example, to screen peptide libraries for inhibitors of XLGolf protein activity, it may be useful to encode a chimeric XLGolf protein that can be recognized by a commercially available antibody. A fusion protein may also be engineered to contain a cleavage site located between the XLGolf protein encoding sequence and the heterologous protein sequence, so that XLGolf protein may be cleaved and purified away from the heterologous moiety.

In another embodiment, the XLGolf protein may be produced using chemical methods to synthesize the amino acid sequence of the XLGolf protein, or a fragment thereof. For example, peptide synthesis can be performed using various solid-phase techniques (Roberge et al., 1995, Science, 269:202-204) and automated synthesis may be achieved, for example, using the ABI 431A Peptide Synthesizer (PerkinElmer). The newly synthesized peptide may be substantially purified by preparative high performance liquid chromatography (e.g., Creighton, T. (1983) Proteins, Structures and Molecular Principles, WH Freeman and Co., New York, N.Y.).

Drug Screening

The present invention provides assays to identify modulators of GPCR activity.

As used herein, the terms “modulate” or “modulates” in reference to GPCR activity include any measurable alteration to the quality and/or quantity and/or intensity of signal generated, including, but not limited to, any measurable alteration to receptor or enzymatic activity. Modulation of receptor activity includes activation, inhibition and potentiation of the activation by an agonist (natural or otherwise) of the receptor. Modulators of GPCR activity include agonists (partial and full), antagonists (orthosteric and allosteric), inverse agonists, and positive modulators. For example, unlike antagonists that block the activity of agonists but produce no activity on their own, an inverse agonist functions as an antagonist in non-constitutively active systems, but has the added property of actively reducing receptor-mediated constitutive activity of GPCR systems (response not resulting from agonist activation but rather spontaneously emanating from the system itself) (Kenakin, 2001, FASEB J., 15:598-611).

Modulators of GPCR activity can include compounds that activate, inhibit, or increase GPCR activity. Assays of the present invention can be used to identify all of these different types of GPCR modulators.

Compounds that inhibit GPCR activity induced by an agonist or ligand include antagonists (including orthosteric and allosteric).

Compounds that increase GPCR activity induced by an agonist or ligand include positive modulators.

Compounds that activate GPCRs include agonists and ligands.

Compounds that inhibit the baseline activity of a GPCR include inverse agonists. Baseline activity is the constitutive activity displayed by a GPCR in the absence of a ligand or agonist. Modulators of baseline acitivity, such as inverse agonists, are identified by a decrease in GPCR activity in their presence.

GPCR activity can be monitored using any of several different methods known to the art. For example, phospholipase C assays may be performed by growing cells in wells of a microtiter plate and then incubating the wells in the presence or absence of test compound, and total inositol phosphates (IP) may then be recovered and measured.

GPCR activity can also be determined based upon a measurement of intracellular calcium concentration. Many types of assays for determining intracellular calcium concentrations are well known to the art and can be employed in the methods of the invention. For example, cells can be grown to confluence on glass cover slides, rinsed, and incubated in the presence of an agent such as Fluo-3, Fluo-4, or FURA-2 AM (Molecular Probes, Eugene, Oreg.). After rinsing and further incubation, calcium displacement can be measured using a photometer.

GPCR activity can be determined by use of many methods known to the art. By way of non-limiting example, GPCR activity can be determined by detection of intracellular phospholipase C (PLC) activity, phospholipase A (PLA) activity, adenylyl cyclase activity, cAMP levels, MAP kinase activity, GDP-GTP exchange, intracellular concentration of calcium in the cell, and opening and closing of ion channels.

GDP-GTP exchange can be determined via the GTPγS binding assay, which is based upon the principle that agonists bind to G-protein coupled receptors and stimulate GDP-GTP exchange at the G-protein associated with the GPCR Since [³⁵S]-GTPγS is a non-hydrolyzable GTP analog, it can be used to provide an index of GDP-GTP exchange and, thus, receptor activation. The GTPγS binding assay therefore provides a quantitative measure of receptor activation. Another method for determining GDT-GTP exchange is the DELFIA GTP assay (PerkinElmer Life Sciences (Boston, Mass.), which uses Europium-GTP (Eu-GTP). This assay uses time-resolved fluorescence to measure binding of the non-radioactive Eu-GTP complex to Gα upon activation of a GPCR, and so does not involve any of the problems associated with the use of radioactivity (Frang et al., 2003, Assay Drug Dev. Technol., 1:275-280).

In general, screening assays include a GPCR and the XLGolf protein or a variant thereof Any GPCR from any source can be screened in the assays of the present invention. GPCRs from any organism may be assayed, for example mammalian GPCRs, including human, rodent, murine, rat, guinea pig, mouse, hamster, rhesus, cynomologous monkey, and porcine.

GPCR sequences are known in the art. For example, known human GPCRs are available from GenBank. For example, the following is a list of 120 human, non-olfactory GPCRs from the major families A, B, and C (includes RefSeqP identifiers, except for the last entry, which is a HUGO gene name):

Platelet-activating factor receptor (NP_{—}000943), NP_{—}005272, NP_{—}001516, NP_{—}000948, NP_{—}000950, NP_{—}114142, NP_{—}031395, NP_{—}005675, NP_{—}005291, NP_{—}067649, NP_{—}000625, NP_{—}005674, NP_{—}006047, NP_{—}064707, GPCR35 (NP_{—}005292), NP_{—}001328, NP_{—}005192, NP_{—}001287, NP_{—}006632, NP_{—}005274, NP_{—}001286, NP_{—}001828, NP_{—}000638, NP_{—}003956, NP_{—}000307, NP_{—}061844, NP_{—}009158, NP_{—}002971, NP_{—}005282, NP_{—}003458, NP_{—}005270, NP_{—}005039, NP_{—}055694, NP_{—}076404, NP_{—}073625, NP_{—}076403, NP_{—}149039, GPCR160 (NP_{—}055188), NP_{—}005281, NP_{—}000379, NP_{—}057641, NP_{—}037440, NP_{—}004876, NP_{—}064552, NP_{—}002053, NP_{—}005275, NP_{—}055441, NP 444508, NP_{—}612200, NP_{—}002368, NP_{—}004358, NP_{—}005290, NP_{—}001496, NP_{—}000814, NP_{—}005286, NP_{—}061843, NP_{—}005276, NP_{—}001392, GPCR21(NP_{—}005285), NP_{—}071429, NP_{—}110401, NP_{—}473373, NP_{—}473372, NP_{—}671732, NP_{—}473371, NP_{—}060960, NP_{—}004769, NP_{—}996880, NP_{—}036325, NP_{—}057624, NP_{—}065133, NP_{—}006134, NP_{—}061124, NP_{—}003970, NP_{—}003658, NP_{—}004063, GPCR12 (NP_{—}005279), NP_{—}570718, NP_{—}005758, NP_{—}065110, NP_{—}005283, NP_{—}003599, NP_{—}003476, NP_{—}037477, NP_{—}057319, NP_{—}004237, NP_{—}001829, NP_{—}061123, NP_{—}002557, NP_{—}005294, NP_{—}005296, NP_{—}005273, NP_{—}000155, NP_{—}060955, NP_{—}001497, NP_{—}002020, NP_{—}001453, NP_{—}002021, NP_{—}005288, NP_{—}001495, cysteinyl leukotriene receptor 1 (NP_{—}006630), NP_{—}005287, NP_{—}055314, NP_{—}473362, NP_{—}004215, NP_{—}001548, NP_{—}005499, NP_{—}000570, neuropeptide Y receptor Y1 (NP_{—}000900), NP_{—}006165, NP_{—}001499, NP_{—}072093, NP_{—}115892, NP_{—}694941, NP_{—}005747, NP_{—}061842, NP_{—}005284, NP_{—}115940, NP_{—}005289, GPR57.

In some embodiments, Gs-coupled GPCRs are screened. Any Gs-coupled GPCR can be screened by the assays of the present invention. Cao et al., 2003, Bioinformatics, 19:234-240 provides an algorithm for predicting the G protein coupling state of GPCRs.

The following 46 GPCRs are reported to and/or predicted to couple to Gs proteins: GPCRs NP_{—}000948, NP_{—}114142, NP_{—}005675, NP_{—}005291, NP_{—}005292, NP_{—}009158, NP_{—}073625, NP_{—}002053, NP_{—}055441, NP_{—}444508, NP_{—}005286, NP_{—}473372, NP_{—}006134, NP_{—}570718, NP_{—}002557, NP_{—}060955, NP_{—}473362, NP_{—}694941, NP_{—}061842, NP_{—}005289, NP_{—}000307, NP_{—}002971, NP_{—}005039, NP_{—}000814, NP_{—}004237, NP_{—}000948, NP_{—}000950, NP_{—}114142, NP_{—}005675, NP_{—}005291, NP_{—}005292, NP_{—}000307, NP_{—}061844, NP_{—}009158, NP_{—}002971, NP_{—}005270, NP_{—}005039, NP_{—}073625, NP_{—}064552, NP_{—}002053, NP_{—}005275, NP_{—}055441, NP_{—}444508, NP_{—}612200, NP_{—}005290, NP_{—}000814, NP_{—}005286, NP_{—}005276, NP_{—}071429, NP_{—}110401, NP_{—}473372, NP_{—}060960, NP_{—}036325, NP_{—}057624, NP_{—}006134, NP_{—}061124, NP_{—}005279, NP_{—}570718, NP_{—}065110, NP_{—}037477, NP_{—}057319, NP_{—}004237, NP_{—}061123, NP_{—}002557, NP_{—}000155, NP_{—}060955, NP_{—}473362, NP_{—}001499, NP_{—}694941, NP_{—}061842, NP_{—}005289.

In some embodiments, GPCRs for use in the assays of the present invention are selected from adenosine A2a receptor (NP_{—}000666), adenosine A2b receptor (NP_{—}000667), dopamine receptor D1 (NP_{—}000785), beta-2 adrenergic receptor (NP_{—}000015), dopamine receptor D5 (NP_{—}000789), histamine receptor H2 (NP_{—}071640), melanocortin 1 receptor (NP_{—}002377), melanocortin 2 receptor (NP_{—}000520), melanocortin 3 receptor (NP_{—}063941), melanocortin 4 receptor (NP_{—}005903), and melanocortin 5 receptor (NP_{—}005904).

In particular embodiments, GPCRs are selected from the dopamine receptor D1, the adenosine A2a receptor, and the adrenergic β2 receptor.

Any XLGolf polypeptide, or a variant thereof, from any source can be used in the assays of the present invention. For example, XLGolf polypeptides from human, mouse, or rat can be used. In some embodiments, a polypeptide comprising the amino acid sequence of SEQ ID NO:2, or a polypeptide having at least 80% sequence identity to SEQ ID NO:2 is used. In some embodiments the XLGolf polypeptide of SEQ ID NO:2 is used. In some embodiments, the mouse XLGolf polypeptide, which is encoded by nucleotide positions 196 -885 of SEQ ID NO:5, is used. In some embodiments, a polypeptide comprising the amino acid sequence of SEQ ID NO:6, or a polypeptide having at least 80% sequence identity to SEQ ID NO:6 is used.

In some embodiments, cells expressing a particular GPCR and XLGolf are used in assays to screen for compounds that modulate GPCR activity. In some embodiments, membranes derived from cells expressing a particular GPCR and XLGolf are used in assays to screen for compounds that modulate GPCR activity. Assays may be performed using either intact cells or membranes prepared from the cells (see e.g., Wang et al., Proc. Natl. Acad. Sci. U.S.A. 90:10230-10234 (1993)).

Any cell type in which a GPCR of interest is expressed or can be engineered to be expressed can be used. By way of non-limiting example, the assay may utilize mammalian cells (including, but not limited to, human, hamster, mouse, rat, or monkey) or non-mammalian cells such as amphibian (e.g., frog), fish, or insect cells. Cell lines that may be used in the assays of the invention include, but are not limited to, HEK-293s (human embryonic kidney), CHO (Chinese hamster ovary), LTk- (murine fibroblasts lacking cytosolic deoxythymidine kinase (dTK)), HeLa, BALB/c-3T3, Xenopus oocytes, melanophores (cells from fish and amphibians), and insect Sf9 cells.

In some embodiments, the assays of the present invention are carried using insect cells or membranes prepared from insect cells. For example, Sf9 cells are derived from the fall armyworm, Spodoptera frugiperda, and express relatively low levels of G proteins with little or no low background response for mammalian-GPCR ligands. Through infection with recombinant baculoviruses, these cells can simultaneously express multiple recombinant proteins including, for example, both mammalian GPCR and G protein subunits (α, β, and γ). Proteins expressed in Sf9 cells undergo posttranslational modification. Fatty acid acylation of G protein subunits and GPCRs occurs in a manner almost identical to mammalian cells. Although the extent of modification and specific glycosylation processes differ from mammalian cells, GPCRs and G proteins expressed in Sf9 cells function similarly to those in the mammalian cells.

Addition benefits of embodiments using Sf9 cells relate to the presence of adenosine deaminase (ADA), an enzyme that metabolizes adenosine. Typically, ADA should be removed from [³⁵S]GTPγS binding reactions order to eliminate the released adenosine from intact heterologous mammalian cells or membrane preparations from native tissue or mammalian cells. In mammalian cell types, the presence of adenosine deaminase (ADA) in the assay can limit testing the action of adenosine (the natural ligand of ADA) or any compound that modulates the action of adenosine. We have found that the potencies of synthetic ADA-resistance adenosine receptor ligands, such as CGS21680, CV1808, and NECA, are comparable in Sf9 cells expressing adenosine A2a receptor, XLGolf, and β1γ2 dimer.

The assays of the present invention can be carried out in a high throughput format to identify compounds that act as agonists on the receptor. For example, agonists or positive modulators can be identified that act at the A2a or D1 receptor using the assays of the present invention. In some embodiments, secondary assays can be carried out following a primary HTS screening campaign using another assay, such as a binding assay (with labeled ligand, for example) or a cyclic AMP (cAMP) stimulation to verify that compounds are acting on the receptor. Binding assays are particularly effective as secondary assays since the readout is close to the receptor itself. Assays with amplification steps like the cAMP induction assay are more likely to produce false positives.

Some embodiments include testing for binding of a test compound and/or ligand. For such binding assays, membranes or whole cells are contacted with a test compound. After binding is complete, the GPCR is separated from the ligand and/or test compound, eg., by filtration, and the amount of binding that has occurred is determined. In some embodiments, the ligand used is detectably labeled with a radioisotope such as, for example, ¹²⁵I. Other types of labels can also be used, including, but not limited to, the following fluorescent labeling compounds: fluoresceinisothiocyanate, rhodamine, phycoerythrin, phycocyanin, allophycocyanin o-phthaldehyde and fluorescamine. Chemiluminescent compounds can also be used with the assays of the invention, including, but not limited to, luminol, isoluminol, theromatic of acridinium ester, imidazole, acridinium salt, and oxalate ester.

In other embodiments, the assays of the present invention can be run as an SAR driving assay. An SAR driving assay is used to guide chemistry efforts when developing small molecules, and they need to be as biologically representative as possible so that the correct functional behavior is optimised. Since the GDP-GTP exchange event is proximal to receptor activation, assays of the present invention wherein GPCR activity is determined by detecting GDP-GTP exchange are particularly useful for generating pharmacologically accurate and relevant parameters, such as potency and efficacy.

Non-radioactive heterogenous or radioactive homogenous assay formats for measuring GTP binding to G alpha subunits have developed in the art, particularly for use in high throughput screening (HTS). The DELFIA GTP assay, a non-radioactive heterogenous assay format, measures the time resolved flourescence of the binding between europium-labled GTP and Ga subunit. However, the requirement of a washing step to separate the unbound Eu-GTP from the bound Eu-GTP/Ga complex is a potential drawback in the high throughput application of this assay. In contrast, the homogenous assay formats that do not require separation of the bound and free [³⁵S]GTPγS are more appropriate for HTS applications. The homogeneous assay is available in two formats, scintillation proximity assay (SPA) bead or flash plate. We have been able to use the homogeneous assay in SPA bead format in the [³⁵S]GTPγS binding assay for DRD1 and A2A receptor co-expressed with XLGolf in Sf9 cells. In our assays, the scintillation proximity beads were coated with wheat germ agglutinin (WGA) that interacted with glycosylated protein. In some embodiments, expression of XLGαolf in Sf9 cells with either DRD1 or A2A receptor are used to perform [³⁵S]-GTPγS binding assay in either heterogenous or homogenous formats.

The test compounds used in the methods described above include compounds such as peptides, peptidomimetics, small molecules, or other drugs.

We have identified a unique expressed sequence, found in mammalian tissue, that encodes a functional splice variant of GNAL designated XLGolf (extra long). Gene expression patterns of XLGolf differ from Golf. We have established expression of a functional XLGolf protein in Sf9 cells. Cell co-expression systems include XLGolf or Golf and a selected GPCR (including DRD1, A2a, etc). Antibodies specific for XLGolf. Nucleic acid probes and primers specific for XLGolf. Methods of using antibodies and nucleic acid probes for diagnosis using human biological samples (ELISA, TaqMan, In situs, Westerns, Imaging—PET, MRI etc.).

Diagnostics

Polymorphisms in XL & Golf and the Use of These Polymorphisms for a Diagnostic

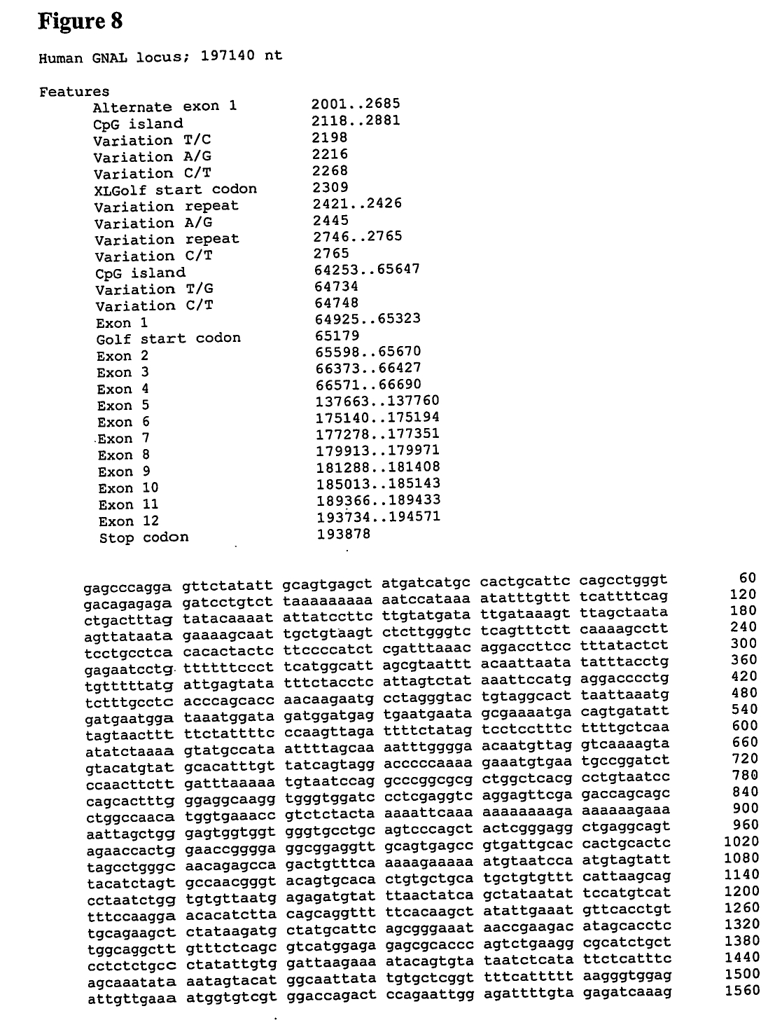

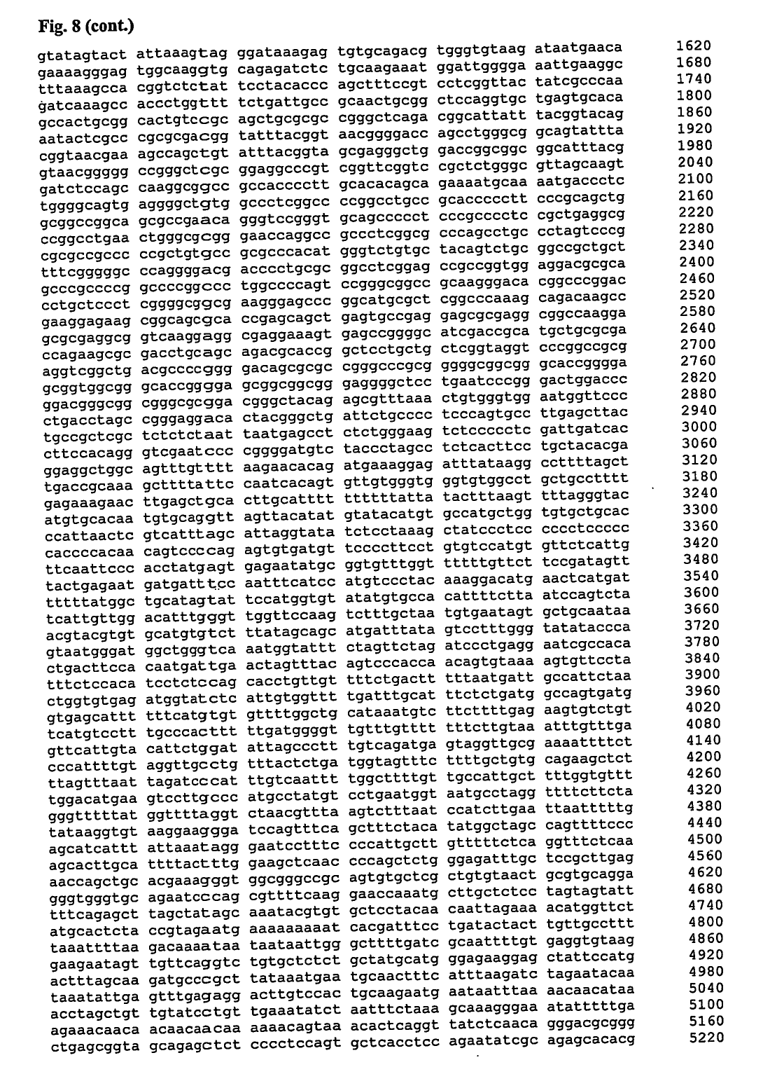

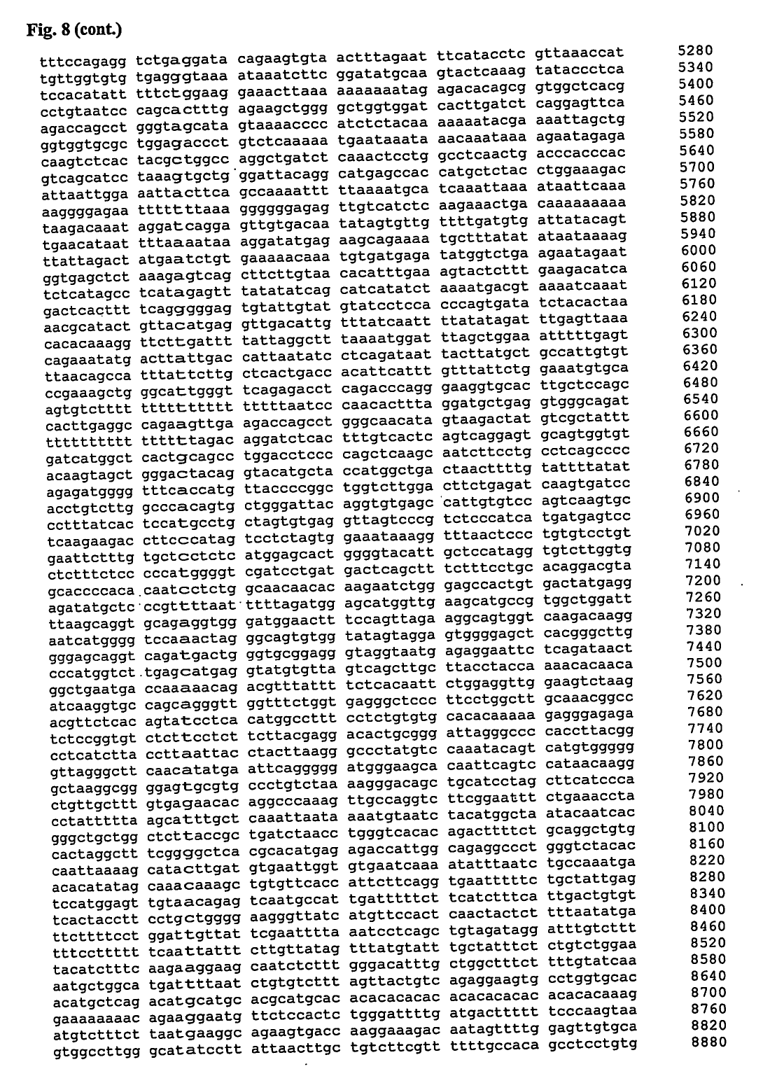

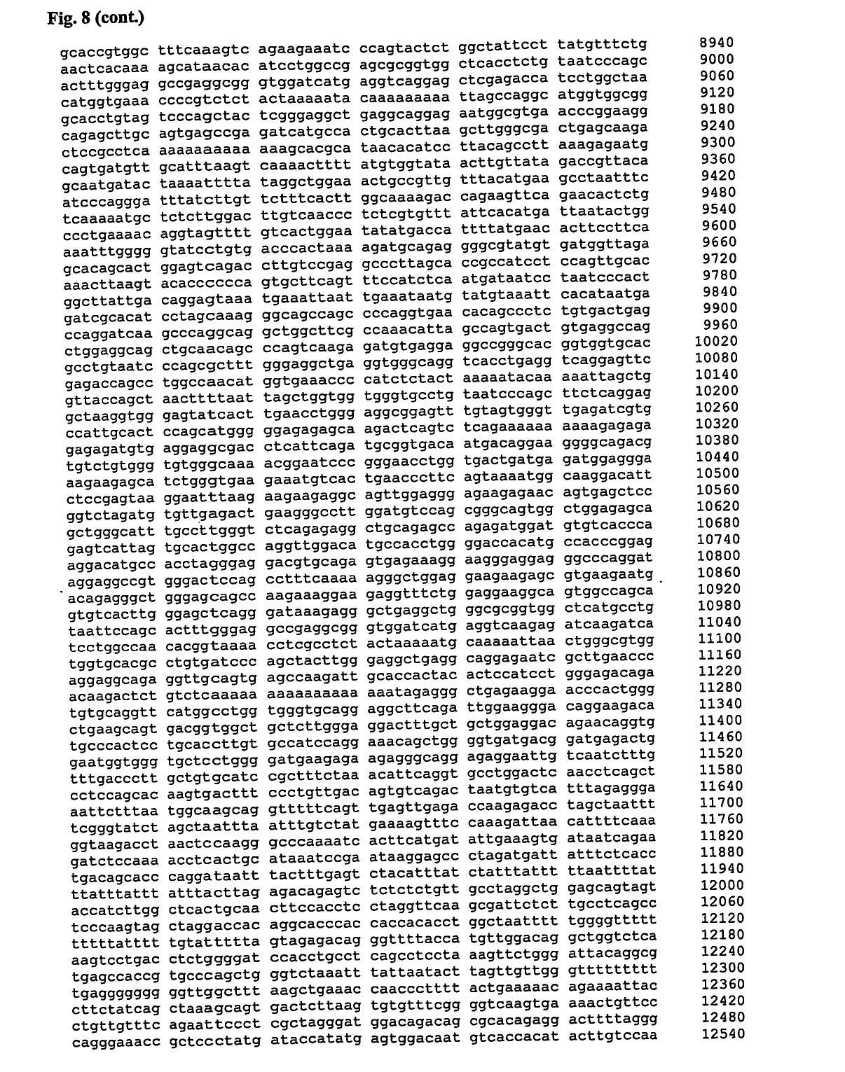

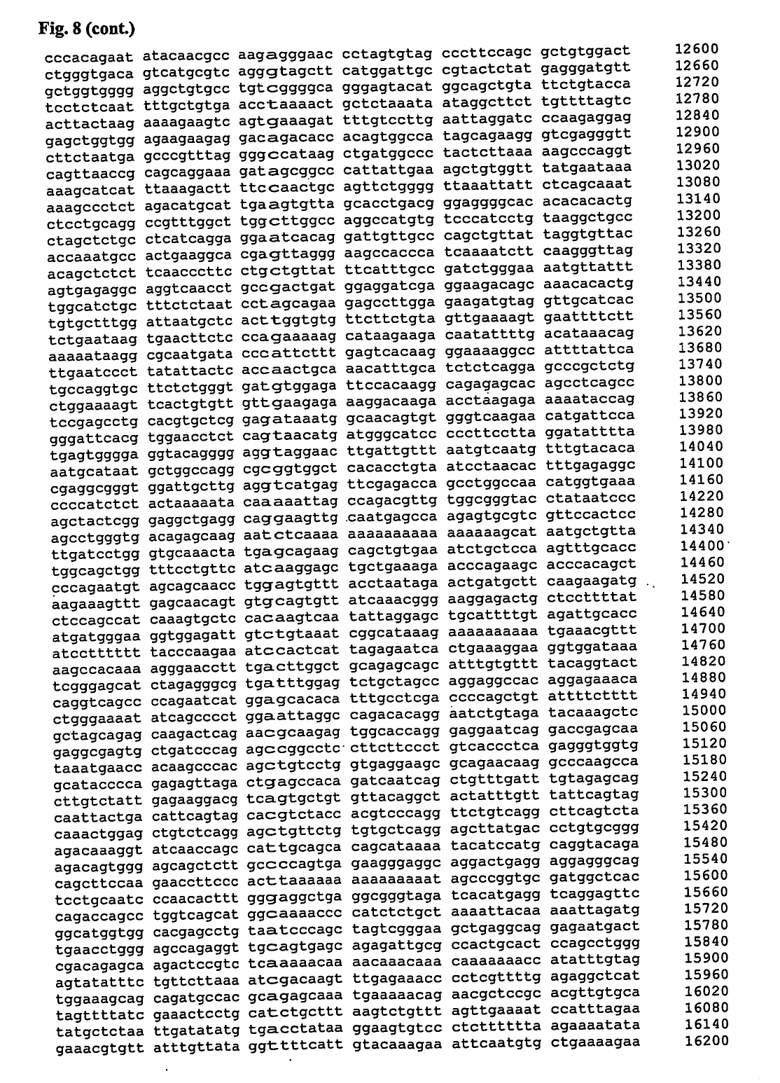

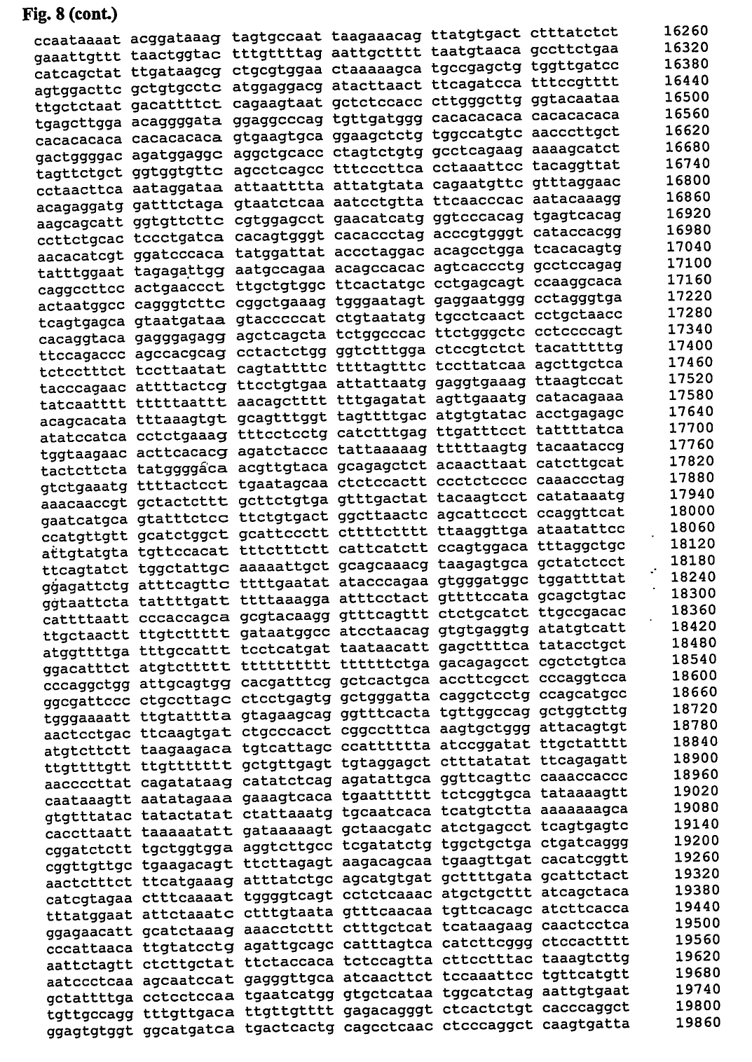

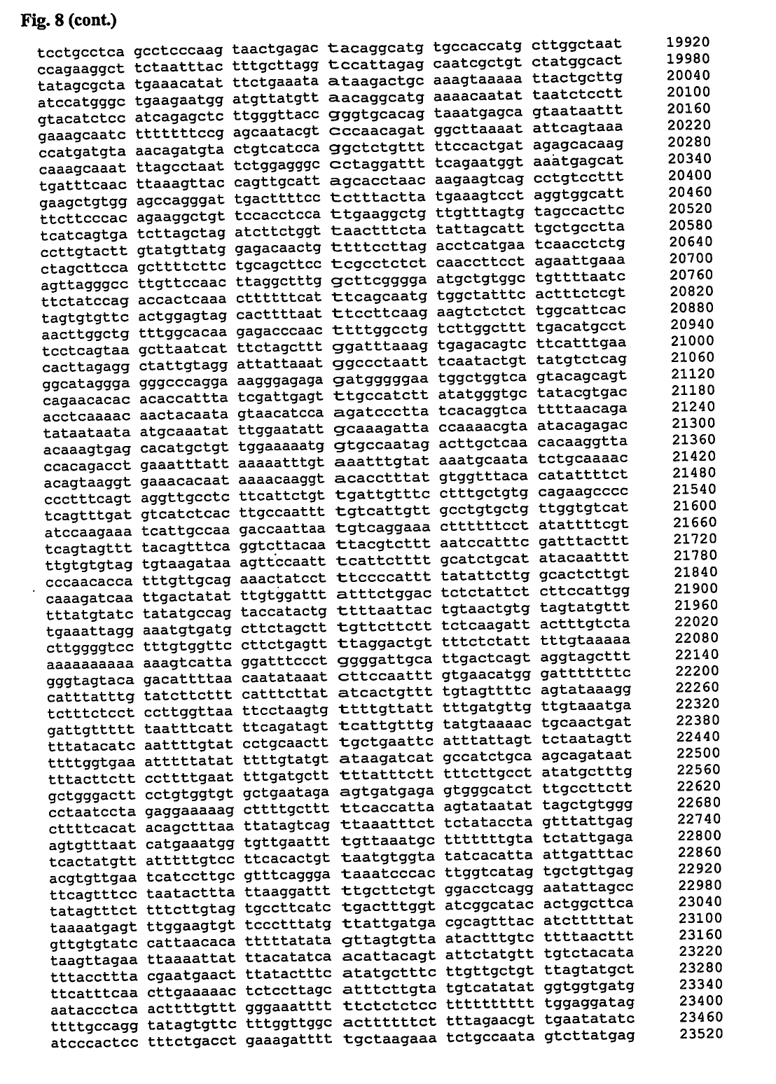

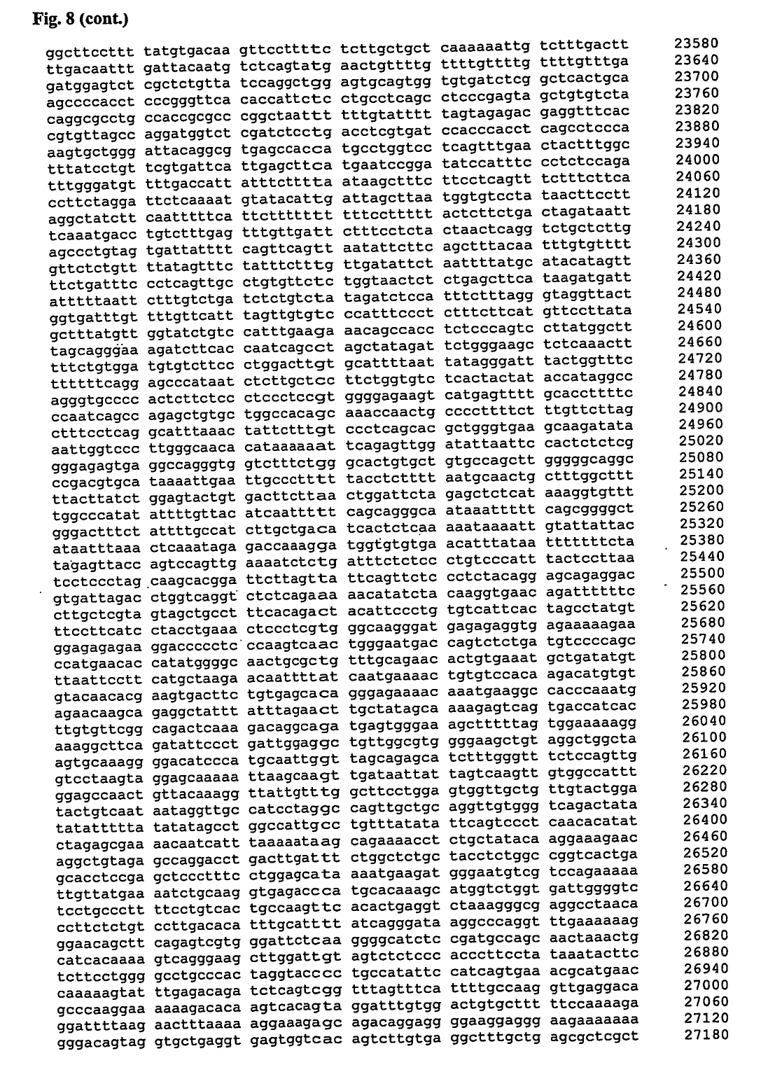

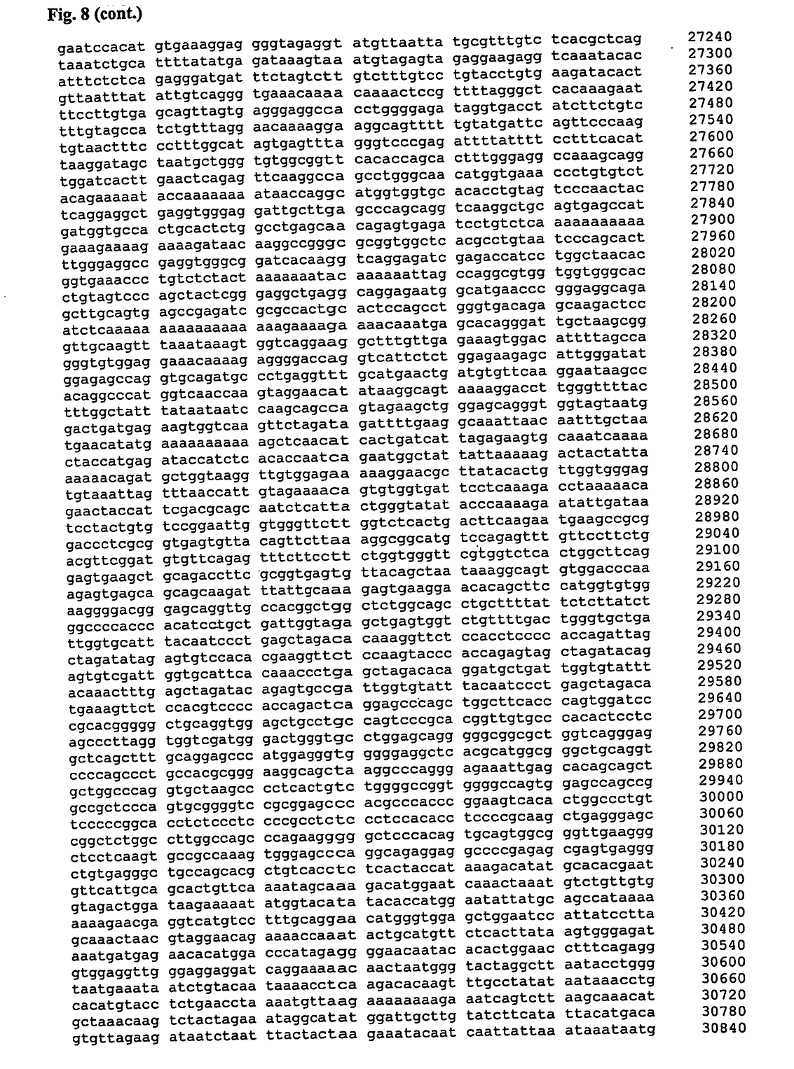

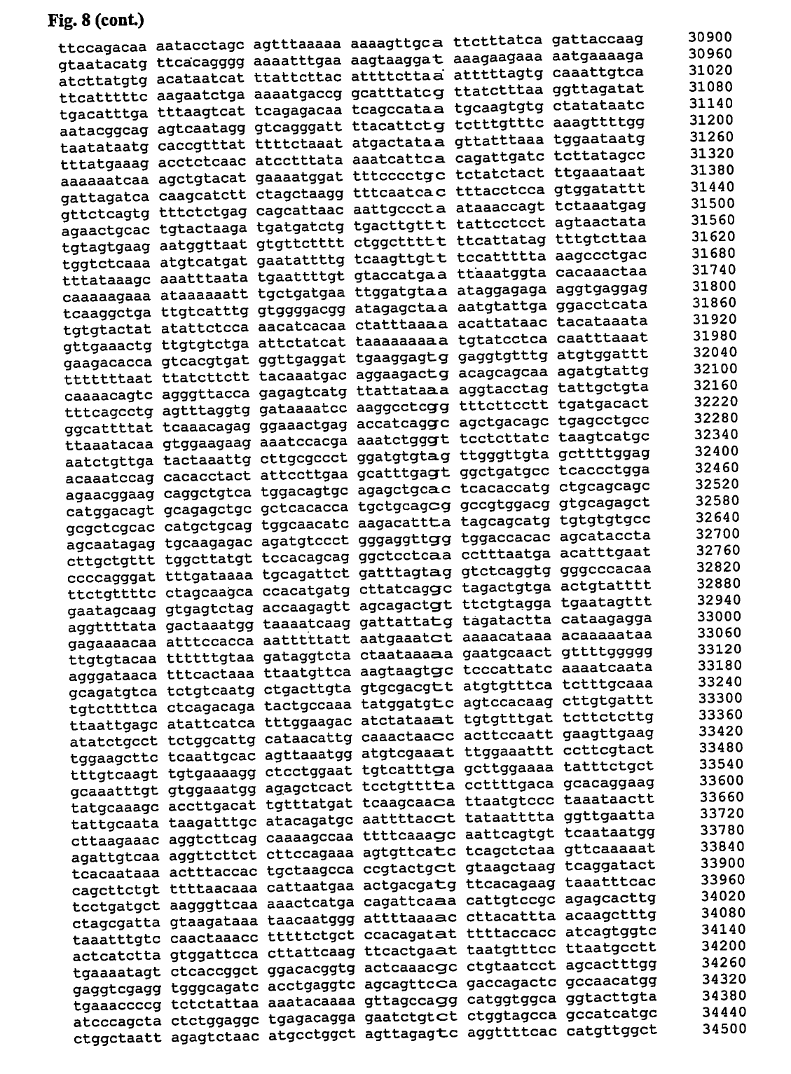

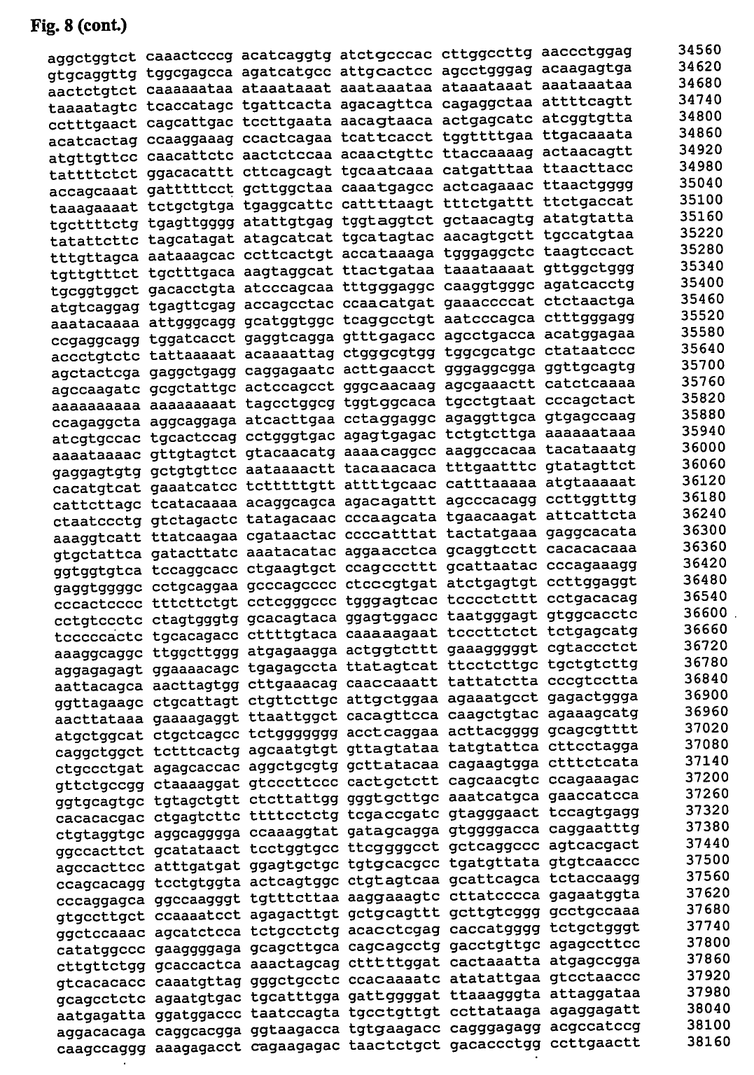

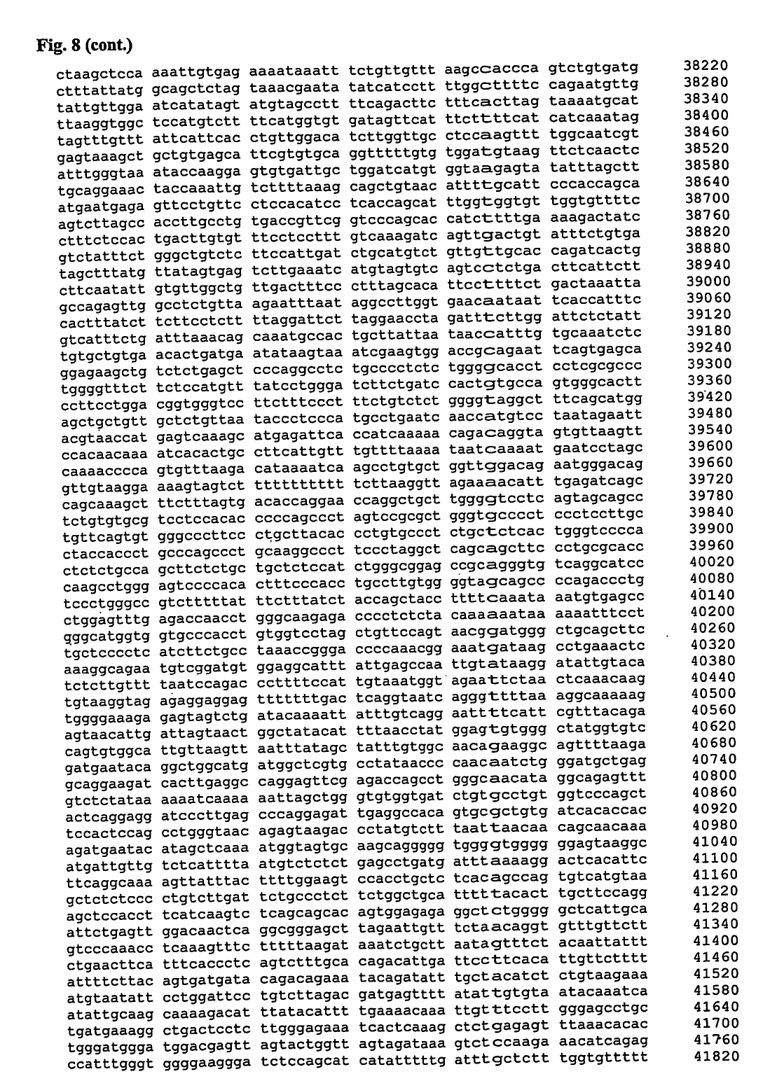

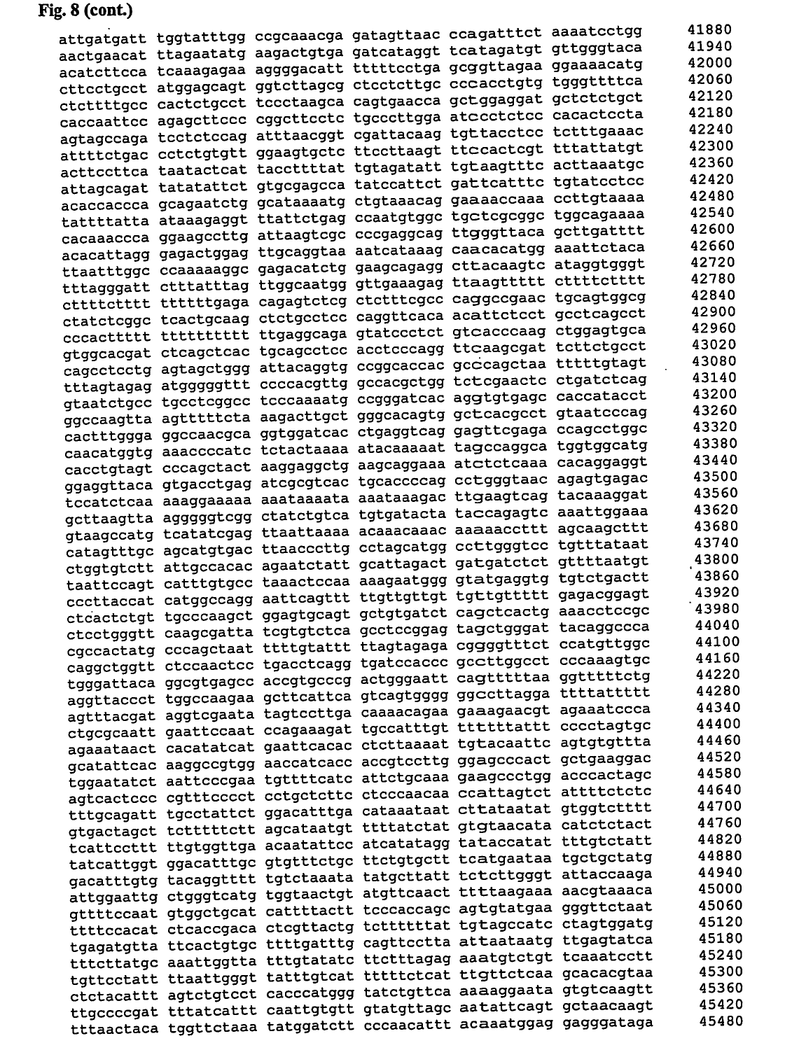

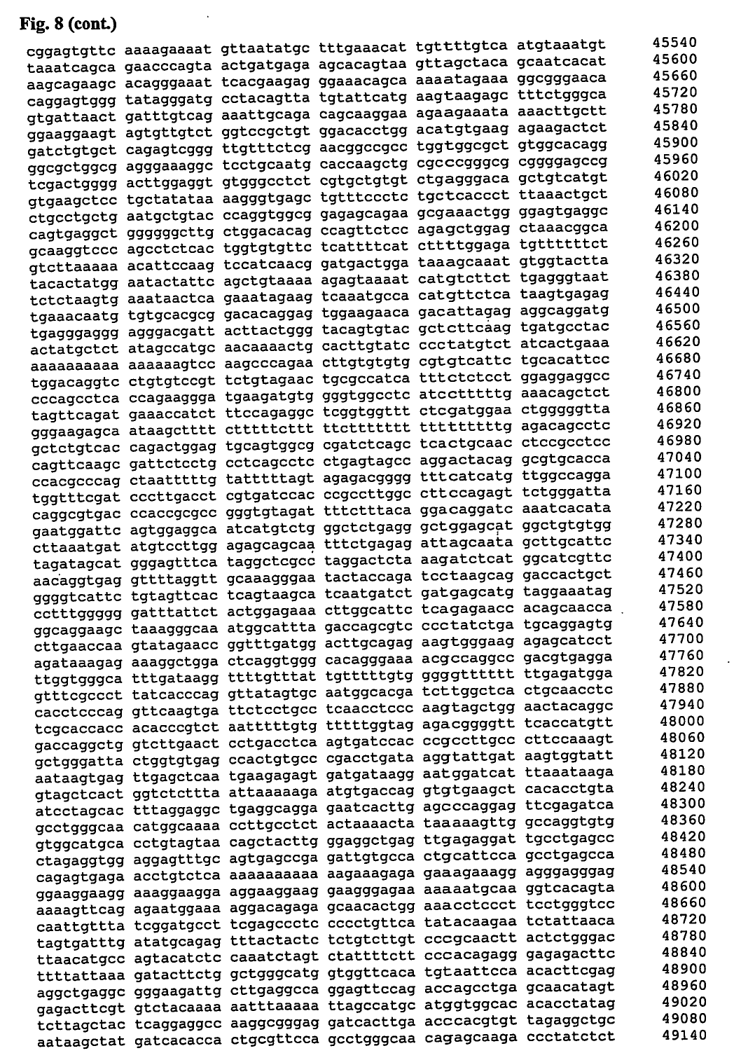

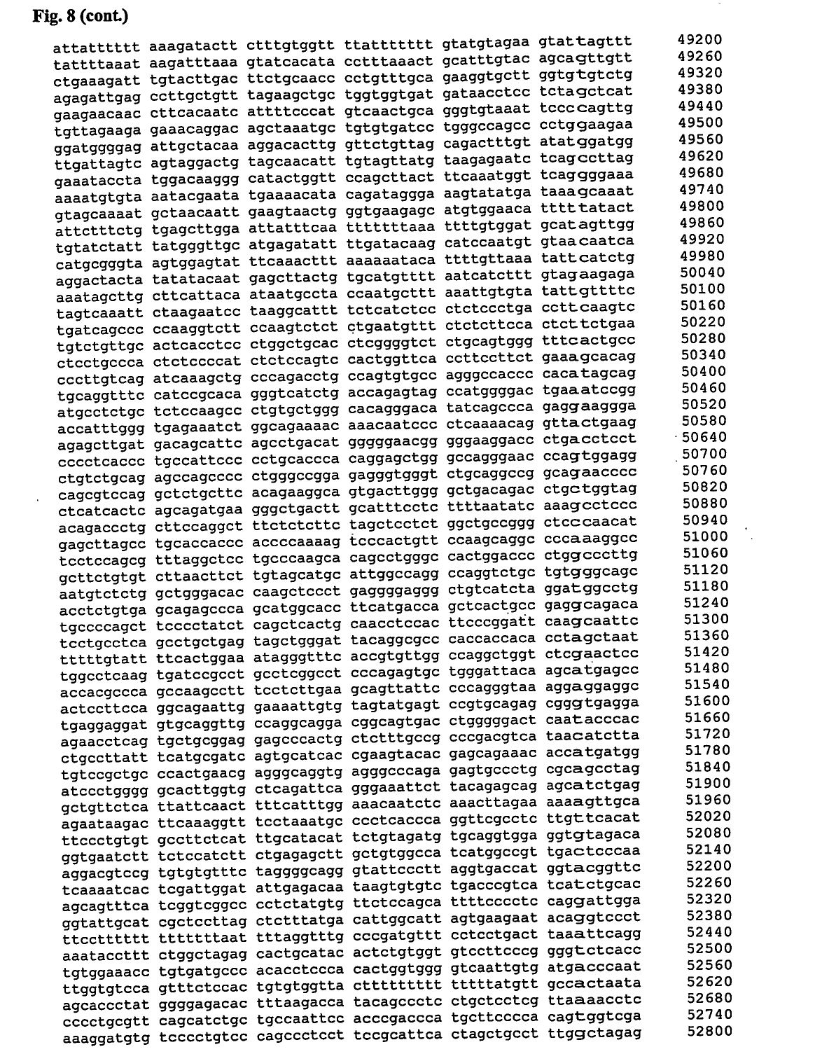

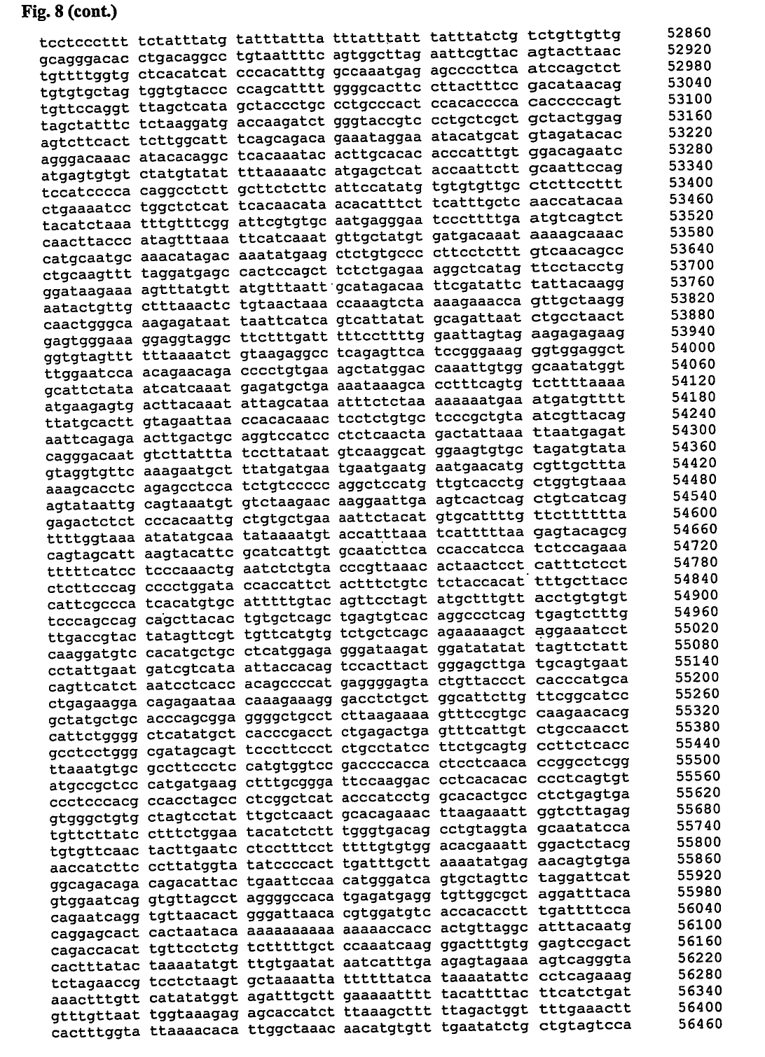

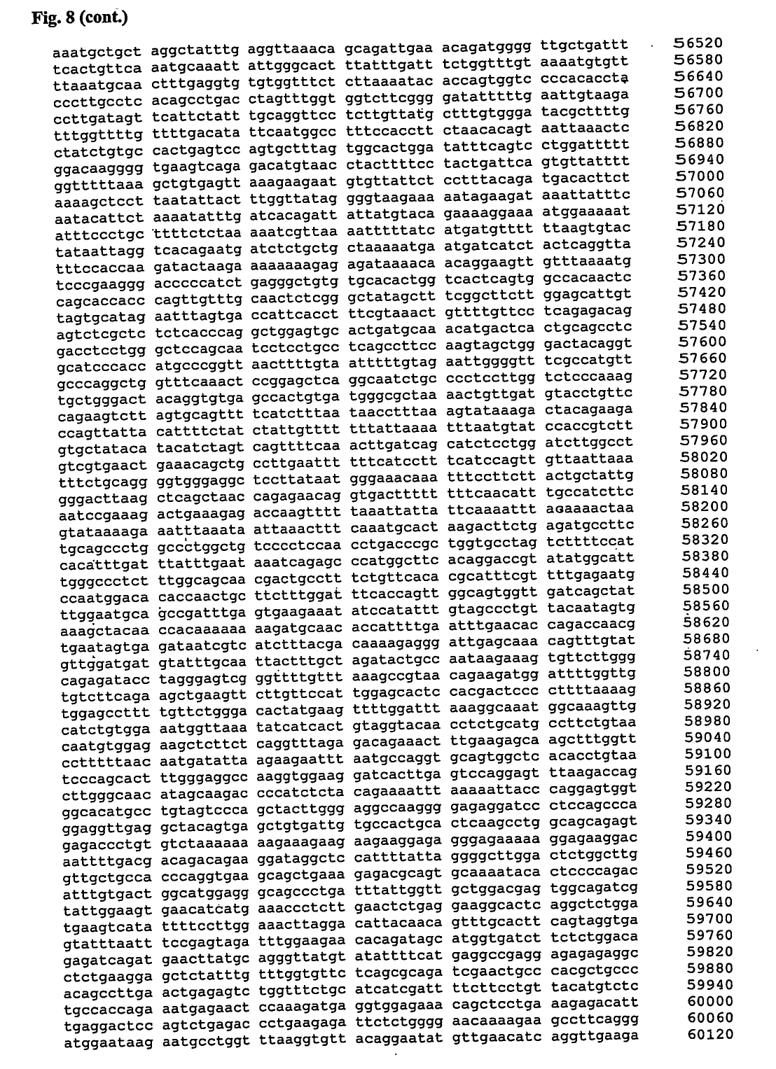

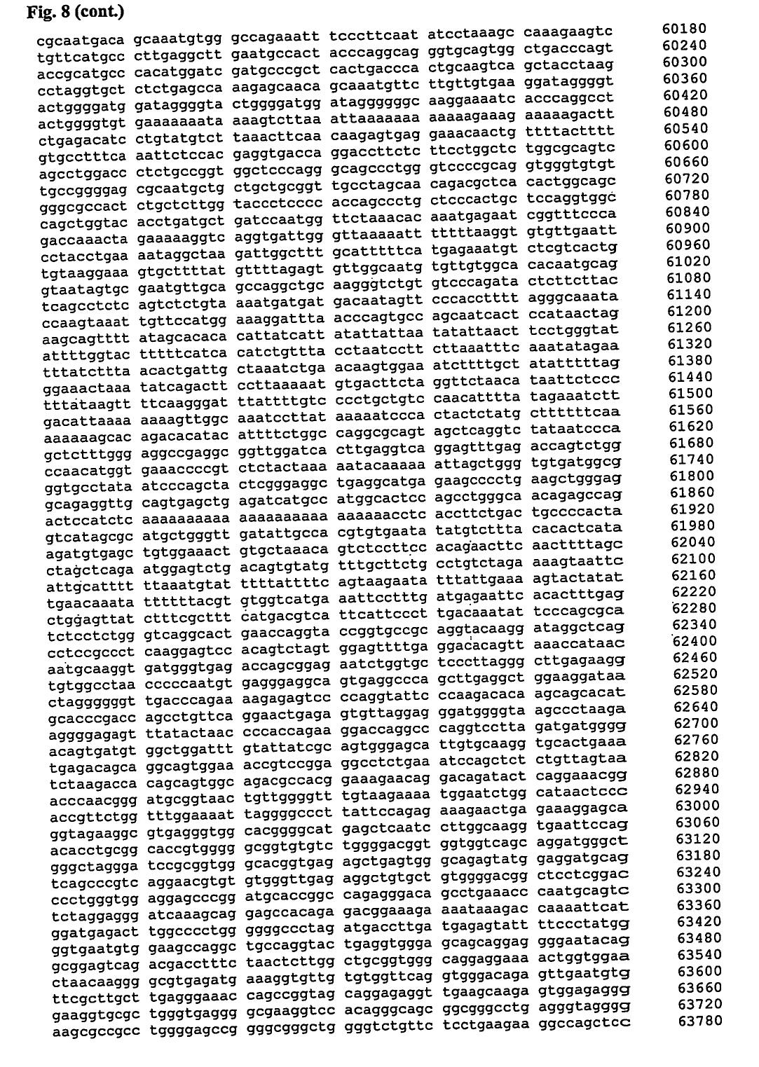

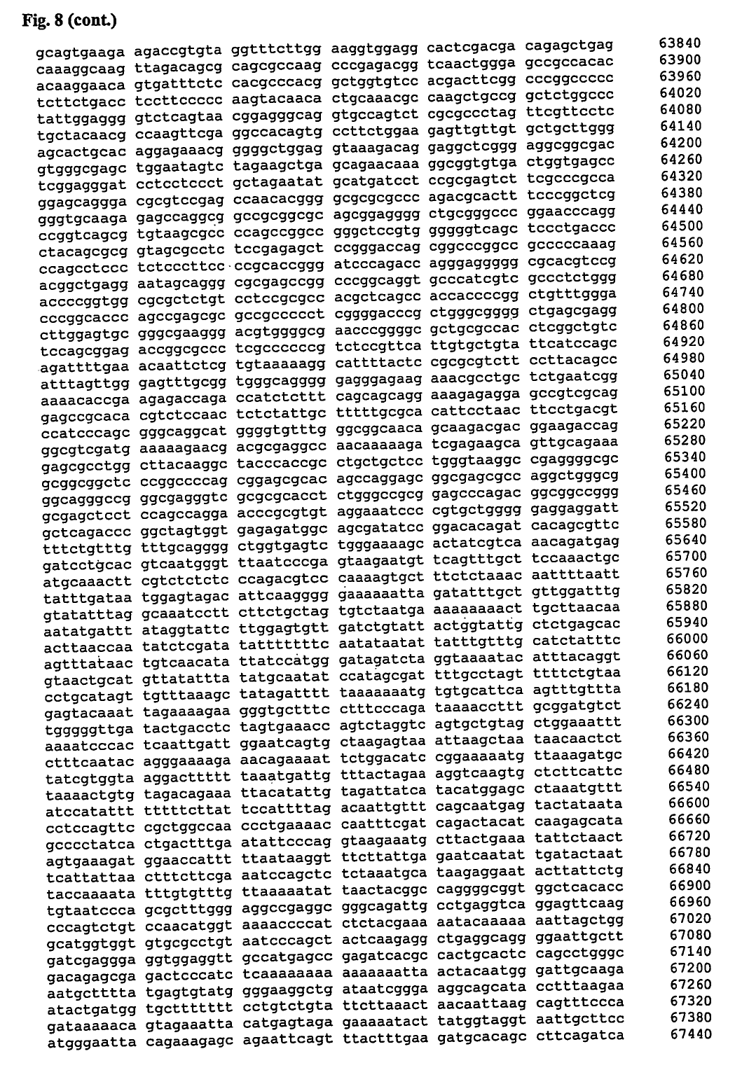

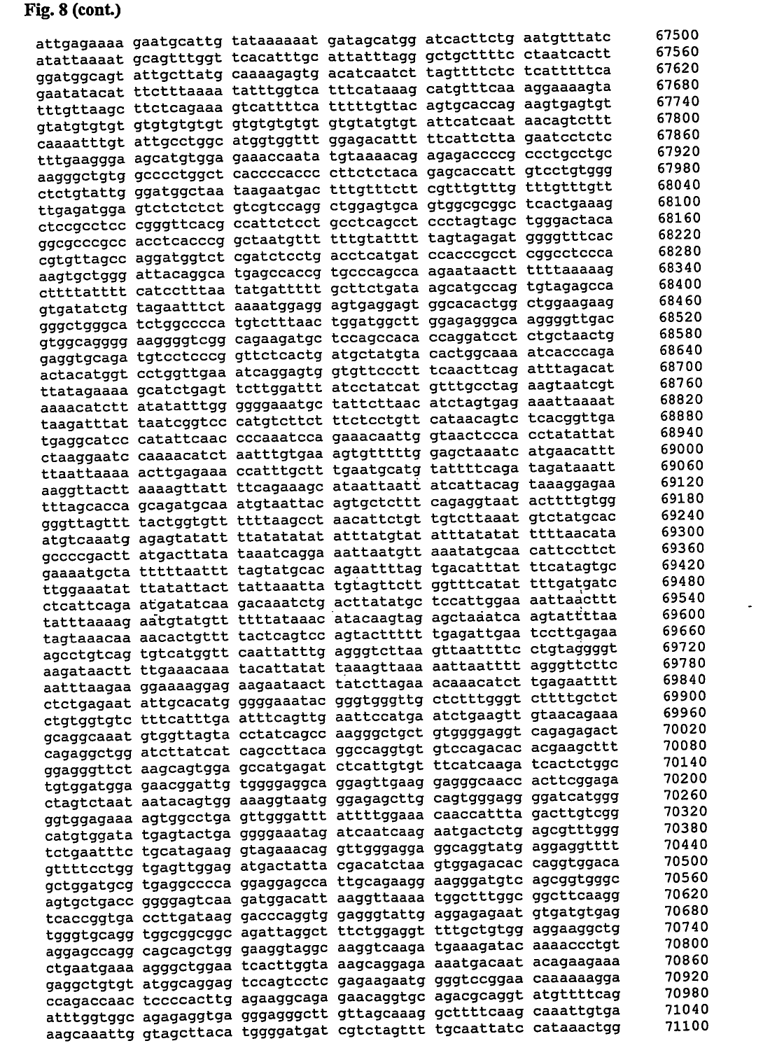

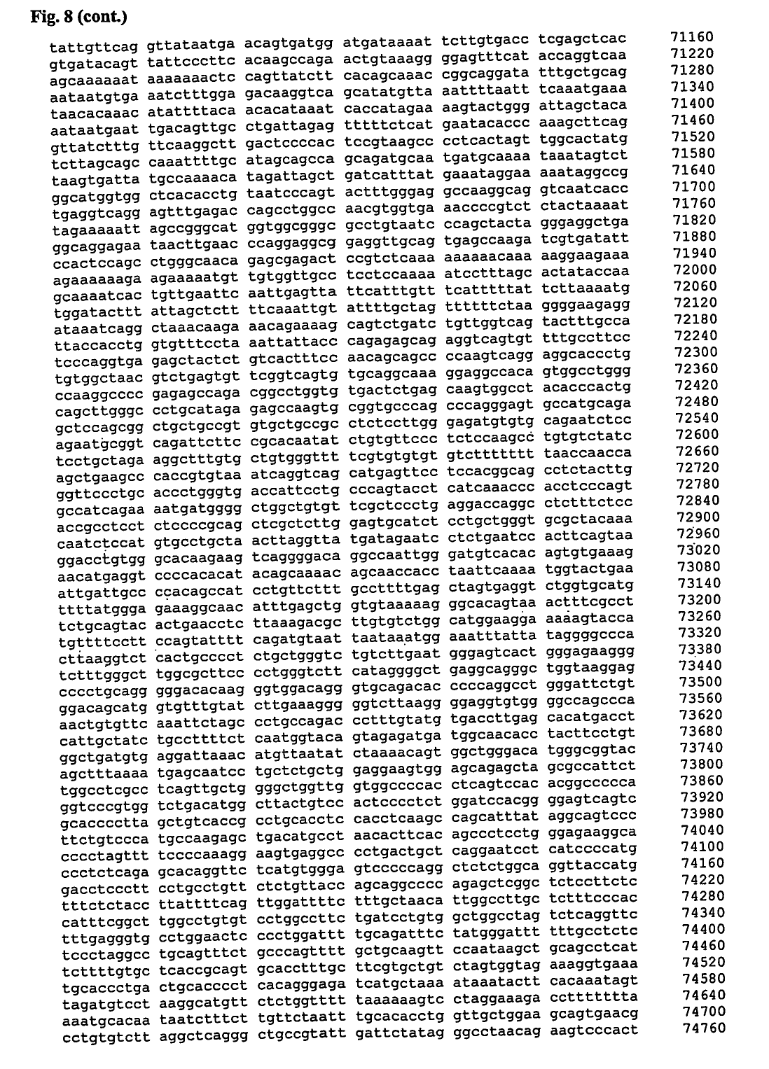

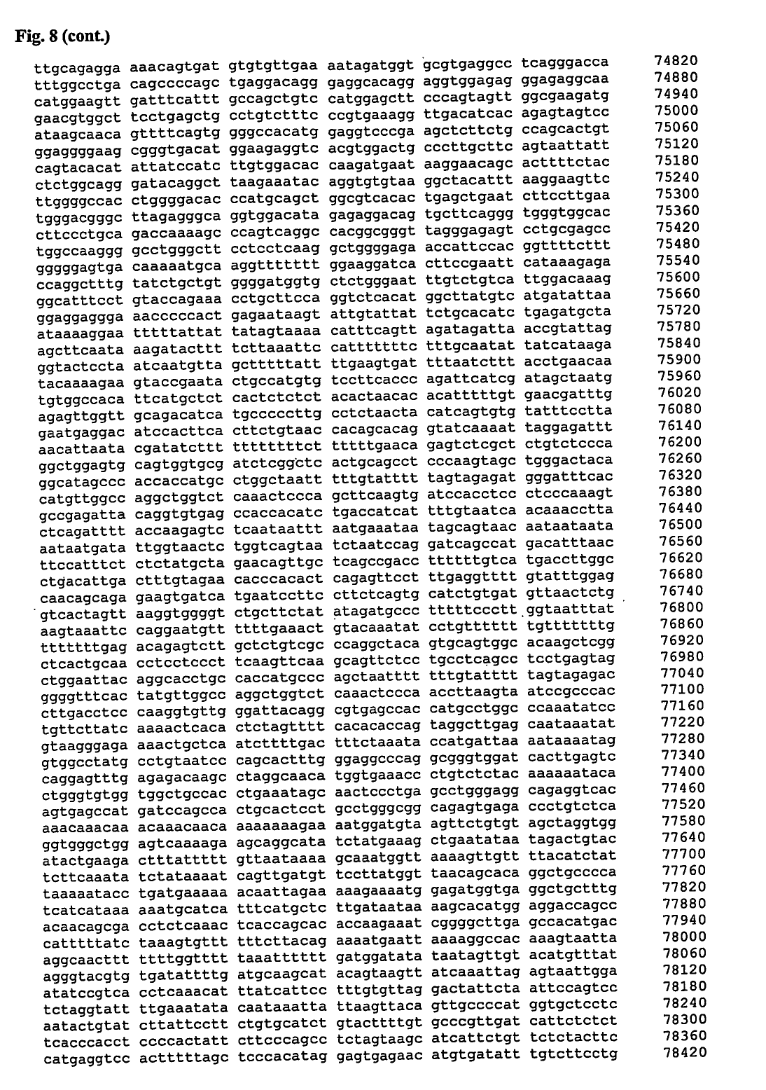

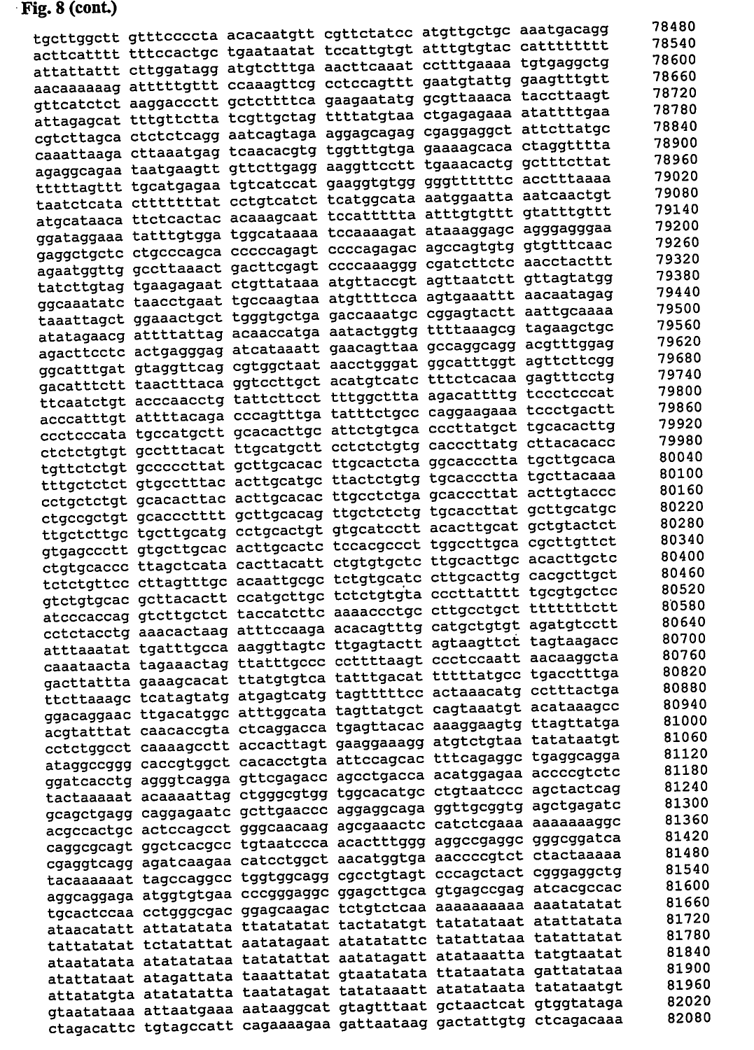

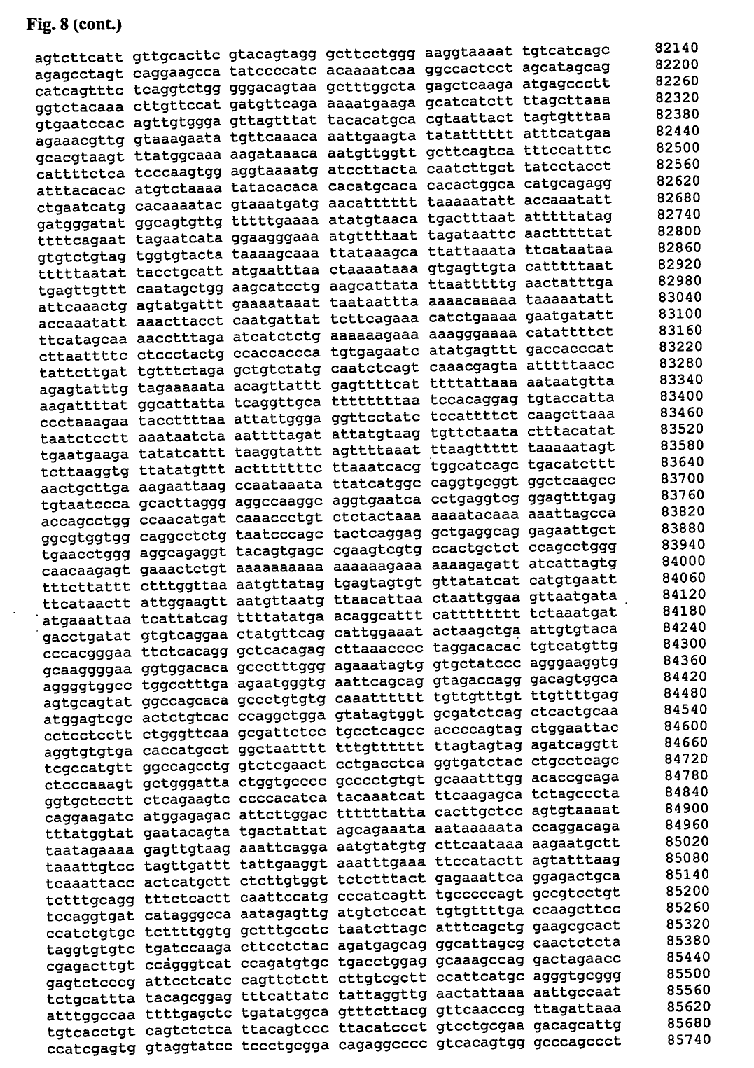

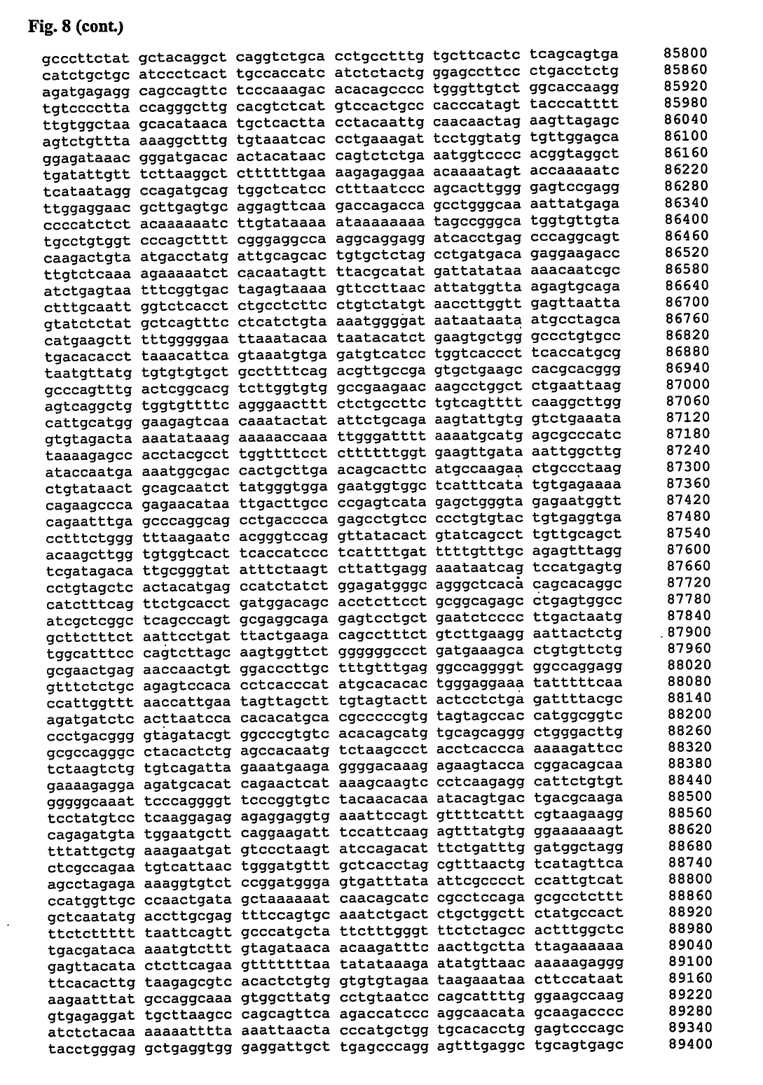

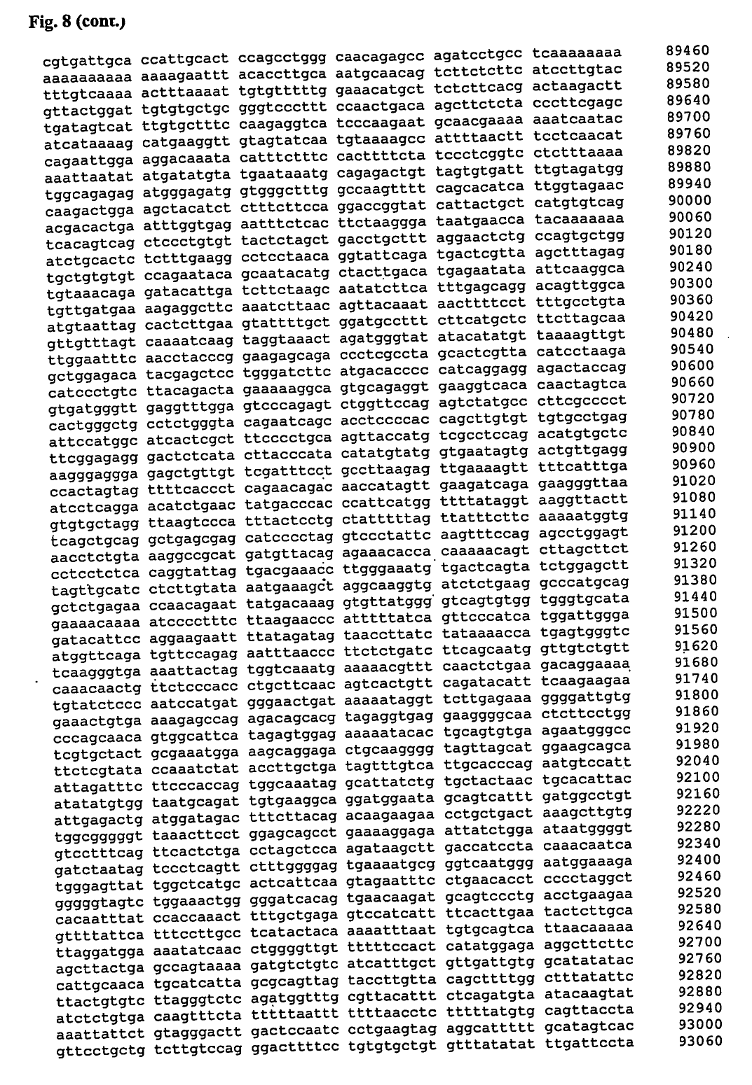

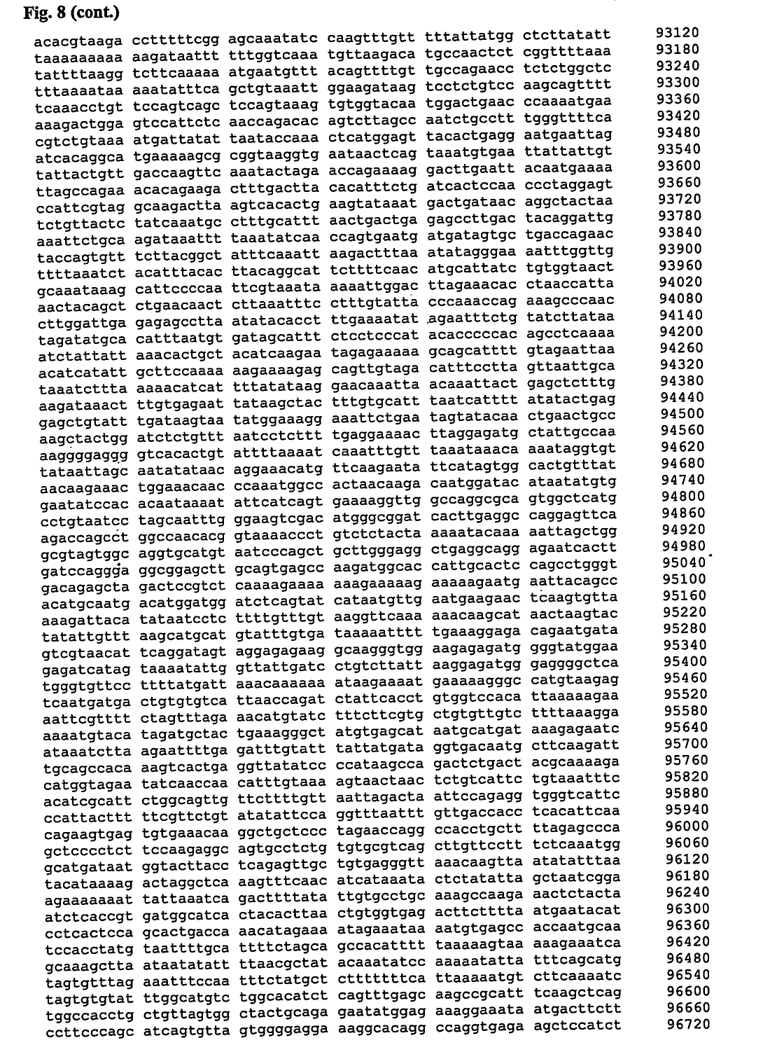

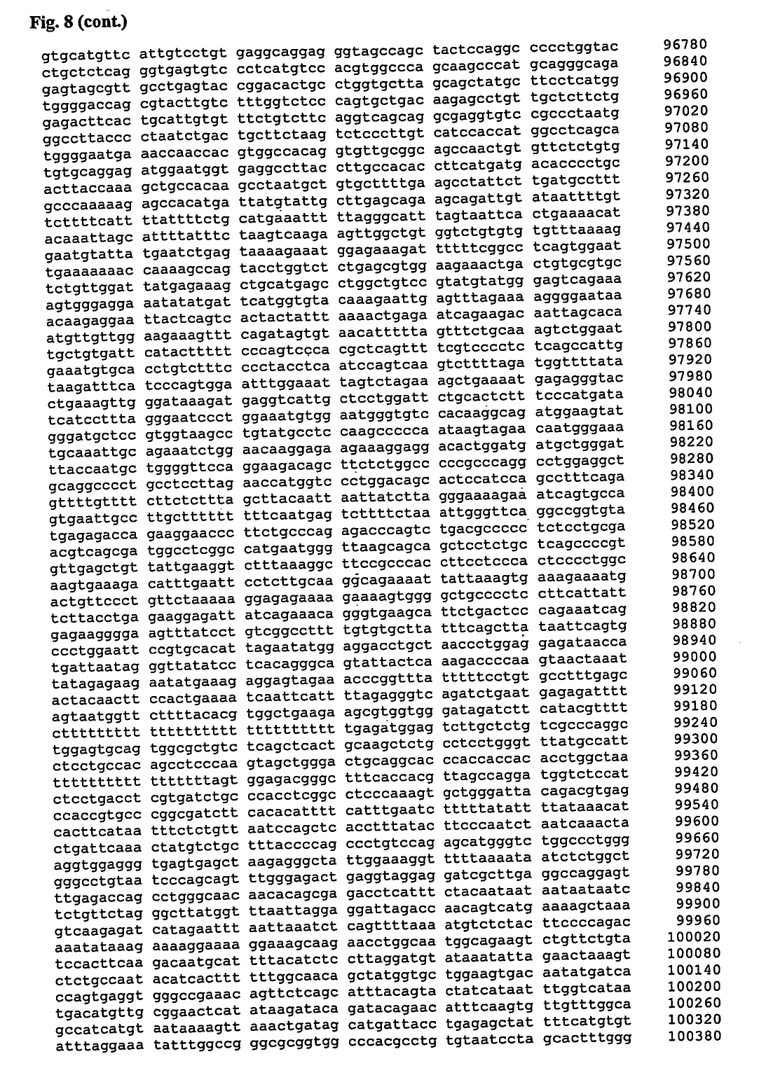

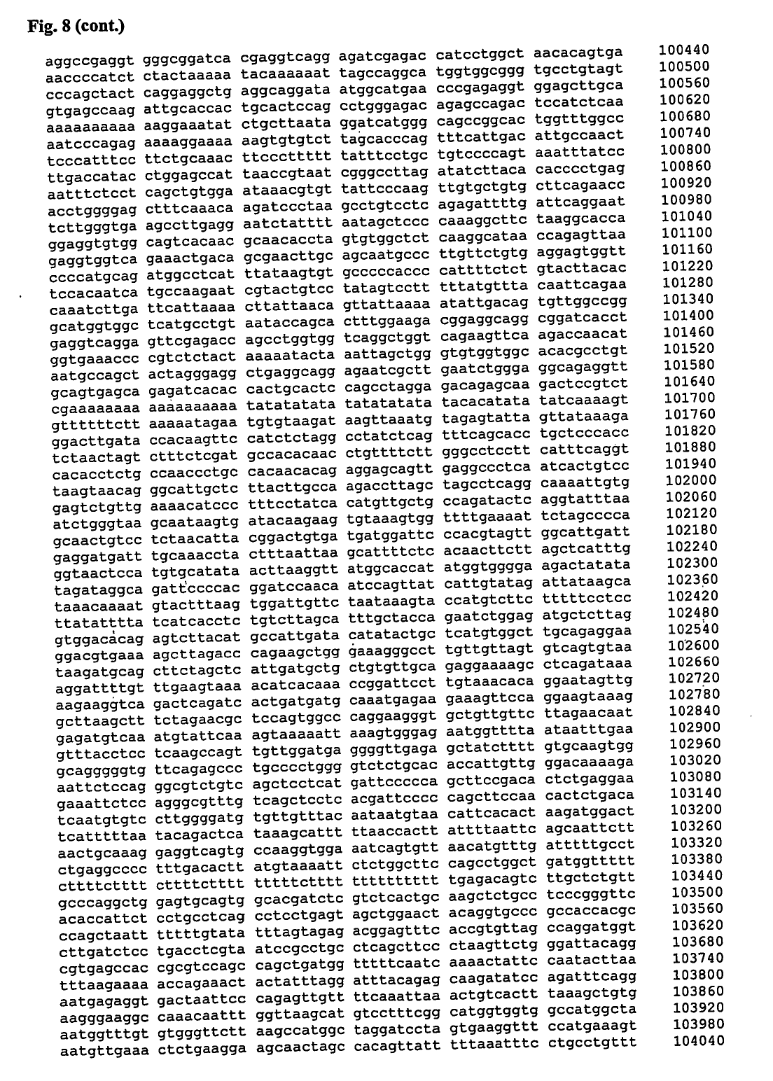

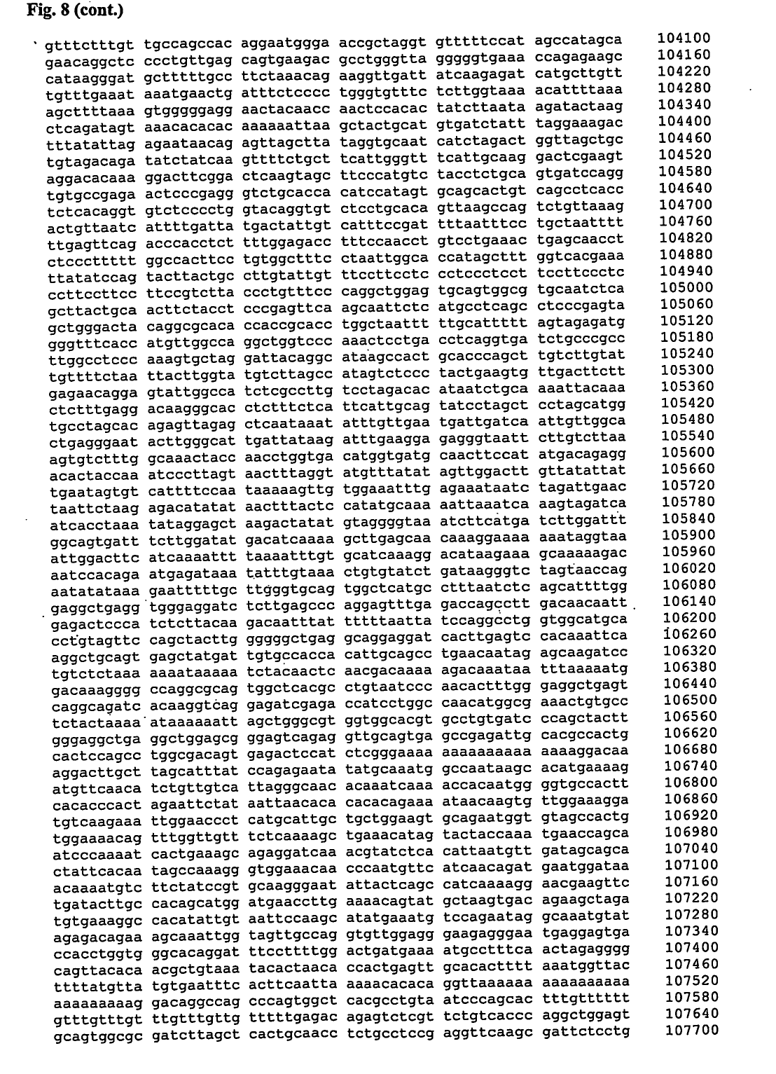

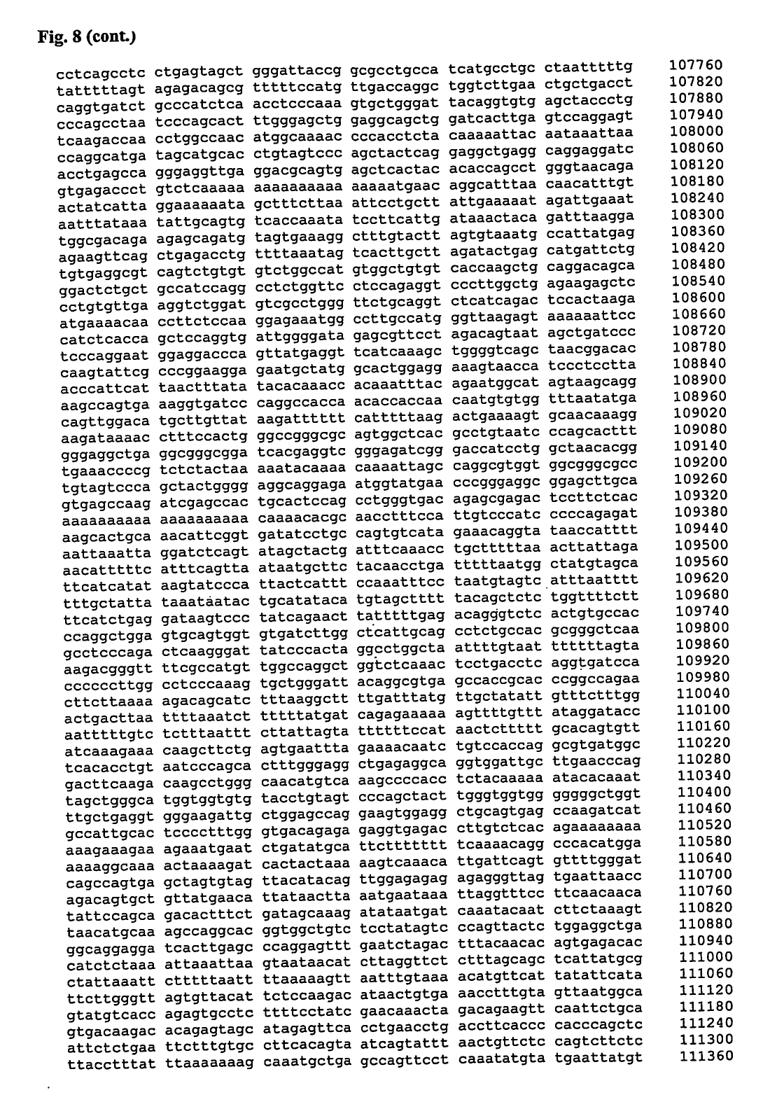

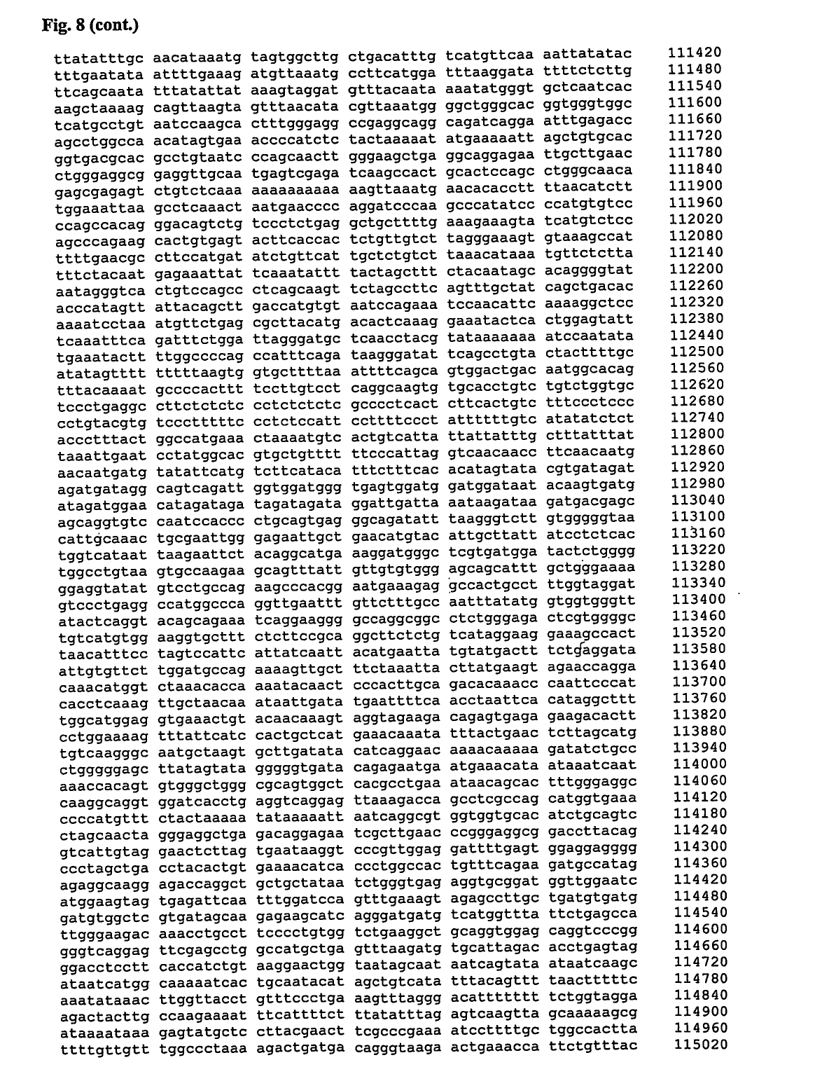

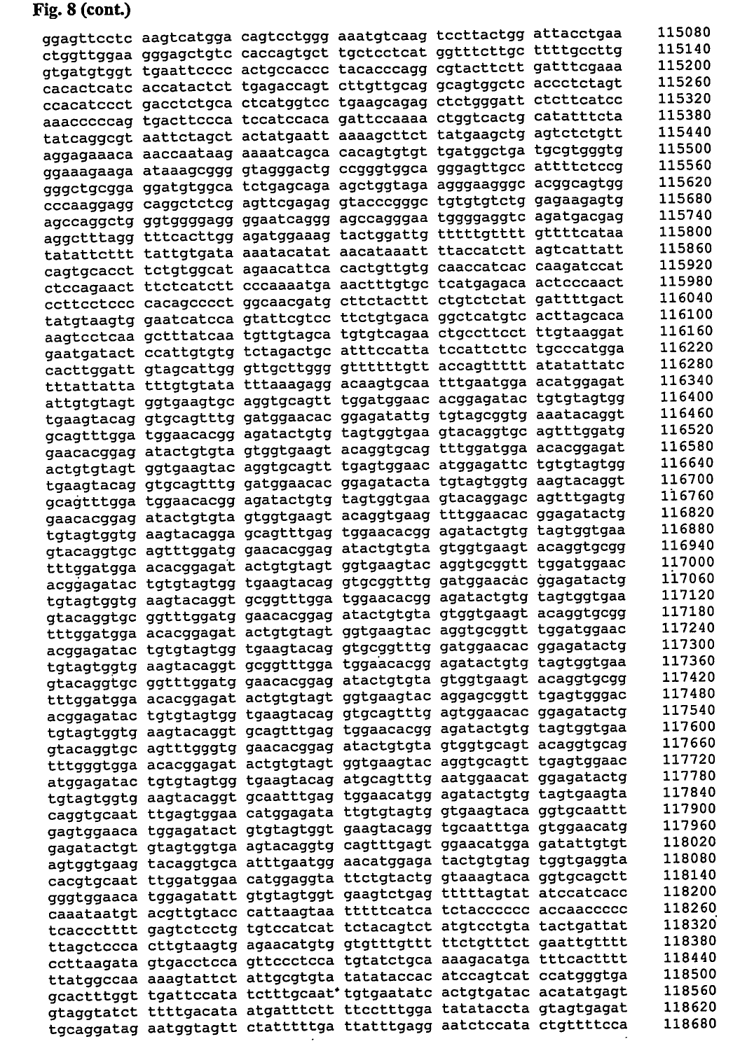

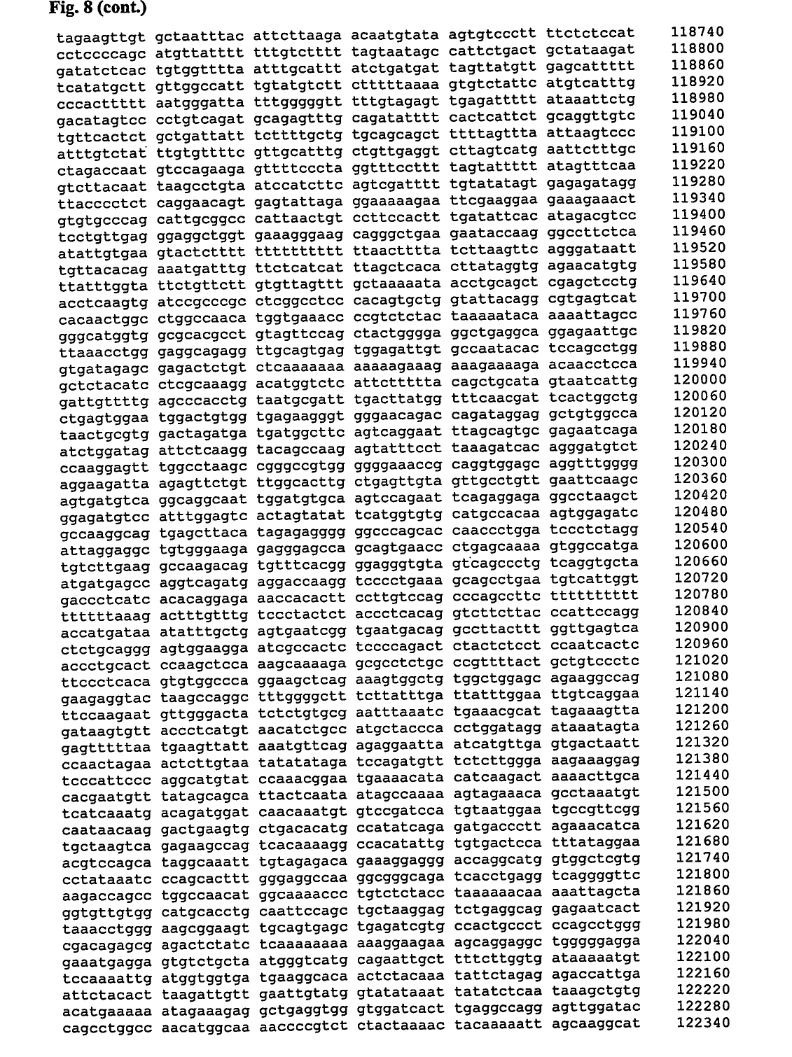

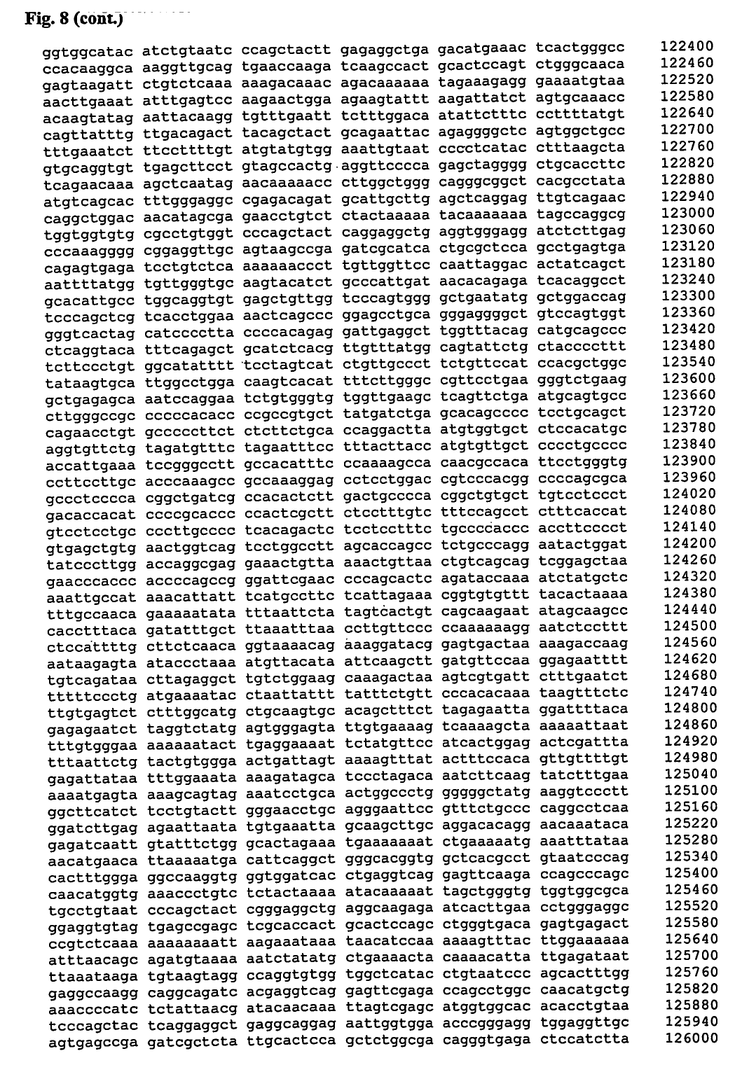

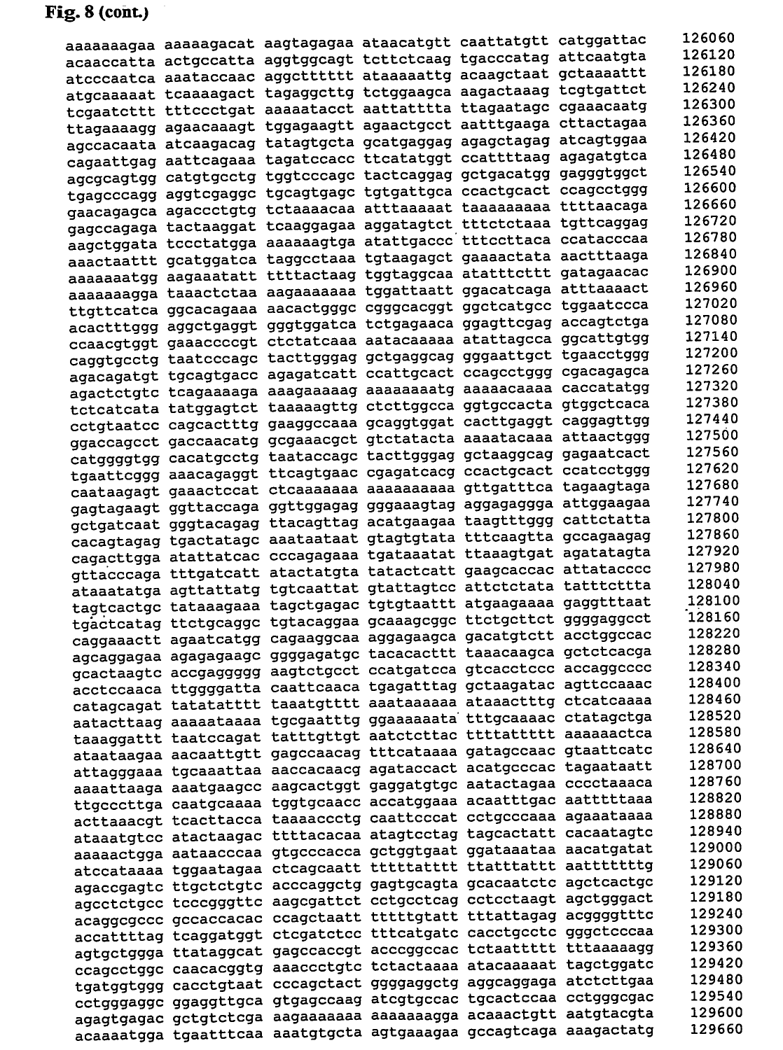

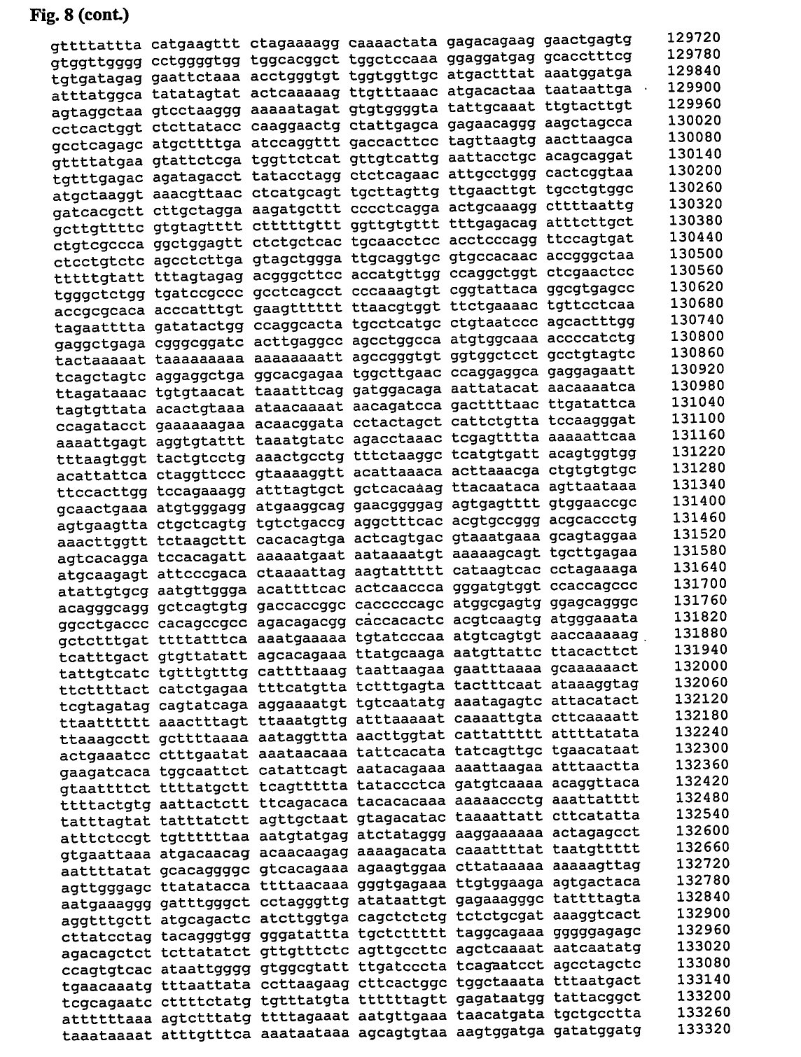

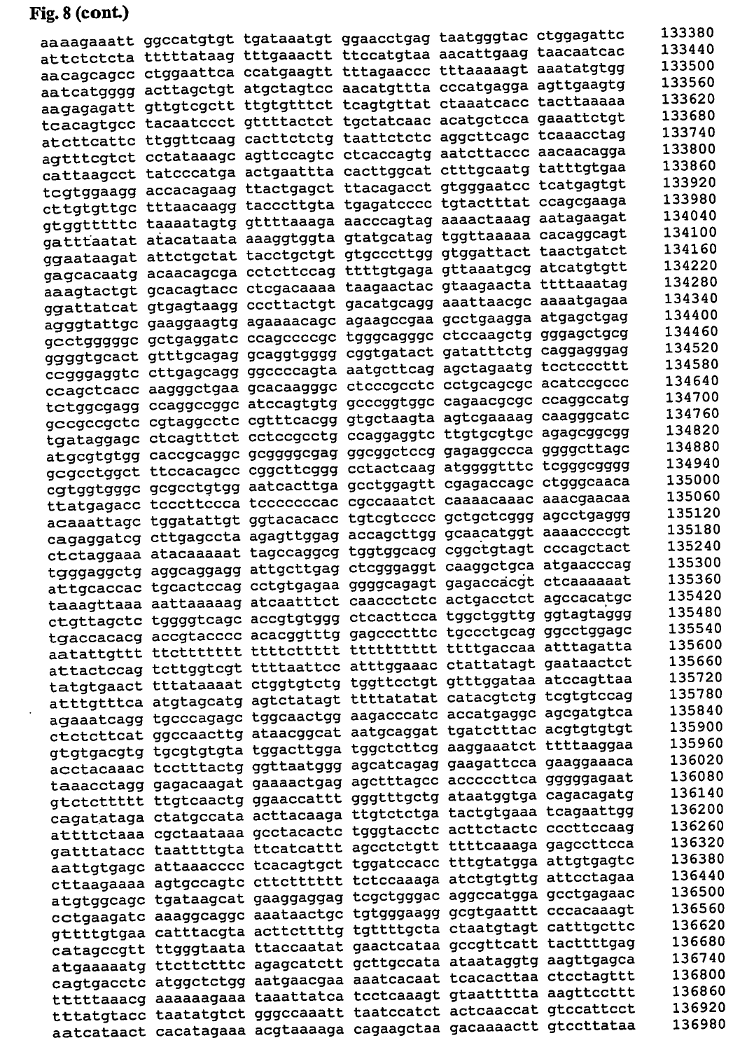

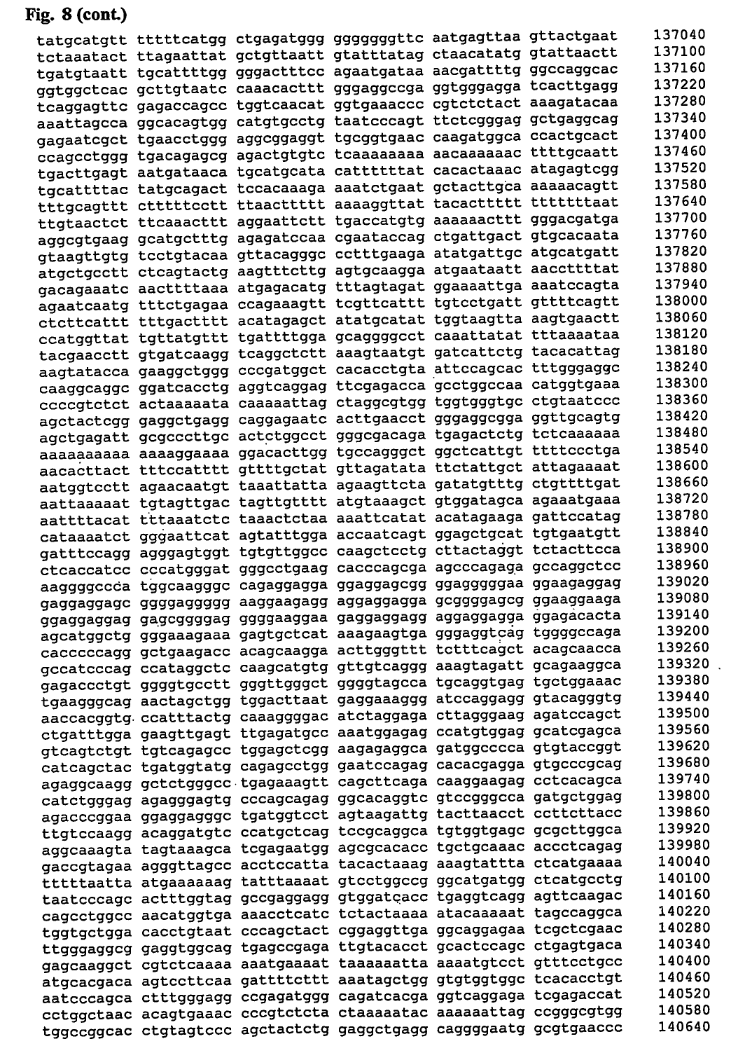

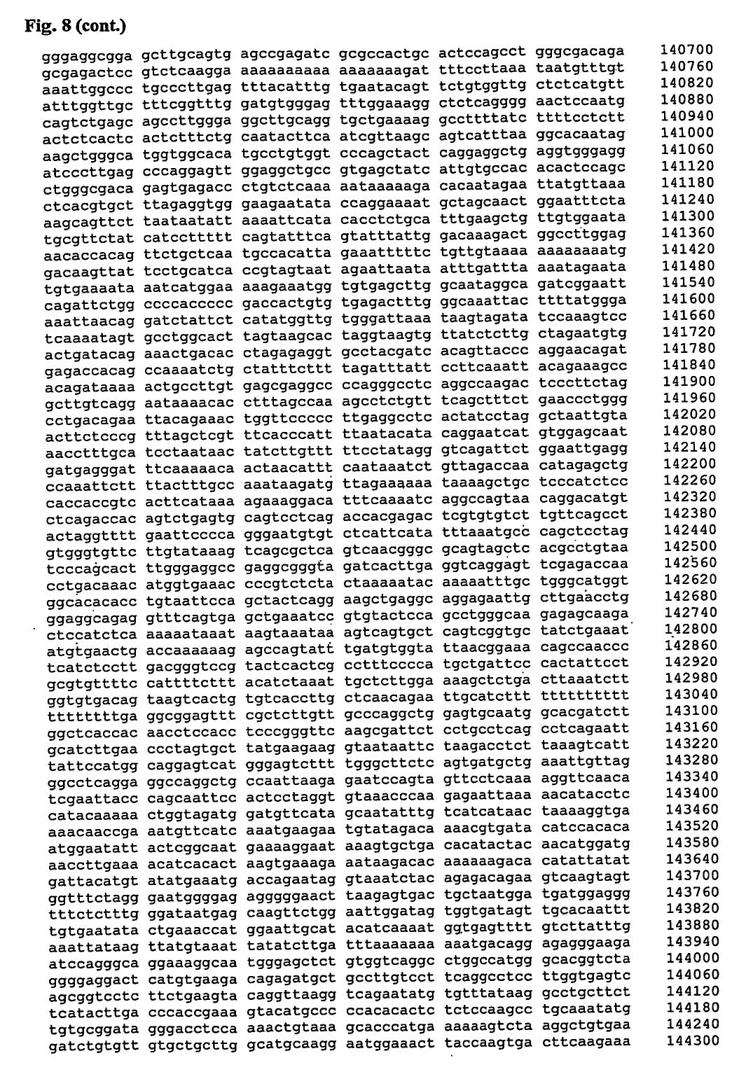

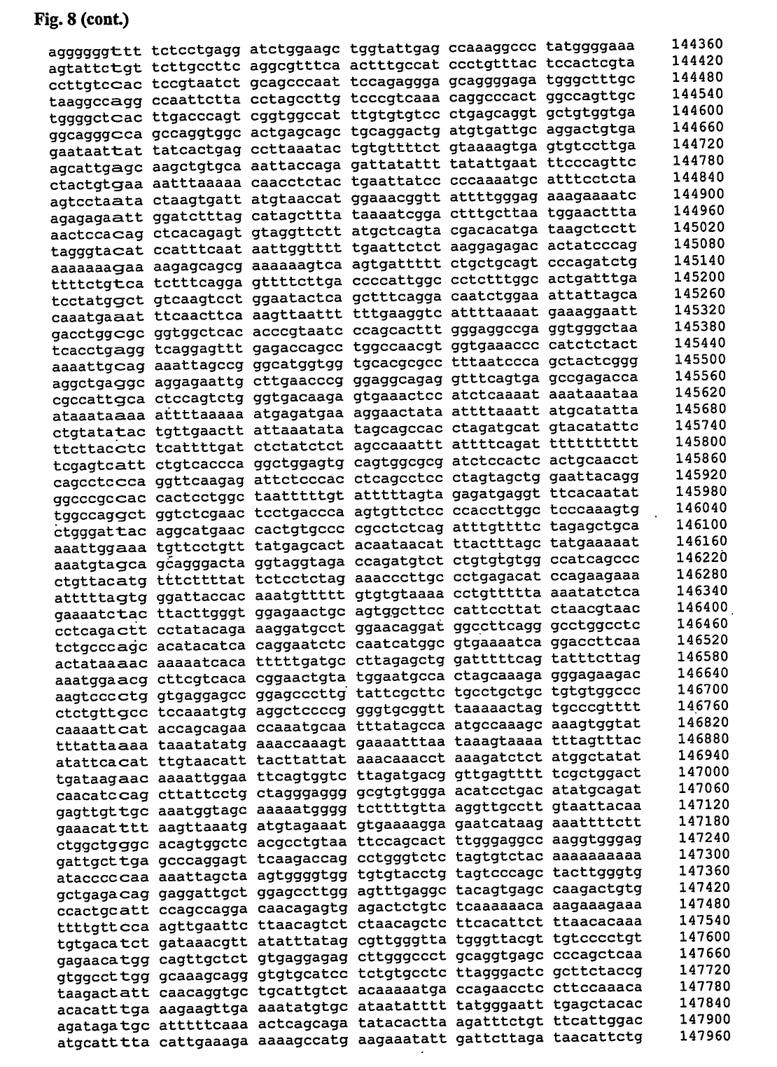

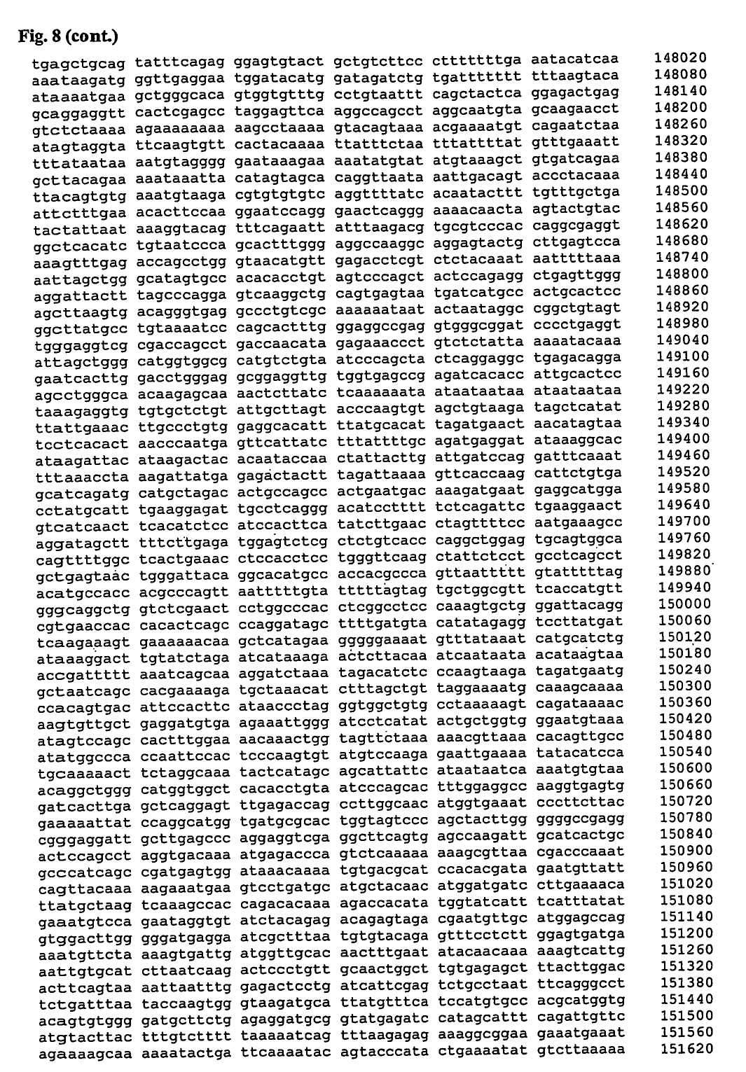

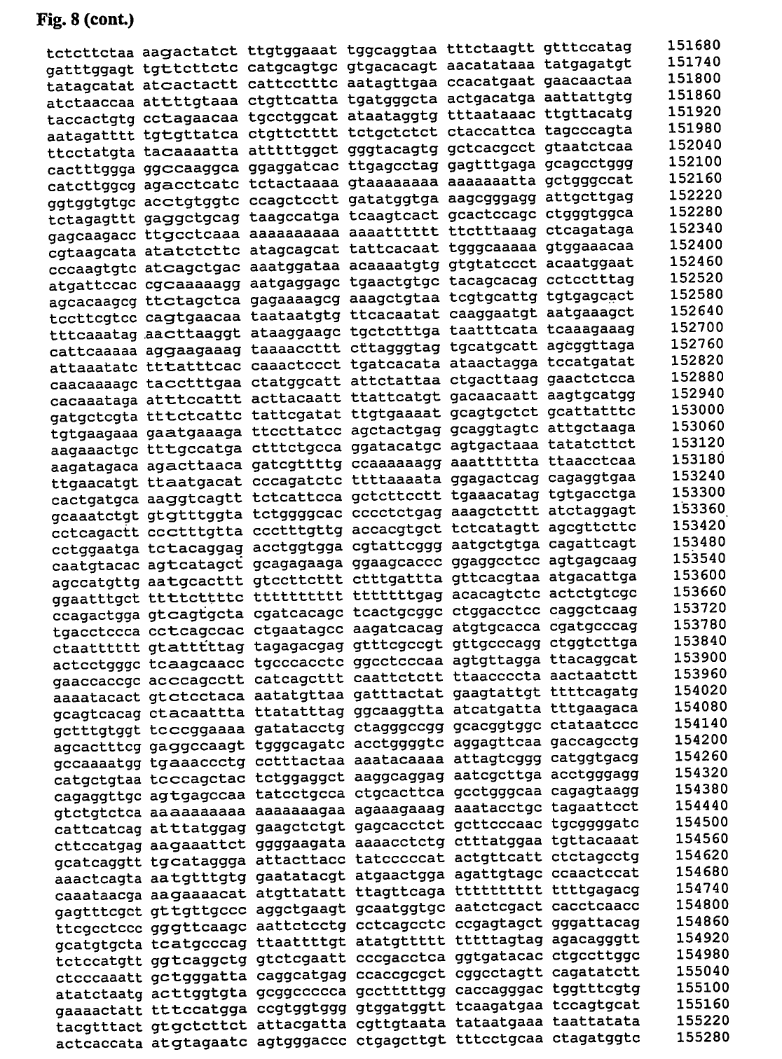

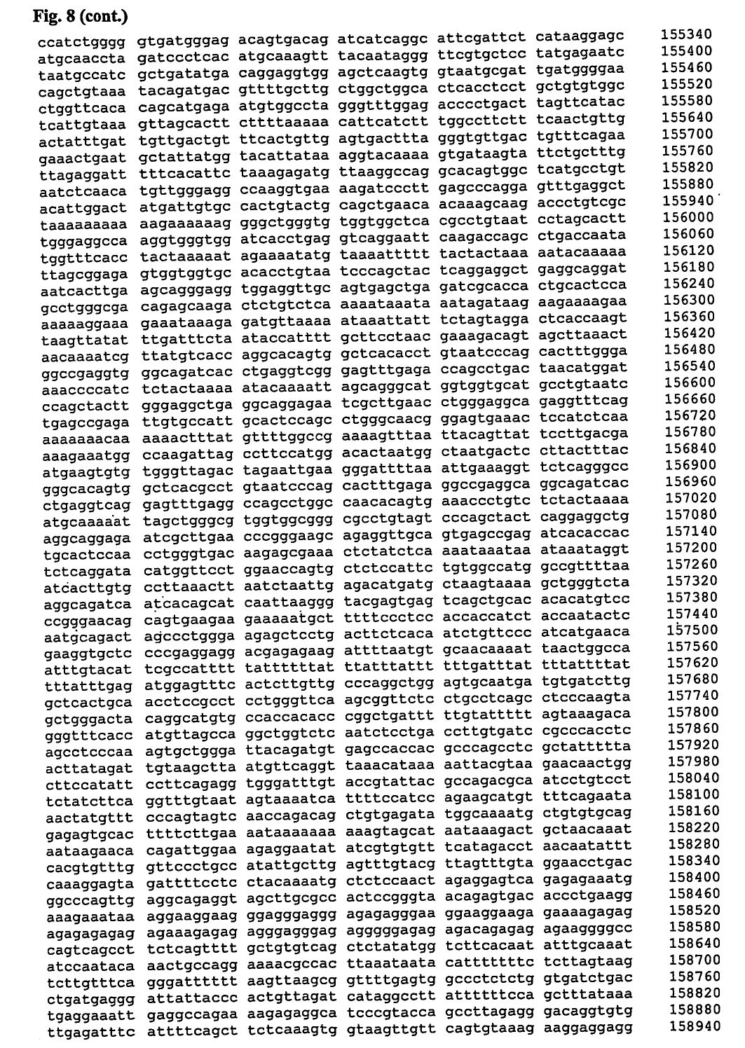

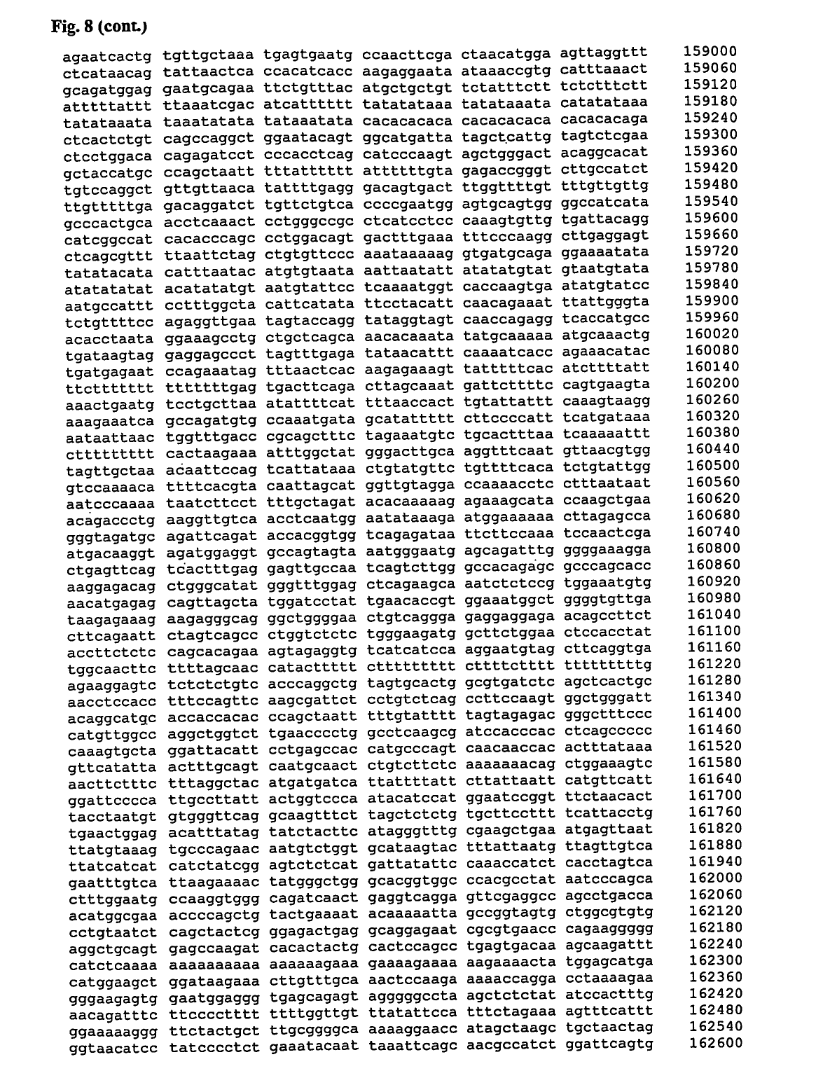

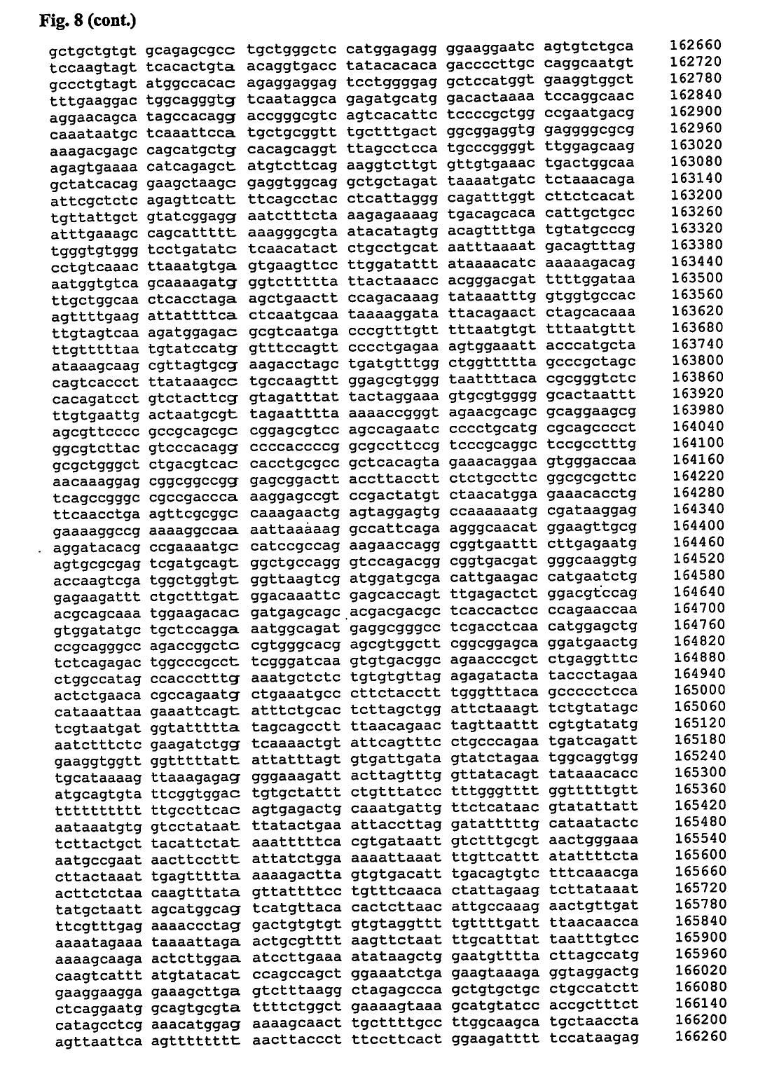

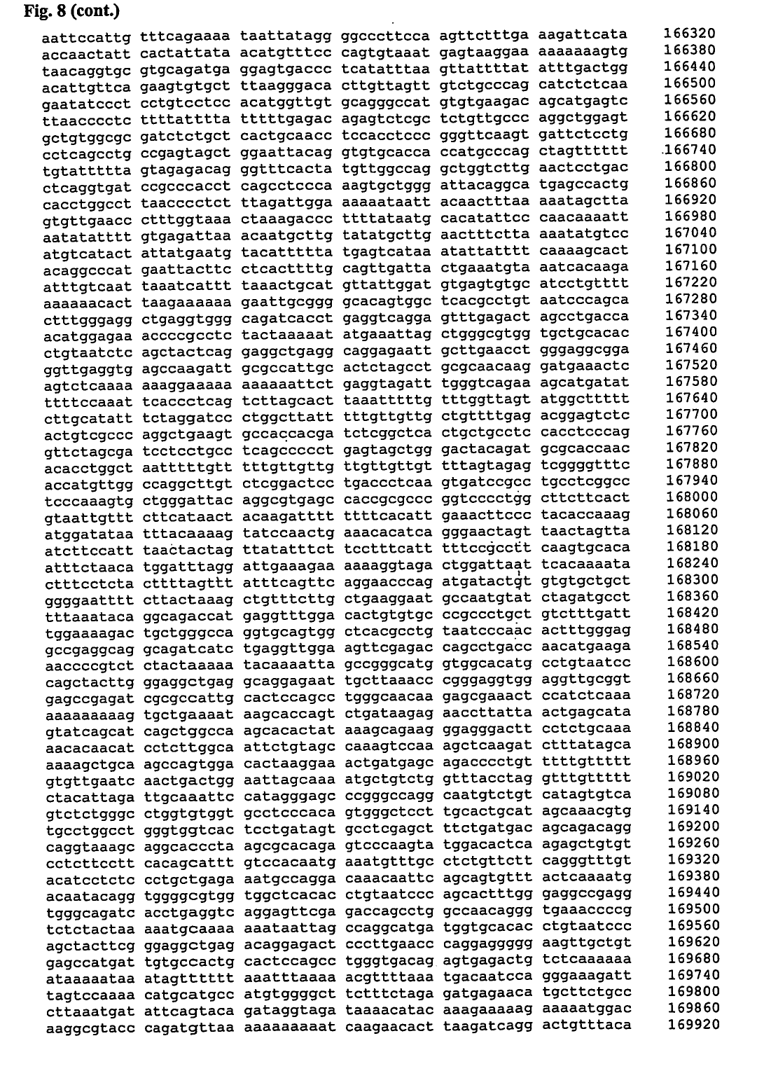

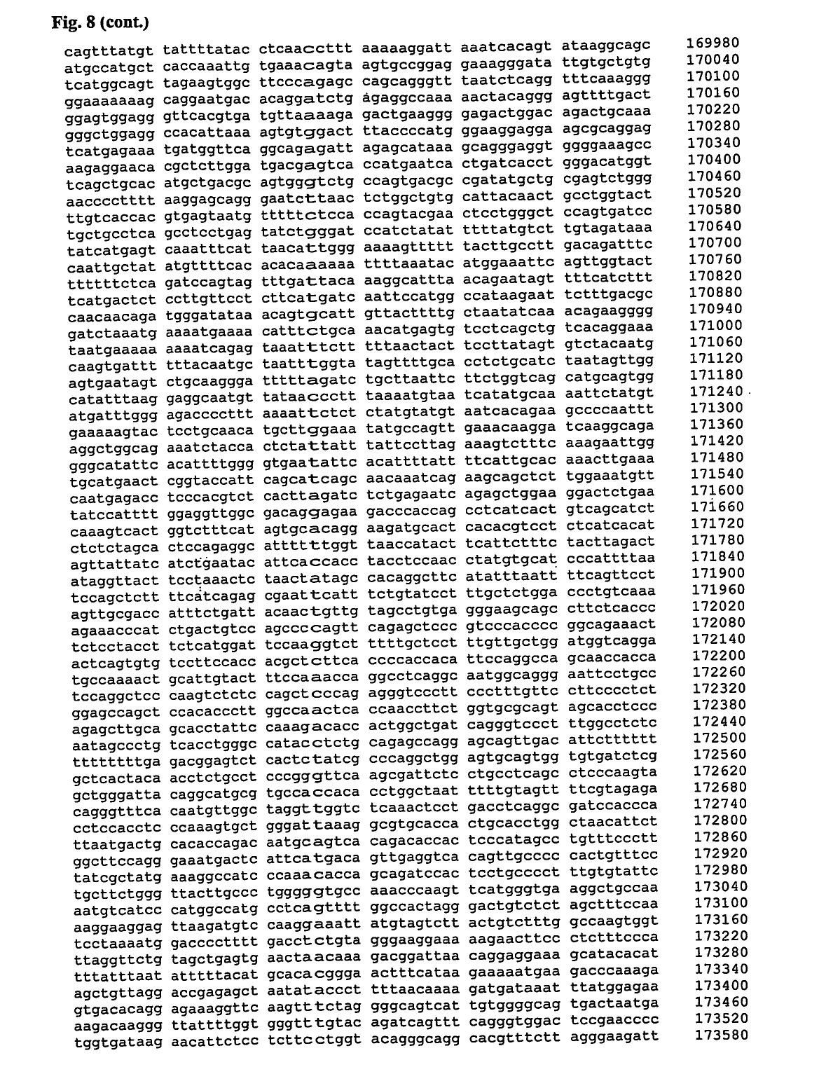

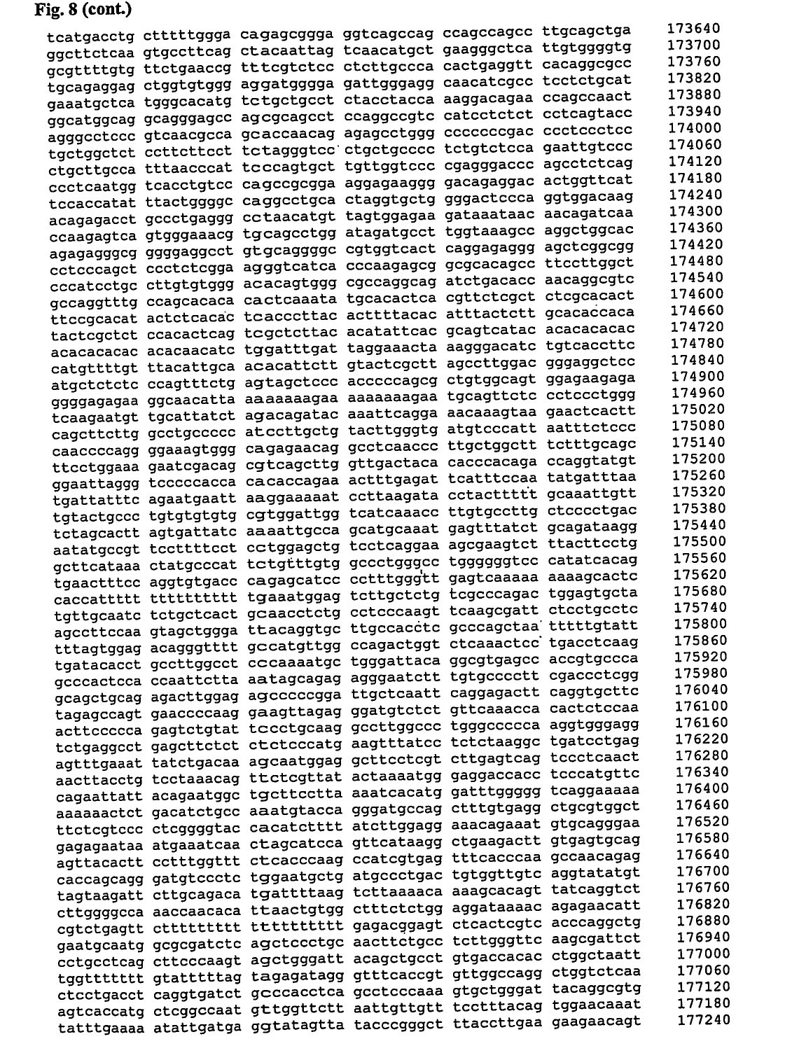

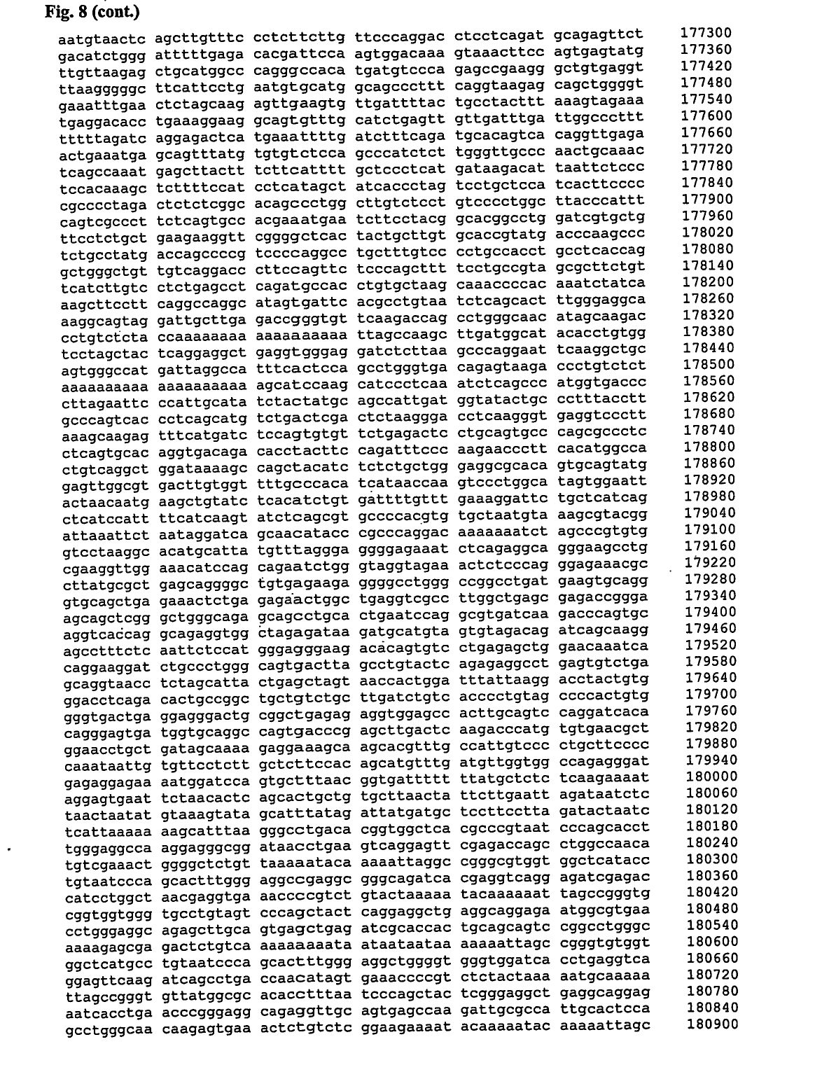

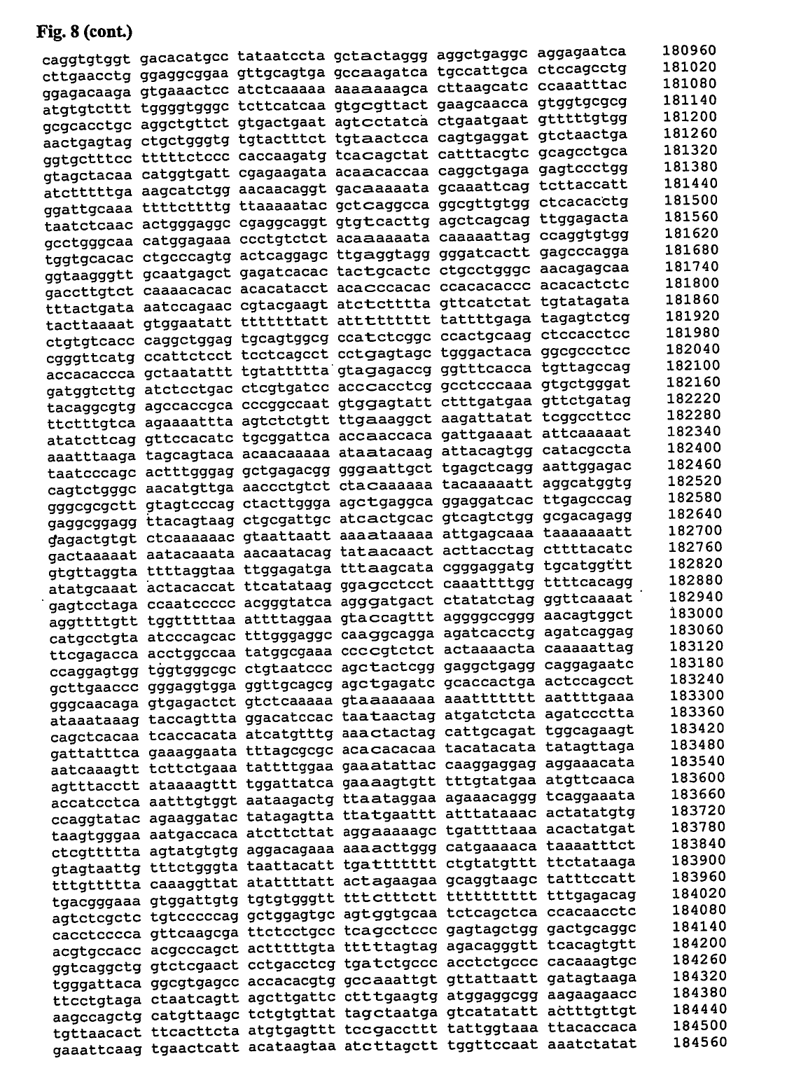

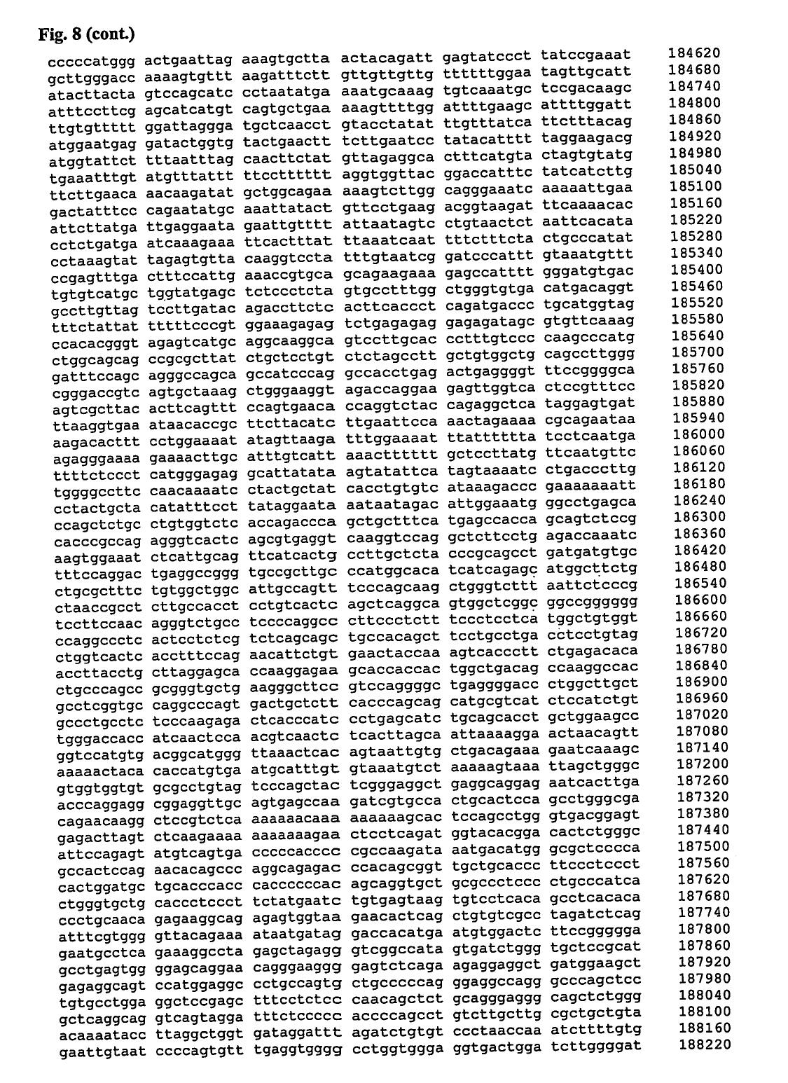

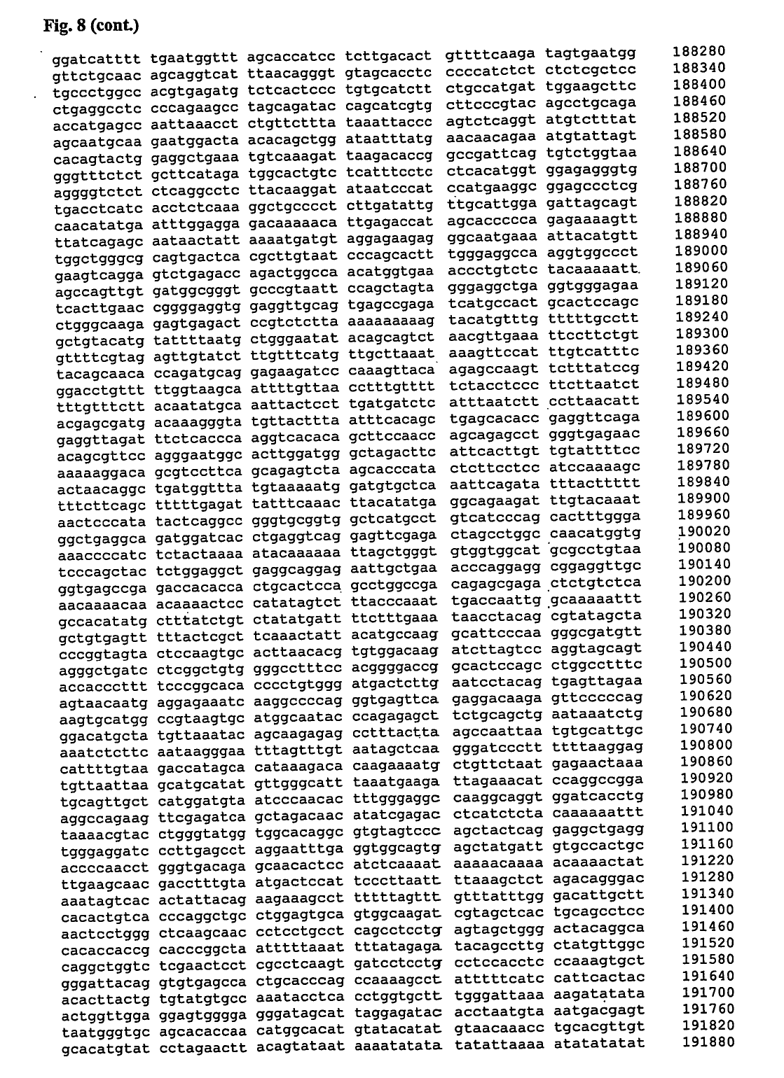

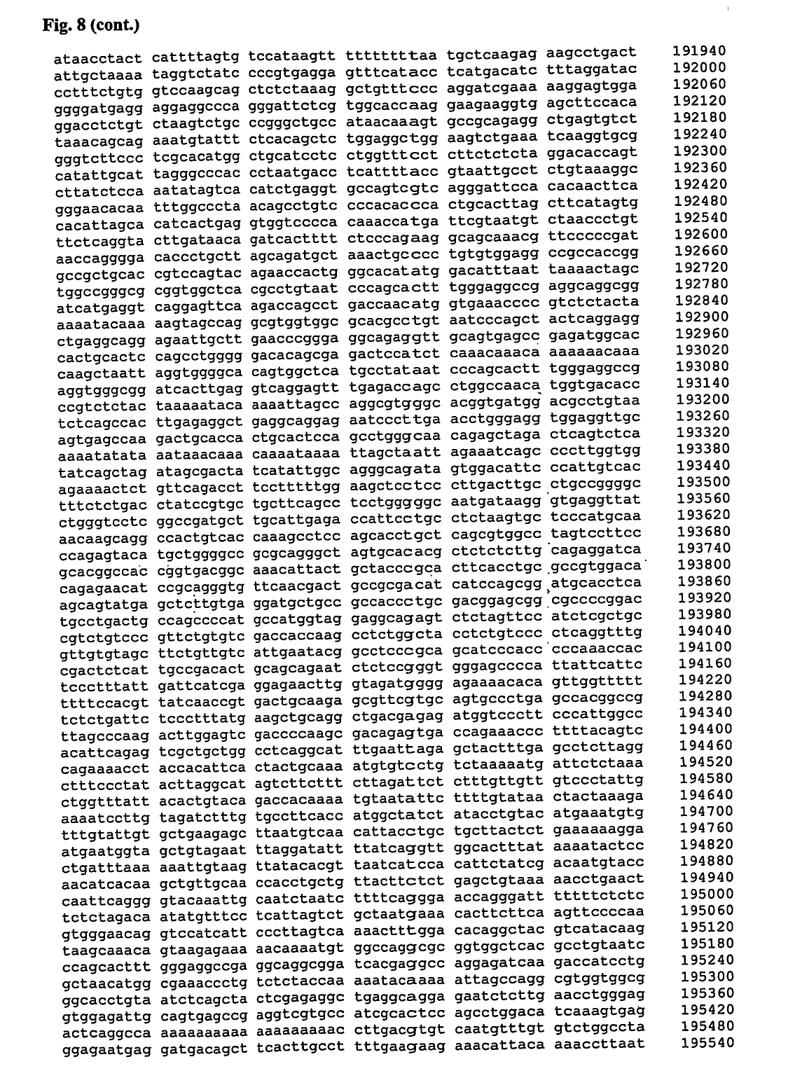

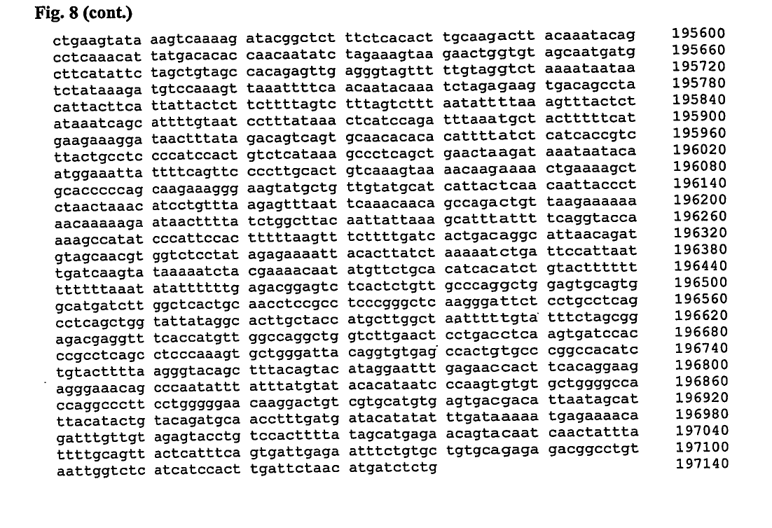

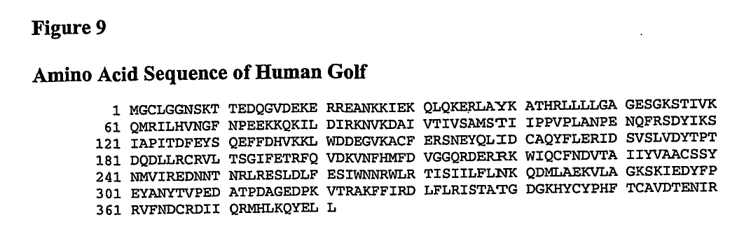

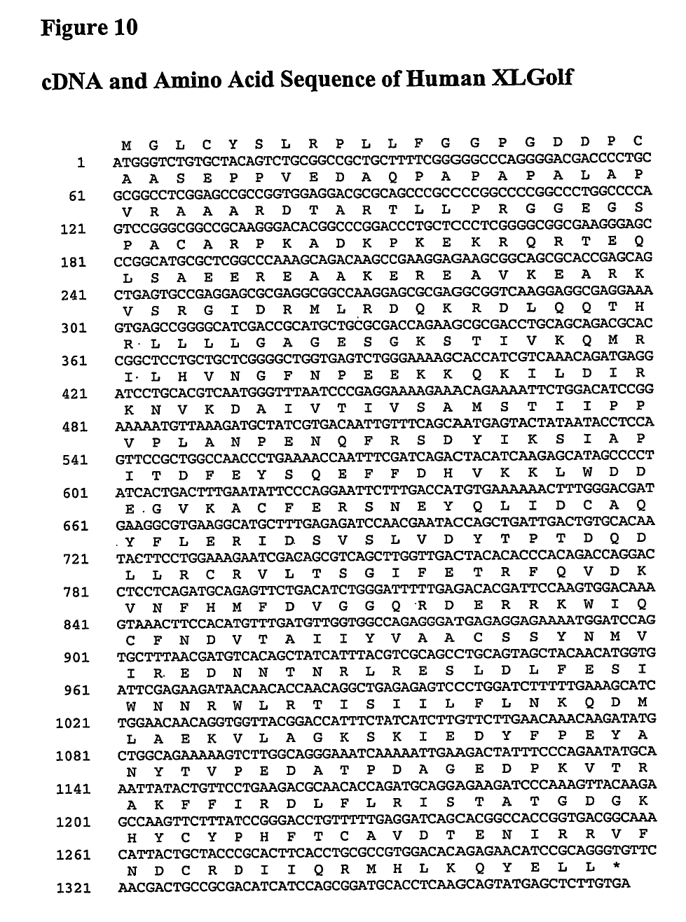

We have discovered new polymorphisms (single nucleotide polymorphisms (SNPs) and insertions) in the GNAL gene that have not been previously described. See annotations in FIG. 8. Some of these polymorphisms are in the alternative exon 1 region and others are in the region of the original exon 1. We also have sequencing evidence that there are differences in the occurrence of these polymorphisms between patients with schizophrenia and those with bipolar disorder. Such genotyping for a single gene can help identify an “at risk” individual. Moreover, since bipolar and schizophrenia are thought to involve multiple genes, such a diagnostic can also be combined with assays for other susceptibility genes.

The methods for genotyping GNAL polymorphisms for diagnostics are standard but can include pyrosequencing, primer extension, denaturing HPLC, Mass Spec, sequencing, or micorarray. In some embodiments, the assays are done using peripheral blood cells. In addition to measuring the occurrence of individual markers as described in the list of new polymorphisms from GNAL, combinations of these markers from GNAL can be used to construct haplotypes, or combinations of markers from the GNAL gene.

Golf and XLGolf Expression and Use in a Diagnostic

We have shown Golf expression is approximately 2× lower in patients with schizophrenia compared to clinically normal individuals using 3 different probes from GeneLogic. Lowered Golf expression is consistent with our overall hypothesis that there may be reduced expression in some individuals due to DNA methylation. Measuring Golf and XLGolf expression in tissues from patients will be useful to predict susceptibility to disease and/or response to treatment. Since obtaining human brain tissue from a live patient for diagnostic purposes is not practical, the use of a surrogate tissue for the diagnostic is desirable. Some examples of tissues that can be used in such diagnostics include, but are not limited to, olfactory tissue (that can be biopsied and has similarities to CNS tissue) or peripheral blood. Measuring expression of Golf, XLGolf and some set of other marker genes or proteins would be carried out. Either mRNA or protein expression can be used in the diagnostic for such expression measurements. Diagnostics for Golf/XLGolf can also be applicable to other disorders beyond psychiatry, most notably cancer.

Measuring mRNA Expression

We have developed TaqMan assays to measure both Golf and XLGolf and these can be used for developing a diagnostic. However, any approach that reliably measures mRNA levels would be suitable. For example, one way to develop this approach would be to measure Golf expression versus XLGolf.

Measuring Protein Expression

Golf and XLGolf protein expression could also be measured as a diagnostic. One method of such measurement is by ELISAs using the antibodies to Golf or XLGolf that we have developed. Any other immunological assay using Golf- or XLGolf-specific antibodies that reliably measures protein levels would also be suitable. A protein measurement assay can also be set up using a mass spectrometry,approach, such as ICAT.

DNA Methylation from GNAL Region [XL(Alternative Exon 1), Golf (Exon 1)]

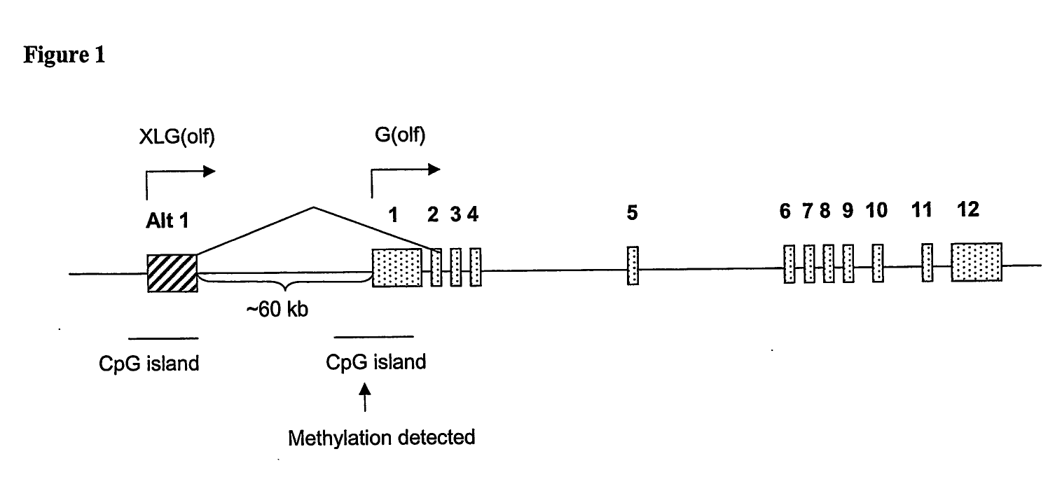

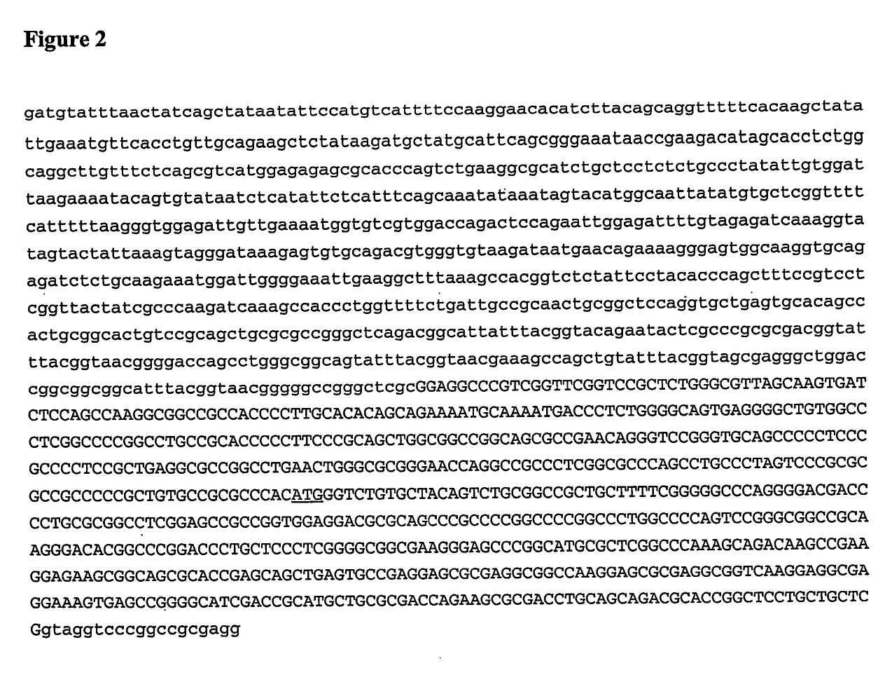

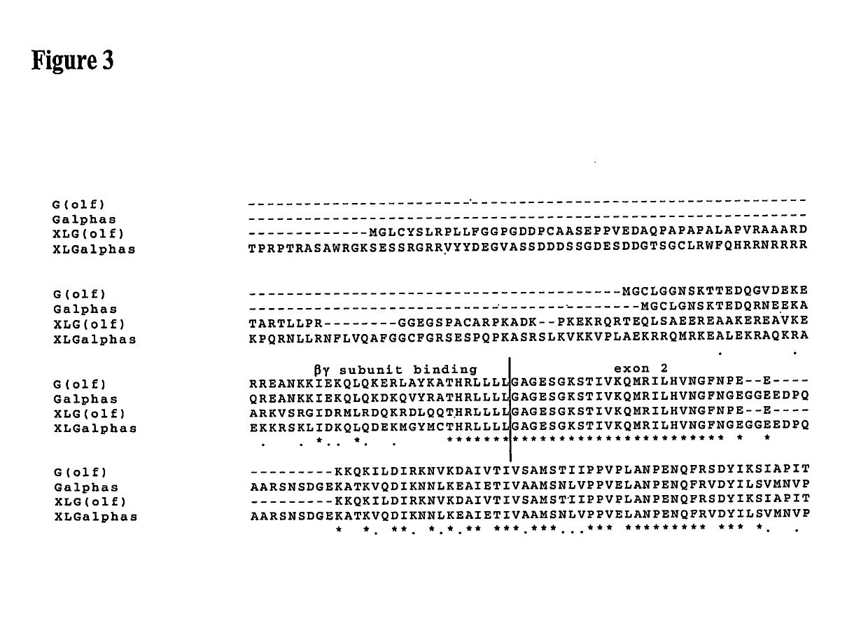

Since imprints consist of cytosine methylation in CpG islands, the GNAL gene was inspected for the presence of CpG islands. We have found CpG islands in the region of both the alternate and original first exons of GNAL. In contrast to the pattern of methylation observed for GNAS, no methylation has been detected in the CpG island encompassing the alternate exon 1. However, methylation of cytosines in the CpG island 5′ to the original exon 1 of GNAL has been detected (FIG. 1). Both methylated and unmethylated alleles were detected in genomic DNA from multiple human brain regions, suggesting that this locus is imprinted. This discovery can be harnessed in screening assays to diagnose heritable schizophrenia.

Measuring DNA methylation from different regions of the GNAL gene can provide information about an individual's risk for developing bipolar disorder or schizophrenia. Our data suggest that methylation patterns for both the alternative exon 1 and original exon 1 regions differ depending on tissue, including different regions of the brain. An assay can be developed based on DNA methylation of GNAL (either alternative exon1 region, exon 1 region or combination) from tissue outside the brain that would be diagnostic for risk of developing the disorders. The assay would typically be carried out on bisulfite treated DNA, and typical assays include, but are not limited to, methylation-specific PCR, denaturing HPLC or sequencing. The specific primers, described herein, that we designed for our studies can be used in such assays.

There is also evidence that methylation of DNA in a genomic region may affect expression of multiple genes in that region. Other genes in the Golf region that may be affected, and can be tested for, include IMPACT, IMPA.

Drug Screening/Therapeutics

The present invention also provides methods for preparing co-expression systems including a GPCR and Golf or XLGolf. We have set up co-expression systems in insect (Sf9) cells co-transfected with Dopamine Receptor D1 and Golf or XLGolf. Similar co-transfection systems can be set up with other GPCRs that interact with Golf (such as Adenosine A2a) or those that co-express with Golf or XLGolf (in the same brain regions) based on in situ analysis, such as, for example, GPR6 or GPR52. We have the ability to measure mRNA expression of Golf and XLGolf in these co-expression systems using the TaqMan assay developed for that purpose. Additional TaqMan assays can be developed to measure GPCR expression.

The invention also provides methods and screening assays to discover new drugs/compounds that modulate the interaction of Golf/XLGolf and a selected GPCR (such as measuring GTPγS, cAMP). There are several ways that our observations can be utilized for drug screening. A straightforward approach is to use the co-transfection system described above in cell lines expressing a GPCR and Golf or XLGolf. The cells expressing the GPCR are treated with a compound and the generation of cAMP is measured, by standard procedures. Ideally, the cell lines used would not express other G-proteins that would interfere with the assay. In some embodiments insect cells are used, but other cell lines (from any source, including mammalian cells) lacking G-protein expression can be used. In addition to carrying out assays using whole cells, screening assays can be carried out using membrane preparations containing the desired GPCR and reconstituted with Golf/XLGolf and the proper co-factors. Procedures for such reconstitution are well known to the art. Specifically, measurements can be taken of agonist-induced GTPγS binding using different Golf isoforms with a GPCR. Similarly, measurements of the effect of test drugs/compounds on activity of purified Golf or XLGolf can be made by measuring GTP/GDP exchange or GTPase activity. GTP/GDP exchange or GTPase activity measurement is well known to the art and there are commercially available kits, for example, a kit is available from Molecular Probes.

The invention also provides methods of analyzing the extent to which drugs act selectively on or through Golf versus XLGolf. Our data indicates that XLGolf has more peripheral expression than Golf. Such distinction will permit screening for drugs that act selectively on or through one or the other isoform. Such screening can be done by measuring the relative activity for any given test drug or compound in Golf versus XLGolf assays as described above.

In addition to methods of screening for compounds that modulate the activity of Golf/XLGolf, we could also screen for compounds that modulate the expression Golf or XLGolf Such screening can be done in cell lines using the methods described above, as well as in tissues harvested from animals. Compounds can similarly be tested for differential effects on the activity and/or expression of Golf and XLGolf.

Therapeutics

Screening for GPCR Agonists

We have discovered that GNAL (Golf) mRNA expression is decreased in the brains of individuals with schizophrenia. We have constructed 2 co-expression systems: 1) Dopamine receptor D1 (DRD1) and Golf; 2) DRD1 and XLGolf To assess signaling through these GPCR/G-protein coupled systems, a typical measure of activity is cAMP production. As a result, one approach for developing new therapies is as follows: set up multiple co-expression systems in cells containing GPCRs that relevant to disease (DRD1, A2A, GPR6, GPR52 or other GPCRs) with different levels of Golf or XLGolf. Test compounds are screened to identify those that, at lowered concentrations of Golf/XLGolf, result in improved signaling.

Compounds That Directly Modulate Activity or Expression of Golf or XLGolf

There is evidence in the literature that Golf expression can be up-regulated following long term exposure to lithium (rat) studies. As G-proteins, by definition, Golf and XLGolf couple to GPCRs to tranduce the receptor signal. However, there are also other G-proteins that perform this role and it is not yet known how selectively Golf/XLGolf couple to specific receptors such as DRD1 or adenosine A2A.

The invention also provides methods of screening for compounds that modulate the activity of DNA methyltransferase. Our evidence suggests that DNA methylation of genomic DNA in the region of Golf is relevant to psychosis. Compounds that selectively modulate the DNA methyltransferase activity on DNA in that region can be used in the treatment of psychosis.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### Novel Splice Form of GNAL

In an attempt to identify novel splice forms of the GNAL gene, the published cDNA sequence was compared to databases of expressed sequence tags (ESTs). A Golf cDNA sequence (GenBank accession number L10665) encompassing the full coding region and some of the 5′ and 3′ UTRs served as the query in a search of human EST databanks using the WU-Blast2 algorithm. All partial length, high identity matches were manually inspected for the presence of DNA sequence that could identify alternate splice forms or novel exons.

Hits with novel sequence were compared to the known GNAL gene structure and the draft human genome sequence. One such variant identified a new GNAL transcript with an alternative first exon spliced to the known exon 2 of GNAL. This new exon maps to human chromosome 18p11.2, approximately 60 kb telomeric to the published exon 1 of GNAL (FIGS. 1 & 2). A full-length transcript containing this alternative first exon and exons 2-12 of GNAL was subsequently verified by RT-PCR from human brain tissue.

A first-strand cDNA comprising XLGolf was synthesized from RNA from human brain tissue (striatum) obtained from Analytical Biological Services (Wilmington, Del.), using the gene specific primer 5′-CCTCACAAGAGCTCATACTGC-3′ (SEQ ID NO:13) and the Superscipt first-strand cDNA synthesis kit from Invitrogen (Carlsbad, Calif.). A full-length cDNA encoding XLGolf was generated by PCR amplification of this cDNA using the primers 5′-CACCATGGGTCTGTGCTACAGTCTG-3′ (SEQ ID NO:14) and 5′-TCACAAGAGCTCATACTGCTT-3′ (SEQ ID NO:15). The XLGolf cDNA was then directionally cloned into the vector pENTR/D-TOPO (Invitrogen (Carlsbad, Calif.)). The cloned PCR product was verified by DNA sequencing.

The identification of an alternative first exon illustrates not only a previously unidentified splice form, but an additional transcriptional start site and presumably a distinct regulatory promoter region. This gene structure is highly similar to the structure of the related GNAS gene that encodes the G-protein alpha subunit Gαs. As the protein encoded by the more 5′ exon 1 of GNAS is longer than the originally identified protein, it was named XLGαs (for eXtra Large). Therefore, we have named the alternative transcript of GNAL “XLG(olf)”. XLGolf and Golf differ only in their first exons, sharing exons 2 through 12.

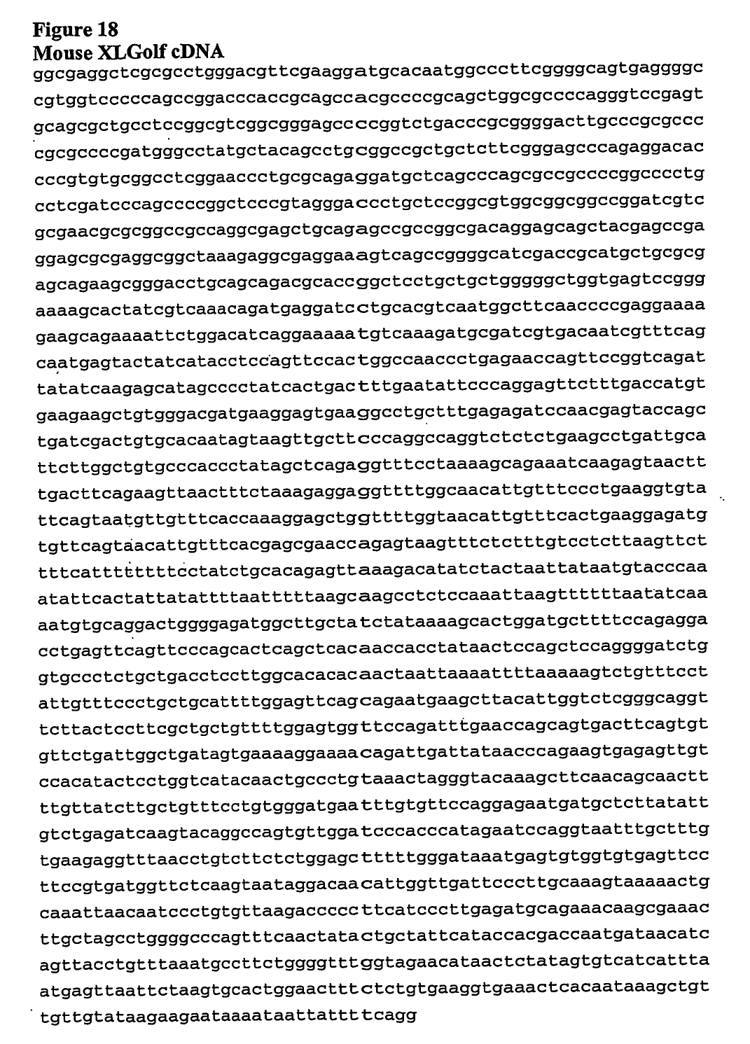

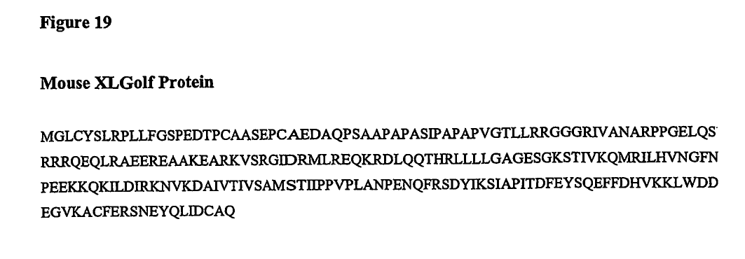

A search of mouse EST data identified a similar splice form (RefseqN NM_{—}177137). The mouse XLGolf cDNA sequence, which includes 5′ and 3′ untranslated regions, (SEQ ID NO:5) is presented in FIG. 18; the coding sequence for the mouse XLGolf protein spans nucleotide positions 196 to 885 of SEQ ID NO:5. The predicted mouse XLGolf protein sequence (SEQ ID NO:6) (RefseqP NP_{—}796111) is shown in FIG. 19.

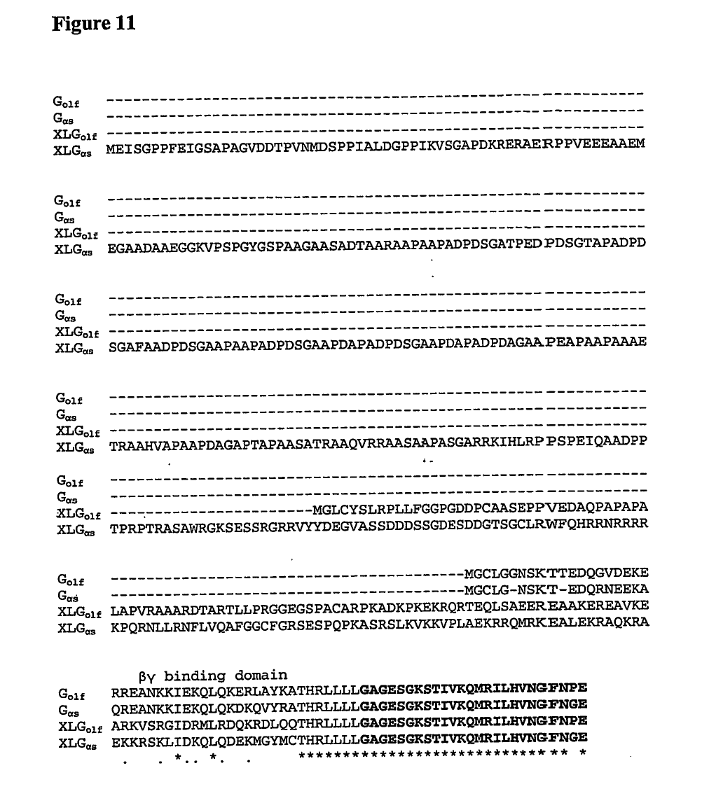

An open reading frame in the alternate exon 1 of GNAL begins with an ATG within a reasonable Kozak consensus sequence, has an upstream in-frame stop codon, and is conserved between human and mouse. Predicted amino acid sequences were aligned with the ClustalW program. While the predicted amino acid sequence of XLG(olf) shares little similarity with the N-terminal region of XLGαs, both alternative first exons share a conserved βγ-subunit binding domain with the originally described proteins (FIG. 11). This evidence supports that the XLG(olf) transcript encodes a functional G-protein alpha subunit.

### Example 2

### Comparison of Golf and XLGolf Sequences

A cDNA encoding Golf was also cloned. An adenine base (A) appears at nucleotide position 135 in the sequence that we cloned for Golf (SEQ ID NO:3). The nucleotide sequence of the human G protein alpha-olf subunit (olfactory) mRNA sequence presented in GenBank (accession number L10665) contains a guanine base (G) at nucleotide position 135. At nucleotide position 171, a thymine base (T) appears in SEQ ID NO:1, while a cytosine base (C) appears at nucleotide position 171 in GenBank sequence L10655. Our clone for Golf (SEQ ID NO:3) and the GenBank sequence L10655 encode identical proteins.

The same base change at nucleotide position 171 (A in place of G) was found in the equivalent position (nucleotide position 402) of our cloned sequence encoding XLGolf (SEQ ID NO:1). In addition, another change was found where a C appears in SEQ ID NO:1, but a T appears in the L10665 sequence at nucleotide position 1185. Both changes are silent.

A nucleotide alignment (PILEUP) was carried out between the cDNA encoding Golf (SEQ ID NO:3) and the cDNA encoding XLGolf (SEQ ID NO:1). The two splice forms are identical after the first exon (position 377 in our alignment; see FIG. 11). Before the splice site, these two sequences are identical at only 62 of 376 base pairs (16% identity over the first exon). Over the entirety of their sequences, SEQ ID NO:3 and SEQ ID NO:1 are identical at 1063 out of 1377 nucleotide positions (77% overall identity).

The XLG(olf) alternative transcript encodes a protein of 458 amino acids in length; 77 amino acids longer than the Golf protein (381 amino acids in length).

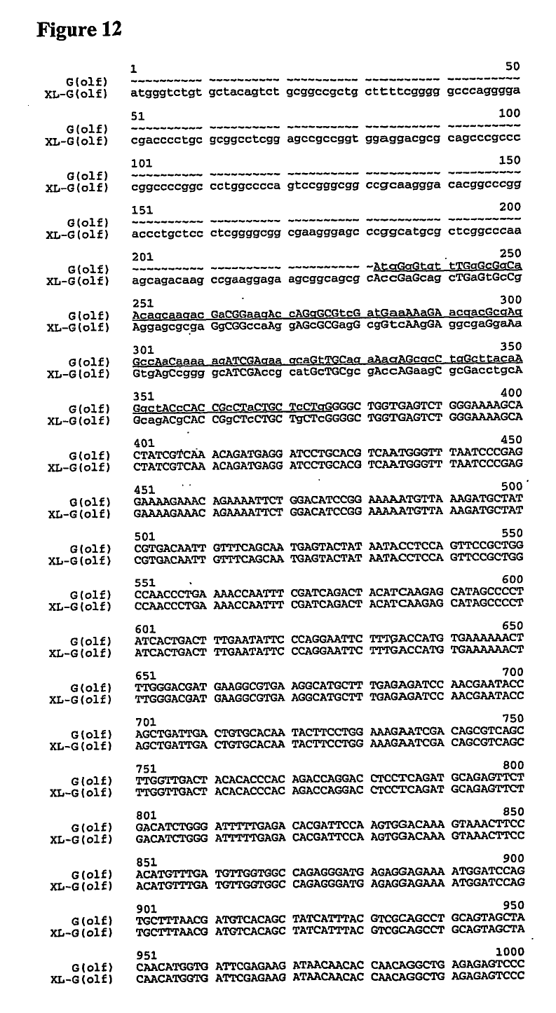

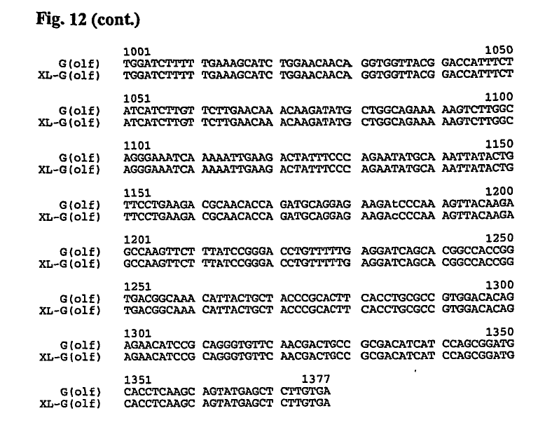

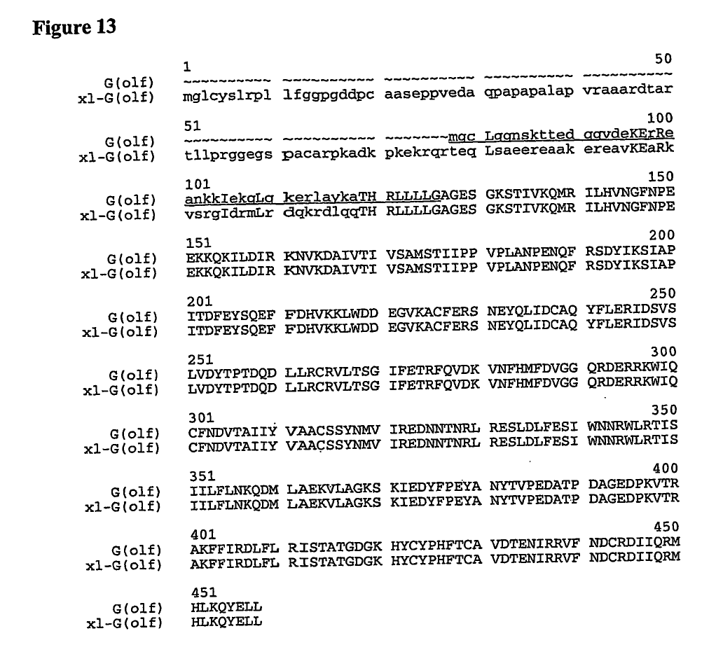

Beginning with the regions of the proteins encoded by second exon and continuing to their C-termini (amino acid positions 127 to 458 of SEQ ID NO:1), the two splice forms are 100% identical at the amino acid level (see FIG. 12). Golf and XLGolf have distinctly different N-termini, encoded by alternate exons, sharing only 14 amino acid residues in their overlapping regions. Comparison (PILEUP) of the full-length proteins reveals overall identity at 347 amino acid residues out of 458 (76% identity).

### Example 3

### Comparison of GNAL and GNAS Transcripts

An N-terminal amino acid alignment of the conceptual translations of the long and short transcripts of GNAL and GNAS (see FIG. 11). The sequence in bold is that encoded by the common second exons. While the coding region of the original first exons are well conserved, there is little conservation in much of the ‘XL’ forms. The exception is the region just N-terminal to the exon 2 coding sequence. This region contains the βγ-binding domain of the alpha subunits. Interestingly, the last 7 amino acids encoded by the first exons are completely conserved. Asterisks denote identity, and dots denote similarity.

### Example 4

### DNA Methylation Analysis Using Methylation Specific PCR (MSP)

Prediction of CpG islands in regions of the alternative first exons was accomplished with the programs CpGPlot/CpGReport (from the EMBOSS suite of sequence analysis software) using a window of 400 nt, an observed/expected ratio of CG dinucleotides of at least 0.6, and a minimum G+C content of 0.5.

Oligos Used for Golf and XLGolf Methylation Specific PCR

Golf and XLGolf MSP of DNA samples from the T98G neuroblastoma cell and from peripheral blood was carried out using the following primer pairs; expected product size is indicated in brackets: Golf primers used were M25+M26 (218 bp), U27+U28 (223 bp), M37+M38 (158 bp), and U39+U40 (162 bp); XLGolf primers used were M1+M2 (178 bp), U3+U4 (183 bp), M2+M5 (186 bp), and U4 +U6 (190 bp).

| | | |
|---|---|---|
| MSP | Primer sequences | SEQ ID NO |

| | | |
|---|---|---|
| XLGolf | | |
| M2 | GAACAACAAAAACCGATACGTC | SEQ ID NO:16 |
| M5 | GTTCGGTTTAAAGTAGATAAGTCGA | SEQ ID NO:17 |
| U4 | TACCAAACAACAAAAACCAATACAT | SEQ ID NO:18 |
| U6 | GTTTGGTTTAAAGTAGATAAGTTGA | SEQ ID NO:19 |
| M1 | TAAAGTAGATAAGTCGAAGGAGAAGC | SEQ ID NO:20 |
| U3 | TTTAAAGTAGATAAGTTGAAGGAGAAGTG | SEQ ID NO:21 |
| Golf | | |
| M25 | TAAGAGAGTTAGGCGGTCGC | SEQ ID NO:22 |
| M26 | CCTAATCTAAAATCCCGATACGAA | SEQ ID NO:23 |
| U27 | GTGTAAGAGAGTTAGGTGGTTGTG | SEQ ID NO:24 |
| U28 | TCCCTAATCTAAAATCCCAATACAA | SEQ ID NO:25 |
| M37 | TTCGTTCGTTAGGAGTAGGGAC | SEQ ID NO:26 |
| M38 | CGACTAAAACGCTTACACGCT | SEQ ID NO:27 |
| U39 | TTTTTGTTTGTTAGGAGTAGGGATG | SEQ ID NO:28 |
| U40 | ACCAACTAAAACACTTACACACT | SEQ ID NO:29 |

Bisulphite DNA Modification Protocol

Genomic DNA extracted from cell lines, peripheral blood or brain were modified using the CpGenome™ DNA Modification Kit (Chemicon International, Temecula, Calif.) purchased from Serologicals Corp. (Norcross, Ga.) (Herman et al., 1996, Proc. Natl. Acad. Sci. U.S.A., 93: 9821-9826).

The protocol used was the same as recommended by the manufacturer with minor modifications. Briefly, 1 ug of genomic DNA in 100 ul of molecular biology grade water was incubated with 200 μM NaOH at 37° C. for 15 minutes. After the incubation 500 μl of DNA Modification reagent I pH5 was added to the DNA that was then incubated at 55° C. for 20 hours. After the 20 hour incubation the completion of the chemical modification and DNA clean up was performed as per the protocol recommended by the manufacturer. Modified DNA was resuspended in 25 μl of 10 mM Tris/0.1 mM EDTA pH7.5 and stored at −20° C.

MSP was carried out on the modified DNA using MSP primers designed using Serologicals Primer Design Software or MethPrimer (Li & Dahiya, 2002, Bioinformatics, 18:1427-1431). Oligonucleotides were purchased from MWG Biotech (High Point, N.C.).

MSP reactions were set up as follows using Amplitaq Gold purchased from PerkinElmer (Boston, Mass.) or Applied Biosystems (Foster City, Calif.) and consisted of the following:
1×PCR reaction Buffer (Serologicals Corp. (Norcross, Ga.)).
2.5 mM dNTP mix
17.5 mM MgCl₂
40 μM MSP primers
1 unit Amplitaq Gold
˜100 ng modified DNA

PCR was carried out using the MJ Research PT-200 DNA Engine using the following general cycling conditions:
Step 1.95° C., 9 minutes
Step 2.95° C., 45 seconds
Step 3.55° C., 45 seconds
Step 4.72° C., 1 minute
Step 5.4° C.

Steps 2-4 were repeated 35 times. Note: annealing temperatures (step 3) were modified depending upon the Tₘ of the PCR oligonucleotide used.

The PCR reactions were then subjected to electrophoresis in a 1% agarose/TEA gel containing 0.5 ug/ml ethidium bromide. Bands were visualized using GeneGenius Bioimaging System (Syngene, Frederick, Md.).

Results

Modified DNA from the human glioblastoma cell line T98G, that is known to be methylated at the Golf locus (Costello et al., 2000, Nat. Genet., 24:32-38), was subjected to MSP using a series of primers designed to amplify both modified-unmethylated DNA (U primers) and modified-methylated DNA (M primers).

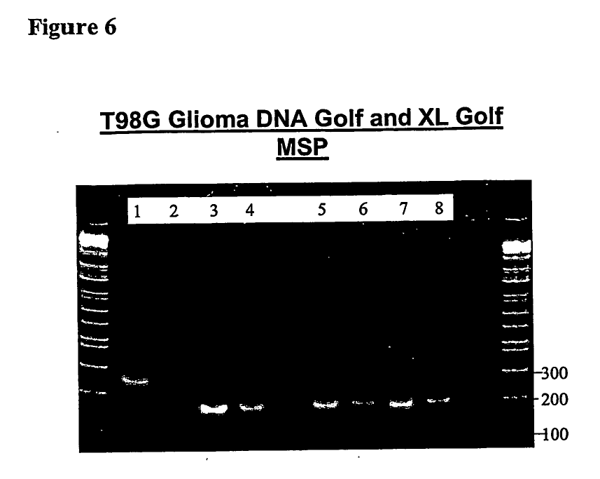

Using the Golf primers (M25 and M26), a DNA fragment of the expected size, 218 bp, was observed. No DNA fragments were observed with the corresponding set of Golf U primers, U27 and U28. A similar result was obtained with another set of M and U primers (37, 38, 39 and 40 respectively) that identified a different region of the Golf CpG island. The size of the DNA fragment for the M primers was 158 bp. See FIG. 6.

A similar set of MSP reactions were carried out on T98G DNA using the XLGolf primer sets (M2+M5) and (U4+U6). Results obtained showed that a DNA fragment of 186 bp for the M primer set and 190 bp for the U primer set were amplified. These were the expected fragment sizes for the methylated and unmethylated products. See FIG. 6.

To confirm that these DNA fragments actually represented methylated forms of Golf the fragments were cloned and subjected to DNA sequencing. Sequence analysis confirmed that these DNA fragments corresponded to regions of the Golf CpG island that are methylated in the cell line T98G. These data indicated that in the T98G cell line the Golf locus appears to be 100% methylated, as observed by Costello et al., 2000, supra. Our observations suggest that the XLGolf locus in the T98G cell line is hemi-methylated.

Similar MSP was then carried out on modified DNA from a number of different brain regions including Hippocampus, Sustantia nigra, Nucleus accumbens and Caudate nucleus, Anterior thalamus, Frontal cortex and peripheral blood from normal individuals.

Results showed that for Golf, methylation at this locus was only seen in Substantia nigra, Nucleus accumbens and peripheral blood. Unmethylated Golf was seen in all brain regions but not in the T98G cell line DNA.

In contrast XLGolf exhibited methylation in all brain regions except Anterior thalamus and also showed methylation in peripheral blood. The only brain region that showed no unmethylated XLGolf was Nucleus accumbens.

The results are summarized as follows.

| | cell line | | | |
|---|---|---|---|---|
| | Primer set | Primer set | Primer set | Primer set |
| | M2 + M5 | U4 + U6 | M25 + M26 | U27 + U28 |
| | XLGolf | XLGolf | Golf | Golf |
| | methyl- | unmethyl- | methyl- | unmethy- |
| Tissue | ated | ated | ated | lated |
| T98G | + | + | + | − |
| Substantia Nigra | + | + | + | + |
| Caudate nucleus | + | + | − | + |
| Hippocampus | + | + | − | + |
| Frontal cortex | + | + | + | + |
| Nucleus accumbens | + | − | − | + |
| Anterior thalamus | − | + | − | + |
| Peripheral blood | − | + | + | + |

(normal)

Using primer set M25+26 and U27+28 bands of approximately 218 bp for the M primers and 223 bp for the U primers were observed when the samples were subjected to electrophoresis in a 10% acrylamide/TBE gel. This result indicated that in normal human brain tissue in the regions tested the Golf locus is hemi-methylated. A similar result was seen using the M37+38 and U39+40 primer sets.

Subsequently, the DNA fragments from these reactions were cloned and sequenced to verify that the resulting amplified DNA fragments were actually from the Golf and XLGolf loci. Sequence analysis verified that both methylated and unmethylated Golf loci are present in the regions of normal brain tissue tested.

Summary

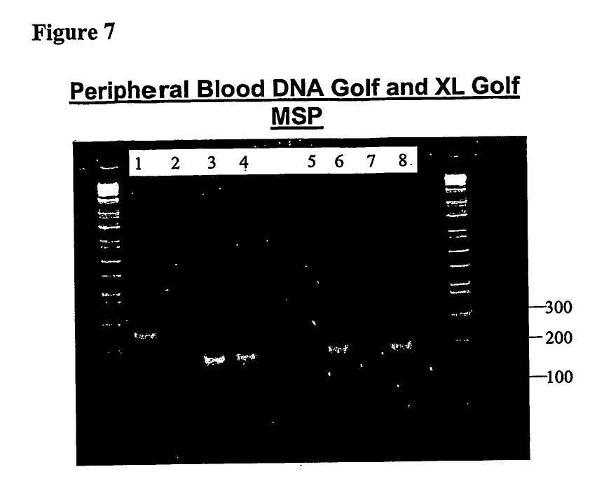

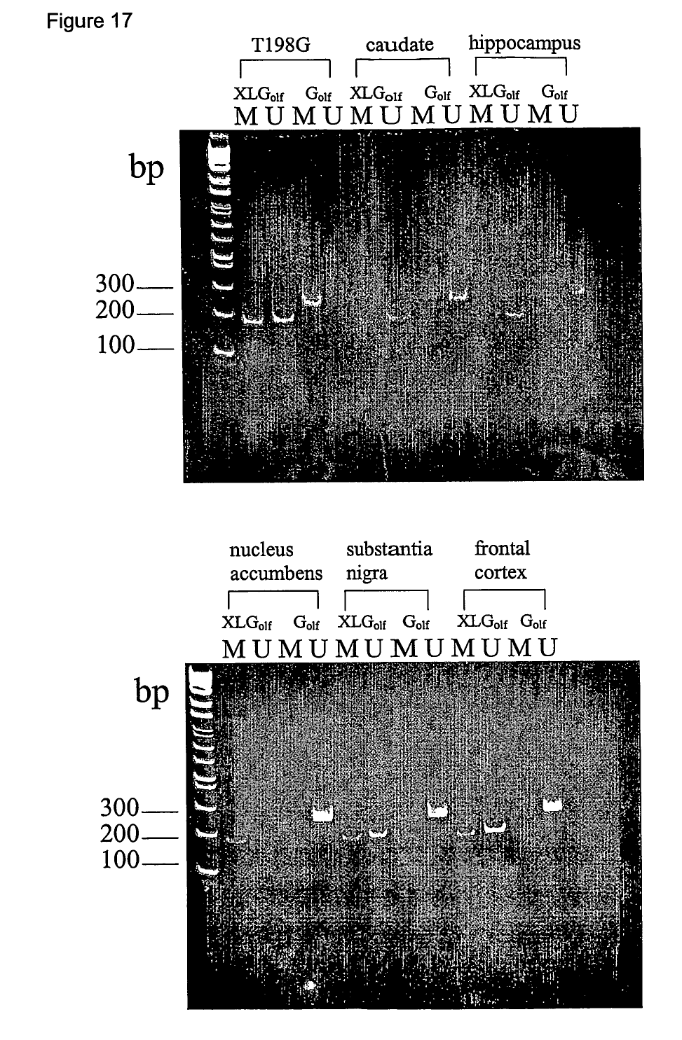

The methylation sensitive NotI restriction site in this region was used to guide the MSP studies, and the T98G glioma cell line served as a positive control for methylated DNA. As expected, only methylated DNA was detected in the T98G cell line by MSP (FIG. 4). However, both methylated and unmethylated DNA were detected in genomic DNA from human frontal cortex, substantia nigra (FIG. 17), and peripheral blood lymphocytes (FIG. 7), suggesting that GNAL is imprinted. Differential methylation of the XLGolf CpG island was detected in the T98G cells, frontal cortex, hippocampus, substantia nigra (FIG. 17), and peripheral blood (FIG. 7). Although failure to detect the methylated or unmethylated state by this method is not definitive, detection of methylation is convincing evidence of epigenetic regulation of a locus. This suggests that Golf and XLGolf CpG islands are methylated in a tissue-specific manner, a phenomenon observed for some other imprinted genes, most notably GNAS.

### Example 5

### Golf and XLGolf in situ Probes

The following DNA sequences from Golf and XLGolf were used for in situ hybridization experiments. These DNA sequences were cloned into the vector pBSKII+ to allow expression of Golf and XL Golf anti-sense RNA.

XLGolf probe 1

(SEQ ID NO:30)

GCGGCCGCAAGGGACACGGCCCGGACCCTGCTCCCTCGGGGCGGCGAAGG
GAGCCCGGCATGCGCTCGGCCCAAAGCAGACAAGCCGAAGGAGAAGCGGC
AGCGCACCGAGCAGCTGAGTGCCGAGGAGCGCGAGGCGGCCAAGGAGCGC
GAGGCGGTCAAGGAGGCGAGGAAAGTGAGCCGGGGCATCGACCGCATGCT
GCGCGACCAGAAGCGCGACCTGCAGCAGACGCACCGGCTCCTGCTGCTCG
XLGolf probe 2

(SEQ ID NO:31)

GATAACAACACCAACAGGCTGAGAGAGTCCCTGGATCTTTTTGAAAGCAT
CTGGAACAACAGGTGGTTACGGACCATTTCTATCATCTTGTTCTTGAACA
AACAAGATATGCTGGCAGAAAAAGTCTTGGCAGGGAAATCAAAAATTGAA
GACTATTTCCCAGAATATGCAAATTATACTGTTCCTGAAGACGCAACACC
AGATGCAGGAGAAGATCCCAAAGTTACAAGAGCCAAGTTCTTTATCCGGG
ACCTGTTTTTGAGGATCAGCACGGCCACCGGTGACGGCAAACATTACTGC
TACCCGCACTTCACCTGCGCCGTGGACACAGAGAACATCCGCAGGGTGTT
CAACGACTGCCGCGACATCATCCAGCGGATGCACCTCAAGCAGTATGAGC
TCTTG
Golf probe

(SEQ ID NO:32)

ATGGGGTGTTTGGGCGGCAACAGCAAGACGACGGAAGACCAGGGCGTCGA
TGAAAAAGAACGACGCGAGGCCAACAAAAAGATCGAGAAGCAGTTGCAGA
AAGAGCGCCTGGCTTACAAGGCTACCCACCGCCTGCTGCTCCTGG

### Example 6

### CNS and Other Tissue Expression of G(olf) and XLG(olf)

PCR primers were designed to amplify both splice forms of the GNAL transcript. cDNA was made from human hippocampus and striatal RNA. PCR reactions amplified both forms of GNAL transcript from each tissue source. DNA sequence analysis confirmed identity of G(olf) and XLG(olf). Distinct expression patterns for Golf and XLGolf were identified.

The results are summarized in the following Tables 1 and 2.

TABLE 1
Relative Expression Levels of Golf and XLGolf in Brain.

| | | |
|---|---|---|
| Golf | Caudate nucleus | ++++ |
| | Hippocampus | +++ |
| | Hypothalamus | +++ |
| | Frontal lobe | + |
| | Temporal lobe | + |
| XLGolf | Spinal cord | +++ |
| | Substantia nigra | ++ |
| | Hypothalamus | +− |

Other brain regions were detectable but low for both Golf and XLGolf.

TABLE 2
XLGolf Peripheral Tissue Expression.

| | |
|---|---|
| Testis | ++++ |
| Brain | +++ |
| Lung | +++ |
| Adrenal gland | +++ |
| Thyroid | ++ |
| Ovary | + |
| Uterus | + |
| Prostate | + |
| Skin | + |
| Fetal brain | + |

All other tissues, low or undetectable expression.

### Example 7

### Real Time PCR

The RNA samples in which Golf, XLGolf and β2-microglobulin levels were determined were obtained from commercial suppliers (Ambion (Austin, Tex.), Stratagene (La Jolla, Calif.), BD Biosciences Clonetech (Palo Alto, Calif.)). Except for the nucleus accumbens (pool of 6 individuals) and the spinal cord (pool of 49 individuals), all of the RNA samples were derived from one tissue sample. The donors were different for each tissue. Reverse transcription was performed using reagents purchased from Invitrogen (Carlsbad, Calif.). For each RNA sample, cDNA was prepared in triplicate.

Controls for use in absolute quantitation were generated by PCR using plasmids containing Golf or XLGolf and the following oligonucleotides: 5′-CAGGATCCTCATCTGTTTGACG (SEQ ID NO:33) (used for Golf and XLGolf), 5′-GGTACCACCATGGGGTGTTTGGGCGGCACC (SEQ ID NO:34) (used for Golf), 5′-CAAGGAGGCGAGGAAAGTGA (SEQ ID NO:35) (used for XLGolf). The PCR products were purified using the QIAquick PCR Purification kit (Qiagen, Valencia, Calif.). The purified control fragments were electrophoresed in ethidium bromide containing agarose gels and the concentrations were determined by comparing the intensity of the bands with a curve constructed using the fluorescence of standards with known concentrations.

Taqman one step PCR mastermix, oligonucleotides and 5′-6FAM/3′-MGBNFQ Taqman probes were purchased from Applied Biosystems (Foster City, Calif.). Taqman assays were performed using the PRISM 7700 Sequence Detection System (Applied Biosystems (Foster City, Calif.)). β2-microglobulin levels were determined using human β2-microglobulin endogenous control predeveloped assay reagents (Applied Biosystems catalog number 4333766F). For quantitative realtime PCR, the following oligonucleotides were used to detect Golf: 5′-AAAGAGCGCCTGGCTTACAAG (SEQ ID NO:36); 5′-GTTTGACGATGGTGCTTTTCC (SEQ ID NO:37) and the following oligonucleotides were used detect XLGolf: 5′-GACGCACCGGCTCCT (SEQ ID NO:38); 5′-GATGGTGCTTTTCCCAGACTCA (SEQ ID NO:39). The sequence of the Golf Taqman probe was 5′-ACCAGCCCCCAGGAG (SEQ ID NO:40) and the sequence of the XLGolf Taqman probe was 5′-CCAGCCCCGAGCAGC (SEQ ID NO:41).

Each cDNA preparation was run in triplicate Taqman QRT-PCR reactions. Golf and XLGolf levels were calculated by comparing the threshold cycle numbers from Taqman reactions with the cDNA samples to standard curves constructed using known copy numbers of Golf or XLGolf purified PCR products (see above). Relative levels of β2-microglobulin were determined by comparing the threshold cycle numbers from Taqman reactions with the cDNA samples to standard curves constructed using diluted cDNA prepared from total human brain RNA. Dividing Golf or XLGolf levels by the β2-microglobulin level normalized the samples.

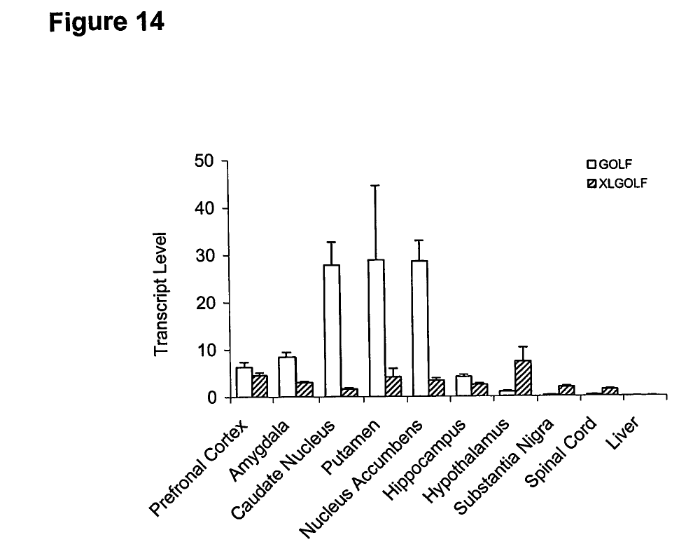

Using a Taqman quantitative PCR assay designed to span the exon ½ junction of each transcript, we determined the relative distributions of Golf and XLGolf in selected human CNS regions. In agreement with previous studies of the rat and mouse genes (Herve et al., 1995, Brain Res. Mol. Brain Res. 32:125-34; Belluscio et al., 1998, Neuron, 20:69-81) the Golf transcript is prominently expressed in the caudate, putamen, and nucleus accumbens. Lower levels of Golf were also detected in prefrontal cortex, amygdala, hippocampus and hypothalamus; whereas the transcript was barely detected or not detected at all in spinal cord, substantia nigra, and liver (FIG. 14). The relative distribution of XLGolf differs markedly from Golf, with the most prominent expression in hypothalamus, prefrontal cortex, and the ventral striatum. In those regions where both transcripts were clearly detected, the absolute levels of XLGolf exceeded Golf only in hypothalamus, substantia nigra and spinal cord.

Both the Golf and XLGolf transcripts are expressed in regions that are relevant to mood and psychosis, such as the nucleus accumbens and prefrontal cortex. Given that, Golf couples to GPCRs that mediate dopaminergic transmission and psychostimulant drug actions in those regions of the brain (namely the D1 and A2a receptors), apparent functional differences between the isoforms suggests that changes in the relative expression levels of Golf and XLGolf alter the pharmacology of the GPCRs that couple to them. Our quantitative assay of expression levels enables the measurement of absolute expression levels of Golf and XLGolf in cells or tissues under different conditions, such as normal and disease states.

### Example 8

### XLGolf Functions as a G Protein Alpha Subunit With DRD1.

Generation of Recombinant Baculoviruses

The XLGolf cDNA was introduced into the cloning vector pENTR/D-TOPO between Not I to Asc I sites. Recombinant baculovirus encoding human XLGolf was generated with the BaculoDirected™ expression kit from Invitrogen (Carlsbad, Calif.) according to the manufacturer's protocol. The titer of the third-passage viral stock was determined by plaque assay and used as the working stock.

Cell Culture and Membrane Preparation

Sf9 cells were suspended in SF 900 II medium containing penicillin (50 unit/mL) and streptomycin (50 μg/mL) and cultured at 28° C. with rotation (125 rpm). Cells were maintained at a density of 2×10⁶ to 4×10⁶ cells/mL. For infection, Sf9 cells at the density of 2×10⁶ cells/mL were infected with baculovirus (≈10⁸ pfu/mL) encoding human dopamine D1A receptor obtained from PerkinElmer Biosignal (Montreal, Canada), human Golf (PerkinElmer Biosignal (Montreal Canada), or human XLGolf at the appropriate multiplicity of infection (MOI). After infection for 48 hours, cells were harvested for membrane preparation. Cells were harvested by centrifugation at 500×g at 4° C. The cell pellets were washed twice with Dulbecco's phosphate-buffered saline (DPBS) at pH 7.4 and suspended in ice-cold 10 mM Tris-HCl with 5 mM EDTA (TE)(pH 7.4) containing a protease inhibitor cocktail (Roche Applied Science (Indianapolis, Ind.)) and sonicated. Following centrifugation at 1000×g, membranes were collected from the supernatant by centrifugation at 20,000×g for 30 min at 4° C. The membrane fraction was stored at −80° C. in TE containing 5% glycerol.

[³H]—SCH 23390 Saturation Binding Assay

Sf9 cell membranes (2 μg per reaction) was incubated with 0.018 to 14.4 nM [³H]—SCH 23390 (Amersham, Piscataway, N.J.) in the binding buffer (50 mM Tris-HCl, pH 7.4, 5 mM KCl, 5 mM MgCl₂, 5 mM EDTA, 1.5 mM CaCl₂) at room temperature for 1 hour. Non-specific binding was determined in the presence of 10 μM (+)-butaclamol (Sigma-Aldrich) in a total volume of 200 μL. Bound radioligand was collected on GF/C filters using a 96-well cell harvester. Filters were washed 5 times with 500 μl of cold 50 mM Tris-HCl buffer (pH 7.4) and filter-bound radioactivity determined by liquid scintillation.

SDS-PAGE and Immunoblot Analysis

Membranes from Sf9 cells expressing the DRD1 alone or DRD1 with Golf variants were solubilized in SDS-sample buffer to a final protein concentration of 1 mg/μl and heated at 80° C. for 5 min. Solubilized proteins were separated using SDS-PAGE and 4% to 12% gradient polyacrylamide gels. Proteins were transferred to polyvinylidene diflouride (PVDF) membranes and probed with rabbit anti-Golf antibody (K-19) from Santa Cruz Reagents (Santa Cruz, Calif.) diluted at (1:5000) and detected with goat anti-rabbit antibody conjugated with horseradish peroxidase (Pierce (Rockford, Ill.)). Immunoreactive bands were visualized by using SuperSignal® West Dura extended-duration substrate (Pierce, (Rockford, Ill.)) according to the manufacturer's instructions.

[³⁵S]-GTPγS Binding Assay

Membranes from Sf9 cells expressing the DRD1 alone or DRD1 with Golf variants were resuspended in the reaction buffer (20 mM HEPES, pH 7.4, 100 mM NaCl, 10 mM MgCl₂, 1 mM EDTA, 1 mM DTT). Agonist-induced [³⁵S]-GTPγS binding assay was performed for 90 min at room temperature in 96 well-microplates with a volume of 200 μl per well, and containing 5 μg of membranes, agonist at a concentration range of 10⁻¹¹ to 10⁻³ M, 10 μM GDP, and 400 pM [³⁵S]-GTPγS. Non-specific binding was determined in the presence of 10 μM unlabeled GTPγS. Radioactivity was measured using a Packard Bioscience Top Count NXT Microplate Scintillation microplate reader.

Data Analysis

Data from [³H]—SCH 23390 saturation binding experiment were fitted to a one-site model to determine the density of dopamine D1 receptor (Bₘₐₓ) and the affinity (K_{d}) for [³H]—SCH 23390 using the GraphPad Prism program (GraphPad Software Inc. (San Diego, Calif.)). For agonist-induced [³⁵S]-GTPγS binding experiments, the EC₅₀ and relative maximum response (Eₘₐₓ) were derived from analysis of the concentration-response curve using non-linear least squares regression fit of the GraphPad Prism program. Statistical significance was assessed by analysis of variance (ANOVA), followed by Tukey post hoc test.

Results and Discussion

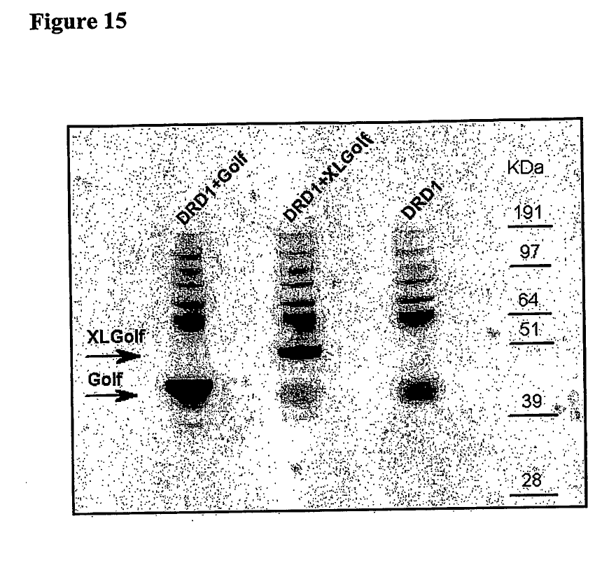

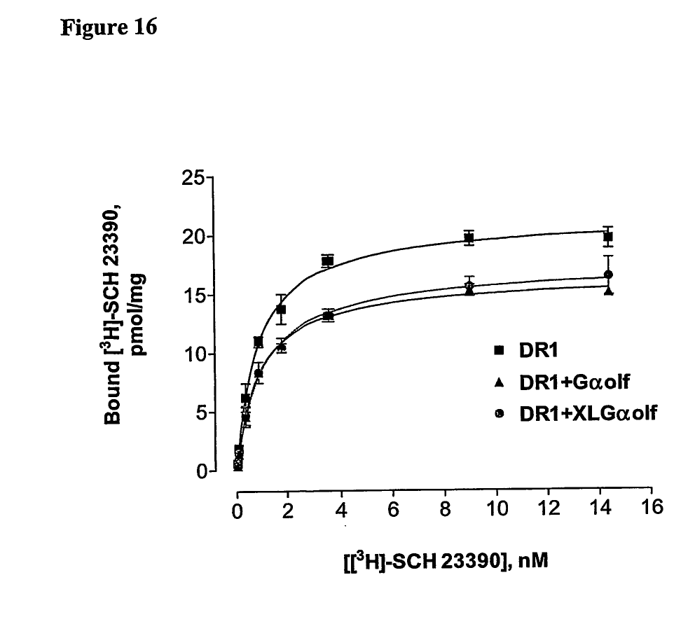

We introduced constructs of Golf variants and the dopamine D1 receptor into Sf9 cells and determined agonist-induced [35S]-GTPγs binding, a measure of G-protein activation. When expressed in Sf9 cells, the apparent molecular weights of Golf and XLGolf were ≈44 kDa and ≈55 kDa, consistent with molecular weights predicted from their amino acid sequences (FIG. 15). The molecular weight for XLGolf expressed in HEK 293E cells was comparable (data not shown). FIG. 16 shows the saturation binding of [3H]—SCH 23390 to DRD1 in Sf9 cells expressing DRD1 alone or DRD1 with Golf or XLGolf. The receptor density (Bₘₐₓ) of Sf9 cells expressing DRD1 alone (21.3±0.7 pmol/mg) was slightly higher than that of Sf9 cells infected with DRD1 plus Golf or DRD1 plus XLGolf (16.4±0.6, and 17.0±0.8 pmol/mg, respectively). However, the affinity (K_{d}) of [3H]—SCH 23390 to DRD1 in these three cell lines was not substantially different (0.89±0.07 for DRD1 alone, 0.91±0.13 for DRD1 plus Golf, and 1.01±0.18 nM for DRD1 plus XLGolf).

Dopamine-activated [³⁵S]-GTPγS binding in Sf9 cells expressing the DRD1 was concentration dependent (FIG. 5). The EC50 for dopamine stimulation of DRD1 in Sf9 cells expressing endogenous Gαs-like G protein, Golf, or XLGolf were 84 nM (95% confidence interval [CI], 36.6 to 192.8 nM), 214 nM (95% CI, 42.3 to 1083.9 nM), and 179 nM (95% CI, 120.8 to 266.7 nM), respectively, and did not differ significantly (P>0.05). The efficacy of dopamine for DRD1 in these co-infection experiments was, however, significantly different (P<0.0001). The efficacies of dopamine for DRD1 in Sf9 cells expressing endogenous Gas-like G proteins, Golf, and XLGolf were 14±2%, 231±4%, and 404±13%, respectively. These results of dopamine-induced [³⁵S]-GTPγS binding demonstrate that XLGolf functionally coupled to the dopamine D1 receptor.

Although the potency of dopamine is the same in all three cell types, its relative efficacy (Eₘₐₓ) differs. Cells expressing XLGolf exhibited greater Eₘₐₓ than cells expressing Golf, which in turn showed greater Eₘₐₓ than cells expressing endogenous Gαs-like protein. Although total receptor number was equivalent in cells expressing either Golf or XLGolf, as reflected by Bₘₐₓ of SCH23390 binding, an increased Eₘₐₓ may reflect a higher ratio of G-protein to DRD1. However, Western (immuno) blot analysis showed that Golf expression was greater than XLGolf expression in the cells used for this study. Nevertheless, increases in agonist efficacy may be due to more efficient coupling of G-protein isoforms to receptor. Alternatively these Gαolf variants may exhibit differences in GDP-GTP exchange rates.

### Example 9

### [³⁵S]GTPγS Assay on A2a Receptor

Generation of Recombinant Baculoviruses

The human adenosine A2a receptor (ADORA2A) (GenBank accession number AY136747) or XLGolf cDNA was introduced into the cloning vector pENTR/D-TOPO between Not I to Asc I sites. Recombinant baculovirus encoding human ADORA2A or XLGolf was generated with the BaculoDirected™ expression kit (Invitrogen, Calif.) according to the manufacturer's protocol. The titer of the third-passage viral stock was determined by plaque assay and used as the working stock.

Cell Culture and Membrane Preparation

Sf-9 cells were suspended in SF 900 II medium containing penicillin (50 unit/mL) and streptomycin (50 μg/mL) and cultured at 28° C. with rotation (125 rpm). Cells were maintained at a density of 2×10⁶ to 4×10⁶ cells/mL. For infection, Sf-9 cells at the density of 2×10⁶ cells/mL were infected with baculovirus (≈108 pfu/mL) encoding human dopamine D1A receptor (PerkinElmer Biosignal, Montreal, Canada), human Gαolf (PerkinElmer), or human XLGαolf at the appropriate multiplicity of infection (MOI). For infection, Sf9 cells at the density of 2×10⁶ cells/mL were infected with baculovirus (≈10⁸ pfu/mL) encoding human adenosine A2a receptor, human XLGolf, human β1 (PerkinElmer Biosignal) and bovine γ2 (PerkinElmer Biosignal) at the multiplicity of infection (MOI) of 1.75:2:2:2. After infection for 48 hours, cells were harvested for membrane preparation. Cells were harvested by centrifugation at 500×g at 4° C. The cell pellets were washed twice with Dulbecco's phosphate-buffered saline (DPBS) at pH 7.4 and suspended in ice-cold 10 mM Tris-HCl with 5 mM EDTA (TE)(pH 7.4) containing a protease inhibitor cocktail (Roche Applied Science, Indianapolis, Ind.) and sonicated. Following centrifugation at 1000×g, membranes were collected from the supernatant by centrifugation at 20,000×g for 30 min at 4° C. The membrane fraction was stored at −80° C. in TE containing 5% glycerol.

[³⁵S]-GTPγS Binding bssay

Membranes from Sf9 cells expressing the ADORA2A, XLGolf, β1, and γ2 subunits were resuspended in the reaction buffer (20 mM HEPES, pH 7.4, 100 mM NaCl, 10 mM MgCl2, 1 mM EDTA, 1 mM DTT) including protease inhibitor. Agonist-induced [³⁵S]-GTPγS binding assay was performed for 90 min at room temperature in 96 well-microplates with a volume of 200 μl per well, and containing 5 μg of membranes, agonist at a concentration range of 10⁻¹² to 10⁻⁴ M, 10 μM GDP, and 400 pM [³⁵S]-GTPγS. Non-specific binding was determined in the presence of 10 μM unlabeled GTPαS. Incubations were terminated by rapid filtration of the samples throught glass fiber filters (Whatman GF/C). For SPA format, 1 mg of WGA-coated SPA bead was included in each well of the reaction. Radioactivity was measured using a Packard Bioscience Top Count NXT Microplate Scintillation microplate reader.

Sf9 cells were co-infected for 48 hours with baculovirus encoding the human A2a receptor, and baculovirus encoding XLGαolf or Gαolf, without or with β1, and γ2 subunits. Two A2a agonists, NECA and CGS21680 (both purchased from Tocris Cookson Inc. (Ellisville, Mo.), were evaluated. Results with NECA are presented in FIG. 4.

### Example 10

### [³⁵S]GTPγS Assay on Adrenergic β2 Receptor

Sf9-Based Platform

The Sf9-based platform, incorporating XLGαolf was used to examine the agonist-induced [³⁵S]-GTPγS binding for the β2 receptor. The pharmacological profile of known β2 receptor ligands (determined using the Sf9 system with β2 receptor and XLGαolf) were compared with their published pharmacologic profiles.

Recombinant baculoviruses for the human β2 adrenergic receptor (GenBank accession number M15169; RefSeqP NP_{—}000015) (PerkinElmer Biosignal, (Montreal, Calif.)), for human Gαs short (PerkinElmer Biosignal, (Montreal, Calif.)), human β1 subunit (PerkinElmer Biosignal), bovine γ2 subunit (PerkinElmer Biosignal), and for XLGolf were produced, and Sf9 cells were infected. Membranes from Sf9 cells expressing β2 and XLGolf were assayed for agonist-induced [³⁵S]-GTPγS binding.

We found that coupling of XLGolf to the β2 receptor improved the signal-to-noise ratio of agonist-induced [³⁵S]-GTPγS binding. We found that the magnitude of (−)-isoproterenol-induced GTPγS binding in membrane from Sf9 cells expressing XLGolf is much higher than that of Sf9 cells expressing Gαs short (≈700% vs ≈350% above the baselline, respectively). This finding shows that the Sf9-cell-based systems using XLGolf with the [³⁵S]-GTPγS binding assay can be used to assay other Gαs-coupled receptors.

Chinese Hamster Ovary (CHO) Cell Assay

The pharmacological profile of known β2 receptor ligands (determined using the CHO cells expressing the β2 receptor and XLGαolf) are compared with their published pharmacologic profiles.

The human β2 receptor and XLGolf are expressed in CHO cells, and membranes from these cells are assayed for agonist-induced [³⁵S]-GTPγS binding.

The foregoing examples are meant to illustrate the invention and are not to be construed to limit the invention in any way. Those skilled in the art will recognize modifications that are within the spirit and scope of the invention.

We claim:

1. An isolated nucleic acid comprising a nucleotide sequence having at least 85% sequence identity to SEQ ID NO:1.

2. An isolated nucleic acid comprising the nucleotide sequence of SEQ ID NO:1.

3. An isolated nucleic acid encoding a polypeptide comprising the amino acid sequence of SEQ ID NO:2, or a polypeptide having at least 80% sequence identity to SEQ ID NO:2.

4. A vector comprising the nucleic acid molecule of any one of claims 1-3.

5. A cell comprising the vector of claim 4.

6. The cell of claim 5, wherein the cell is selected from mammalian, prokaryotic and insect cells.

7. A purified polypeptide comprising an amino acid sequence having at least 85% sequence identity to SEQ ID NO:2.

8. The purified polypeptide of claim 12, wherein the amino acid sequence comprises SEQ ID NO:2.

9. A method for producing a polypeptide comprising:
a) culturing a cell expressing a nucleic acid comprising a nucleotide sequence having at least 85% sequence identity to SEQ ID NO:1; and
b) isolating the polypeptide.

10. The method of claim 9, wherein the nucleic acid comprises SEQ ID NO:1.

11. A method for producing a polypeptide comprising:
a) culturing a cell expressing a nucleic acid comprising a nucleotide sequence encoding a polypeptide comprising the amino acid sequence of SEQ ID NO:2, or a polypeptide having at least 85% sequence identity to SEQ ID NO:2; and
b) isolating the polypeptide.

12. A method for identifying compounds that modulate G protein coupled receptor (GPCR) activity comprising:
a) providing a GPCR and a polypeptide comprising the amino acid sequence of SEQ ID NO:2, or a polypeptide having at least 80% sequence identity to SEQ ID NO:2;
b) contacting the GPCR with a test compound; and
c) determining GPCR activity, wherein a change in GPCR activity in the presence of said compound as compared with GPCR activity in the absence of said compound indicates that said compound modulates GPCR activity.

13. The method of claim 12, wherein the GPCR is a Gs coupled GPCR.

14. The method of claim 12, wherein the GPCR is selected from dopamine receptor D1, adenosine A2a receptor, and adrenergic β2 receptor.

15. The method of claim 12, wherein the GPCR and the polypeptide are provided as cells expressing the GPCR and the polypeptide, or are provided as membranes prepared from said cells.

16. The method of claim 15, wherein the cells are selected from mammalian, prokaryotic and insect cells.

17. The method of claim 16, wherein GPCR activity is determined by detecting intracellular phospholipase C (PLC) activity, phospholipase A (PLA) activity, adenylyl cyclase activity, cAMP levels, MAP kinase activity, GDP-GTP exchange, intracellular concentration of calcium in the cell, or opening and closing of ion channels.

18. The method of claim 16, wherein GPCR activity is determined by detecting GDP-GTP exchange.

19. The method of claim 18, wherein GDP-GTP exchange is determined by GTPγS binding or Eu-GTP binding.

20. The method of claim 16, wherein the GPCR is contacted with a ligand.

21. A method for identifying compounds that inhibit G protein coupled receptor (GPCR) activity comprising:
a) providing a GPCR, a GPCR ligand, and a polypeptide comprising the amino acid sequence of SEQ ID NO:2, or a polypeptide having at least 80% sequence identity to SEQ ID NO:2;
b) contacting the GPCR with a test compound; and
c) determining GPCR activity, wherein a decrease in GPCR activity in the presence of said compound as compared with GPCR activity in the absence of said compound indicates that said compound inhibits GPCR activity.

22. A method for identifying G protein coupled receptor (GPCR) positive modulators comprising:
a) providing a GPCR, a GPCR ligand, and a polypeptide comprising the amino acid sequence of SEQ ID NO:2, or a polypeptide having at least 80% sequence identity to SEQ ID NO:2;
b) contacting the GPCR with a test compound; and
c) determining GPCR activity, wherein an increase in GPCR activity in the presence of said compound as compared with GPCR activity in the absence of said compound indicates that said compound is a positive modulator of the GPCR.

23. A method for identifying compounds that activate a G protein coupled receptor (GPCR) comprising:
a) providing a GPCR and a polypeptide comprising the amino acid sequence of SEQ ID NO:2, or a polypeptide having at least 80% sequence identity to SEQ ID NO:2;
b) contacting the GPCR with a test compound; and
c) determining GPCR activity, wherein an increase in GPCR activity in the presence of said compound as compared with GPCR activity in the absence of said compound indicates that said compound activates the GPCR.

24. A method for identifying compounds that inhibit baseline G protein coupled receptor (GPCR) activity comprising:
a) providing a GPCR and a polypeptide comprising the amino acid sequence of SEQ ID NO:2, or a polypeptide having at least 80% sequence identity to SEQ ID NO:2;
b) contacting the GPCR with a test compound; and
c) determining GPCR activity, wherein a decrease in GPCR activity in the presence of said compound as compared with GPCR activity in the absence of said compound indicates that said compound inhibits GPCR activity.

25. A method for identifying compounds that modulate G protein coupled receptor (GPCR) activity comprising:
a) providing a GPCR and a polypeptide comprising the amino acid sequence of SEQ ID NO:6, or a polypeptide having at least 80% sequence identity to SEQ ID NO:6;
b) contacting the GPCR with a test compound; and
c) determining GPCR activity, wherein a change in GPCR activity in the presence of said compound as compared with GPCR activity in the absence of said compound indicates that said compound modulates GPCR activity.


SEQUENCE LISTING (45 sequences)

SEQ ID NO: 1 - DNA, 1377 bp, Homo sapiens
atgggtctgtgctacagtctgcggccgctgcttttcgggggcccaggggacgacccctgcgcggcctcggagccgccggtggaggacgcgcagcccgccccggccccggccctggccccagtccgggcggccgcaagggacacggcccggaccctgctccctcggggcggcgaagggagcccggcatgcgctcggcccaaagcagacaagccgaaggagaagcggcagcgcaccgagcagctgagtgccgaggagcgcgaggcggccaaggagcgcgaggcggtcaaggaggcgaggaaagtgagccggggcatcgaccgcatgctgcgcgaccagaagcgcgacctgcagcagacgcaccggctcctgctgctcggggctggtgagtctgggaaaagcactatcgtcaaacagatgaggatcctgcacgtcaatgggtttaatcccgaggaaaagaaacagaaaattctggacatccggaaaaatgttaaagatgctatcgtgacaattgtttcagcaatgagtactataatacctccagttccgctggccaaccctgaaaaccaatttcgatcagactacatcaagagcatagcccctatcactgactttgaatattcccaggaattctttgaccatgtgaaaaaactttgggacgatgaaggcgtgaaggcatgctttgagagatccaacgaataccagctgattgactgtgcacaatacttcctggaaagaatcgacagcgtcagcttggttgactacacacccacagaccaggacctcctcagatgcagagttctgacatctgggatttttgagacacgattccaagtggacaaagtaaacttccacatgtttgatgttggtggccagagggatgagaggagaaaatggatccagtgctttaacgatgtcacagctatcatttacgtcgcagcctgcagtagctacaacatggtgattcgagaagataacaacaccaacaggctgagagagtccctggatctttttgaaagcatctggaacaacaggtggttacggaccatttctatcatcttgttcttgaacaaacaagatatgctggcagaaaaagtcttggcagggaaatcaaaaattgaagactatttcccagaatatgcaaattatactgttcctgaagacgcaacaccagatgcaggagaagaccccaaagttacaagagccaagttctttatccgggacctgtttttgaggatcagcacggccaccggtgacggcaaacattactgctacccgcacttcacctgcgccgtggacacagagaacatccgcagggtgttcaacgactgccgcgacatcatccagcggatgcacctcaagcagtatgagctcttgtga

SEQ ID NO: 2 - protein, 458 aa, Homo sapiens
MGLCYSLRPLLFGGPGDDPCAASEPPVEDAQPAPAPALAPVRAAARDTARTLLPRGGEGSPACARPKADKPKEKRQRTEQLSAEEREAAKEREAVKEARKVSRGIDRMLRDQKRDLQQTHRLLLLGAGESGKSTIVKQMRILHVNGFNPEEKKQKILDIRKNVKDAIVTIVSAMSTIIPPVPLANPENQFRSDYIKSIAPITDFEYSQEFFDHVKKLWDDEGVKACFERSNEYQLIDCAQYFLERIDSVSLVDYTPTDQDLLRCRVLTSGIFETRFQVDKVNFHMFDVGGQRDERRKWIQCFNDVTAIIYVAACSSYNMVIREDNNTNRLRESLDLFESIWNNRWLRTISIILFLNKQDMLAEKVLAGKSKIEDYFPEYANYTVPEDATPDAGEDPKVTRAKFFIRDLFLRISTATGDGKHYCYPHFTCAVDTENIRRVFNDCRDIIQRMHLKQYELL

SEQ ID NO: 3 - DNA, 1146 bp, Homo sapiens
atggggtgtttgggcggcaacagcaagacgacggaagaccagggcgtcgatgaaaaagaacgacgcgaggccaacaaaaagatcgagaagcagttgcagaaagagcgcctggcttacaaggctacccaccgcctactgctcctgggggctggtgagtctgggaaaagcactatcgtcaaacagatgaggatcctgcacgtcaatgggtttaatcccgaggaaaagaaacagaaaattctggacatccggaaaaatgttaaagatgctatcgtgacaattgtttcagcaatgagtactataatacctccagttccgctggccaaccctgaaaaccaatttcgatcagactacatcaagagcatagcccctatcactgactttgaatattcccaggaattctttgaccatgtgaaaaaactttgggacgatgaaggcgtgaaggcatgctttgagagatccaacgaataccagctgattgactgtgcacaatacttcctggaaagaatcgacagcgtcagcttggttgactacacacccacagaccaggacctcctcagatgcagagttctgacatctgggatttttgagacacgattccaagtggacaaagtaaacttccacatgtttgatgttggtggccagagggatgagaggagaaaatggatccagtgctttaacgatgtcacagctatcatttacgtcgcagcctgcagtagctacaacatggtgattcgagaagataacaacaccaacaggctgagagagtccctggatctttttgaaagcatctggaacaacaggtggttacggaccatttctatcatcttgttcttgaacaaacaagatatgctggcagaaaaagtcttggcagggaaatcaaaaattgaagactatttcccagaatatgcaaattatactgttcctgaagacgcaacaccagatgcaggagaagatcccaaagttacaagagccaagttctttatccgggacctgtttttgaggatcagcacggccaccggtgacggcaaacattactgctacccgcacttcacctgcgccgtggacacagagaacatccgcagggtgttcaacgactgccgcgacatcatccagcggatgcacctcaagcagtatgagctcttgtga

SEQ ID NO: 4 - protein, 381 aa, Homo sapiens
MGCLGGNSKTTEDQGVDEKERREANKKIEKQLQKERLAYKATHRLLLLGAGESGKSTIVKQMRILHVNGFNPEEKKQKILDIRKNVKDAIVTIVSAMSTIIPPVPLANPENQFRSDYIKSIAPITDFEYSQEFFDHVKKLWDDEGVKACFERSNEYQLIDCAQYFLERIDSVSLVDYTPTDQDLLRCRVLTSGIFETRFQVDKVNFHMFDVGGQRDERRKWIQCFNDVTAIIYVAACSSYNMVIREDNNTNRLRESLDLFESIWNNRWLRTISIILFLNKQDMLAEKVLAGKSKIEDYFPEYANYTVPEDATPDAGEDPKVTRAKFFIRDLFLRISTATGDGKHYCYPHFTCAVDTENIRRVFNDCRDIIQRMHLKQYELL

SEQ ID NO: 5 - DNA, 2267 bp, Mus musculus
ggcgaggctcgcgcctgggacgttcgaaggatgcacaatggcccttcggggcagtgaggggccgtggtcccccagccggacccaccgcagccacgccccgcagctggcgccccagggtccgagtgcagcgctgcctccggcgtcggcgggagccccggtctgacccgcggggacttgcccgcgccccgcgccccgatgggcctatgctacagcctgcggccgctgctcttcgggagcccagaggacaccccgtgtgcggcctcggaaccctgcgcagaggatgctcagcccagcgccgccccggcccctgcctcgatcccagccccggctcccgtagggaccctgctccggcgtggcggcggccggatcgtcgcgaacgcgcggccgccaggcgagctgcagagccgccggcgacaggagcagctacgagccgaggagcgcgaggcggctaaagaggcgaggaaagtcagccggggcatcgaccgcatgctgcgcgagcagaagcgggacctgcagcagacgcaccggctcctgctgctgggggctggtgagtccgggaaaagcactatcgtcaaacagatgaggatcctgcacgtcaatggcttcaaccccgaggaaaagaagcagaaaattctggacatcaggaaaaatgtcaaagatgcgatcgtgacaatcgtttcagcaatgagtactatcatacctccagttccactggccaaccctgagaaccagttccggtcagattatatcaagagcatagcccctatcactgactttgaatattcccaggagttctttgaccatgtgaagaagctgtgggacgatgaaggagtgaaggcctgctttgagagatccaacgagtaccagctgatcgactgtgcacaatagtaagttgcttcccaggccaggtctctctgaagcctgattgcattcttggctgtgcccaccctatagctcagaggtttcctaaaagcagaaatcaagagtaactttgacttcagaagttaactttctaaagaggaggttttggcaacattgtttccctgaaggtgtattcagtaatgttgtttcaccaaaggagctggttttggtaacattgtttcactgaaggagatgtgttcagtaacattgtttcacgagcgaaccagagtaagtttctctttgtcctcttaagttcttttcattttttttcctatctgcacagagttaaagacatatctactaattataatgtacccaaatattcactattatattttaatttttaagcaagcctctccaaattaagttttttaatatcaaaatgtgcaggactggggagatggcttgctatctataaaagcactggatgcttttccagaggacctgagttcagttcccagcactcagctcacaaccacctataactccagctccaggggatctggtgccctctgctgacctccttggcacacacaactaattaaaattttaaaaagtctgtttcctattgtttccctgctgcattttggagttcagcagaatgaagcttacattggtctcgggcaggttcttactccttcgctgctgttttggagtggttccagatttgaaccagcagtgacttcagtgtgttctgattggctgatagtgaaaaggaaaacagattgattataacccagaagtgagagttgtccacatactcctggtcatacaactgccctgtaaactagggtacaaagcttcaacagcaacttttgttatcttgctgtttcctgtgggatgaatttgtgttccaggagaatgatgctcttatattgtctgagatcaagtacaggccagtgttggatcccacccatagaatccaggtaatttgctttgtgaagaggtttaacctgtcttctctggagctttttgggataaatgagtgtggtgtgagttccttccgtgatggttctcaagtaataggacaacattggttgattcccttgcaaagtaaaaactgcaaattaacaatccctgtgttaagacccccttcatcccttgagatgcagaaacaagcgaaacttgctagcctggggcccagtttcaactatactgctattcataccacgaccaatgataacatcagttacctgtttaaatgccttctggggtttggtagaacataactctatagtgtcatcatttaatgagttaattctaagtgcactggaactttctctgtgaaggtgaaactcacaataaagctgttgttgtataagaagaataaaataattattttcagg

SEQ ID NO: 6 - protein, 230 aa, Mus musculus
MGLCYSLRPLLFGSPEDTPCAASEPCAEDAQPSAAPAPASIPAPAPVGTLLRRGGGRIVANARPPGELQSRRRQEQLRAEEREAAKEARKVSRGIDRMLREQKRDLQQTHRLLLLGAGESGKSTIVKQMRILHVNGFNPEEKKQKILDIRKNVKDAIVTIVSAMSTIIPPVPLANPENQFRSDYIKSIAPITDFEYSQEFFDHVKKLWDDEGVKACFERSNEYQLIDCAQ

SEQ ID NO: 7 - DNA, 1540 bp, Homo sapiens
gatgtatttaactatcagctataatattccatgtcattttccaaggaacacatcttacagcaggtttttcacaagctatattgaaatgttcacctgttgcagaagctctataagatgctatgcattcagcgggaaataaccgaagacatagcacctctggcaggcttgtttctcagcgtcatggagagagcgcacccagtctgaaggcgcatctgctcctctctgccctatattgtggattaagaaaatacagtgtataatctcatattctcatttcagcaaatataaatagtacatggcaattatatgtgctcggttttcatttttaagggtggagattgttgaaaatggtgtcgtggaccagactccagaattggagattttgtagagatcaaaggtatagtactattaaagtagggataaagagtgtgcagacgtgggtgtaagataatgaacagaaaagggagtggcaaggtgcagagatctctgcaagaaatggattggggaaattgaaggctttaaagccacggtctctattcctacacccagctttccgtcctcggttactatcgcccaagatcaaagccaccctggttttctgattgccgcaactgcggctccaggtgctgagtgcacagccactgcggcactgtccgcagctgcgcgccgggctcagacggcattatttacggtacagaatactcgcccgcgcgacggtatttacggtaacggggaccagcctgggcggcagtatttacggtaacgaaagccagctgtatttacggtagcgagggctggaccggcggcggcatttacggtaacgggggccgggctcgcggaggcccgtcggttcggtccgctctgggcgttagcaagtgatctccagccaaggcggccgccaccccttgcacacagcagaaaatgcaaaatgaccctctggggcagtgaggggctgtggccctcggccccggcctgccgcacccccttcccgcagctggcggccggcagcgccgaacagggtccgggtgcagccccctcccgcccctccgctgaggcgccggcctgaactgggcgcgggaaccaggccgccctcggcgcccagcctgccctagtcccgcgcgccgcccccgctgtgccgcgcccacatgggtctgtgctacagtctgcggccgctgcttttcgggggcccaggggacgacccctgcgcggcctcggagccgccggtggaggacgcgcagcccgccccggccccggccctggccccagtccgggcggccgcaagggacacggcccggaccctgctccctcggggcggcgaagggagcccggcatgcgctcggcccaaagcagacaagccgaaggagaagcggcagcgcaccgagcagctgagtgccgaggagcgcgaggcggccaaggagcgcgaggcggtcaaggaggcgaggaaagtgagccggggcatcgaccgcatgctgcgcgaccagaagcgcgacctgcagcagacgcaccggctcctgctgctcggtaggtcccggccgcgagg

SEQ ID NO: 8 - protein, 125 aa, Homo sapiens
MGCLGGNSKTTEDQGVDEKERREANKKIEKQLQKERLAYKATHRLLLLGAGESGKSTIVKQMRILHVNGFNPEEKKQKILDIRKNVKDAIVTIVSAMSTIIPPVPLANPENQFRSDYIKSIAPIT

SEQ ID NO: 9 - protein, 138 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVP

SEQ ID NO: 10 - protein, 240 aa, Homo sapiens
TPRPTRASAWRGKSESSRGRRVYYDEGVASSDDDSSGDESDDGTSGCLRWFQHRRNRRRRKPQRNLLRNFLVQAFGGCFGRSESPQPKASRSLKVKKVPLAEKRRQMRKEALEKRAQKRAEKKRSKLIDKQLQDEKMGYMCTHRLLLLGAGESGKSTIVKQMRILHVNGFNGEGGEEDPQAARSNSDGEKATKVQDIKNNLKEAIETIVAAMSNLVPPVELANPENQFRVDYILSVMNVP

SEQ ID NO: 11 - protein, 202 aa, Homo sapiens
MGLCYSLRPLLFGGPGDDPCAASEPPVEDAQPAPAPALAPVRAAARDTARTLLPRGGEGSPACARPKADKPKEKRQRTEQLSAEEREAAKEREAVKEARKVSRGIDRMLRDQKRDLQQTHRLLLLGAGESGKSTIVKQMRILHVNGFNPEEKKQKILDIRKNVKDAIVTIVSAMSTIIPPVPLANPENQFRSDYIKSIAPIT

SEQ ID NO: 12 - DNA, 197140 bp, Homo sapiens
gagcccaggagttctatattgcagtgagctatgatcatgccactgcattccagcctgggtgacagagagagatcctgtcttaaaaaaaaaaatccataaaatatttgttttcattttcagctgactttagtatacaaaatattatccttcttgtatgatattgataaagtttagctaataagttataatagaaaagcaattgctgtaagtctcttgggtctcagtttcttcaaaagcctttcctgcctcacacactactcttccccatctcgatttaaacaggaccttcctttatactctgagaatcctgttttttcccttcatggcattagcgtaatttacaattaatatatttacctgtgtttttatgattgagtatatttctacctcattagtctataaattccatgaggacccctgtctttgcctcacccagcaccaacaagaatgcctagggtactgtaggcacttaattaaatggatgaatggataaatggatagatggatgagtgaatgaatagcgaaaatgacagtgatatttagtaactttttctattttcccaagttagattttctatagtcctcctttcttttgctcaaatatctaaaagtatgccataattttagcaaaatttggggaacaatgttaggtcaaaagtagtacatgtatgcacatttgttatcagtaggacccccaaaagaaatgtgaatgccggatctccaacttcttgatttaaaaatgtaatccaggcccggcgcgctggctcacgcctgtaatcccagcactttgggaggcaaggtgggtggatccctcgaggtcaggagttcgagaccagcagcctggccaacatggtgaaaccgtctctactaaaaattcaaaaaaaaaaagaaaaaaagaaaaattagctgggagtggtggtgggtgcctgcagtcccagctactcgggaggctgaggcagtagaaccactggaaccggggaggcggaggttgcagtgagccgtgattgcaccactgcactctagcctgggcaacagagccagactgtttcaaaaagaaaaaatgtaatccaatgtagtatttacatctagtgccaacgggtacagtgcacactgtgctgcatgctgtgtttcattaagcagcctaatctggtgtgttaatgagagatgtatttaactatcagctataatattccatgtcattttccaaggaacacatcttacagcaggtttttcacaagctatattgaaatgttcacctgttgcagaagctctataagatgctatgcattcagcgggaaataaccgaagacatagcacctctggcaggcttgtttctcagcgtcatggagagagcgcacccagtctgaaggcgcatctgctcctctctgccctatattgtggattaagaaaatacagtgtataatctcatattctcatttcagcaaatataaatagtacatggcaattatatgtgctcggttttcatttttaagggtggagattgttgaaaatggtgtcgtggaccagactccagaattggagattttgtagagatcaaaggtatagtactattaaagtagggataaagagtgtgcagacgtgggtgtaagataatgaacagaaaagggagtggcaaggtgcagagatctctgcaagaaatggattggggaaattgaaggctttaaagccacggtctctattcctacacccagctttccgtcctcggttactatcgcccaagatcaaagccaccctggttttctgattgccgcaactgcggctccaggtgctgagtgcacagccactgcggcactgtccgcagctgcgcgccgggctcagacggcattatttacggtacagaatactcgcccgcgcgacggtatttacggtaacggggaccagcctgggcggcagtatttacggtaacgaaagccagctgtatttacggtagcgagggctggaccggcggcggcatttacggtaacgggggccgggctcgcggaggcccgtcggttcggtccgctctgggcgttagcaagtgatctccagccaaggcggccgccaccccttgcacacagcagaaaatgcaaaatgaccctctggggcagtgaggggctgtggccctcggccccggcctgccgcacccccttcccgcagctggcggccggcagcgccgaacagggtccgggtgcagccccctcccgcccctccgctgaggcgccggcctgaactgggcgcgggaaccaggccgccctcggcgcccagcctgccctagtcccgcgcgccgcccccgctgtgccgcgcccacatgggtctgtgctacagtctgcggccgctgcttttcgggggcccaggggacgacccctgcgcggcctcggagccgccggtggaggacgcgcagcccgccccggccccggccctggccccagtccgggcggccgcaagggacacggcccggaccctgctccctcggggcggcgaagggagcccggcatgcgctcggcccaaagcagacaagccgaaggagaagcggcagcgcaccgagcagctgagtgccgaggagcgcgaggcggccaaggagcgcgaggcggtcaaggaggcgaggaaagtgagccggggcatcgaccgcatgctgcgcgaccagaagcgcgacctgcagcagacgcaccggctcctgctgctcggtaggtcccggccgcgaggtcggctgacgccccggggacagcgcgccgggcccgcgggggcggcgggcaccggggagcggtggcgggcaccggggagcggcggcgggaggggctcctgaatcccgggactggacccggacgggcggcgggcgcggacgggctacagagcgtttaaactgtgggtggaatggttcccctgacctagccgggaggacactacgggctgattctgcccctcccagtgccttgagcttactgccgctcgctctctctaattaatgagcctctctgggaagtctccccctcgattgatcaccttccacagggtcgaatccccggggatgtctaccctagcctctcacttcctgctacacgaggaggctggcagtttgttttaagaacacagatgaaaggagatttataaggccttttagcttgaccgcaaagcttttattccaatcacagtgttgtgggtgggtgtggcctgctgccttttgagaaagaacttgagctgcacttgcattttttttttattatactttaagttttagggtacatgtgcacaatgtgcaggttagttacatatgtatacatgtgccatgctggtgtgctgcacccattaactcgtcatttagcattaggtatatctcctaaagctatccctcccccctccccccaccccacaacagtccccagagtgtgatgttccccttcctgtgtccatgtgttctcattgttcaattcccacctatgagtgagaatatgcggtgtttggttttttgttcttccgatagtttactgagaatgatgatttccaatttcatccatgtccctacaaaggacatgaactcatgattttttatggctgcatagtattccatggtgtatatgtgccacattttcttaatccagtctatcattgttggacatttgggttggttccaagtctttgctaatgtgaatagtgctgcaataaacgtacgtgtgcatgtgtctttatagcagcatgatttatagtcctttgggtatatacccagtaatgggatggctgggtcaaatggtatttctagttctagatccctgaggaatcgccacactgacttccacaatgattgaactagtttacagtcccaccaacagtgtaaaagtgttcctatttctccacatcctctccagcacctgttgttttctgactttttaatgattgccattctaactggtgtgagatggtatctcattgtggttttgatttgcatttctctgatggccagtgatggtgagcattttttcatgtgtgttttggctgcataaatgtcttcttttgagaagtgtctgttcatgtcctttgcccactttttgatggggttgtttgtttttttcttgtaaatttgtttgagttcattgtacattctggatattagccctttgtcagatgagtaggttgcgaaaattttctcccattttgtaggttgcctgtttactctgatggtagtttcttttgctgtgcagaagctctttagtttaattagatcccatttgtcaattttggcttttgttgccattgcttttggtgttttggacatgaagtccttgcccatgcctatgtcctgaatggtaatgcctaggttttcttctagggtttttatggttttaggtctaacgtttaagtctttaatccatcttgaattaatttttgtataaggtgtaaggaagggatccagtttcagctttctacatatggctagccagttttcccagcatcatttattaaatagggaatcctttccccattgcttgtttttctcaggtttctcaaagcacttgcattttactttggaagctcaaccccagctctgggagatttgctccgcttgagaaccagctgcacgaaagggtggcgggccgcagtgtgctcgctgtgtaactgcgtgcaggagggtgggtgcagaatcccagcgttttcaaggaaccaaatgcttgctctcctagtagtatttttcagagcttagctatagcaaatacgtgtgctcctacaacaattagaaaacatggttctatgcactctaccgtagaatgaaaaaaaaatcacgatttcctgatactacttgttgccttttaaattttaagacaaaataataataattgggcttttgatcgcaattttgtgaggtgtaaggaagaatagttgttcaggtctgtgctctctgctatgcatgggagaaggagctattccatgactttagcaagatgcccgcttataaatgaatgcaactttcatttaagatctagaatacaataaatattgagtttgagaggacttgtccactgcaagaatgaataatttaaaacaacataaacctagctgttgtatcctgttgaaatatctaatttctaaagcaaagggaaatatttttgaagaaacaacaacaacaacaaaaaacagtaaacactcaggttatctcaacagggacgcgggctgagcggtagcagagctctcccctccagtgctcacctccagaatatcgcagagcacacgtttccagaggtctgaggatacagaagtgtaactttagaatttcatacctcgttaaaccattgttggtgtgtgagggtaaaataaatcttcggatatgcaagtactcaaagtataccctcatccacatatttttctggaaggaaacttaaaaaaaaaatagagacacagcggtggctcacgcctgtaatcccagcactttgagaagctggggctggtggatcacttgatctcaggagttcaagaccagcctgggtagcatagtaaaaccccatctctacaaaaaaatacgaaaattagctgggtggtgcgctggagaccctgtctcaaaaatgaataaataaacaaataaaagaatagagacaagtctcactacgctggccaggctgatctcaaactcctggcctcaactgacccacccacgtcagcatcctaaagtgctgggattacaggcatgagccaccatgctctacctggaaagacattaattggaaattacttcagccaaaatttttaaaatgcatcaaattaaaataattcaaaaaggggagaatttttttaaaggggggagagttgtcatctcaagaaactgacaaaaaaaaataagacaaataggatcaggagttgtgacaatatagtgttgttttgatgtgattatacagttgaacataattttaaaataaaggatatgagaagcagaaaatgctttatatataataaaagttattagactatgaatctgtgaaaaacaaatgtgatgagatatggtctgaagaatagaatggtgagctctaaagagtcagcttcttgtaacacatttgaaagtactctttgaagacatcatctcatagcctcatagagtttatatatcagcatcatatctaaaatgacgtaaaatcaaatgactcacttttcagggggagtgtattgtatgtatcctccacccagtgatatctacactaaaacgcatactgttacatgaggttgacattgtttatcaattttatatagatttgagttaaacacacaaaggttcttgattttattaggctttaaaatggatttagctggaaatttttgagtcagaaatatgacttattgaccattaatatcctcagataattacttatgctgccattgtgtttaacagccatttattcttgctcactgaccacattcatttgtttattctggaaatgtgcaccgaaagctgggcattgggttcagagacctcagacccagggaaggtgcacttgctccagcagtgtctttttttttttttttttttaatcccaacactttaggatgctgaggtgggcagatcacttgaggccagaagttgaagaccagcctgggcaacatagtaagactatgtcgctatttttttttttttttttttagacaggatctcactttgtcactcagtcaggagtgcagtggtgtgatcatggctcactgcagcctggacctccccagctcaagcaatcttcctgcctcagccccacaagtagctgggactacaggtacatgctaccatggctgactaacttttgtattttatatagagatggggtttcaccatgttaccccggctggtcttggacttctgagatcaagtgatccacctgtcttggcccacagtgctgggattacaggtgtgagccattgtgtccagtcaagtgccctttatcactccatgcctgctagtgtgaggttagtcccgtctcccatcatgatgagtcctcaagaagaccttcccatagtcctctagtggaaataaaggtttaactccctgtgtcctgtgaattctttgtgctcctctcatggagcactggggtacattgctccataggtgtcttggtgctctttctcccccatggggtcgatcctgatgactcagctttctttcctgcacaggacgtagcaccccacacaatcctctggcaacaacacaagaatctgggagccactgtgactatgaggagatatgctcccgttttaatttttagatggagcatggttgaagcatgccgtggctggattttaagcaggtgcagaggtgggatggaactttccagttagaaggcagtggtcaagacaaggaatcatggggtccaaactagggcagtgtggtatagtaggagtggggagctcacgggcttggggagcaggtcagatgactgggtgcggagggtaggtaatgagaggaattctcagataactcccatggtcttgagcatgaggtatgtgttagtcagcttgcttacctaccaaaacacaacaggctgaatgaccaaaaacagacgtttattttctcacaattctggaggttggaagtctaagatcaaggtgccagcagggttggtttctggtgagggctcccttcctggcttgcaaacggccacgttctcacagtatcctcacatggcctttcctctgtgtgcacacaaaaagagggagagatctccggtgtctcttcctcttcttacgaggacactgcgggattagggccccaccttacggcctcatcttaccttaattacctacttaagggccctatgtccaaatacagtcatgtggggggttagggcttcaacatatgaattcagggggatgggaagcacaattcagtccataacaagggctaaggcggggagtgcgtgccctgtctaaaagggacagctgcatcctagcttcatcccactgttgctttgtgagaacacaggcccaaagttgccaggtcttcggaatttctgaaacctacctatttttaagcatttgctcaaattaataaaatgtaatctacatggctaatacaatcacgggctgctggctcttaccgctgatctaacctgggtcacacagacttttctgcaggctgtgcactaggctttcggggctcacgcacatgagagaccattggcagaggccctgggtctacaccaattaaaagcatacttgatgtgaattggtgtgaatcaaaatatttaatctgccaaatgaacacatatagcaaacaaagctgtgttcaccattcttcaggtgaatttttctgctattgagtccatggagttgtaacagagtcaatgccattgatttttcttcatctttcattgactgtgttcactaccttcctgctggggaagggttatcatgttccactcaactactcttttaatatgattcttttcctggattgttattcgaattttaaatcctcagctgtagataggatttgtcttttttccttttttcaattatttcttgttatagtttatgtatttgctatttctctgtctggaatacatctttcaagaaggaagcaatctctttgggacatttgctggctttcttttgtatcaaaatgctggcatgattttaatctgtgtctttagttactgtcagaggaagtgcctggtgcacacatgctcagacatgcatgcacgcatgcacacacacacacacacacacacacacacaaaggaaaaaaaacagaaggaatgttctccactctgggattttgatgacttttttcccaagtaaatgtctttcttaatgaaggcagaagtgaccaaggaaagacaatagttttggagttgtgcagtggccttgggcatatccttattaacttgctgtcttcgttttttgccacagcctcctgtggcaccgtggctttcaaagtcagaagaaatcccagtactctggctattccttatgtttctgaactcacaaaagcataacacatcctggccgagcgcggtggctcacctctgtaatcccagcactttgggaggccgaggcgggtggatcatgaggtcaggagctcgagaccatcctggctaacatggtgaaaccccgtctctactaaaaatacaaaaaaaaattagccaggcatggtggcgggcacctgtagtcccagctactcgggaggctgaggcaggagaatggcgtgaacccggaaggcagagcttgcagtgagccgagatcatgccactgcacttaagcttgggcgactgagcaagactccgcctcaaaaaaaaaaaaaagcacgcataacacatccttacagccttaaagagaatgcagtgatgttgcatttaagtcaaaacttttatgtggtataacttgttatagaccgttacagcaatgatactaaaattttataggctggaaactgccgttgtttacatgaagcctaatttcatcccagggatttatcttgttctttcacttggcaaaagaccagaagttcagaacactctgtcaaaaatgctctcttggacttgtcaaccctctcgtgtttattcacatgattaatactggccctgaaaacaggtagttttgtcactggaatatatgaccattttatgaacacttccttcaaaatttgggggtatcctgtgacccactaaaagatgcagaggggcgtatgtgatggttagagcacagcactggagtcagaccttgtccgaggcccttagcaccgccatcctccagttgcacaaacttaagtacaccccccagtgcttcagtttccatctcaatgataatcctaatcccactggcttattgacaggagtaaatgaaattaattgaaataatgtatgtaaattcacataatgagatcgcacatcctagcaaagggcagccagccccaggtgaacacagccctctgtgactgagccaggatcaagcccaggcaggctggcttcgccaaacattagccagtgactgtgaggccagctggaggcagctgcaacagcccagtcaagagatgtgaggaggccgggcacggtggtgcacgcctgtaatcccagcgctttgggaggctgaggtgggcaggtcacctgaggtcaggagttcgagaccagcctggccaacatggtgaaaccccatctctactaaaaatacaaaaattagctggttaccagctaacttttaattagctggtggtgggtgcctgtaatcccagcttctcaggaggctaaggtgggagtatcacttgaacctgggaggcggagtttgtagtgggttgagatcgtgccattgcactccagcatgggggagagagcaagactcagtctcagaaaaaaaaaagagagagagagatgtgaggaggcgacctcattcagatgcggtgacaatgacaggaaggggcagacgtgtctgtgggtgtgggcaaaacggaatcccgggaacctggtgactgatgagatggagggaaagaagagcatctgggtgaagaaatgtcactgaacccttcagtaaaatggcaaggacattctccgagtaaggaatttaagaagaagaggcagttggagggagaagagaacagtgagctccggtctagatgtgttgagactgaagggccttggatgtccagcgggcagtggctggagagcagctgggcatttgccttgggtctcagagaggctgcagagccagagatggatgtgtcacccagagtcattagtgcactggccaggttggacatgccacctggggaccacatgccacccggagaggacatgccacctagggaggacgtgcagagtgagaaaggaagggaggagggcccaggataggaggccgtgggactccagcctttcaaaaagggctggaggaagaagagcgtgaagaatgacagagggctgggagcagccaagaaaggaagaggtttctggaggaaggcagtggccagcagtgtcacttgggagctcagggataaagagggctgaggctgggcgcggtggctcatgcctgtaattccagcactttgggaggccgaggcgggtggatcatgaggtcaagagatcaagatcatcctggccaacacggtaaaacctcgcctctactaaaaatgcaaaaattaactgggcgtggtggtgcacgcctgtgatcccagctacttgggaggctgaggcaggagaatcgcttgaacccaggaggcagaggttgcagtgagccaagattgcaccactacactccatcctgggagacagaacaagactctgtctcaaaaaaaaaaaaaaaaaatagagggctgagaaggaacccactgggtgtgcaggttcatggcctggtgggtgcaggaggcttcagattggaagggacaggaagacactgaagcagtgacggtggctgctcttgggaggactttgctgctggaggacagaacaggtgtgcccactcctgcaccttgtgccatccaggaaacagctggggtgatgacggatgagactggaatggtgggtgctcctggggatgaagagaagagggcaggagaggaattgtcaatctttgtttgacccttgctgtgcatccgctttctaaacattcaggtgcctggactcaacctcagctcctccagcacaagtgactttccctgttgacagtgtcagactaatgtgtcatttagagggaaattctttaatggcaagcaggtttttcagttgagttgagaccaagagacctagctaattttcgggtatctagctaatttaatttgtctatgaaaagtttccaaagattaacattttcaaaggtaagacctaactccaagggcccaaaatcacttcatgatattgaaagtgataatcagaagatctccaaaacctcactgcataaatccgaataaggagccctagatgattatttctcacctgacagcacccaggataatttactttgagtctacatttatctatttatttttaattttatttatttatttatttacttagagacagagtctctctctgttgcctaggctggagcagtagtaccatcttggctcactgcaacttccacctcctaggttcaagcgattctcttgcctcagcctcccaagtagctaggaccacaggcacccaccaccacacctggctaatttttggggttttttttttatttttgtatttttagtagagacagggttttaccatgttggacaggctggtctcaaagtcctgacctctggggatccacctgcctcagcctcctaaagttctgggattacaggcgtgagccaccgtgcccagctgggtctaaatttattaatacttagttgttgggttttttttttgagggggggggttggctttaagctgaaaccaacccttttactgaaaaacagaaaattaccttctatcagctaaagcagtgactcttaagtgtgtttcggggtcaagtgaaaactgttccctgttgtttcagaattccctcgctagggatggacagacagcgcacagaggacttttagggcagggaaaccgctccctatgataccatatgagtggacaatgtcaccacatacttgtccaacccacagaatatacaacgccaagagggaaccctagtgtagcccttccagcgctgtggactctgggtgacagtcatgcgtcagggtagcttcatggattgccgtactctatgagggatgttgctggtggggaggctgtgcctgtcggggcagggagtacatggcagctgtattctgtaccatcctctcaattttgctgtgaacctaaaactgctctaaataataggcttcttgttttagtcacttactaagaaaagaagtcagtgaaagattttgtccttgaattaggatcccaagaggaggagctggtggagaagaagaggacagacaccacagtggccatagcagaagggtcgagggttcttctaatgagcccgtttaggggccataagctgatggccctactcttaaaaagcccaggtcagttaaccgcagcaggaaagatagcggcccattattgaaagctgtggtttatgaataaaaaagcatcatttaaagacttttccaactgcagttctggggttaaattattctcagcaaataaagccctctagacatgcattgaagtgttagcacctgacgggaggggcacacacacactgctcctgcaggccgtttggcttggcttggccaggccatgtgtcccatcctgtaaggctgccctagctctgcctcatcaggaggaatcacaggattgttgcccagctgttattaggtgttacaccaaatgccactgaaggcacgagttagggaagccacccatcaaaatcttcaagggttagacagctctcttcaacccttcctgctgttatttcatttgccgatctgggaaaatgttatttagtgagaggcaggtcaacctgccgactgatggaggatcgaggaagacagcaaacacactgtggcatctgctttctctaatcctagcagaagagccttggagaagatgtaggttgcatcactgtgctttggattaatgctcacttggtgtgttcttctgtagttgaaaagtgaattttctttctgaataagtgaacttctcccagaaaaagcataagaagacaatattttgacataaacagaaaaataaggcgcaatgatacccattctttgagtcacaagggaaaaggccattttattcattgaatcccttatattactcaccaactgcaaacatttgcatctctcaggagcccgctctgtgccaggtgcttctctgggtgatgtggagattccacaaggcagagagcacagcctcagccctggaaaagttcactgtgttgttgaagagaaaggacaagaacctaagagaaaaataccagtccgagcctgcacgtgctcggagataaatggcaacagtgtgggtcaagaacatgattccagggattcacgtggaacctctcagtaacatgatgggcatcccccttccttaggatattttatgagtggggaggtacaggggaggtaggaacttgattgtttaatgtcaatgtttgtacacaaatgcataatgctggccaggcgcggtggctcacacctgtaatcctaacactttgagaggccgaggcgggtggattgcttgaggtcatgagttcgagaccagcctggccaacatggtgaaaccccatctctactaaaaatacaaaaattagccagacgttgtggcgggtacctataatcccagctactcgggaggctgaggcaggaagttgcaatgagccaagagtgcgtcgttccactccagcctgggtgacagagcaagaatctcaaaaaaaaaaaaaaaaaaaagcataatgctgttattgatcctgggtgcaaactatgagcagaagcagctgtgaaatctgctccaagtttgcacctggcagctggtttcctgttcatcaaggagctgctgaaagaacccagaagcacccacagctcccagaatgtagcagcaacctggagtgtttacctaatagaactgatgcttcaagaagatgaagaaagtttgagcaacagtgtgcagtgttatcaaacgggaaggagactgctccttttatctccagccatcaaagtgctccacaagtcaatattaggagctgcattttgtagattgcaccatgatgggaaggtggagattgtctgtaaatcggcataaagaaaaaaaaaatgaaacgtttatcctttttttacccaagaaatccactcattagagaatcactgaaaggaaggtggataaaaagccacaaaagggaacctttgacttggctgcagagcagcatttgtgttttacaggtacttcgggagcatctagagggcgtgatttggagtctgctagccaggaggccacaggagaaacacaggtcagccccagaatcatggagcacacatttgcctcgaccccagctgtattttcttttctgggaaaatatcagcccctggaattaggccagacacaggaatctgtagatacaaagctcgctagcagagcaagactcagaacgcaagagtggcaccagggaggaatcaggaccgagcaagaggcgagtgctgatcccagagccggcctccttcttccctgtcaccctcagagggtggtgtaaatgaaccacaagcccacagctgtcctggtgaggaagcgcagaacaaggcccaagccagcataccccagagagttagactgagccacagatcaatcagctgtttgatttgtagagcagcttgtctattgagaaggacgtcagtgctgtgttacaggctactatttgtttattcagtagcaattactgacattcagtagcacgtctaccacgtcccaggttctgtcaggcttcagtctacaaactggagctgtctcaggagctgttctgtgtgctcaggagcttatgaccctgtgcgggagacaaaggtatcaaccagccattgcagcacagcataaaatacatccatgcaggtacagaagacagtgggagcagctcttgccccagtgagaagggaggcaggactgaggaggagggcagcagcttccaagaaccttcccacttaaaaaaaaaaaaaaatagcccggtgcgatggctcactcctgcaatcccaacactttgggaggctgaggcgggtagatcacatgaggtcaggagttccagaccagcctggtcagcatggcaaaaccccatctctgctaaaattacaaaaattagatgggcatggtggcacgagcctgtaatcccagctagtcgggaagctgaggcaggagaatgacttgaacctgggagccagaggttgcagtgagcagagattgcgccactgcactccagcctgggcgacagagcaagactccgtctcaaaaacaaaacaaacaaacaaaaaaaccatatttgtagagtatatttctgttcttaaaatcgacaagtttgagaaacccctcgttttgagaggctcattggaaagcagcagatgccacgcagagcaaatgaaaaacagaacgctccgcacgttgtgcatagttttatcgaaactcctgcatctgctttaagtctgtttagttgaaaatccatttagaatatgctctaattgatatatgtgacctataaggaagtgtccctcttttttaagaaaatatagaaacgtgttatttgttataggttttcattgtacaaagaaattcaatgtgctgaaaagaaccaataaaatacggataaagtagtgccaattaagaaacagttatgtgactctttatctctgaaattgttttaactggtactttgttttagaattgctttttaatgtaacagccttctgaacatcagctatttgataagcgctgcgtggaactaaaaagcatgccgagctgtggttgatccagtggacttcgctgtgcctcatggaggacgatacttaactttcagatccatttccgttttttgctctaatgacattttctcagaagtaatgctctccacccttgggcttgggtacaataatgagcttggaacaggggataggaggcccagtgttgatgggcacacacacacacacacacacacacacacacacacacacagtgaagtgcaggaagctctgtggccatgtcaacccttgctgactggggacagatggaggcaggctgcaccctagtctgtggcctcagaagaaaagcatcttagttctgctggtggtgttcagcctcagcctttcccttcacctaaattcctacaggttatcctaacttcaaataggataaattaattttaattatgtatacagaatgttcgtttaggaacacagaggatggatttctagagtaatctcaaaatcctgttattcaacccacaatacaaaggaagcagcattggtgttcttccgtggagcctgaacatcatgggtcccacagtgagtcacagccttctgcactccctgatcacacagtgggtcacaccctagacccgtgggtcataccacggaacacatcgtggatcccacatatggattataccctaggacacagcctggatcacacagtgtatttggaattagagattggaatgccagaaacagccacacagtcaccctggcctccagagcaggccttccactgaaccctttgctgtggcttcactatgccctgagcagtccaaggcacaactaatggcccagggtcttccggctgaaagtgggaatagtgaggaatgggcctagggtgatcagtgagcagtaatgataagtacccccatctgtaatatgtgcctcaactcctgctaacccacaggtacagagggagaggagctcagctatctggcccacttctgggctccctccccagtttccagacccagccacgcagcctactctggggtctttggactccgtctcttacatttttgtctcctttcttccttaatatcagtattttcttttagtttctccttatcaaagcttgctcatacccagaacattttactcgttcctgtgaaattattaatggaggtgaaagttaagtccattatcaatttttttttaatttaacagctttttttgagatatagttgaaatgcatacagaaaacagcacatatttaaagtgtgcagtttggttagttttgacatgtgtatacacctgagagcatatccatcacctctgaaagtttcctcctgcatctttgagttgatttccttattttatcatggtaagaacacttcacacgagatctaccctattaaaaagtttttaagtgtacaataccgtactcttctatatggggacaacgttgtacagcagagctctacaacttaatcatcttgcatgtctgaaatgttttactccttgaatagcaactctccacttccctctcccccaaaccctagaaacaaccgtgctactctttgcttctgtgagtttgactattacaagtcctcatataaatggaatcatgcagtatttctccttctgtgactggcttaactcagcattccctccaggttcatccatgttgttgcatctggctgcattcccttcttttcttttttaaggttgaataatattccattgtatgtatgttccacattttctttcttcattcatcttccagtggacatttaggctgcttcagtatcttggctattgcaaaaattgctgcagcaaacgtaagagtgcagctatctcctggagattctgatttcagttcttttgaatatatacccagaagtgggatggctggattttatggtaattctatattttgattttttaaaggaatttcctactgttttccatagcagctgtaccattttaattcccaccagcagcgtacaagggtttcagtttctctgcatctttgccgacacttgctaacttttgtctttttgataatggccatcctaacaggtgtgaggtgatatgtcattatggttttgatttgccattttcctcatgattaataacattgagcttttcatatacctgctggacatttctatgtctttttttttttttttttttttctgagacagagcctcgctctgtcacccaggctggattgcagtggcacgatttcggctcactgcaaccttcgcctcccaggtccaggcgattcccctgccttagcctcctgagtggctgggattacaggctcctgccagcatgcctgggaaaattttgtattttagtagaagcagggtttcactatgttggccaggctggtcttgaactcctgacttcaagtgatctgcccacctcggcctttcaaagtgctgggattacagtgtatgtcttctttaagaagacatgtcattagcccattttttaatccggatatttgctattttttgttttgttttgtttttttgctgttgagttgtaggagctctttatatatttcagagattaaccccttatcagatataagcatatctcagagatattgcaggttcagttccaaaccaccccaataaagttaatatagaaagaaagtcacatgaatttttttctcggtgcatataaaagttgtgtttatactatactatatctattaaatgtgcaatcacatcatgtcttaaaaaaaagcacaccttaatttaaaaatattgataaaaagtgctaacgatcatctgagccttcagtgagtccggatctctttgctggtggaaggtcttgcctcgatatctgtggctgctgactgatcagggcggttgttgctgaagacagtttcttagagtaagacagcaatgaagttgatcacatcggttaactctttctttcatgaaagatttatctgcagcatgtgatgcttttgatagcattctactcatcgtagaactttcaaaattggggtcagtcctctcaaacatgctgctttatcagctacatttatggaatattctaaatcctttgtaatagtttcaacaatgttcacagcatcttcaccaggagaacattgcatctaaagaaacctctttctttgctcattcataagaagcaactcctcacccattaacattgtatcctgagattgcagccatttagtcacatcttcgggctccacttttaattctagttctcttgctatttctaccacatctccagttacttcctttactaaagtcttgaatccctcaaagcaatccatgagggttgcaatcaacttcttccaaattcctgttcatgttgctattttgacctcctccaatgaatcatgggtgctcataatggcatctagaattgtgaattgttgccaggtttgttgacattgttgttttgagacagggtctcactctgtcacccaggctggagtgtggtggcatgatcatgactcactgcagcctcaacctcccaggctcaagtgattatcctgcctcagcctcccaagtaactgagactacaggcatgtgccaccatgcttggctaatccagaaggcttctaatttactttgcttaggtccattagagcaatcgctgtctatggcacttatagcgctatgaaacatatttctgaaataataagactgcaaagtaaaaattactgcttgatccatgggctgaagaatggatgttatgttaacaggcatgaaaacaatattaatctccttgtacatctccatcagagctcttgggttaccgggtgcacagtaaatgagcagtaataatttgaaagcaatctttttttccgagcaatacgtcccaacagatggcttaaaatattcagtaaaccatgatgtaaacagatgtactgtcatccaggctctgtttttccactgatagagcacaagcaaagcaaatttagcctaattctggagggccctaggattttcagaatggtaaatgagcattgatttcaacttaaagttaccagttgcattagcacctaacaagaagtcagcctgtcctttgaagctgtggagccagggattgacttttcctctttacttatgaaagtcctaggtggcattttcttcccacagaaggctgttccacctccattgaaggctgttgtttagtgtagccacttctcatcagtgatcttagctagatcttctggttaactttctatattagcatttgctgccttaccttgtacttgtatgttatggagacaactgttttccttagacctcatgaatcaacctctgctagcttccagcttttcttctgcagcttcctcgcctctctcaaccttcctagaattgaaaagttagggccttgttccaacttaggctttggcttcggggaatgctgtggctgttttaatcttctatccagaccactcaaacttttttcatttcagcaatgtggctatttcactttctcgttagtgtgttcactggagtagcacttttaatttccttcaagaagtctctcttggcattcacaacttggctgtttggcacaagagacccaacttttggcctgtcttggcttttgacatgccttcctcagtaagcttaatcatttctagctttggatttaaagtgagacagtcttcatttgaacacttagaggctattgtaggattattaaatggccctaatttcaatactgttatgtctcagggcatagggagggcccaggaaagggagagagatgggggaatggctggtcagtacagcagtcagaacacacacaccatttatcgattgagtttgccatcttatatgggtgctatacgtgacacctcaaaacaactacaatagtaacatccaagatcccttatcacaggtcattttaacagatataataataatgcaaatatttggaatattgcaaagattaccaaaacgtaatacagagacacaaagtgagcacatgctgttggaaaaatggtgccaatagacttgctcaacacaaggttaccacagacctgaaatttattaaaaatttgtaaatttgtataaatgcaatatctgcaaaacacagtaaggtgaaacacaataaaacaaggtacacctttatgtggtttacacatattttctccctttcagtaggttgcctcttcattctgttgattgtttcctttgctgtgcagaagcccctcagtttgatgtcatctcacttgccaattttgtcattgttgcctgtgctgttggtgtcatatccaagaaatcattgccaagaccaattaatgtcaggaaacttttttcctatattttcgttcagtagttttacagtttcaggtcttacaattacgtctttaatccatttcgatttactttttgtgtgtagtgtaagataaagttccaatttcattcttttgcatctgcatatacaattttcccaacaccatttgttgcagaaactatcctttccccattttatattcttggcactcttgtcaaagatcaattgactatatttgtggatttatttctggactctctattctcttccattggtttatgtatctatatgccagtaccatactgttttaattactgtaactgtgtagtatgttttgaaattaggaaatgtgatgcttctagctttgttcttctttctcaagattactttgtctacttggggtcctttgtggttccttctgagttttaggactgttttctctatttttgtaaaaaaaaaaaaaaaaaagtcattaggatttccctggggattgcattgactcagtaggtagctttgggtagtacagacattttaacaatataaatcttccaatttgtgaacatgggatttttttccatttatttgtatcttctttcatttcttatatcactgttttgtagttttcagtataaaggtctttctcctccttggttaattcctaagtgttttgttatttttgatgttgttgtaaatgagattgttttttaatttcattttcagatagttcattgtttgtatgtaaaactgcaactgattttatacatcaattttgtatcctgcaactttgctgaattcatttattagttctaatagttttttggtgaaatttttatatttttgtatgtataagatcatgccatctgcaagcagataattttacttcttccttttgaattttgatgctttttatttctttttcttgcctatatgctttggctgggacttcctgtggtgtgctgaatagaagtgatgagagtgggcatctttgccttcttcctaatcctagaggaaaaagcttttgctttttcaccattaagtataatattagctgtgggcttttcacatacagctttaattatagtcagttaaatttcttctatacctagtttattgagagtgtttaatcatgaaatggtgttgaattttgttaaatgctttttttgtatctattgagatcactatgttatttttgtccttcacactgttaatgtggtatatcacattaattgatttacacgtgttgaatcatccttgcgtttcagggataaatcccacttggtcatagtgctgttgagttcagtttcctaatactttattaaggatttttgcttctgtggacctcaggaatattagcctatagtttcttttcttgtagtgccttcatctgactttggtatcggcatacactggcttcataaaatgagtttggaagtgttccctttatgttattgatgacgcagtttacatctttttatgttgtgtatccattaacacatttttatatagttagtgttaatactttgtcttttaacttttaagttagaattaaaattatttacatatcaacattacagtattctatgtttgtctacatatttacctttacgaatgaactttatactttcatatgctttcttgttgctgtttagtatgctttcatttcaacttgaaaaactctccttagcatttcttgtatgtcatatatggtggtgatgaataccctcaacttttgtttgggaaattttttctctctcctttttttttttggaggatagttttgccaggtatagtgttctttggttggcacttttttcttttagaacgttgaatatatcatcccactcctttctgacctgaaagatttttgctaagaaatctgccaatagtcttatgagggcttccttttatgtgacaagttccttttctcttgctgctcaaaaaattgtctttgacttttgacaatttgattacaatgtctcagtatgaactgttttgttttgttttgttttgtttgagatggagtctcgctctgttatccaggctggagtgcagtggtgtgatctcggctcactgcaagccccacctcccgggttcacaccattctcctgcctcagcctcccgagtagctgtgtctacaggcgcctgccaccgcgcccggctaattttttgtatttttagtagagacgaggtttcaccgtgttagccaggatggtctcgatctcctgacctcgtgatccacccacctcagcctcccaaagtgctgggattacaggcgtgagccaccatgcctggtcctcagtttgaactactttggctttatcctgttcgtgattcattgagcttcatgaatccggatatccatttccctctccagatttgggatgttttgaccattatttcttttaataagctttcttcctcagtttctttcttcaccttctaggattctcaaaatgtatacattgattagcttaatggtgtcctataacttccttaggctatcttcaatttttcattcttttttttttcctttttactcttctgactagataatttcaaatgacctgtctttgagtttgttgattctttcctctactaactcaggtctgctcttgagccctgtagtgattattttcagttcagttaatattcttcagctttacaatttgtgttttgttctctgttttatagtttctatttctttgttgatattctaattttatgcatacatagttttctgatttccctcagttgcctgtgttctctggtaactctctgagcttcataagatgattatttttaattctttgtctgatctctgtctatagatctccatttctttagggtaggttactggtgatttgttttgttcatttagttgtgtcccatttccctctttcttcatgttccttatagctttatgttggtatctgtccatttgaagaaacagccacctctcccagtccttatggctttagcagggaaagatcttcaccaatcagcctagctatagattctgggaagctctcaaactttttctgtggatgtgtcttccctggacttgtgcattttaattatagggatttactggtttcttttttcaggagcccataatctcttgctccttctggtgtctcactactataccataggccagggtgccccactcttctccctccctccgtggggagaagtcatgagttttgcaccttttcccaatcagccagagctgtgctggccacagcaaaccaactgccccttttctttgttcttagctttcctcaggcatttaaactattctttgtccctcagcacgctgggtgaagcaagatataaattggtcccttgggcaacacataaaaaattcagagttggatattaattccactctctcggggagagtgaggccagggtggtctttctgggcactgtgctgtgccagcttgggggcaggcccgacgtgcataaaattgaattgccctttttacctcttttaatgcaactgctttggctttttacttatctggagtactgtgacttcttaactggattctagagctctcataaaggtgttttggcccatatattttgttacatcaatttttcagcagggcaataaatttttcagcggggctgggactttctattttgccatcttgctgacatcactctcaaaaataaaattgtattattacataatttaaactcaaatagagaccaaaggatggtgtgtgaacatttataatttttttctatagagttaccagtccagttgaaaatctctgatttctctccctgtcccatttactccttaatcctccctagcaagcacggattcttagttattcagttctccctctacaggagcagaggacgtgattagacctggtcaggtctctcagaaaaacatatctacaaggtgaacagattttttccttgctcgtagtagctgcctttcacagactacattccctgtgtcattcactagcctatgtttccttcatcctacctgaaactccctcgtgggcaagggatgagagaggtgagaaaaagaaggagagagaaggaccccctcccaagtcaactgggaatgaccagtctctgatgtccccagcccatgaacaccatatggggcaactgcgctgtttgcagaacactgtgaaatgctgatatgtttaattccttcatgctaagaacaattttatcaatgaaaactgtgtccacaagacatgtgtgtacaacacgaagtgacttctgtgagcacagggagaaaacaaatgaaggccacccaaatgagaacaagcagaggctatttatttagaacttgctatagcaaaagagtcagtgaccatcacttgtgttcggcagactcaaagacaggcagatgagtgggaaagctttttagtggaaaaaggaaaggcttcagatattccctgattggaggctgttggcgtggggaagctgtaggctggctaagtgcaaaggggacatcccatgcaattggttagcagagcatctttgggtttctccagttggtcctaagtaggagcaaaaattaagcaagttgataattattagtcaagttgtggccatttggagccaactgttacaaaggttattgtttggcttcctggagtggttgctgttgtactggatactgtcaataataggttgccatcctaggccagttgctgcaggttgtgggtcagactatatatatttttatatatagcctggccattgcctgtttatatattcagtccctcaacacatatctagagcgaaaacaatcatttaaaaataagcagaaaacctctgctatacaaggaaagaacaggctgtagagccaggacctgacttgatttctggctctgctacctctggccggtcactgagcacctccgagctccctttcctggagcataaaatgaagatgggaatgtcgtccagaaaaattgttatgaaaatctgcaaggtgagacccatgcacaaagcatggtctggtgattggggtctcctgcccttttcctgtcactgccaagttcacactgaggtctaaagggcgaggcctaacaccttctctgtccttgacacatttgcattttatcagggataaggcccaggtttgaaaaaagggaacagcttcagagtcgtgggattctcaaggggcatctccgatgccagcaactaaactgcatcacaaaagtcagggaagcttggattgtagtctctcccacccttcctataaatacttctcttcctggggcctgcccactaggtacccctgccatattccatcagtgaaacgcatgaaccaaaaagtatttgagacagatctcagtcggtttagtttcattttgccaaggttgaggacagcccaaggaaaaaagacacaagtcacagtaggatttgtggactgtgctttttccaaaagaggattttaagaactttaaaaaggaaagagcagacaggaggggaaggagggaagaaaaaaagggacagtaggtgctgaggtgagtggtcacagtcttgtgaggctttgctgagcgctcgctgaatccacatgtgaaaggaggggtagaggtatgttaattatgcgtttgtctcacgctcagtaaatctgcattttatatgagataaagtaaatgtagagtagaggaagaggtcaaatacacatttctctcagagggatgatttctagtcttgtctttgtcctgtacctgtgaagatacactgttaatttatattgtcagggtgaaacaaaacaaaactccgttttagggctcacaaagaatttccttgtgagcagttagtgagggaggccacctggggagataggtgacctatcttctgtctttgtagccatctgtttaggaacaaaaggaaggcagtttttgtatgattcagttcccaagtgtaactttccctttggcatagtgagtttagggtcccgagattttattttcctttcacattaaggatagctaatgctgggtgtggcggttcacaccagcactttgggaggccaaagcaggtggatcacttgaactcagagttcaaggccagcctgggcaacatggtgaaaccctgtgtctacagaaaaataccaaaaaaaataaccaggcatggtggtgcacacctgtagtcccaactactcaggaggctgaggtgggaggattgcttgagcccagcaggtcaaggctgcagtgagccatgatggtgccactgcactctggcctgagcaacagagtgagatcctgtctcaaaaaaaaaaagaaagaaaagaaaagataacaaggccgggcgcggtggctcacgcctgtaatcccagcactttgggaggccgaggtgggcggatcacaaggtcaggagatcgagaccatcctggctaacacggtgaaaccctgtctctactaaaaaaatacaaaaaattagccaggcgtggtggtgggcacctgtagtcccagctactcgggaggctgaggcaggagaatggcatgaacccgggaggcagagcttgcagtgagccgagatcgcgccactgcactccagcctgggtgacagagcaagactccatctcaaaaaaaaaaaaaaaaaagaaaagaaaacaaatgagcacagggattgctaagcgggttgcaagtttaaataaagtggtcaggaaggctttgttgagaaagtggacattttagccagggtgtggaggaaacaaaagaggggaccaggtcattctctggagaagagcattgggatatggagagccaggtgcagatgccctgaggtttgcatgaactgatgtgttcaaggaataagccacaggcccatggtcaaccaagtaggaacatataaggcagtaaaaggaccttgggttttactttggctatttataataatccaagcagccagtagaagctgggagcagggtggtagtaatggactgatgagaagtggtcaagttctagatagattttgaaggcaaattaacaatttgctaatgaacatatgaaaaaaaaaaagctcaacatcactgatcattagagaagtgcaaatcaaaactaccatgagataccatctcacaccaatcagaatggctattattaaaaagactactattaaaaaacagatgctggtaaggttgtggagaaaaaggaacgcttatacactgttggtgggagtgtaaattagtttaaccattgtagaaaacagtgtggtgattcctcaaagacctaaaaacagaactaccattcgacgcagcaatctcattactgggtatatacccaaaagaatattgataatcctactgtgtccggaattggtgggttcttggtctcactgacttcaagaatgaagccgcggaccctcgcggtgagtgttacagttcttaaaggcggcatgtccagagtttgttccttctgacgttcggatgtgttcagagtttcttccttctggtgggttcgtggtctcactggcttcaggagtgaagctgcagaccttcgcggtgagtgttacagctaataaaggcagtgtggacccaaagagtgagcagcagcaagatttattgcaaagagtgaaggaacacagcttccatggtgtggaaggggacgggagcaggttgccacggctggctctggcagcctgcttttattctcttatctggccccacccacatcctgctgattggtagagctgagtggtctgttttgactgggtgctgattggtgcatttacaatccctgagctagacacaaaggttctccacctccccaccagattagctagatatagagtgtccacacgaaggttctccaagtacccaccagagtagctagatacagagtgtcgattggtgcattcacaaaccctgagctagacacaggatgctgattggtgtatttacaaactttgagctagatacagagtgccgattggtgtatttacaatccctgagctagacatgaaagttctccacgtccccaccagactcaggagcccagctggcttcacccagtggatcccgcacgggggctgcaggtggagctgcctgccagtcccgcacggttgtgcccacactcctcagcccttaggtggtcgatgggactgggtgcctggagcagggggcggcgctggtcagggaggctcagctttgcaggagcccatggagggtgggggaggctcacgcatggcgggctgcaggtccccagccctgccacgcgggaaggcagctaaggcccagggagaaattgagcacagcagctgctggcccaggtgctaagcccctcactgtctggggccggtggggccagtggagccagccggccgctcccagtgcggggtccgcggagcccacgcccacccggaagtcacactggccctgttcccccggcacctctccctccccgcctctccctccacacctccccgcaagctgagggagccggctctggccttggccagcccagaagggggctcccacagtgcagtggcgggttgaagggctcctcaagtgccgccaaagtgggagcccaggcagaggaggccccgagagcgagtgagggctgtgagggctgccagcacgctgtcacctctcactaccataaagacatatgcacacgaatgttcattgcagcactgttcaaaatagcaaagacatggaatcaaactaaatgtctgttgtggtagactggataagaaaaatatggtacatatacaccatggaatattatgcagccataaaaaaaagaacgaggtcatgtcctttgcaggaacatgggtggagctggaatccattatccttagcaaactaacgtaggaacagaaaaccaaatactgcatgttctcacttataagtgggagataaatgatgagaacacatggacccatagaggggaacaatacacactggaacctttcagagggtggaggttgggaggaggatcaggaaaaacaactaatgggtactaggcttaatacctgggtaatgaaataatctgtacaataaaacctcaagacacaagtttgcctatataataaacctgcacatgtacctctgaacctaaaatgttaagaaaaaaaagaaatcagtcttaagcaaacatgctaaacaagtctactagaaataggcatatggattgcttgtatcttcatattacatgacagtgttagaagataatctaatttactactaagaaatacaatcaattattaaataaataatgttccagacaaaatacctagcagtttaaaaaaaaagttgcattctttatcagattaccaaggtaatacatgttcacaggggaaaatttgaaaagtaaggataaagaagaaaaatgaaaagaatcttatgtgacataatcatttattcttacattttcttaaatttttagtgcaaattgtcattcatttttcaagaatctgaaaaatgaccggcatttatcgttatctttaaggttagatattgacatttgatttaagtcattcagagacaatcagccataatgcaagtgtgctatataatcaatacggcagagtcaatagggtcagggattttacattctgtctttgtttcaaagttttggtaatataatgcaccgtttatttttctaaatatgactataagttatttaaatggaataatgtttatgaaagacctctcaacatcctttataaaatcattcacagattgatctcttatagccaaaaaatcaaagctgtacatgaaaatggattttcccctgctctatctactttgaaataatgattagatcacaagcatcttctagctaaggtttcaatcactttacctccagtggatatttgttctcagtgtttctctgagcagcattaacaattgccctaataaaccagttctaaatgagagaactgcactgtactaagatgatgatctgtgacttgttttattcctcctagtaactatatgtagtgaagaatggttaatgtgttcttttctggctttttttcattatagtttgtcttaatggtctcaaaatgtcatgatgaatattttgtcaagttgtttccatttttaaagccctgactttataaagcaaatttaatatgaattttgtgtaccatgaattaaatggtacacaaactaacaaaaagaaaataaaaaatttgctgatgaattggatgtaaataggagagaaggtgaggagtcaaggctgattgtcatttggtggggacggatagagctaaaatgtattgaggacctcatatgtgtactatatattctccaaacatcacaactatttaaaaacattataactacataaatagttgaaactgttgtgtctgaattctatcattaaaaaaaaatgtatcctcacaatttaaatgaagacaccagtcacgtgatggttgaggattgaaggagtggaggtgtttgatgtggattttttttttaatttatcttctttacaaatgacaggaagactgacagcagcaaagatgtattgcaaaacagtcagggttaccagagagtcatgttattataaaaggtacctagtattgctgtatttcagcctgagtttaggtggataaaatccaaggcctcggtttcttcctttgatgacactggcattttattcaaacagagggaaactgagaccatcaggcagctgacagctgagcctgccttaaatacaagtggaagaagaaatccacgaaaatctgggttcctcttatctaagtcatgcaatctgttgatactaaattgcttgcgccctggatgtgtagttgggttgtagcttttggagacaaatccagcacacctactattccttgaagcatttgagtggctgatgcctcaccctggaagaacggaagcaggctgtcatggacagtgcagagctgcactcacaccatgctgcagcagccatggacagtgcagagctgcgctcacaccatgctgcagcggccgtggacggtgcagagctgcgctcgcaccatgctgcagtggcaacatcaagacatttatagcagcatgtgtgtgtgccagcaatagagtgcaagagacagatgtccctgggaggttggtggaccacacagcatacctacttgctgttttggcttatgttccacagcagggctcctcaacctttaatgaacatttgaatccccagggattttgataaaatgcagattctgatttagtaggtctcaggtggggcccacaattctgttttcctagcaagcaccacatgatgcttatcaggctagactgtgaactgtattttgaatagcaaggtgagtctagaccaagagttagcagactgtttctgtaggatgaatagtttaggttttatagactaaatggtaaaatcaaggattattatgtagatacttacataagaggagagaaaacaaatttccaccaaatttttattaatgaaatctaaaacataaaacaaaaataattgtgtacaattttttgtaagataggtctactaataaaaagaatgcaactgttttgggggagggataacatttcactaaattaatgttcaaagtaagtgctcccattatcaaaatcaatagcagatgtcatctgtcaatgctgacttgtagtgcgacgttatgtgtttcatctttgcaaatgtcttttcactcagacagatactgccaaatatggatgtcagtccacaagcttgtgatttttaattgagcatattcatcatttggaagacatctataaattgtgtttgattcttctcttgatatctgccttctggcattgcataacattgcaaactaaccacttccaattgaagttgaagtggaagcttctcaattgcacagttaaatggatgtcgaaatttggaaatttccttcgtacttttgtcaagttgtgaaaaggctcctggaattgtcatttgagcttggaaaatatttctgctgcaaatttgtgtggaaatggagagctcacttcctgttttaccttttgacagcacaggaagtatgcaaagcaccttgacattgtttatgattcaagcaacattaatgtccctaaataactttattgcaatataagatttgcatacagatgcaattttaccttataattttaggttgaattacttaagaaacaggtcttcagcaaaagccaattttcaaagcaattcagtgttcaataatggagattgtcaaaggttcttctcttccagaaaagtgttcatctcagctctaagttcaaaaattcacaataaaactttaccactgctaagccaccgtactgctgtaagctaagtcaggatactcagcttctgtttttaacaaacattaatgaaactgacgatgttcacagaagtaaatttcactcctgatgctaagggttcaaaaactcatgacagattcaaacattgtccgcagagcacttgctagcgattagtaagataaataacaatgggattttaaaaccttacatttaacaagctttgtaaatttgtccaactaaacctttttctgctccacagatatttttaccaccatcagtggtcactcatcttagtggattccacttattcaagttcactgaattaatgtttccttaatgcctttgaaaatagtctcaccggctggacacggtgactcaaacgcctgtaatcctagcactttgggaggtcgaggtgggcagatcacctgaggtcagcagttccagaccagactcgccaacatggtgaaaccccgtctctattaaaaatacaaaagttagccaggcatggtggcaggtacttgtaatcccagctactctggaggctgagacaggagaatctgtctctggtagccagccatcatgcctggctaattagagtctaacatgcctggctagttagagtcaggttttcaccatgttggctaggctggtctcaaactcccgacatcaggtgatctgcccaccttggccttgaaccctggaggtgcaggttgtggcgagccaagatcatgccattgcactccagcctgggagacaagagtgaaactctgtctcaaaaaataaataaataaataaataaataaataaataaataaataaataataaaatagtctcaccatagctgattcactaagacagttcacagaggctaaattttcagttcctttgaactcagcattgactccttgaataaacagtaacaactgagcatcatcggtgttaacatcactagccaaggaaagccactcagaatcattcaccttggttttgaattgacaaataatgttgttcccaacattctcaactctccaaacaactgttcttaccaaaagactaacagtttattttctctggacacatttcttcagcagttgcaatcaaacatgatttaattaacttaccaccagcaaatgatttttcctgcttggctaacaaatgagccactcagaaacttaactggggtaaagaaaattctgctgtgatgaggcattccattttaagttttctgatttttctgaccattgcttttctgtgagttggggatattgtgagtggtaggtctgctaacagtgatatgtattatatattcttctagcatagatatagcatcattgcatagtacaacagtgctttgccatgtaatttgttagcaaataaagcacccttcactgtaccataaagatgggaggctctaagtccacttgttgtttcttgctttgacaaagtaggcatttactgataataaataaaatgttggctgggtgcggtggctgacacctgtaatcccagcaatttgggaggccaaggtgggcagatcacctgatgtcaggagtgagttcgagaccagcctacccaacatgatgaaaccccatctctaactgaaaatacaaaaattgggcagggcatggtggctcaggcctgtaatcccagcactttgggaggccgaggcaggtggatcacctgaggtcaggagtttgagaccagcctgaccaacatggagaaaccctgtctctattaaaaatacaaaattagctgggcgtggtggcgcatgcctataatcccagctactcgagaggctgaggcaggagaatcacttgaacctgggaggcggaggttgcagtgagccaagatcgcgctattgcactccagcctgggcaacaagagcgaaacttcatctcaaaaaaaaaaaaaaaaaaaaaaattagcctggcgtggtggcacatgcctgtaatcccagctactccagaggctaaggcaggagaatcacttgaacctaggaggcagaggttgcagtgagccaagatcgtgccactgcactccagcctgggtgacagagtgagactctgtcttgaaaaaaataaaaaaataaaacgttgtagtctgtacaacatgaaaacaggccaaggccacaatacataaatggaggagtgtggctgtgttccaataaaactttacaaacacatttgaatttcgtatagttctcacatgtcatgaaatcatcctctttttgttattttgcaaccatttaaaaaatgtaaaaatcattcttagctcatacaaaaacaggcagcaagacagatttagcccacaggccttggtttgctaatccctggtctagactctatagacaaccccaagcatatgaacaagatattcattctaaaaggtcattttatcaagaacgataactacccccatttattactatgaaagaggcacatagtgctattcagatacttatcaaatacatacaggaacctcagcaggtccttcacacacaaaggtggtgtcatccaggcaccctgaagtgctccagccctttgcattaataccccagaaagggaggtggggccctgcaggaagcccagccccctcccgtgatatctgagtgtccttggaggtcccactcccctttcttctgtcctcgggccctgggagtcactcccctctttcctgacacagcctgtccctcctagtgggtggcacagtacaggagtggacctaatgggagtgtggcacctctcccccactctgcacagacccttttgtacacaaaaagaattcccttctcttctgagcatgaaaggcaggcttggcttgggatgagaaggaactggtctttgaaagggggtcgtaccctctaggagagagtggaaaacagctgagagcctattatagtcatttcctcttgctgctgtcttgaattacagcaaacttagtggcttgaaacagcaaccaaatttattatcttacccgtccttaggttagaagcctgcattagtctgttcttgcattgctggaaagaaatgcctgagactgggaaacttataaagaaaagaggtttaattggctcacagttccacaagctgtacagaaagcatgatgctggcatctgctcagcctctgggggggacctcaggaaacttacgggggcagcgttttcaggctggcttctttcactgagcaatgtgtgttagtataatatgtattcacttcctaggactgccctgatagagcaccacaggctgcgtggcttatacaacagaagtggactttctcatagttctgccggctaaaaggatgtcccttccccactgctcttcagcaacgtcccagaaagacggtgcagtgctgtagctgttctcttattgggggtgcttgcaaatcatgcagaaccatccacacacacgacctgagtcttcttttcctctgtcgaccgatcgtagggaacttccagtgaggctgtaggtgcaggcaggggaccaaaggtatgatagcaggagtggggaccacaggaatttgggccacttctgcatataacttcctggtgccttcggggcctgctcaggcccagtcacgactagccacttccatttgatgatggagtgctgctgtgcacgcctgatgttatagtgtcaacccccagcacaggtcctgtggtaactcagtggcctgtagtcaagcattcagcatctaccaaggcccaggagcaggccaagggttgtttcttaaaaggaaagtccttatccccagagaatggtagtgccttgctccaaaatcctagagacttgtgctgcagtttgcttgtcggggcctgccaaaggctccaaacagcatctccatctgcctctgacacctcgagcaccatggggtctgctgggtcatatggcccgaaggggagagcagcttgcacagcagcctggacctgttgcagagccttcccttgttctgggcaccactcaaaactagcagctttttggatcactaaattaatgagccggagtcacacacccaaatgttaggggctgcctcccacaaaatcatatattgaagtcctaacccgcagcctctcagaatgtgactgcatttggagattggggatttaaagggtaattaggataaaatgagattaggatggaccctaatccagtatgcctgttgtccttataagaagaggagattaggacacagacaggcacggaggtaagaccatgtgaagacccagggagaggacgccatccgcaagccagggaaagagacctcagaagagactaactctgctgacaccctggccttgaacttctaagctccaaaattgtgagaaaataaatttctgttgtttaagccacccagtctgtgatgctttattatggcagctctagtaaacgaatatatcatccttttggcttttccagaatgttgtattgttggaatcatatagtatgtagccttttcagacttctttcacttagtaaaatgcatttaaggtggctccatgtcttttcatggtgtgatagttcatttcttttcatcatcaaatagtagtttgtttattcattcacctgttggacatcttggttgcctccaagttttggcaatcgtgagtaaagctgctgtgagcattcgtgtgcaggtttttgtgtggatgtaagttctcaactcatttgggtaaataccaaggagtgtgattgctggatcatgtggtaagagtatatttagctttgcaggaaactaccaaattgtcttttaaagcagctgtaacattttgcattcccaccagcaatgaatgagagttcctgttcctccacatcctcaccagcatttggtggtgttggtgttttcagtcttagccaccttgcctgtgaccgttcggtcccagcaccatcttttgaaaagactatcctttctccactgacttgtgtttcctcctttgtcaaagatcagttgactgtatttctgtgagtctatttctgggctgtctcttccattgatctgcatgtctgttgttgcaccagatcactgtagctttatgttatagtgagtcttgaaatcatgtagtgtcagtcctctgacttcattcttcttcaatattgtgttggctgttgactttccctttagcacattccttttctgactaaattagccagagttggcctctgttaagaatttaataggccttggtgaacaataattcaccatttccactttatcttcttcctcttttaggattcttaggaacctagatttcttggattctctattgtcatttctgatttaaacagcaaatgccactgcttattaataaccatttgtgcaaatctctgtgctgtgaacactgatgaatataagtaaatcgaagtggaccgcagaattcagtgagcaggagaagctgtctctgagctcccaggcctctgcccctctctggggcacctcctcgcgccctggggtttcttctccatgtttatcctgggatcttctgatccactgtgccagtgggcacttccttcctggacggtgggtccttctttccctttctgtctctggggtaggctttcagcatggagctgctgttgctctgttaataccctcccatgcctgaatcaaccatgtcctaatagaattacgtaaccatgagtcaaagcatgagattcaccatcaaaaacagacaggtagtgttaagttccacaacaaaatcacactgccttcattgtttgttttaaaataatcaaaatgaatcctagccaaaaccccagtgtttaagacataaaatcaagcctgtgctggttggacagaatgggacaggttgtaaggaaaagtagtcttttttttttttcttaaggttagaaaacatttgagatcagccagcaaagctttctttagtgacaccaggaaccaggctgcttggggtcctcagtagcagcctctgtgtgcgtcctccacacccccagccctagtccgcgctgggtgcccctccctccttgctgttcagtgtgggcccttccctgcttacaccctgtgccctctgctctcactgggtccccactaccaccctgcccagccctgcaaggcccttccctaggctcagcagcttccctgcgcaccctctctgccagcttctctgctgctctccatctgggcggagccgcagggtgtcaggcatcccaagcctgggagtccccacactttcccacctgccttgtggggtagcagccccagaccctgtccctgggccgtctttttatttctttatctaccagctaccttttcaaataaatgtgagccctggagtttgagaccaacctgggcaagagacccctctctacaaaaaataaaaaatttcctgggcatggtggtgcccacctgtggtcctagctgttccagtaacggatgggctgcagcttctgctcccctcatcttctgcctaaaccgggaccccaaacggaaatgataagcctgaaactcaaaggcagaatgtcggatgtggaggcatttattgagccaattgtataaggatattgtacatctcttgttttaatccagacccttttccattgtaaatggtagaattctaactcaaacaagtgtaaggtagagaggaggagtttttttgactcaggtaatcagggttttaaaggcaaaaagtggggaaagagagtagtctgatacaaaattatttgtcaggaattttcattcgtttacagaagtaacattgattagtaactggctatacattttaacctatggagtgtgggctatggtgtccagtgtggcattgttaagttaatttatagctatttgtggcaacagaaggcagttttaagagatgaatacaggctggcatgatggctcgtgcctataaccccaacaatctgggatgctgaggcaggaagatcacttgaggccaggagttcgagaccagcctgggcaacataggcagagtttgtctctataaaaaatcaaaaaattagctgggtgtggtgatctgtgcctgtggtcccagctactcaggaggatcccttgagcccaggagattgaggccacagtgcgctgtgatcacaccactccactccagcctgggtaacagagtaagaccctatgtctttaattaacaacagcaacaaaagatgaatacatagctcaaaatggtagtgcaagcagggggtggggtggggggagtaaggcatgattgttgtctcattttaatgtctctctgagcctgatgatttaaaaggactcacattcttcaggcaaaagttatttacttttggaagtccacctgctctcacagccagtgtcatgtaagctctctcccctgtcttgattctgccctcttctggctgcatttttacacttgcttccaggagctccaccttcatcaagtctcagcagcacagtggagagaggctctgggggctcattgcaattctgagttggacaactcaggcgggagcttagaattgtttctaacaggtgtttgttcttgtcccaaacctcaaagtttctttttaagataaatctgcttaatagtttctacaattatttctgaacttcatttcaccctcagtctttgcacagacattgattccttcacattgttcttttattttcttacagtgatgatacagacagaaatacagatatttgctacatctctgtaagaaaatgtaatattcctggattcctgtcttagacgatgagttttatattgtgtaatacaaatcaatattgcaagcaaaagacatttatacattttgaaaacaaattgtttccttgggagcctgctgatgaaaggctgactcctcttgggagaaatcactcaaagctctgagagtttaaacacactgggatgggatggacgagttagtactggttagtagataaagtctccaagaaacatcagagccatttgggtggggaagggatctccagcatcatatttttgatttgctctttggtgtttttattgatgatttggtatttggccgcaaacgagatagttaacccagatttctaaaatcctggaactgaacatttagaatatgaagactgtgagatcataggttcatagatgtgttgggtacaacatcttccatcaaagagaaaggggacatttttttcctgagcggttagaaggaaaacatgcttcctgcctatggagcagtggtcttagcgctcctcttgccccacctgtgtgggttttcactcttttgcccactctgccttccctaagcacagtgaaccagctggaggatgctctctgctcaccaattccagagcttccccggcttcctctgcccttggaatccctctcccacactcctaagtagccagatcctctccagatttaacggtcgattacaagtgttacctcctctttgaaacattttctgaccctctgtgttggaagtgctcttccttaagtttccactcgttttattatgtacttccttcataatactcattaccttttattgtagatatttgtaagtttcacttaaatgcattagcagattatatattctgtgcgagccatatccattctgattcatttctgtatcctccacaccacccagcagaatctggcataaaatgctgtaaacaggaaaaccaaaccttgtaaaatattttattaataaagaggtttattctgagccaatgtggctgctcgcggctggcagaaaacacaaacccaggaagccttgattaagtcgccccgaggcagttgggttacagcttgattttacacattagggagactggagttgcaggtaaaatcataaagcaacacatggaaattctacattaatttggcccaaaaaggcgagacatctggaagcagaggcttacaagtcataggtgggttttagggattctttatttagttggcaatgggttgaaagagttaagtttttcttttcttttcttttcttttttttttgagacagagtctcgctctttcgcccaggccgaactgcagtggcgctatctcggctcactgcaagctctgcctcccaggttcacaacattctcctgcctcagcctcccactttttttttttttttttgaggcagagtatccctctgtcacccaagctggagtgcagtggcacgatctcagctcactgcagcctccacctcccaggttcaagcgattcttctgcctcagcctcctgagtagctgggattacaggtgccggcaccacgcccagctaatttttgtagttttagtagagatgggggtttccccacgttggccacgctggtctcgaactcctgatctcaggtaatctgcctgcctcggcctcccaaaatgccgggatcacaggtgtgagccaccatacctggccaagttaagtttttctaaagacttgctgggcacagtggctcacgcctgtaatcccagcactttgggaggccaacgcaggtggatcacctgaggtcaggagttcgagaccagcctggccaacatggtgaaaccccatctctactaaaaatacaaaaattagccaggcatggtggcatgcacctgtagtcccagctactaaggaggctgaagcaggaaaatctctcaaacacaggaggtggaggttacagtgacctgagatcgcgtcactgcaccccagcctgggtaacagagtgagactccatctcaaaaaggaaaaaaaataaaataaaataaagacttgaagtcagtacaaaggatgcttaagttaagggggtcggctatctgtcatgtgatactataccagagtcaaattggaaagtaagccatgtcatatcgagttaattaaaaacaaacaaacaaaaacctttagcaagctttcatagtttgcagcatgtgacttaacccttgcctagcatggccttgggtcctgtttataatctggtgtcttattgccacacagaatctattgcattagactgatgatctctgttttaatgttaattccagtcatttgtgcctaaactccaaaaagaatggggtatgaggtgtgtctgacttcccttaccatcatggccaggaattcagtttttgttgttgttgttgtttttgagacggagtctcactctgttgcccaagctggagtgcagtgctgtgatctcagctcactgaaacctccgcctcctgggttcaagcgattatcgtgtctcagcctccggagtagctgggattacaggcccacgccactatgcccagctaatttttgtatttttagtagagacggggtttctccatgttggccaggctggttctccaactcctgacctcaggtgatccacccgccttggcctcccaaagtgctgggattacaggcgtgagccaccgtgcccgactgggaattcagtttttaaggtttttctgaggttacccttggccaagaagcttcattcagtcagtggggggccttaggattttatttttagtttacgataggtcgaatatagtccttgacaaaacagaagaaagaacgtagaaatcccactgcgcaattgaattccaatccagaaagattgccatttgtttttttatttcccctagtgcagaaataactcacatatcatgaattcacacctcttaaaattgtacaattcagtgtgtttagcatattcacaaggccgtggaaccatcaccaccgtccttgggagcccactgctgaaggactggaatatctaattcccgaatgttttcatcattctgcaaagaagccctggacccactagcagtcactccccgtttcccctcctgctcttcctcccaacaaccattagtctattttctctctttgcagatttgcctattctggacatttgacataaataatcttataatatgtggtcttttgtgactagcttctttttcttagcataatgtttttatctatgtgtaacatacatctctacttcattcctttttgtggttgaacaatattccatcatataggtataccatattttgtctatttatcattggtggacatttgcgtgtttctgcttctgtgctttcatgaataatgctgctatggacatttgtgtacaggtttttgtctaaatatatgcttatttctcttgggtattaccaagaattggaattgctgggtcatgtggtaactgtatgttcaactttttaagaaaaacgtaaacagttttccaatgtggctgcatcattttactttcccaccagcagtgtatgaagggttctaatttttccacatctcaccgacactcgttactgtcttttttattgtagccatcctagtggatgtgagatgttattcactgtgcttttgatttgcagttccttaattaataatgttgagtatcatttcttatgcaaattggttatttgtatatcttctttagagaaatgtctgttcaaatcctttgttcctattttaattgggttatttgtcattttttctcatttgttctcaagcacacgtaactctacatttagtctgtcctcacccatgggtatctgttcaaaaaggaatagtgtcaagttttgccccgattttatcatttcaattgtgttgtatgttagcaatattcagtgctaacaagttttaactacatggttctaaatatggatcttcccaacatttacaaatggaggagggatagacggagtgttcaaaagaaaatgttaatatgctttgaaacattgttttgtcaatgtaaatgttaaatcagcagaacccagtaactgatgagaagcacagtaagttagctacagcaatcacataagcagaagcacagggaaattcacgaagagggaaacagcaaaaatagaaaggcgggaacacaggagtgggtatagggatgcctacagttatgtattcatgaagtaagagctttctgggcagtgattaactgatttgtcagaaattgcagacagcaaggaaagaagaaataaaacttgcttggaaggaagtagtgttgtctggtccgctgtggacacctggacatgtgaagagaagactctgatctgtgctcagagtcgggttgtttctcgaacggccgcctggtggcgctgtggcacaggggcgctggcgagggaaaggctcctgcaatgcaccaagctgcgcccgggcgcggggagccgtcgactggggacttggaggtgtgggcctctcgtgctgtgtctgagggacagctgtcatgtgtgaagctcctgctatataaaagggtgagctgtttccctctgctcaccctttaaactgctctgcctgctgaatgctgtacccaggtggcggagagcagaagcgaaactggggagtgaggccagtgaggctggggggcttgctggacacagccagttctccagagctggagctaaacggcagcaaggtcccagcctctcactggtgtgttctcattttcatcttttggagatgttttttctgtcttaaaaaacattccaagtccatcaacggatgactggataaagcaaatgtggtacttatacactatggaatactattcagctgtaaaaagagtaaaatcatgtcttcttgagggtaattctctaagtgaaataactcagaaatagaagtcaaatgccacatgttctcataagtgagagtgaaacaatgtgtgcacgcggacacaggagtggaagaacagacattagagaggcaggatgtgagggagggagggacgattacttactgggtacagtgtacgctcttcaagtgatgcctacactatgctctatagccatgcaacaaaactgcacttgtatcccctatgtctatcactgaaaaaaaaaaaaaaaaaaagtccaagcccagaacttgtgtgtgcgtgtcattctgcacattcctggacaggtcctgtgtccgttctgtagaactgcgccatcatttctctcctggaggaggcccccagcctcaccagaagggatgaagatgtggggtggcctcatcctttttgaaacagctcttagttcagatgaaaccatctttccagaggctcggtggtttctcgatggaactgggggttagggaagagcaataagcttttctttttctttttcttttttttttttttttgagacagcctcgctctgtcaccagactggagtgcagtggcgcgatctcagctcactgcaacctccgcctcccagttcaagcgattctcctgcctcagcctcctgagtagccaggactacaggcgtgcaccaccacgcccagctaatttttgtatttttagtagagacggggtttcatcatgttggccaggatggtttcgatcccttgacctcgtgatccacccgccttggccttccagagttctgggattacaggcgtgacccaccgcgccgggtgtagattttctttacaggacaggatcaaatcacatagaatggattcagtggaggcaatcatgtctgggctctgagggctggagcatggctgtgtggcttaaatgatatgtccttggagagcagcaatttctgagagattagcaatagcttgcattctagatagcatgggagtttcataggctcgcctaggactctaaagatctcatggcatcgttcaacaggtgaggttttaggttgcaaagggaatactaccagatcctaagcaggaccactgctggggtcattctgtagttcactcagtaagcatcaatgatctgatgagcatgtaggaaatagcctttggggggatttattctactggagaaacttggcattctcagagaaccacagcaaccaggcaggaagctaaagggcaaatggcatttagaccagcgtcccctatctgatgcaggagtgcttgaaccaagtatagaaccggtttgatggacttgcagagaagtgggaagagagcatcctagataaagagaaaggctggactcaggtgggcacagggaaaacgccaggccgacgtgaggattggtgggcatttgataaggttttgtttattgtttttgtgggggttttttttgagatggagtttcgcccttatcacccaggttatagtgcaatggcacgatcttggctcactgcaacctccacctcccaggttcaagtgattctcctgcctcaacctcccaagtagctggaactacaggctcgcaccaccacacccgtctaatttttgtgtttttggtagagacggggtttcaccatgttgaccaggctggtcttgaactcctgacctcaagtgatccacccgccttgcccttccaaagtgctgggattactggtgtgagccactgtgcccgacctgataaggtattgataagtggtattaataagtgagttgagctcaatgaagagagtgatgataaggaatggatcatttaaataagagtagctcactggtctctttaattaaaaagaatgtgaccaggtgtgaagctcacacctgtaatcctagcactttaggaggctgaggcaggagaatcacttgagcccaggagttcgagatcagcctgggcaacatggcaaaaccttgcctctactaaaactataaaaagttggccaggtgtggtggcatgcacctgtagtaacagctacttgggaggctgagttgagaggattgcctgagccctagaggtggaggagtttgcagtgagccgagattgtgccactgcattccagcctgagccacagagtgagaacctgtctcaaaaaaaaaaaaagaaagagagaaagaaaggagggagggagggaaggaaggaaaggaaggaaggaaggaaggaagggagaaaaaaatgcaaggtcacagtaaaaagttcagagaatggaaaaggacagagagcaacactggaaacctcccttcctgggtcccaattgtttatcggatgccttcgagccctccccctgttcatatacaagaatctattaacatagtgatttgatatgcagagtttactactctctgtcttgtcccgcaacttactctgggacttaacatgccagtacatctccaaatctagtctattttcttcccacagagggagagacttcttttattaaagatacttctggctgggcatggtggttcacatgtaattccaacacttcgagaggctgaggcgggaagattgcttgaggccaggagttccagaccagcctgagcaacatagtgagacttcgtgtctacaaaaaatttaaaaattagccatgcatggtggcacacacctatagtcttagctactcaggaggccaaggcgggaggatcacttgaacccacgtgttagaggctgcaataagctatgatcacaccactgcgttccagcctgggcaacagagcaagaccctatctctattattttttaaagatacttctttgtggttttatttttttgtatgtagaagtattagttttattttaaataagatttaaagtatcacatacctttaaactgcatttgtacagcagttgttctgaaagatttgtacttgacttctgcaacccctgtttgcagaaggtgcttggtgtgtctgagagattgagccttgctgtttagaagctgctggtggtgatgataacctcctctagctcatgaagaacaaccttcacaatcattttcccatgtcaactgcagggtgtaaattccccagttgtgttagaagagaaacaggacagctaaatgctgtgtgatcctgggccagcccctggaagaaggatggggagattgctacaaaggacacttggttctgttagcagactttgtatatggatggttgattagtcagtaggactgtagcaacatttgtagttatgtaagagaatctcagccttaggaaatacctatggacaagggcatactggttccagcttactttcaaatggttcaggggaaaaaaatgtgtaaatacgaatatgaaaacatacagatagggaaagtatatgataaagcaaatgtagcaaaatgctaacaattgaagtaactgggtgaagagcatgtggaacatttttatactattctttctgtgagcttggaattatttcaatttttttaaattttgtggatgcatagttggtgtatctatttatgggttgcatgagatattttgatacaagcatccaatgtgtaacaatcacatgcgggtaagtggagtatttcaaactttaaaaaatacattttgttaaatattcatctgaggactactatatatacaatgagcttactgtgcatgttttaatcatctttgtagaagagaaaatagcttgcttcattacaataatgcctaccaatgctttaaattgtgtatattgttttctagtcaaattctaagaatcctaaggcattttctcatctccctctccctgaccttcaagtctgatcagcccccaaggtcttccaagtctctctgaatgtttctctcttccactcttctgaatgtctgttgcactcacctccctggctgcacctcggggtctctgcagtgggtttcactgccctcctgcccactctccccatctctccagtccactggttcaccttccttctgaaagcacagcccttgtcagatcaaagctgcccagacctgccagtgtgccagggccaccccacatagcagtgcaggtttccatccgcacagggtcatctgaccagagtagccatggggactgaaatccggatgcctctgctctccaagccctgtgctgggcacagggacatatcagcccagaggaagggaaccatttgggtgagaaatctggcagaaaacaaacaatcccctcaaaacaggttactgaagagagcttgatgacagcattcagcctgacatgggggaacgggggaaggaccctgacctcctcccctcaccctgccattccccctgcacccacaggagctgggccagggaacccagtggaggctgtctgcagagccagccccctgggccggagagggtgggtctgcaggccggcagaacccccagcgtccaggctctgcttcacagaaggcagtgacttggggctgacagacctgctggtagctcatcactcagcagatgaagggctgacttgcatttcctcttttaatatcaaagcctcccacagaccctgcttccaggctttctctcttctagctcctctggctgccgggctcccaacatgagcttagcctgcaccacccaccccaaaagtcccactgttccaagcaggccccaaaggcctcctccagcgtttaggctcctgcccaagcacagcctgggccactggacccctggcccttggcttctgtgtcttaacttcttgtagcatgcattggccaggccaggtctgctgtgggcagcaatgtctctggctgggacaccaagctccctgaggggagggctgtcatctaggatggcctgacctctgtgagcagagcccagcatggcaccttcatgaccagctcactgccgaggcagacatgccccagcttcccctatctcagctcactgcaacctccacttcccggattcaagcaattctcctgcctcagcctgctgagtagctgggattacaggcgcccaccaccacacctagctaattttttgtattttcactggaaatagggtttcaccgtgttggccaggctggtctcgaactcctggcctcaagtgatccgcctgcctcggcctcccagagtgctgggattacaagcatgagccaccacgcccagccaagcctttcctcttgaagcagttattccccagggtaaaggaggaggcactccttccaggcagaattggaaaattgtgtagtatgagtccgtgcagagcgggtgaggatgaggaggatgtgcaggttgccaggcaggacggcagtgacctgggggactcaatacccacagaacctcagtgctgcggaggagcccactgctctttgccgcccgacgtcataacatcttactgccttatttcatgcgatcagtgcatcaccgaagtacacgagcagaaacaccatgatggtgtccgctgcccactgaacgagggcaggtgagggcccagagagtgccctgcgcagcctagatccctgggggcacttggtgctcagattcagggaaattcttacagagcagagcatctgaggctgttctcattattcaacttttcatttggaaacaatctcaaacttagaaaaaagttgcaagaataagacttcaaaggtttcctaaatgcccctcacccaggttcgcctcttgttcacatttccctgtgtgccttctcatttgcatacattctgtagatgtgcaggtggaggtgtagacaggtgaatctttctccatcttctgagagcttgctgtggccatcatggccgttgactcccaaaggacgtccgtgtgtgtttctaggggcagggtattcccttaggtgaccatggtacggttctcaaaatcactcgattggatattgagacaataagtgtgtctgacccgtcatcatctgcacagcagtttcatcggtcggcccctctatgtgttctccagcattttcccctccaggattggaggtattgcatcgctccttagctctttatgacattggcattagtgaagaatacaggtccctttcctttttttttttttaattttaggtttgcccgatgtttcctcctgacttaaattcaggaaatacctttctggctagagcactgcatacactctgtggtgtccttcccggggtctcacctgtggaaacctgtgatgcccacacctcccacactggtggggtcaattgtgatgacccaatttggtgtccagtttctccactgtgtggttacttttttttttttttatgttgccactaataagcaccctatggggagacactttaagaccatacagccctcctgctcctcgttaaaacctccccctgcgttcagcatctgctgccaattccacccgacccatgcttccccacagtggtcgaaaaggatgtgtcccctgtcccagccctccttccgcattcactagctgcctttggctagagtcctcccttttctatttatgtatttatttatttatttatttatttatctgtctgttgttggcagggacacctgacaggcctgtaattttcagtggcttagaattcgttacagtacttaactgttttggtgctcacatcatcccacatttggccaaatgagagccccttcaatccagctcttgtgtgctagtggtgtacccccagcattttggggcacttccttactttccgacataacagtgttccaggtttagctcatagctaccctgccctgcccactccacaccccacacccccagttagctatttctctaaggatgaccaagatctgggtaccgtccctgctcgctgctactggagagtcttcacttcttggcatttcagcagacagaaataggaaatacatgcatgtagatacacagggacaaacatacacaggctcacaaatacacttgcacacacccatttgtggacagaatcatgagtgtgtctatgtatattttaaaaatcatgagctcataccaattcttgcaattccagtccatccccacaggcctcttgcttctcttcattccatatgtgtgtgttgcctcttcctttctgaaaatcctggctctcattcacaacataacacatttcttcatttgctcaaccatacaatacatctaaatttgtttcggattcgtgtgcaatgagggaatcccttttgaatgtcagtctcaacttacccatagtttaaattcatcaaatgttgctatgtgatgacaaataaaagcaaaccatgcaatgcaaacatagacaaatatgaagctctgtgccccttcctctttgtcaacagccctgcaagttttaggatgagccactccagcttctctgagaaaggctcatagttcctacctgggataagaaaagtttatgttatgtttaattgcatagacaattcgatattctattacaaggaatactgttgctttaaactctgtaactaaaccaaagtctaaaagaaaccagttgctaaggcaactgggcaaagagataattaattcatcagtcattatatgcagattaatctgcctaactgagtgggaaaggaggtaggcttctttgatttttccttttggaattagtagaagagagaagggtgtagttttttaaaatctgtaagaggcctcagagttcatccgggaaagggtggaggctttggaatccaacagaacagacccctgtgaaagctatggaccaaattgtgggcaatatggtgcattctataatcatcaaatgagatgctgaaaataaagcacctttcagtgtcttttaaaaatgaagagtgacttacaaatattagcataaatttctctaaaaaaaatgaaatgatgttttttatgcacttgtagaattaaccacacaaactcctctgtgctcccgctgtaatcgttacagaattcagagaacttgactgcaggtccatccctctcaactagactattaaattaatgagatcagggacaatgtcttatttatccttataatgtcaaggcatggaagtgtgctagatgtatagtaggtgttcaaagaatgctttatgatgaatgaatgaatgaatgaacatgcgttgctttaaaagcacctcagagcctccatctgtcccccaggctccatgttgtcacctgctggtgtaaaagtataattgcagtaaatgtgtctaagaacaaggaattgaagtcactcagctgtcatcaggagactctctcccacaattgctgtgctgaaaattctacatgtgcattttgttcttttttattttggtaaaatatatgcaatataaaatgtaccatttaaatcatttttaagagtacagcgcagtagcattaagtacattcgcatcattgtgcaatcttcaccaccatccatctccagaaatttttcatcctcccaaactgaatctctgtacccgttaaacactaactcctcatttctcctctcttcccagcccctggataccaccattctactttctgtctctaccacattttgcttacccattcgcccatcacatgtgcatttttgtacagttcctagtatgctttgttacctgtgtgttcccagccagcagcttacactgtgctcagctgagtgtcacaggccctcagtgagtctttgttgaccgtactatagttcgttgttcatgtgtctgctcagcagaaaaagctaggaaatcctcaaggatgtccacatgctgcctcatggagagggataagatggatatatattagttctattcctattgaatgatcgtcataattaccacagtccacttactgggagcttgatgcagtgaatcagttcatctaatcctcaccacagccccatgaggggagtactgttaccctcacccatgcactgagaaggacagagaataacaaagaaagggacctctgctggcattcttgttcggcatccgctatgctgcacccagcggaggggctgcctcttaagaaaagtttccgtgccaagaacacgcattctggggctcatatgctcacccgacctctgagactgagtttcattgtctgccaacctgcctcctgggcgatagcagttcccttccctctgcctatccttctgcagtgccttctcaccttaaatgtgcgccttccctccatgtggtccgaccccaccactcctcaacaccggcctcggatgccgctcccatgatgaagctttgcgggattccaaggaccctcacacacccctcagtgtccctcccacgccacctagccctcggctcatacccatcctggcacactgccctctgagtgagtgggctgtgctagtcctatttgctcaactgcacagaaacttaagaaattggtcttagagtgttcttatcctttctggaatacatctctttgggtgacagcctgtaggtagcaatatccatgtgttcaactacttgaatcctcctttcctttttgtgtggacacgaaattggactctacgaaccatcttcccttatggtatatccccacttgatttgcttaaaatatgagaacagtgtgaggcagacagacagacattactgaattccaacatgggatcagtgctagttctaggattcatgtggaatcaggtgttagcctaggggccacatgagatgaggtgttggcgctaggatttacacagaatcaggtgttaacactgggattaacacgtggatgtcaccacacctttgattttccacaggagcactcactaatacaaaaaaaaaaaaaaaaccaccactgttaggcatttacaatgcagaccacattgttcctctgtctttttgctccaaatcaagggactttgtggagtccgactcactttatactaaaatatgtttgtgaatataatcatttgaagagtagaaaagtcagggtatctagaaccgtcctctaagtgctaaaattattttttatcataaaatattccctcagaaagaaactttgttcatatatggtagatttgcttgaaaaatttttacattttacttcatctgatgtttgttaattggtaaagagagcaccatctttaaagctttttagactggttttgaaacttcactttggtattaaaacacattggctaaacaacatgtgtttgaatatctgctgtagtccaaaatgctgctaggctatttgaggttaaacagcagattgaaacagatggggttgctgattttcactgttcaaatgcaaattattgggcactttatttgatttctggtttgtaaaatgtgttttaaatgcaactttgaggtgtgtggtttctcttaaaatacaccagtggtccccacacctacccttgcctcacagcctgacctagtttggtggtcttcggggatatttttgaattgtaagaccttgatagttcattctatttgcaggttcctcttgttatgctttgtgggatacgcttttgtttggttttgttttgacatattcaatggcctttccaccttctaacacagtaattaaactcctatctgtgccactgagtccagtgctttagtggcactggatatttcagtcctggatttttggacaaggggtgaagtcagagacatgtaacctacttttcctactgattcagtgttattttggtttttaaagctgtgagttaaagaagaatgtgttattctcctttacagatgacacttctaaaagctccttaatattactttggttataggggtaagaaaaatagaagataaattatttcaatacattctaaaatatttgatcacagattattatgtacagaaaaggaaaatggaaaaatatttccctgcttttctctaaaaatcgttaaaatttttatcatgatgttttttaagtgtactataattaggtcacagaatgatctctgctgctaaaaatgaatgatcatctactcaggttatttccaccaagatactaagaaaaaaaagagagataaaacaacaggaagttgtttaaaatgtcccgaagggacccccatctgagggctgtgtgcacactggtcactcagtggccacaactccagcaccacccagttgtttgcaactctcgggctatagctttcggcttcttggagcattgttagtgcatagaatttagtgaccattcacctttcgtaaactgttttgttcctcagagacagagtctcgctctctcacccaggctggagtgcactgatgcaaacatgactcactgcagcctcgacctcctgggctccagcaatcctcctgcctcagccttccaagtagctgggactacaggtgcatcccaccatgcccggttaacttttgtaatttttgtagaattggggtttcgccatgttgcccaggctggtttcaaactccggagctcaggcaatctgcccctccttggtctcccaaagtgctgggactacaggtgtgagccactgtgatgggcgctaaaactgttgatgtacctgttccagaagtcttagtgcagttttcatctttaataacctttaaagtataaagactacagaagaccagttattacattttctatctattgtttttttattaaaatttaatgtatccaccgtcttgtgctatacatacatctagtcagttttcaaacttgatcagcatctcctggatcttggcctgtcgtgaactgaaacagctgccttgaattttttcatcctttcatccagttgttaattaaatttctgcaggggtgggaggctccttataatgggaaacaaatttccttcttactgctattggggacttaagctcagctaaccagagaacaggtgacttttttttcaacatttgccatcttcaatccgaaagactgaaagagaccaagtttttaaattattattcaaaatttacaaaactaagtataaaagaaatttaaataattaaactttcaaatgcactaagacttctgagatgccttctgcagccctggccctggctgtcccctccaacctgacccgctggtgcctagtcttttccatcacatttgatttatttgaataaatcagagcccatggcttcacaggaccgtatatggcatttgggccctctttggcagcaacgactgcctttctgttcacacgcatttcgttttgagaatgccaatggacacaccaactgcttctttggatttcaccagttggcagtggttgatcagctatttggaatgcagccgatttgagtgaagaaatatccatatttgtagccctgttacaatagtgaaagctacaaccacaaaaaaaagatgcaacaccattttgaatttgaacaccagaccaacgtgaatagtgagataatcgtcatctttacgacaaaagagggattgagcaaacagtttgtatgttggatgatgtatttgcaattactttgctagatactgccaataagaaagtgttcttgggcagagatacctagggagtcgggttttgtttaaagccgtaacagaagatggattttggttgtgtcttcagaagctgaagttcttgttccattggagcactccacgactccccttttaaaagtggagccttttgttctgggacactatgaagttttggatttaaaggcaaatggcaaagttgcatctgtggaaatggttaaatatcatcactgtaggtacaacctctgcatgccttctgtaacaatgtggagaagctcttctcaggtttagagacagaaactttgaagagcaagctttggttcctttttaacaatgatattaagaagaatttaatgccaggtgcagtggctcacacctgtaatcccagcactttgggaggccaaggtggaaggatcacttgagtccaggagtttaagaccagcttgggcaacatagcaagaccccatctctacagaaaatttaaaaattacccaggagtggtggcacatgcctgtagtcccagctacttgggaggccaaggggagaggatccctccagcccaggaggttgaggctacagtgagctgtgattgtgccactgcactcaagcctggcagcagagtgagaccctgtgtctaaaaaaaagaaagaagaagaaggagagggagaaaaaggagaaggacaattttgacgacagacagaaggataggctccattttattaggggcttggactctggcttggttgctgccacccaggtgaagcagctgaaagagacgcagtgcaaaatacactccccagacatttgtgactggcatggagggcagccctgatttattggttgctggacgagtggcagatcgtattggaagtgaacatcatgaaaccctcttgaactctgaggaaggcactcaggctctggatgaagtcatattttccttggaaacttaggacattacaacagtttgcacttcagtaggtgagtatttaatttccgagtagatttggaagaacacagatagcatggtgatcttctctggacagagatcagatgaacttatgcagggttatgtatattttcatgaggccgaggagagagaggcctctgaaggagctctatttgtttggtgttctcagcgcagatcgaactgcccacgctgcccacagccttgaactgagagtctggtttctgcatcatcgattttcttcctgttacatgtctctgccaccagaaatgagaactccaaagatgaggtggagaaacagctcctgaaagagacatttgaggactccagtctgagaccctgaagagattctctggggaacaaaagaagccttcagggatggaataagaatgcctggtttaaggtgttacaggaatatgttgaacatcaggttgaagacgcaatgacagcaaatgtgggccagaaatttcccttcaatatcctaaagccaaagaagtctgttcatgcccttgaggcttgaatgccactacccaggcagggtgcagtggctgacccagtaccgcatgcccacatggatcgatgcccgctcactgacccactgcaagtcagctacctaagcctaggtgctctctgagccaaagagcaacagcaaatgttcttgttgtgaaggataggggtactggggatggataggggtactggggatggataggggggcaaggaaaatcacccaggcctactggggtgtgaaaaaaataaaagtcttaaattaaaaaaaaaaaagaaagaaaaagacttctgagacatcctgtatgtcttaaacttcaacaagagtgaggaaacaactgttttacttttgtgcctttcaaattctccacgaggtgaccaggaccttctcttcctggctctggcgcagtcagcctggaccctctgccggtggctcccagggcagccctgggtccccgcaggtgggtgtgttgccggggagcgcaatgctgctgctgcggttgcctagcaacagacgctcacactggcagcgggcgccactctgctcttggtaccctccccaccagccctgctcccactgctccaggtggccagctggtacacctgatgctgatccaatggttctaaacacaaatgagaatcggtttcccagaccaaactagaaaaaggtcaggtgattgggttaaaaatttttttaaggtgtgttgaattcctacctgaaaataggctaagattggctttgcatttttcatgagaaatgtctcgtcactgtgtaaggaaagtgcttttatgttttagagtgttggcaatgtgttgtggcacacaatgcaggtaatagtgcgaatgttgcagccaggctgcaagggtctgtgtcccagatactcttcttactcagcctctcagtctctgtaaaatgatgatgacaatagttcccaccttttagggcaaataccaagtaaattgttccatggaaaggatttaacccagtgccagcaatcactccataactagaagcagttttatagcacacacattatcattatattattaatatattaacttcctgggtatattttggtactttttcatcacatctgtttacctaatccttcttaaatttcaaatatagaatttatctttaacactgattgctaaatctgaacaagtggaaatcttttgctatatttttagggaaactaaatatcagacttccttaaaaatgtgacttctaggttctaacataattctccctttataagttttcaagggatttattttgtcccctgctgtccaacattttatagaaatcttgacattaaaaaaaagttggcaaatccttataaaaatcccactactctatgcttttttcaaaaaaaagcacagacacatacattttctggccaggcgcagtagctcaggtctataatcccagctctttgggaggccgaggcggttggatcacttgaggtcaggagtttgagaccagtctggccaacatggtgaaaccccgtctctactaaaaatacaaaaaattagctgggtgtgatggcgggtgcctataatcccagctactcgggaggctgaggcatgagaagcccctgaagctgggaggcagaggttgcagtgagctgagatcatgccatggcactccagcctgggcaacagagccagactccatctcaaaaaaaaaaaaaaaaaaaaaaaaaacctcaccttctgactgccccactagtcatagcgcatgctgggttgatattgccacgtgtgaatatatgtctttacacactcataagatgtgagctgtggaaactgtgctaaacagtctccttccacagaacttcaacttttagcctagctcagaatggagtctgacagtgtatgtttgcttctgcctgtctagaaaagtaattcattgcattttttaaatgtatttttattttcagtaagaatatttattgaaaagtactatattgaacaaatattttttacgtgtggtcatgaaattcctttgatgagaattcacactttgagctggagttatctttcgctttcatgacgtcattcattcccttgacaaatattcccagcgcatctcctctgggtcaggcactgaaccaggtaccggtgccgcaggtacaaggataggctcagcctccgccctcaaggagtccacagtctagtggagttttgaggacacagttaaaccataacaatgcaaggtgatgggtgagaccagcggagaatctggtgctcccttagggcttgagaaggtgtggcctaacccccaatgtgagggaggcagtgaggcccagcttgaggctggaaggataactaggggggttgacccagaaaagagagtccccaggtattcccaagacacaagcagcacatgcacccgaccagcctgttcaggaactgagagtgttaggagggatggggtaagccctaagaaggggagagtttatactaaccccaccagaaggaccaggcccaggtccttagatgatggggacagtgatgtggctggatttgtattatcgcagtgggagcattgtgcaaggtgcactgaaatgagacagcaggcagtggaaaccgtccggaggcctctgaaatccagctctctgttagtaatctaagaccacagcagtggcagacgccacggaaagaacaggacagatactcaggaaacggacccaacgggatgcggtaactgttggggtttgtaagaaaatggaatctggcataactcccaccgttctggtttggaaaattaggggcccttattccagagaaagaactgagaaaggagcaggtagaaggcgtgagggtggcacggggcatgagctcaatccttggcaaggtgaattccagacacctgcggcaccgtgggggcggtgtgtctggggacggtggtggtcagcaggatgggctgggctagggatccgcggtgggcacggtgagagctgagtgggcagagtatggaggatgcagtcagcccgtcaggaacgtgtgtgggttgagaggctgtgctgtggggacggctcctcggacccctgggtggaggagcccggatgcaccggccagagggacagcctgaaacccaatgcagtctctaggagggatcaaagcaggagccacagagacggaaagaaaataaagaccaaaattcatggatgagacttggcccctggggggccctagatgaccttgatgagagtattttccctatggggtgaatgtggaagccaggctgccaggtactgaggtgggagcagcaggaggggaatacaggcggagtcagacgacctttctaactcttggctgcggtgggcaggaggaaaactggtggaactaacaaggggcgtgagatgaaaggtgttgtgtggttcaggtgggacagagttgaatgtgttcgcttgcttgagggaaaccagccggtagcaggagaggttgaagcaagagtggagaggggaaggtgcgctgggtgaggggcgaaggtccacagggcagcggcgggcctgagggtaggggaagcgccgcctggggagccggggcgggctggggtctgttctcctgaagaaggccagctccgcagtgaagaagaccgtgtaggtttcttggaaggtggaggcactcgacgacagagctgagcaaaggcaagttagacagcgcagcgccaagcccgagacggtcaactgggagccgccacacacaaggaacagtgatttctccacgcccacggctggtgtccacgacttcggcccggccccctcttctgacctccttcccccaagtacaacactgcaaacgccaagctgccggctctggccctattggaggggtctcagtaacggagggcaggtgccagtctcgcgccctagttcgttcctctgctacaacgccaagttcgaggccacagtgccttctggaagagttgttgtgctgcttgggagcactgcacaggagaaacgggggctggaggtaaagacaggaggctcgggaggcggcgacgtgggcgagctggaatagtctagaagctgagcagaacaaaggcggtgtgactggtgagcctcggagggatcctcctccctgctagaatatgcatgatcctccgcgagtcttcgcccgccaggagcagggacgcgtccgagccaacacggggcgcgcgcccagacgcactttcccggctcggggtgcaagagagccaggcggccgcggcgcagcggaggggctgcgggcccggaacccaggccggtcagcgtgtaagcgccccagccggccgggctccgtggggggtcagctccctgacccctacagcgcggtagcgcctctccgagagctccgggaccagcggcccggccgcccccaaagccagcctccctctcccttccccgcaccgggatcccagaccagggagggggcgcacgtccgacggctgaggaatagcagggcgcgagccggcccggcaggtgcccatcgtcgccctctgggaccccggtggcgcgctctgtcctccgcgccacgctcagccaccaccccggctgtttgggacccggcacccagccgagcgcgccgccccctcggggacccgctgggcggggctgagcgaggcttggagtgcgggcgaagggacgtggggcgaacccggggcgctgcgccacctcggctgtctccagcggagaccggcgccctcgccccccgtctccgttcattgtgctgtattcatccagcagattttgaaacaattctcgtgtaaaaaggcattttactccgcgcgtcttccttacagccatttagttgggagtttgcggtgggcagggggagggagaagaaacgcctgctctgaatcggaaaacaccgaagagaccagaccatctctttcagcagcaggaaagagaggagccgtcgcaggagccgcacacgtctccaactctctattgctttttgcgcacattcctaacttcctgacgtccatcccagcgggcaggcatggggtgtttgggcggcaacagcaagacgacggaagaccagggcgtcgatgaaaaagaacgacgcgaggccaacaaaaagatcgagaagcagttgcagaaagagcgcctggcttacaaggctacccaccgcctgctgctcctgggtaaggccgaggggcgcgcggcggctcccggccccagcggagcgcacagccaggagcggcgagcgccaggctgggcgggcagggccgggcgagggtcgcgcgcacctctgggccgcggagcccagacggcggccggggcgagctcctccagccaggaacccgcgtgtaggaaatccccgtgctgggggaggaggattgctcagacccggctagtggtgagagatggcagcgatatccggacacagatcacagcgttctttctgtttgtttgcaggggctggtgagtctgggaaaagcactatcgtcaaacagatgaggatcctgcacgtcaatgggtttaatcccgagtaagaatgttcagtttgcttccaaactgcatgcaaacttcgtctctctcccagacgtcccaaaagtgctttctctaaacaattttaatttatttgataatggagtagacattcaagggggaaaaaattagatatttgctgttggatttggtatatttaggcaaatccttcttctgctagtgtctaatgaaaaaaaaacttgcttaacaaaatatgatttataggtattcttggagtgttgatctgtattactggtattgctctgagcacacttaaccaatatctcgatatatttttttcaatataatattatttgtttgcatctatttcagtttataactgtcaacatattatccatgggatagatctaggtaaaatacatttacaggtgtaactgcatgttatatttatatgcaatatccatagcgattttgcctagtttttctgtaacctgcatagttgtttaaagctatagatttttaaaaaaatgtgtgcattcaagtttgtttagagtacaaattagaaaagaagggtgctttcctttcccagataaaacctttgcggatgtcttgggggttgatactgacctctagtgaaaccagtctaggtcagtgctgtagctggaaatttaaaatcccactcaattgattggaatcagtgctaagagtaaattaagctaataacaactctctttcaatacagggaaaagaaacagaaaattctggacatccggaaaaatgttaaagatgctatcgtggtaaggacttttttaaatgattgtttactagaaaggtcaagtgctcttcattctaaaactgtgtagacagaaattacatattgtagattatcatacatggagcctaaatgtttatccatattttttttcttattccattttagacaattgtttcagcaatgagtactataatacctccagttccgctggccaaccctgaaaaccaatttcgatcagactacatcaagagcatagcccctatcactgactttgaatattcccaggtaagaaatgcttactgaaatattctaactagtgaaagatggaaccatttttaataaggtttcttattgagaatcaatattgatactaattcattattaactttcttcgaaatccagctctctaaatgcataagaggaatacttattctgtaccaaaatatttgtgtttgttaaaaatattaactacggccaggggcggtggctcacacctgtaatcccagcgctttgggaggccgaggcgggcagattgcctgaggtcaggagttcaagcccagtctgtccaacatggtaaaaccccatctctacgaaaaatacaaaaaaattagctgggcatggtggtgtgcgcctgtaatcccagctactcaagaggctgaggcaggggaattgcttgatcgagggaggtggaggttgccatgagccgagatcacgccactgcactccagcctgggcgacagagcgagactcccatctcaaaaaaaaaaaaaaattaactacaatgggattgcaagaaatgcttttatgagtgtatggggaaggctgataatcgggaaggcagcatacctttaagaaatactgatggtgctttttttcctgtctgtattcttaaactaacaattaagcagtttcccagataaaaacagtagaaattacatgagtagagaaaaatacttatggtaggtaattgcttccatgggaattacagaaagagcagaattcagtttactttgaagatgcacagccttcagatcaattgagaaaagaatgcattgtataaaaaatgatagcatggatcacttctgaatgtttatcatattaaaatgcagtttggttcacatttgcattatttagggctgcttttcctaatcacttggatggcagtattgcttatgcaaaagagtgacatcaatcttagttttctctcatttttcagaatatacatttctttaaaatatttggtcatttcataaagcatgtttcaaaggaaaagtatttgttaagcttctcagaaagtcattttcatttttgttacagtgcaccagaagtgagtgtgtatgtgtgtgtgtgtgtgtgtgtgtgtgtgtgtatgtgtattcatcaataacagtctttcaaaatttgtattgcctggcatggtggtttggagacatttttcattcttagaatcctctctttgaagggaagcatgtggagaaaccaatatgtaaaacagagagaccccgccctgcctgcaagggctgtggcccctggctcaccccaccccttctctacagagcaccattgtcctgtgggctctgtattgggatggctaataagaatgactttgtttcttcgtttgtttgtttgtttgttttgagatggagtctctctctgtcgtccaggctggagtgcagtggcgcggctcactgaaagctccgcctcccgggttcacgccattctcctgcctcagcctccctagtagctgggactacaggcgcccgccacctcacccggctaatgttttttgtatttttagtagagatggggtttcaccgtgttagccaggatggtctcgatctcctgacctcatgatccacccgcctcggcctcccaaagtgctgggattacaggcatgagccaccgtgcccagccaagaataacttttttaaaaagcttttattttcatcctttaatatgatttttgcttctgataagcatgccagtgtagagccagtgatatctgtagaatttctaaaatggaggagtgaggagtggcacactggctggaagaaggggctgggcatctggccccatgtctttaactggatggcttggagagggcaaggggttgacgtggcaggggaaggggtcggcagaagatgctccagccacaccaggatcctctgctaactggaggtgcagatgtcctcccggttctcactgatgctatgtacactggcaaaatcacccagaactacatggtcctggttgaaatcaggagtggtgttccctttcaacttcagatttagacatttatagaaaagcatctgagttcttggatttatcctatcatgtttgcctagaagtaatcgtaaaacatcttatatatttggggggaaatgctattcttaacatctagtgagaaattaaaattaagatttattaatcggtcccatgtcttctttctcctgttcataacagtctcacggttgatgaggcatcccatattcaaccccaaatccagaaacaattggtaactcccacctatattatctaaggaatccaaaacatctaatttgtgaaagtgtttttggagctaaatcatgaacatttttaattaaaaacttgagaaaccatttgctttgaatgcatgtattttcagatagataaattaaggttacttaaaagttattttcagaaagcataattaattatcattacagtaaaggagaatttagcaccagcagatgcaaatgtaattacagtgctctttcagaggtaatacttttgtgggggttagttttactggtgttttttaagcctaacattctgttgtcttaaatgtctatgcacatgtcaaatgagagtatattttatatatatatttatgtatatttatatatttttaacatagccccgacttatgacttatataaatcaggaaattaatgttaaatatgcaacattccttctgaaaatgctatttttaattttagtatgcacagaattttagtgacatttatttcatagtgcttggaaatatttatattacttattaaattatgtagttcttggtttcatattttgatgatcctcattcagaatgatatcaagacaaatctgacttatatgctccattggaaaattaacttttatttaaaagaatgtatgttttttataaacatacaagtagagctaaatcaagtattttaatagtaaacaaaacactgttttactcagtccagtacttttttgagattgaatccttgagaaagcctgtcagtgtcatggttcaattatttgagggtcttaagttaattttcctgtaggggtaagataacttttgaaacaaatacattatattaaagttaaaaattaattttagggttcttcaatttaagaaggaaaaggagaagaataacttatcttagaaacaaacatcttgagaattttctctgagaatattgcacatgggggaaatacgggtgggttgctctttgggtcttttgctctctgtggtgtctttcatttgaatttcagttgaattccatgaatctgaagttgtaacagaaagcaggcaaatgtggttagtacctatcagccaagggctgctgtggggaggtcagagagactcagaggctggatcttatcatcagccttacaggccaggtgtgtccagacacacgaagctttggagggttctaagcagtggagccatgagatctcattgtgtttcatcaagatcactctggctgtggatggagaacggattgtggggaggcaggagttgaaggagggcaaccacttcggagactagtctaataatacagtggaaaggtaatgggagagcttgcagtgggaggggatcatgggggtggagaaaagtggcctgagttgggatttattttggaaacaaccatttagacttgtcggcatgtggatatgagtactgaggggaaatagatcaatcaagaatgactctgagcgtttgggtctgaatttctgcatagaaggtagaaacaggttgggaggaggcaggtatgaggaggttttgttttcctggtgagttggagatgactattacgacatctaagtggagacaccaggtggacagctggatgcgtgaggccccaggaggagccattgcagaaggaagggatgtcagcggtgggcagtgctgaccggggagtcaagatggacattaaggttaaaatggctttggcggcttcaaggtcaccggtgaccttgataaggacccaggtggagggtattgaggagagaatgtgatgtgagtgggtgcaggtggcggcggcagattaggctttctggaggttttgctgtggaggaaggctgaggagccaggcagcagctgggaaggtaggcaaggtcaagatgaaagatacaaaaccctgtctgaatgaaaagggctggaatcacttggtaaagcaggagaaaatgacaatacagaagaaagaggctgtgtatggcaggagtccagtcctcgagaagaatggggtccggaacaaaaaaggaccagaccaactccccacttgagaaggcagagaacaggtgcagacgcaggtatgttttcagatttggtggcagagaggtgagggagggcttgttagcaaaggcttttcaagcaaattgtgaaagcaaattggtagcttacatggggatgatcgtctagttttgcaattatccataaactggtattgttcaggttataatgaacagtgatggatgataaaattcttgtgacctcgagctcacgtgatacagttattcccttcacaagccagaactgtaaaggggagtttcataccaggtcaaagcaaaaaataaaaaaactccagttatcttcacagcaaaccggcaggatatttgctgcagaataatgtgaaatctttggagacaaggtcagcatatgttaaattttaatttcaaatgaaataacacaaacatattttacaacacataaatcaccatagaaaagtactgggattagctacaaataatgaattgacagttgcctgattagagtttttctcatgaatacacccaaagcttcaggttatctttgttcaaggcttgactccccactccgtaagcccctcactagttggcactatgtcttagcagccaaattttgcatagcagccagcagatgcaatgatgcaaaataaatagtcttaagtgattatgccaaaacatagattagctgatcatttatgaaataggaaaaataggccgggcatggtggctcacacctgtaatcccagtactttgggaggccaaggcaggtcaatcacctgaggtcaggagtttgagaccagcctggccaacgtggtgaaaccccgtctctactaaaattagaaaaattagccgggcatggtggcgggcgcctgtaatcccagctactagggaggctgaggcaggagaataacttgaacccaggaggcggaggttgcagtgagccaagatcgtgatattccactccagcctgggcaacagagcgagactccgtctcaaaaaaaaacaaaaaggaagaaaagaaaaaagaagaaaaatgttgtggttgcctcctccaaaaatcctttagcactataccaagcaaaatcactgttgaattcaattgagttattcatttgtttcatttttattcttaaaatgtggatactttattagctcttttcaaattgtattttgctagttttttctaaggggaagaggataaatcaggctaaacaagaaacagaaaagcagtctgatctgttggtcagtactttgccattaccacctggtgtttcctaaattattacccagagagcagaggtcagtgttttgccttcctcccaggtgagagctactctgtcactttccaacagcagccccaagtcaggaggcaccctgtgtggctaacgtctgagtgttcggtcagtgtgcaggcaaaggaggccacagtggcctgggccaaggccccgagagccagacggcctggtgtgactctgagcaagtggcctacacccactgcagcttgggccctgcatagagagccaagtgcggtgcccagcccagggagtgccatgcagagctccagcggctgctgccgtgtgctgccgcctctccttgggagatgtgtgcagaatctccagaatgcggtcagattcttccgcacaatatctgtgttccctctccaagcctgtgtctatctcctgctagaaggctttgtgctgtgggttttcgtgtgtgtgtcttttttttaaccaaccaagctgaagcccaccgtgtaaatcaggtcagcatgagttcctccacggcagcctctacttgggttccctgcaccctgggtgaccattcctgcccagtacctcatcaaacccacctcccagtgccatcagaaaatgatggggctggctgtgttcgctccctgaggaccaggcctctttctccaccgcctcctctccccgcagctcgctcttggagtgcatctcctgctgggtgcgctacaaacaatctccatgtgcctgctaacttaggttatgatagaatcctctgaatccacttcagtaaggacctgtgggcacaagaagtcaggggacaggccaattgggatgtcacacagtgtgaaagaacatgaggtccccacacatacagcaaaacagcaaccacctaattcaaaatggtactgaaattgattgccccacagccatcctgttctttgccttttgagctagtgaggtctggtgcatgttttatgggagaaaggcaacatttgagctggtgtaaaaagggcacagtaaactttcgccttctgcagtacactgaacctcttaaagacgcttgtgtctggcatggaaggaaaaagtaccatgttttccttccagtattttcagatgtaattaataaatggaaatttattataggggcccacttaaggtctcactgcccctctgctgggtctgtcttgaatgggagtcactgggagaagggtctttgggcttggcgcttcccctgggtcttcataggggctgaggcagggctggtaaggagcccctgcagggggacacaagggtggacagggtgcagacacccccaggcctgggattctgtggacagcatggtgtttgtatcttgaaagggggtcttaaggggaggtgtggggccagcccaaactgtgttcaaattctagccctgccagaccctttgtatgtgaccttgagcacatgacctcattgctatctgccttttctcaatggtacagtagagatgatggcaacacctacttcctgtggctgatgtgaggattaaacatgttaatatctaaaacagtggctgggacatgggcggtacagctttaaaatgagcaatcctgctctgctggaggaagtggagcagagctagcgccattcttggcctcgcctcagttgctggggctggttggtggccccacctcagtccacacggcccccaggtcccgtggtctgacatggcttactgtccactcccctctggatccacggggagtcagtcgcaccccttagctgtcaccgcctgcacctccacctcaagccagcatttataggcagtcccttctgtcccatgccaagagctgacatgcctaacacttcacagccctcctgggagaaggcacccctagttttccccaaaggaagtgaggcccctgactgctcaggaatcctcatccccatgccctctcagagcacaggttctcatgtgggagtcccccaggctctctggcaggttaccatggacctcccttcctgcctgttctctgttaccagcaggccccagagctcggctctccttctctttctctaccttattttcagttggattttctttgctaacattggccttgctctttcccaccatttcggcttggcctgtgtcctggccttctgatcctgtggctggcctagtctcaggttctttgagggtgcctggaactcccctggattttgcagatttctatgggattttttgcctctctccctaggcctgcagtttctgcccagttttgctgcaagttccaataagctgcagcctcattcttttgtgctcaccgcagtgcacctttgcttcgtgctgtctagtggtagaaaggtgaaatgcaccctgactgcacccctcacagggagatcatgctaaaataaatacttcacaaatagttagatgtcctaaggcatgttctctggtttttaaaaaagtcctaggaaagacctttttttaaaatgcacaataatctttcttgttctaatttgcacacctggttgctggaagcagtgaacgcctgtgtcttaggctcagggctgccgtattgattctatagggcctaacagaagtcccactttgcagaggaaaacagtgatgtgtgttgaaaatagatggtgcgtgaggcctcagggaccatttggcctgacagccccagctgaggacagggaggcacaggaggtggagagggagaggcaacatggaagttgatttcatttgccagctgtccatggagcttcccagtagttggcgaagatggaacgtggcttcctgagctgcctgtctttcccgtgaaaggttgacatcacagagtagtccataagcaacagttttcagtggggccacatggaggtcccgaagctcttctgccagcactgtggaggggaagcgggtgacatggaagaggtcacgtggactgcccttgcttcagtaattattcagtacacatattatccatcttgtggacaccaagatgaataaggaacagcacttttctacctctggcagggatacaggcttaagaaatacaggtgtgtaaggctacatttaaggaagttcttggggccacctggggacacccatgcagctggcgtcacactgagctgaatcttccttgaatgggacgggcttagagggcaggtggacatagagaggacagtgcttcagggtgggtggcaccttccctgcagaccaaaagcccagtcaggccacggcgggttagggagagtcctgcgagcctggccaaggggcctgggcttcctcctcaaggctggggagaaccattccacggttttctttgggggagtgacaaaaatgcaaggtttttttggaaggatcacttccgaattcataaagagaccaggctttgtatctgctgtggggatggtgctctgggaatttgtctgtcattggacaaagggcatttcctgtaccagaaacctgcttccaggtctcacatggcttatgtcatgatattaaggaggagggaaacccccactgagaataagtattgtattattctgcacatctgagatgctaataaaaggaatttttattattatagtaaaacatttcagttagatagattaaccgtattagagcttcaataaagatacttttcttaaattccatttttttctttgcaatattatcataagaggtactcctaatcaatgttagctttttattttgaagtgattttaatctttacctgaacaatacaaaagaagtaccgaatactgccatgtgtccttcacccagattcatcgatagctaatgtgtggccacattcatgctctcactctctctacactaacacacatttttgtgaacgatttgagagttggttgcagacatcatgcccccttgcctctaactacatcagtgtgtatttccttagaatgaggacatccacttcacttctgtaaccacagcacaggtatcaaaattaggagatttaacattaatacgatatctttttttttttcttttttgaacagagtctcgctctgtctcccaggctggagtgcagtggtgcgatctcggctcactgcagcctcccaagtagctgggactacaggcatagcccaccaccatgcctggctaatttttgtatttttagtagagatgggatttcaccatgttggccaggctggtctcaaactcccagcttcaagtgatccacctccctcccaaagtgccgagattacaggtgtgagccaccacatctgaccatcattttgtaatcaacaaaccttactcagattttaccaagagtctcaataatttaatgaaataatagcagtaacaataataataaataatgatattggtaactctggtcagtaatctaatccaggatcagccatgacatttaacttccatttctctctatgctagaacagttgctcagccgaccttttttgtcatgaccttggcctgacattgactttgtagaacacccacactcagagttcctttgaggttttgtatttggagcaacagcagagaagtgatcatgaatccttccttctcagtgcatctgtgatgttaactctggtcactagttaaggtggggtctgcttctatatagatgccctttttcccttggtaatttataagtaaattccaggaatgttttttgaaactgtacaaatatcctgtttttttgtttttttgtttttttgagacagagtcttgctctgtcgcccaggctacagtgcagtggcacaagctcggctcactgcaacctcctcccttcaagttcaagcagttctcctgcctcagcctcctgagtagctggaattacaggcacctgccaccatgcccagctaatttttttgtatttttagtagagacggggtttcactatgttggccaggctggtctcaaactcccaaccttaagtaatccgcccaccttgacctcccaaggtgttgggattacaggcgtgagccaccatgcctggcccaaatatcctgttcttatcaaaactcacactctagttttcacacaccagtaggcttgagcaataaatatgtaagggagaaaactgctcaatcttttgactttctaaataccatgattaaaataaaataggtggcctatgcctgtaatcccagcactttgggaggcccaggcgggtggatcacttgagtccaggagtttgagagacaagcctaggcaacatggtgaaaccctgtctctacaaaaaatacactgggtgtggtggctgccacctgaaatagcaactccctgagcctgggaggcagaggtcacagtgagccatgatccagccactgcactcctgcctgggcggcagagtgagaccctgtctcaaaacaaacaaacaaacaacaaaaaaaagaaaatggatgtaagttctgtgtagctaggtggggtgggctggagtcaaaagaagcaggcatatctatgaaagctgaatataatagactgtacatactgaagactttatttttgttaataaaagcaaatggttaaaagttgttttacatctattcttcaaatatctataaaatcagttgatgttccttatggttaacagcacaggctgccccataaaaatacctgatgaaaaaacaattagaaaaagaaaatggagatggtgaggctgctttgtcatcataaaaaatgcatcatttcatgctcttgataataaaagcacatggaggaccagccacaacagcgacctctcaaactcaccagcacaccaagaaatcggggcttgagccacatgaccatttttatctaaagtgttttttcttacagaaaatgaattaaaaggccacaaagtaattaaggcaactttttttggtttttaaattttttgatggatatataatagttgtacatgtttatagggtacgtgtgatattttgatgcaagcatacagtaagttatcaaattagagtaattggaatatccgtcacctcaaacatttatcattcctttgtgttaggactattctaattccagtcctctaggtattttgaaatatacaataaattattaagttacagttgccccatggtgctcctcaatactgtatcttattccttctgtgcatctgtacttttgtgcccgttgatcattctctcttcacccacctccccactattcttcccagcctctagtaagcatcattctgttctctacttccatgaggtccactttttagctcccacataggagtgagaacatgtgatatttgtcttcctgtgcttggcttgtttcccctaacacaatgttcgttctatccatgttgctgcaaatgacaggacttcatttttttccactgctgaataatattccattgtgtatttgtgtaccattttttttattattatttcttggataggatgtctttgaaacttcaaatcctttgaaaatgtgaggctgaacaaaaaagatttttgtttccaaagttcgcctccagtttgaatgtattggaagtttgttgttcatctctaaggacccttgctcttttcagaagaatatggcgttaaacataccttaagtattagagcattttgttcttatcgttgctagttttatgtaactgagagaaaatattttgaacgtcttagcactctctcaggaatcagtagaaggagcagagcgaggaggctattcttatgccaaattaagacttaaatgagtcaacacgtgtggtttgtgagaaaagcacactaggttttaagaggcagaataatgaagttgttcttgaggaaggttcctttgaaacactggctttcttattttttagttttgcatgagaatgtcatccatgaaggtgtgggggttttttcacctttaaaataatctcatactttttttatcctgtcatcttcatggcataaatggaattaaatcaactgtatgcataacattctcactacacaaagcaattccatttttaatttgtgtttgtatttgtttggataggaaatatttgtggatggcataaaatccaaaagatataaaggagcagggagggaagaggctgctcctgcccagcacccccagagtccccagagacagccagtgtggtgtttcaacagaatggttggccttaaactgacttcgagtccccaaagggcgatcttctcaacctacttttatcttgtagtgaagagaatctgttataaaatgttaccgtagttaatcttgttagtatggggcaaatatctaacctgaattgccaagtaaatgttttccaagtgaaatttaacaatagagtaaattagctggaaactgcttgggtgctgagaccaaatgccggagtacttaattgcaaaaatatagaacgattttattagacaaccatgaaatactggtgttttaaagcgtagaagctgcagacttcctcactgagggagatcataaattgaacagttaagccaggcaggacgtttggagggcatttgatgtaggttcagcgtggctaataacctgggatggcatttggtagttcttcgggacatttctttaactttacaggtccttgctacatgtcatctttctcacaagagtttcctgttcaatctgtacccaacctgtattcttccttttggctttaagacattttgtccctcccatacccatttgtattttacagacccagtttgatatttctgcccaggaagaaatccctgacttccctcccatatgccatgcttgcacacttgcattctgtgcacccttatgcttgcacacttgctctctgtgtgcctttacatttgcatgcttcctctctgtgcacccttatgcttacacacctgttctctgtgcccccttatgcttgcacacttgcactctaggcacccttatgcttgcacatttgctctctgtgcctttacacttgcatgcttactctgtgtgcacccttatgcttacaaacctgctctgtgcacacttacacttgcacacttgcctctgagcacccttatacttgtacccctgccgctgtgcacccttttgcttgcacagttgctctctgtgcaccttatgcttgcatgcttgctcttgctgcttgcatgcctgcactgtgtgcatccttacacttgcatgctgtactctgtgagcccttgtgcttgcacacttgcactctccacgcccttggccttgcacgcttgttctctgtgcacccttagctcatacacttacattctgtgtgctcttgcacttgcacacttgctctctctgttcccttagtttgcacaattgcgctctgtgcatccttgcacttgcacgcttgctgtctgtgcacgcttacacttccatgcttgctctctgtgtacccttatttttgcgtgctccatcccaccaggtcttgctcttaccatcttcaaaaccctgccttgcctgcttttttttcttcctctacctgaaacactaagatttccaagaacacagtttgcatgctgtgtagatgtccttatttaaatattgatttgccaaaggttagtcttgagtacttagtaagttcttagtaagacccaaataactatagaaactagttatttgcccccttttaagtccctccaattaacaaggctagacttatttagaaagcacatttatgtgtcatatttgacattttttatgcctgacctttgattcttaaagctcatagtatgatgagtcatgtagtttttccactaaacatgcctttactgaggacaggaacttgacatggcatttggcatatagttatgctcagtaaatgtacataaagccacgtatttatcaacaccgtactcaggaccatgagttacacaaaggaagtgttagttatgacctctggcctcaaaagccttaccacttagtgaaggaaaggatgtctgtaatatataatgtataggccgggcaccgtggctcacacctgtaattccagcactttcagaggctgaggcaggaggatcacctgagggtcaggagttcgagaccagcctgaccaacatggagaaaccccgtctctactaaaaatacaaaattagctgggcgtggtggcacatgcctgtaatcccagctactcaggcagctgaggcaggagaatcgcttgaacccaggaggcagaggttgcggtgagctgagatcacgccactgcactccagcctgggcaacaagagcgaaactccatctcgaaaaaaaaaaggccaggcgcagtggctcacgcctgtaatcccaacactttgggaggccgaggcgggcggatcacgaggtcaggagatcaagaacatcctggctaacatggtgaaaccccgtctctactaaaaatacaaaaaattagccaggcctggtggcaggcgcctgtagtcccagctactcgggaggctgaggcaggagaatggtgtgaacccgggaggcggagcttgcagtgagccgagatcacgccactgcactccaacctgggcgacggagcaagactctgtctcaaaaaaaaaaaaaaatatatatataacatattattatatatattatatatattactatatgttatatataatatattatatatattatatattctatattataatatagaatatatatattctatattataatatattatatataatatataatatatataatatatattataatatagattatataaattatatgtaatatatattataatatagattatataaattatatgtaatatatattataatatagattatataaattatatgtaatatatattataatatagattatataaattatatataatatatataatgtgtaatataaaattaatgaaaaataaggcatgtagtttaatgctaactcatgtggtatagactagacattctgtagccattcagaaaagaagattaataaggactattgtgctcagacaaaagtcttcattgttgcacttcgtacagtagggcttcctgggaaggtaaaattgtcatcagcagagcctagtcaggaagccatatccccatcacaaaatcaaggccactcctagcatagcagcatcagtttctcaggtctgggggacagtaagctttggctagagctcaagaatgagcccttggtctacaaacttgttccatgatgttcagaaaaatgaagagcatcatcttttagcttaaagtgaatccacagttgtgggagttagtttattacacatgcacgtaattacttagtgtttaaagaaacgttggtaaagaatatgttcaaacaaattgaagtatatattttttatttcatgaagcacgtaagtttatggcaaaaagataaacaaatgttggttgcttcagtcatttccatttccattttctcatcccaagtggaggtaaaatgatccttactacaatcttgcttatcctacctatttacacacatgtctaaaatatacacacacacatgcacacacactggcacatgcagaggctgaatcatgcacaaaatacgtaaatgatgaacatttttttaaaaatattaccaaatattgatgggatatggcagtgttgtttttgaaaaatatgtaacatgactttaatatttttatagttttcagaattagaatcataggaagggaaaatgttttaattagataattcaactttttatgtgtctgtagtggtgtactataaaagcaaattataaagcattattaaatattcataataatttttaatattacctgcattatgaatttaactaaaataaagtgagttgtacatttttaattgagttgtttcaatagctggaagcatcctgaagcattatattaatttttgaactatttgaattcaaactgagtatgatttgaaaataaattaataatttaaaaacaaaaataaaaatattaccaaatattaaacttacctcaatgattattcttcagaaacatctgaaaagaatgatattttcatagcaaaacctttagaatcatctctgaaaaaagaaaaaagggaaaacatattttctcttaattttcctccctactgccaccacccatgtgagaatcatatgagtttgaccacccattattcttgattgtttctagagctgtctatgcaatctcagtcaaacgagtaatttttaaccagagtatttgtagaaaaataacagttatttgagttttcatttttattaaaaataatgttaaagattttatggcattattatcaggttgcattttttttaatccacaggagtgtaccattaccctaaagaataccttttaaattattgggaggttcctatctccattttctcaagcttaaataatctccttaaataatctaaattttagatattatgtaagtgttctaatactttacatattgaatgaagatatatcattttaaggtatttagttttaaatttaagttttttaaaaatagttcttaaggtgttatatgtttactttttttcttaaatcacgtggcatcagctgacatctttaactgcttgaaagaattaagccaataaatattatcatggccaggtgcggtggctcaagcctgtaatcccagcacttagggaggccaaggcaggtgaatcacctgaggtcgggagtttgagaccagcctggccaacatgatcaaaccctgtctctactaaaaaaatacaaaaaattagccaggcgtggtggcaggcctctgtaatcccagctactcaggaggctgaggcaggagaattgcttgaacctgggaggcagaggttacagtgagccgaagtcgtgccactgctctccagcctgggcaacaagagtgaaactctgtaaaaaaaaaaaaaaaagaaaaaaagagattatcattagtgtttcttatttctttggttaaaatgttatagtgagtagtgtgttatatcatcatgtgaattttcataacttattggaagttaatgttaatgttaacattaactaattggaagttaatgataatgaaattaatcattatcagttttatatgaacaggcatttcatttttttttctaaatgatgacctgatatgtgtcaggaactatgttcagcattggaaatactaagctgaattgtgtacacccacgggaattctcacagggctcacagagcttaaacccctaggacacactgtcatgttggcaaggggaaggtggacacagccctttgggagaaatagtggtgctatcccagggaaggtgaggggtggcctggcctttgaagaatgggtgaattcagcaggtagaccagggacagtggcaagtgcagtatggccagcacagccctgtgtgcaaatttttttgttgtttgtttgttttgagatggagtcgcactctgtcacccaggctggagtatagtggtgcgatctcagctcactgcaacctcctccttctgggttcaagcgattctcctgcctcagccaccccagtagctggaattacaggtgtgtgacaccatgcctggctaatttttttgttttttttagtagtagagatcaggtttcgccatgttggccagcctggtctcgaactcctgacctcaggtgatctacctgcctcagcctcccaaagtgctgggattactggtgccccgcccctgtgtgcaaatttggacaccgcagaggtgctccttctcagaagtcccccacatcatacaaatcatttcaagagcatctagccctacaggaagatcatggagagacattcttggacttttttattacacttgctccagtgtaaaattttatggtatgaatacagtatgactattatagcagaaataaataaaaataccaggacagataatagaaaagagttgtaagaaattcaggaaatgtatgtgcttcaataaaaagaatgctttaaattgtcctagttgattttattgaaggtaaatttgaaattccatacttagtatttaagtcaaattaccactcatgcttctcttgtggttctctttactgagaaattcaggagactgcatctttgcaggtttctcacttcaattccatgcccatcagtttgcccccagtgccgtcctgttccaggtgatcatagggccaaatagagttgatgtctccattgtgttttgaccaagcttccccatctgtgctcttttggtggctttgcctctaatcttagcatttcagctggaagcgcacttaggtgtgtctgatccaagacttcctctacagatgagcagggcattagcgcaactctctacgagacttgtccagggtcatccagatgtgctgacctggaggcaaagccaggactagaaccgagtctcccgattcctcatccagttctcttcttgtcgcttccattcatgcagggtgcgggtctgcatttatacagcggagtttcattatctattaggttgaactattaaaaattgccaatatttggccaattttgagctctgatatggcagtttcttacggttcaacccgttagattaaatgtcacctgtcagtctctcattacagtcccttacatccctgtcctgcgaagacagcattgccatcgagtggtaggtatcctccctgcggacagaggccccgtcacagtgggcccagccctgcccttctatgctacaggctcaggtctgcacctgcctttgtgcttcactctcagcagtgacatctgctgcatccctcacttgccaccatcatctctactgggagccttccctgacctctgagatgagaggcagccagttctcccaaagacacacagcccctgggttgtctggcaccaaggtgtccccttaccagggcttgcacgtctcatgtccactgcccacccatagttacccattttttgtggctaagcacataacatgctcacttacctacaattgcaacaactagaagttagagcagtctgtttaaaaggctttgtgtaaatcaccctgaaagattcctggtatgtgttggagcaggagataaacgggatgacacactacataaccagtctctgaaatggtccccacggtaggcttgatattgtttcttaaggctcttttttgaaaagagaggaaacaaaatagtaccaaaaatctcataataggccagatgcagtggctcatccctttaatcccagcacttggggagtccgaggttggaggaacgcttgagtgcaggagttcaagaccagaccagcctgggcaaaattatgagaccccatctctacaaaaaatcttgtataaaaataaaaaaaatagccgggcatggtgttgtatgcctgtggtcccagcttttcgggaggccaaggcaggaggatcacctgagcccaggcagtcaagactgtaatgacctatgattgcagcactgtgctctagcctgatgacagaggaagaccttgtctcaaaagaaaaatctcacaatagttttacgcatatgattatataaaaacaatcgcatctgagtaatttcggtgactagagtaaaagttccttaacattatggttaagagtgcagactttgcaattggtctcacctctgcctcttcctgtctatgtaaccttggttgagttaattagtatctctatgctcagtttcctcatctgtaaaatggggataataataataatgcctagcacatgaagctttttgggggaattaaatacaataatacatctgaagtgctgggccctgtgcctgacacaccttaaacattcagtaaatgtgagatgtcatcctggtcacccttcaccatgcgtaatgttatgtgtgtgtgctgccttttcagacgttgccgagtgctgaagccacgcacggggcccagtttgactcggcacgtcttggtgtggccgaagaacaagcctggctctgaattaagagtcaggctgtggtgttttcagggaactttctctgccttctgtcagttttcaaggcttggcattgcatgggaagagtcaacaaatactatattctgcagaaagtattgtggtctgaaatagtgtagactaaaatataaagaaaaaccaaattgggattttaaaatgcatgagcgcccatctaaaagagccacctacgccttggttttcctcttttttggtgaagttgataaattggcttgataccaatgaaaatggcgaccactgcttgaacagcacttcatgccaagaactgccctaagctgtataactgcagcaatcttatgggtggagaatggtggctcatttcatatgtgagaaaacagaagcccagagaacataattgacttgccccgagtcatagagctgggtagagaatggttcagaatttgagcccaggcagcctgaccccagagcctgtccccctgtgtactgtgaggtgacctttctgggtttaagaatcacgggtccaggttatacactgtatcagccttgttgcagctacaagcttggtgtggtcacttcaccatccctcattttgatttttgtttgcagagtttaggtcgatagacattgcgggtatatttctaagtcttattgaggaaataatcagtccatgagtgcctgtagctcactacatgagccatctatctggagatgggcagggctcacacagcacaggccatctttcagttctgcacctgatggacagcacctcttcctgcggcagagcctgagtggccatcgctcggctcagcccagtgcgaggcagagagtcctgctgaatctccccttgactaatggcttctttctaattcctgatttactgaagacagcctttctgtcttgaaggaattactctgtggcatttcccagtcttagcaagtggttctggggggccctgatgaaagcactgtgttctggcgaactgagaaccaactgtggacccttgctttgtttgagggccaggggtggccaggagggtttctctgcagagtccacacctcacccatatgcacacactgggaggaaatatttttcaaccattggtttaaccattgaatagttagctttgtagtacttactcctctgagattttacgcagatgatctcacttaatccacacacatgcacgcccccgtgtagtagccaccatggcggtcccctgacggggtagatacgtggcccgtgtcacacagcatgtgcagcagggctgggacttggcgccagggcctacactctgagccacaatgtctaagccctacctcacccaaaaagattcctctaagtctgtgtcagattagaaatgaagaggggacaaagagaagtaccacggacagcaagaaaagaggaagatgcacatcagaactcataaagcaagtccctcaagaggcattctgtgtgggggcaaattcccaggggttcccggtgtctacaacacaaatacagtgactgacgcaagatcctatgtcctcaaggagagagaggaggtgaaattccagtgttttcatttcgtaagaaggcagagatgtatggaatgcttcaggaagatttccattcaagagtttatgtgggaaaaaagttttattgctgaaagaatgatgtccctaagtatccagacatttctgatttggatggctaggctcgccagaatgtcattaactgggatgtttgctcacctagcgtttaactgtcatagttcaagcctagagaaaaggtgtctccggatgggagtgatttataattcgcccctccattgtcatccatggttgcccaactgatagctaaaaaatcaacagcatccgcctccagagcgcctctttgctcaatatgaccttgcgagtttccagtgcaaatctgactctgctggcttctatgccactttctcttttttaattcagttgcccatgctattctttgggtttctctagccactttggctctgacgatacaaaatgtctttgtagataacaacaagatttcaacttgcttattagaaaaaagagttacatactcttcagaagtttttttaatatataaagaatatgttaacaaaaagagggttcacacttgtaagagcgtcacactctgtggtgtgtagaataagaaataacttccataataagaatttatgccaggcaaagtggcttatgcctgtaatcccagcattttgggaagccaaggtgagaggattgcttaagcccagcagttcaagaccatcccaggcaacatagcaagaccccatctctacaaaaaaattttaaaattaactacccatgctggtgcacacctggagtcccagctacctgggaggctgaggtgggaggattgcttgagcccaggagtttgaggctgcagtgagccgtgattgcaccattgcactccagcctgggcaacagagccagatcctgcctcaaaaaaaaaaaaaaaaaaaaaagaatttacaccttgcaaatgcaacagtcttctcttcatccttgtactttgtcaaaaactttaaaattgtgtttttggaaacatgcttctcttcacgactaagacttgttactggattgtgtgctgcgggtccctttccaactgacaagcttctctacccttcgagctgatagtcatttgtgctttccaagaggtcatcccaagaatgcaacgaaaaaaatcaatacatcataaaagcatgaaggttgtagtatcaatgtaaaagccattttaactttcctcaacatcagaattggaaggacaaatacatttctttccacttttctatccctcggtcctctttaaaaaaattaatatatgatatgtatgaataaatgcagagactgttagtgtgattttgtagatggtggcagagagatgggagatggtgggctttggccaagttttcagcacatcattggtagaaccaagactggaagctacatctctttcttccaggaccggtatcattactgctcatgtgtcagacgacactgaatttggtgagaatttctcacttctaagggataatgaaccatacaaaaaaatcacagtcagctccctgtgttactctagctgacctgctttaggaactctgccagtgctggatctgcactctctttgaaggcctcctaacaggtattcagatgactcgttaagctttagagtgctgtgtgtccagaatacagcaatacatgctacttgacatgagaatataattcaaggcatgtaaacagagatacattgatcttctaagcaatatcttcatttgagcaggacagttggcatgttgatgaaaagaggcttcaaatcttaacagttacaaataacttttccttttgcctgtaatgtaattagcactcttgaagtattttgctggatgcctttcttcatgctcttcttagcaagttgtttagtcaaaatcaagtaggtaaactagatgggtatatacatatgttaaaagttgtttggaatttcaacctacccggaagagcagaccctcgcctagcactcgttacatcctaagagctggagacatacgagctcctgggatcttcatgacacccccatcaggaggagactaccagcatccctgtcttacagactagaaaaaggcagtgcagaggtgaaggtcacacaactagtcagtgatgggttgaggtttggagtcccagagtctggttccagagtctatgcccttcgcccctcactgggctgcctctgggtacagaatcagcacctccccaccagcttgtgttgtgcctgagattccatggcatcactcgctttcccctgcaagttaccatgtcgcctccagacatgtgctcttcggagagggactctcatacttacccatacatatgtatggtgaatagtgactgttgaggaagggagggagagctgttgttcgatttcctgccttaagagttgaaaagtttttcatttgaccactagtagttttcaccctcagaacagacaaccatagttgaagatcagagaagggttaaatcctcaggaacatctgaactatgacccacccattcatggttttataggtaaggttacttgtgtgctaggttaagtcccatttactcctgctatttttagttatttcttcaaaaatggtgtcagctgcaggctgagcgagcatcccctaggtccctattcaagtttccagagcctggagtaacctctgtaaaggccgcatgatgttacagagaaacaccacaaaaacagtcttagcttctcctcctctcacaggtattagtgacgaaaccttgggaaatgtgactcagtatctggagctttagttgcatcctcttgtataaatgaaagctaggcaaggtgatctctgaaggcccatgcaggctctgagaaccaacagaattatgacaaaggtgttatggggtcagtgtggtgggtgcatagaaaacaaaaatcccctttcttaagaacccatttttatcagttcccatcatggattgggagatacattccaggaagaattttatagatagtaaccttatctataaaaccatgagtgggtcatggttcagatgttccagagaatttaacccttctctgatcttcagcaatggttgtctgtttcaagggtgaaaattactagtggtcaaatgaaaaacgtttcaactctgaagacaggaaaacaaacaactgttctcccaccctgcttcaacagtcactgttcagatacatttcaagaagaatgtatctcccaatccatgatgggaactgataaaaataggttcttgagaaaggggattgtggaaactgtgaaaagagccagagacagcacgtagaggtgaggaaggggcaactcttcctggcccagcaacagtggcattcatagagtggagaaaaatacactgcagtgtgaagaatgggcctcgtgctactgcgaaatggaaagcaggagactgcaaggggtagttagcatggaagcagcattctcgtataccaaatctataccttgctgatagtttgtcattgcacccagaatgtccattattagatttcttcccaccagtggcaaataggcattatctgtgctactaactgcacattacatatatgtggtaatgcagattgtgaaggcaggatggaatagcagtcatttgatggcctgtattgagactgatggatagactttcttacagacaagaagaacctgctgactaaagcttgtgtggcgggggttaaacttcctggagcagcctgaaaaggagaattatctggaataatggggtgtcctttcagttcactctgacctagctccaagataagcttgaccatcctacaaacaatcagatctaatagtccctcagttctttggggagtgaaaatgcgggtcaatgggaatggaaagatgggagttattggctcatgcactcattcaagtagaatttcctgaacacctcccctaggctgggggtagtctggaaactgggggatcacagtgaacaagatgcagtccctgacctgaagaacacaatttatccaccaaacttttgctgagagtccatcattttcacttgaatactcttgcagttttattcatttccttgcctcatactacaaaaatttaattgtgcagtcattaacaaaaattaggatggaaaatatcaacctggggttgttttttccactcatatggagaaggcttcttcagcttactgagccagtaaaagatgtctgtcatcatttgctgttgattgtggcatatataccattgcaacatgcatcattagcgcagttagtaccttgttacagcttttggctttatattcttactgtgtcttagggtctcagatggtttgcgttacatttctcagatgtaatacaagtatatctctgtgacaagtttctatttttaatttttttaacctctttttatgtgcagttacctaaaattattctgtagggacttgactccaatccctgaagtagaggcatttttgcatagtcacgttcctgctgtcttgtccagggacttttcctgtgtgctgtgtttatatatttgattcctaacacgtaagacctttttcggagcaaatatccaagtttgtttttattatggctcttatatttaaaaaaaaaaagataattttttggtcaaatgttaagacatgccaactctcggttttaaatattttaaggtcttcaaaaaatgaatgtttacagttttgttgccagaacctctctggctctttaaaataaaaatatttcagctgtaaattggaagataagtcctctgtccaagcagtttttcaaacctgttccagtcagctccagtaaagtgtggtacaatggactgaacccaaaatgaaaaagactggagtccattctcaaccagacacagtcttagccaatctgcctttgggttttcacgtctgtaaaatgattatattaataccaaactcatggagttacactgaggaatgaattagatcacaggcatgaaaaagcgcggtaaggtgaataactcagtaaatgtgaattattattgttattactgttgaccaagttcaaatactagaaccagaaaaggacttgaattacaatgaaaattagccagaaacacagaagactttgacttacacatttctgatcactccaaccctaggagtccattcgtaggcaagacttaagtcacactgaagtataaatgactgataacaggctactaatctgttactctatcaaatgcctttgcatttaactgactgagagccttgactacaggattgaaattctgcaagataaattttaaatatcaaccagtgaatgatgatagtgctgaccagaactaccagtgtttcttacggctatttcaaattaagactttaaatatagggaaaatttggttgttttaaatctacatttacacttacaggcattcttttcaacatgcattatctgtggtaactgcaaataaagcattccccaattcgtaaataaaaattggacttagaaacacctaaccattaaactacagctctgaacaactcttaaatttcctttgtattacccaaaccagaaagcccaaccttggattgagagagccttaatatacacctttgaaaatatagaatttctgtatcttataatagatatgcacatttaatgtgatagcatttctcctcccatacacccccacagcctcaaaaatctattattaaacactgctacatcaagaatagagaaaaagcagcattttgtagaattaaacatcatattgcttccaaaaaagaaaagagcagttgtagacatttccttagttaattgcataaatctttaaaaacatcattttatataaggaacaaattaacaaattactgagctctttgaagataaactttgtgagaattataagctactttgtgcatttaatcattttatatactgaggagctgtatttgataagtaatatggaaaggaaattctgaatagtatacaactgaactgccaagctactggatctctgtttaatcctcttttgaggaaaacttaggagatgctattgccaaaaggggaggggtcacactgtattttaaaatcaaatttgtttaaataaacaaaataggtgttataattagcaatatataacaggaaacatgttcaagaatattcatagtggcactgtttataacaagaaactggaaacaacccaaatggccactaacaagacaatggatacataatatgtggaatatccacacaataaaatattcatcagtgaaaaggttggccaggcgcagtggctcatgcctgtaatcctagcaatttgggaagtcgacatgggcggatcacttgaggccaggagttcaagaccagcctggccaacacggtaaaaccctgtctctactaaaaatacaaaaattagctgggcgtagtggcaggtgcatgtaatcccagctgcttgggaggctgaggcaggagaatcacttgatccagggaggcggagcttgcagtgagccaagatggcaccattgcactccagcctgggtgacagagctagactccgtctcaaaagaaaaaaagaaaaagaaaaagaatgaattacagccacatgcaatgacatggatggatctcagtatcataatgttgaatgaagaactcaagtgttaaaagattacatataatcctcttttgtttgtaaggttcaaaaaacaagcataactaagtactatattgtttaagcatgcatgtatttgtgataaaaatttttgaaaggagacagaatgatagtcgtaacattcaggatagtaggagagaaggcaagggtggaagagagatggggtatggaagagatcatagtaaaatattggttattgatcctgtcttattaaggagatgggaggggctcatgggtgttccttttatgattaaacaaaaaaataagaaaatgaaaaagggccatgtaagagtcaatgatgactgtgtgtcattaaccagatctattcacctgtggtccacattaaaaagaaaattcgttttctagtttagaaacatgtatctttcttcgtgctgtgttgtcttttaaaggaaaaatgtacatagatgctactgaaagggctatgtgagcataatgcatgataaagagaatcataaatcttaagaattttgagatttgtatttattatgataggtgacaatgcttcaagatttgcagccacaaagtcactgaggttatatccccataagccagactctgactacgcaaaagacatggtagaatatcaaccaacatttgtaaaagtaactaactctgtcattctgtaaatttcacatcgcattctggcagttgttcttttgttaattagactaattccagaggtgggtcattcccattactttttcgttctgtatatattccaggtttaatttgttgaccacctcacattcaacagaagtgagtgtgaaacaaggctgctccctagaaccaggccacctgcttttagagcccagctcccctcttccaagaggcagtgcctctgtgtgcgtcagcttgttcctttctcaaatgggcatgataatggtacttacctcagagttgctgtgagggttaaacaagttaatatatttaatacataaaagactaggctcaaagtttcaacatcataaatactctatattagctaatcggaagaaaaaaattattaaatcagacttttatattgtgcctgcaaagccaagaaactctactaatctcaccgtgatggcatcactacacttaactgtggtgagacttcttttaatgaatacatcctcactccagcactgaccaaacatagaaaatagaaataaaatgtgagccaccaatgcaatccacctatgtaattttgcattttctagcagccacatttttaaaaagtaaaaagaaatcagcaaagcttaataatatattttaacgctatacaaatatccaaaaatattatttcagcatgtagtgtttagaaatttccaatttctatgctctttttttcattaaaaatgtcttcaaaatctagtgtgtatttggcatgtctggcacatctcagtttgagcaagccgcatttcaagctcagtggccacctgctgttagtggctactgcagagaatatggagaaaggaaataatgacttcttccttcccagcatcagtgttagtggggaggaaaggcacaggccaggtgagaagctccatctgtgcatgttcattgtcctgtgaggcaggagggtagccagctactccaggccccctggtacctgctctcagggtgagtgtccctcatgtccacgtggcccagcaagcccatgcagggcagagagtagcgttgcctgagtaccggacactgcctggtgcttagcagctatgcttcctcatggtggggaccagcgtacttgtctttggtctcccagtgctgacaagagcctgttgctcttctggagacttcactgcattgtgtttctgtcttcaggtcagcaggcgaggtgtccgccctaatgggccttacccctaatctgactgcttctaagtctcccttgtcatccaccatggcctcagcatggggaatgaaaccaaccacgtggccacaggtgttgcggcagccaactgtgttctctgtgtgtgcaggagatggaatggtgaggccttaccttgccacaccttcatgatgacacccctgcacttaccaaagctgccacaagcctaatgctgtgcttttgaagcctattcttgatgcctttgcccaaaaagagccacatgattatgtattgcttgagcagaagcagattgtataattttgttcttttcattttattttctgcatgaaatttttagggcatttagtaattcactgaaaacatacaaattagcattttatttctaagtcaagaagttggctgtggtctgtgtgtgtttaaaaggaatgtattatgaatctgagtaaaagaaatggagaaagattttttcggcctcagtggaattgaaaaaaaccaaaagccagtacctggtctctgagcgtggaagaaactgactgtgcgtgctctgttggattatgagaaagctgcatgagcctggctgtccgtatgtatgggagtcagaaaagtgggaggaaatatatgattcatggtgtacaaagaattgagtttagaaaaggggaataaacaagaggaattactcagtcactactatttaaaactgagaatcagaagacaattagcacaatgttgttggaagaaagtttcagatagtgtaacatttttagtttctgcaaagtctggaattgctgtgattcatactttttcccagtcccacgctcagttttcgtcccctctcagccattggaaatgtgcacctgtctttcccctacctcaatccagtcaagtcttttagatggttttatataagatttcatcccagtggaatttggaaattagtctagaaagctgaaaatgagagggtacctgaaagttgggataaagatgaggtcattgctcctggattctgcactctttcccatgatatcatcctttagggaatccctggaaatgtggaatgggtgtccacaaggcagatggaagtatgggatgctccgtggtaagcctgtatgcctccaagcccccaataagtagaacaatgggaaatgcaaattgcagaaatctggaacaaggagaagaaaggaggacactggatgatgctgggatttaccaatgctggggttccaggaagacagcttctctggcccccgcccaggcctggaggctgcaggcccctgcctccttagaaccatggtccctggacagcactccatccagcctttcagagttttgttttcttctctttagcttacaattaattatcttagggaaaagaaatcagtgccagtgaattgccttgctttttttttcaatgagtcttttctaaattgggttcaggccggtgtatgagagaccagaaggaacccttctgcccagagacccagtctgacgccccctctcctgcgaacgtcagcgatggcctcggccatgaatgggttaagcagcagctcctctgctcagccccgtgttgagctgttattgaaggtctttaaaggcttccgcccaccttcctcccactcccctggcaagtgaaagacatttgaattcctcttgcaaggcagaaaattattaaagtgaaagaaaatgactgttccctgttctaaaaaggagagaaaagaaaagtggggctgcccctccttcattatttcttacctgagaaggagattatcagaaacagggtgaagcattctgactcccagaaatcaggagaaggggaagtttatcctgtcggccttttgtgtgcttatttcagcttataattcagtgccctggaattccgtgcacattagaatatggaggacctgctaaccctggaggagataaccatgattaatagggttatatcctcacagggcagtattactcaaagaccccaagtaactaaattatagagaagaatatgaaagaggagtagaaacccggtttatttttcctgtgcctttgagcactacaacttccactgaaaatcaattcattttagagggtcagatctgaatgagagattttagtaatggttcttttacacgtggctgaagaagcgtggtgggatagatcttcatacgttttcttttttttttttttttttttttttttttttgagatggagtcttgctctgtcgcccaggctggagtgcagtggcgctgtctcagctcactgcaagctctgcctcctgggtttatgccattctcctgccacagcctcccaagtagctgggactgcaggcacccaccaccacacctggctaatttttttttttttttttagtggagacgggctttcaccacgttagccaggatggtctccatctcctgacctcgtgatctgcccacctcggcctcccaaagtgctgggattacagacgtgagccaccgtgcccggcgatcttcacacattttcatttgaatctttttatattttataaacatcacttcataatttctctgttaatccagctcacctttatacttcccaatctaatcaaactactgattcaaactatgtctgctttaccccagccctgtccagagcatgggtctggccctgggaggtggagggtgagtgagctaagagggctattggaaaggtttttaaaataatctctggctgggcctgtaatcccagcagtttgggagactgaggtaggaggatcgcttgaggccaggagtttgagaccagcctgggcaacaacacagcgagacctcatttctacaataataataataatctctgttctagggcttatggtttaattaggaggattagaccaacagtcatgaaaagctaaagtcaagagatcatagaatttaattaaatctcagttttaaaatgtctctacttccccagacaaatataaagaaaaggaaaaggaaagcaagaacctggcaatggcagaagtctgttctgtatccacttcaagacaatgcattttacatctccttaggatgtataaatattagaactaaagtctctgccaatacatcacttttttggcaacagctatggtgctggaagtgacaatatgatcaccagtgaggtgggccgaaacagttctcagcatttacagtactatcataatttggtcataatgacatgttgcggaactcatataagatacagatacagaacatttcaagtgttgtttggcagccatcatgtaataaaagttaaactgatagcatgattacctgagagctattttcatgtgtatttaggaaatatttggccgggcgcggtggcccacgcctgtgtaatcctagcactttgggaggccgaggtgggcggatcacgaggtcaggagatcgagaccatcctggctaacacagtgaaaccccatctctactaaaaatacaaaaaattagccaggcatggtggcgggtgcctgtagtcccagctactcaggaggctgaggcaggataatggcatgaacccgagaggtggagcttgcagtgagccaagattgcaccactgcactccagcctgggagacagagccagactccatctcaaaaaaaaaaaaaaggaaatatctgcttaataggatcatgggcagccggcactggtttggccaatcccagagaaaaggaaaaaagtgtgtcttagcacccagtttcattgacattgccaacttcccatttccttctgcaaacttcccttttttatttcctgctgtccccagtaaatttatccttgaccatacctggagccattaaccgtaatcgggccttagatatcttacacacccctgagaatttctcctcagctgtggaataaacgtgttattcccaagttgtgctgtgcttcagaaccacctggggagctttcaaacaagatccctaagcctgtcctcagagattttgattcaggaattcttgggtgaagccttgaggaatctattttaatagctccccaaaggcttctaaggcaccaggaggtgtggcagtcacaacgcaacacctagtgtggctctcaaggcataaccagagttaagaggtggtcagaaactgacagcgaacttgcagcaatgcccttgttctgtgaggagtggttccccatgcagatggcctcatttataagtgtgcccccaccccattttctctgtacttacactccacaatcatgccaagaatcgtactgtcctatagtcctttttatgtttacaattcagaacaaatcttgattcattaaaacttattaacagttattaaaaatattgacagtgttggccgggcatggtggctcatgcctgtaataccagcactttggaagacggaggcaggcggatcacctgaggtcaggagttcgagaccagcctggtggtcaggctggtcagaagttcaagaccaacatggtgaaaccccgtctctactaaaaatactaaattagctgggtgtggtggcacacgcctgtaatgccagctactagggaggctgaggcaggagaatcgcttgaatctgggaggcagaggttgcagtgagcagagatcacaccactgcactccagcctaggagacagagcaagactccgtctcgaaaaaaaaaaaaaaaaaatatatatatatatatatatatacacatatatatcaaaagtgttttttcttaaaaatagaatgtgtaagataagttaaatgtagagtattagttataaagaggacttgataccacaagttccatctctaggcctatctcagtttcagcacctgctcccacctctaactagtctttctcgatgccacacaacctgttttcttgggcctccttcatttcaggtcacacctctgccaaccctgccacaacacagaggagcagttgaggccctcaatcactgtcctaagtaacagggcattgctcttacttgccaagaccttagctagcctcaggcaaaattgtggagtctgttgaaaacatccctttcctatcacatgttgctgccagatactcaggtatttaaatctgggtaagcaataagtgatacaagaagtgtaaagtggttttgaaaattctagccccagcaactgtcctctaacattacggactgtgatgatggattcccacgtagttggcattgattgaggatgatttgcaaacctactttaattaagcattttctcacaacttcttagctcatttgggtaactccatgtgcatataacttaaggttatggcaccatatggtggggaagactatatatagataggcagattccccacggatccaacaatccagttatcattgtatagattataagcataaacaaaatgtactttaagtggattgttctaataaagtaccatgtcttctttttcctccttatattttatcatcacctctgtcttagcatttgctaccagaatctggagatgctcttaggtggacacagagtcttacatgccattgatacatatactgctcatgtggcttgcagaggaaggacgtgaaaagcttagacccagaagctgggaaagggccttgttgttagtgtcagtgtaataagatgcagcttctagctcattgatgctgctgtgttgcagaggaaaagcctcagataaaaggattttgtttgaagtaaaacatcacaaaccggattccttgtaaacacaggaatagttgaagaaggtcagactcagatcactgatgatgcaaatgagaagaaagttccaggaagtaaaggcttaagctttctagaacgctccagtggcccaggaagggtgctgttgttcttagaacaatgagatgtcaaatgtattcaaagtaaaaattaaagtgggagaatggttttaataatttgaagtttacctcctcaagccagttgttggatgaggggttgagagctatcttttgtgcaagtgggcagggggtgttcagagccctgcccctggggtctctgcacaccattgttgggacaaaagaaattctccagggcgtctgtcagctcctcatgattcccccagcttccgacactctgaggaagaaattctccagggcgtttgtcagctcctcacgattcccccagcttccaacactctgacatcaatgtgtccttggggatgtgttgtttacaataatgtaacattcacactaagatggacttcatttttaatacagactcataaagcatttttaaccacttattttaattcagcaattcttaactgcaaaggaggtcagtgccaaggtggaaatcagtgttaacatgtttgatttttgcctctgaggcccctttgacacttatgtaaaattctctggcttccagcctggctgatggtttttcttttcttttcttttctttttttttctttttttttttttttgagacagtcttgctctgttgcccaggctggagtgcagtggcacgatctcgtctcactgcaagctctgcctcccgggttcacaccattctcctgcctcagcctcctgagtagctggaactacaggtgcccgccaccacgcccagctaatttttttgtatatttagtagagacggagtttcaccgtgttagccaggatggtcttgatctcctgacctcgtaatccgcctgcctcagcttccctaagttctgggattacaggcgtgagccaccgcgtccagccagctgatggtttttcaatcaaaactattccaatacttaatttaagaaaaaccagaaactactatttaggatttacagagcaagatatccagatttcaggaatgagaggtgactaattcccagagttgttttcaaattaaactgtcactttaaagctgtgaagggaaggccaaacaatttggttaagcatgtcctttcggcatggtggtggccatggctaaatggtttgtgtgggttcttaagccatggctaggatcctagtgaaggtttccatgaaagtaatgttgaaactctgaaggaagcaactagccacagttatttttaaatttcctgcctgtttgtttctttgttgccagccacaggaatgggaaccgctaggtgtttttccatagccatagcagaacaggctcccctgttgagcagtgaagacgcctgggttagggggtgaaaccagagaagccataagggatgctttttgccttctaaacagaaggttgattatcaagagatcatgcttgtttgtttgaaataaatgaactgatttctcccctgggtgtttctcttggtaaaacattttaaaagcttttaaagtgggggaggaactacaaccaactccacactatcttaataagatactaagctcagatagtaaacacacacaaaaaattaagctactgcatgtgatctatttaggaaagactttatattagagaataacagagttagcttataggtgcaatcatctagactggttagctgctgtagacagatatctatcaagttttctgcttcattgggtttcattgcaaggactcgaagtaggacacaaaggacttcggactcaagtagcttcccatgtctacctctgcagtgatccaggtgtgccgagaactcccgagggtctgcaccacatccatagtgcagcactgtcagcctcacctctcacaggtgtctcccctggtacaggtgtctcctgcacagttaagccagtctgttaaagactgttaatcattttgattatgactattgtcatttccgattttaatttcctgctaattttttgagttcagacccacctcttttggagacctttccaacctgtcctgaaactgagcaacctctccctttttggccacttcctgtggctttcctaattggcaccatagctttggtcacgaaattatatccagtacttactgccttgtattgtttccttcctccctccctccttccttccctcccttccttccttccgtcttaccctgtttcccaggctggagtgcagtggcgtgcaatctcagcttactgcaacttctacctcccgagttcaagcaattctcatgcctcagcctcccgagtagctgggactacaggcgcacaccaccgcacctggctaatttttgcatttttagtagagatggggtttcaccatgttggccaggctggtcccaaactcctgacctcaggtgatctgcccgccttggcctcccaaagtgctaggattacaggcataagccactgcacccagcttgtcttgtattgttttctaattacttggtatgtcttagccatagtctccctactgaagtgttgacttcttgagaacaggagtattggccatctcgccttgtcctagacacataatctgcaaaattacaaactctttgaggacaagggcacctctttctcattcattgcagtatcctagctcctagcatggtgcctagcacagagttagagctcaataaatatttgttgaatgattgatcaattgttggcactgagggaatacttgggcattgattataagatttgaaggagagggtaattcttgtcttaaagtgtctttggcaaactaccaacctggtgacatggtgatgcaacttccatatgacagaggacactaccaaatcccttagtaactttaggtatgtttatatagttggacttgttatattattgaatagtgtcattttccaataaaaagttgtggaaatttgagaaataatctagattgaactaattctaagagacatatataactttactccatatgcaaaaattaaatcaaagtagatcaatcacctaaatataggagctaagactatatgtaggggtaaatcttcatgatcttggatttggcagtgatttcttggatatgacatcaaaagcttgagcaacaaaggaaaaaaataggtaaattggacttcatcaaaattttaaaatttgtgcatcaaaggacataagaaagcaaaaagacaatccacagaatgagataaatatttgtaaactgtgtatctgataagggtctagtaaccagaatatataaagaatttttgcttgggtgcagtggctcatgcctttaatctcagcattttgggaggctgaggtgggaggatctcttgagcccaggagtttgagaccagccttgacaacaattgagactcccatctcttacaagacaatttattttttaattatccaggcctggtggcatgcacctgtagttccagctacttggggggctgaggcaggaggatcacttgagtccacaaattcaaggctgcagtgagctatgattgtgccaccacattgcagcctgaacaatagagcaagatcctgtctctaaaaaaataaaaatctacaactcaacgacaaaaagacaaataatttaaaaatggacaaaggggccaggcgcagtggctcacgcctgtaatcccaacactttgggaggctgagtcaggcagatcacaaggtcaggagatcgagaccatcctggccaacatggcgaaactgtgcctctactaaaaataaaaaattagctgggcgtggtggcacgtgcctgtgatcccagctacttgggaggctgaggctggagcgggagtcagaggttgcagtgagccgagattgcacgccactgcactccagcctggcgacagtgagactccatctcgggaaaaaaaaaaaaaaaaaaggacaaaggacttgcttagcatttatccagagaatatatgcaaatggccaataagcacatgaaaagatgttcaacatctgttgtcattagggcaacacaaatcaaaaccacaatggggtgccacttcacacccactagaattctataattaacacacacacagaaaataacaagtgttggaaaggatgtcaagaaattggaaccctcatgcattgctgctggaagtgcagaatggtgtagccactgtggaaaacagtttggttgtttctcaaaagctgaaacatagtactaccaaatgaaccagcaatcccaaaatcactgaaagcagaggatcaaacgtatctcacattaatgttgatagcagcactattcacaatagccaaagggtggaaacaacccaatgttcatcaacagatgaatggataaacaaaatgtcttctatccgtgcaagggaatattactcagccatcaaaaggaacgaagttctgatacttgccacagcatggatgaaccttgaaaacagtatgctaagtgacagaagctagatgtgaaaggccacatattgtaattccaagcatatgaaatgtccagaataggcaaatgtatagagacagaaagcaaattggtagttgccaggtgttggagggaagagggaatgaggagtgaccacctggtgggcacaggatttccttttggactgatgaaaatgcctttcaactagaggggcagttacacaacgctgtaaatacactaacaccactgagttgcacacttttaaatggttacttttatgttatgtgaatttcacttcaattaaaaacacacaggttaaaaaaaaaaaaaaaaaaaaaaaaaggacaggccagcccagtggctcacgcctgtaatcccagcactttgttttttgtttgtttgtttgtttgttgtttttgagacagagtctcgttctgtcacccaggctggagtgcagtggcgcgatcttagctcactgcaacctctgcctccgaggttcaagcgattctcctgcctcagcctcctgagtagctgggattaccggcgcctgccatcatgcctgcctaatttttgtatttttagtagagacagcgtttttccatgttgaccaggctggtcttgaactgctgacctcaggtgatctgcccatctcaacctcccaaagtgctgggattacaggtgtgagctaccctgcccagcctaatcccagcactttgggagctggaggcagctggatcacttgagtccaggagttcaagaccaacctggccaacatggcaaaaccccacctctacaaaaattacaataaattaaccaggcatgatagcatgcacctgtagtcccagctactcaggaggctgaggcaggaggatcacctgagccagggaggttgaggacgcagtgagctcactacacaccagcctgggtaacagagtgagaccctgtctcaaaaaaaaaaaaaaaaaaaatgaacaggcatttaacaacatttgtactatcattaggaaaaaatagctttcttaaattcctgcttattgaaaaatagattgaaataatttataaatattgcagtgtcaccaaatatccttcattgataaactacagatttaaggatggcgacagaagagcagatgtagtgaaaggctttgtacttagtgtaaatgccattatgagagaagttcagctgagacctgttttaaatagtcacttgcttagatactgagcatgattctgtgtgaggcgtcagtctgtgtgtctggccatgtggctgtgtcaccaagctgcaggacagcaggactctgctgccatccaggcctctggttcctccagaggtcccttggctgagaagagctccctgtgttgaaggtctggatgtcgcctgggttctgcaggtctcatcagactccactaagaatgaaaacaaccttctccaaggagaaatggccttgccatgggttaagagtaaaaaattcccatctcaccagctccaggtgattggggatagagcgttcctagacagtaatagctgatccctcccaggaatggaggacccagttatgaggttcatcaaagctggggtcagctaacggacaccaagtattcgcccggaaggagaatgctatggcactggaggaaagtaaccatccctccttaacccattcattaactttatatacacaaaccacaaatttacagaatggcatagtaagcaggaagccagtgaaaggtgatcccaggccaccaacaccaccaacaatgtgtggtttaatatgacagttggacatgcttgttataagattttttcatttttaagactgaaaagtgcaacaaaggaagataaaacctttccactgggccgggcgcagtggctcacgcctgtaatcccagcactttgggaggctgaggcgggcggatcacgaggtcgggagatcgggaccatcctggctaacacggtgaaaccccgtctctactaaaaatacaaaacaaaattagccaggcgtggtggcgggcgcctgtagtcccagctactggggaggcaggagaatggtatgaacccgggaggcggagcttgcagtgagccaagatcgagccactgcactccagcctgggtgacagagcgagactccttctcacaaaaaaaaaaaaaaaaaaaacaaaacacgcaacctttccattgtcccatcccccagagataagcactgcaaacattcggtgatatcctgccagtgtcatagaaacaggtataaccattttaattaaattaggatctcagtatagctactgatttcaaacctgctttttaaacttattagaaacatttttcatttcagttaataatgcttctacaacctgatttttaatggctatgtagcattcatcatataagtatcccattactcatttccaaatttcctaatgtagtcatttaatttttttgctattataaataatactgcatatacatgtagctttttacagctctctggttttcttttcatctgaggataagtccctatcagaacttatttttgagacagggtctcactgtgccacccaggctggagtgcagtggtgtgatcttggctcattgcagcctctgccacgcgggctcaagcctcccagactcaagggattatcccactaggcctggctaattttgtaatttttttagtaaagacgggttttcgccatgttggccaggctggtctcaaactcctgacctcaggtgatccaccccccttggcctcccaaagtgctgggattacaggcgtgagccaccgcacccggccagaacttcttaaaaagacagcatctttaaggcttttgatttatgttgctatattgtttctttggactgacttaattttaaatcttttttatgatcagagaaaaaagttttgtttataggataccaatttttgtctctttaatttcttattagtattttttccataactctttttgcacagtgttatcaaagaaacaagcttctgagtgaatttagaaaacaatctgtccaccaggcgtgatggctcacacctgtaatcccagcactttgggaggctgagaggcaggtggattgcttgaacccaggacttcaagacaagcctgggcaacatgtcaaagccccacctctacaaaaaatacacaaattagctgggcatggtggtgtgtacctgtagtcccagctacttgggtggtgggggggctggtttgctgaggtgggaagattgctggagccaggaagtggaggctgcagtgagccaagatcatgccattgcactcccctttgggtgacagagagaggtgagaccttgtctcacagaaaaaaaaaaagaaagaaagaaatgaatctgatatgcattcttttttttcaaaacaggcccacatggaaaaaggcaaaactaaaagatcactactaaaaagtcaaacattgattcagtgttttgggatcagccagtgagctagtgtagttacatacagttggagagagagagggttagtgaattaaccagacagtgctgttatgaacattataacttaaatgaataaattaggtttccttcaacaacatattccagcagacactttctgatagcaaagatataatgatcaaatacaatcttctaaagttaacatgcaaagccaggcacggtggctgtctcctatagtcccagttactctggaggctgaggcaggaggatcacttgagcccaggagtttgaatctagactttacaacacagtgagacaccatctctaaaattaaattaagtaataacatcttaggttctctttagcagctcattatgcgctattaaattctttttaattttaaaaagttaatttgtaaaacatgttcattatattcatattcttgggttagtgttacattctccaagacataactgtgaaacctttgtagttaatggcagtatgtcaccagagtgcctcttttcctatcgaacaaactagacagaagttcaattctgcagtgacaagacacagagtagcatagagttcacctgaacctgaccttcaccccacccagctcattctctgaattctttgtgccttcacagtaatcagtatttaactgttctccagtcttctcttacctttatttaaaaaaagcaaatgctgagccagttcctcaaatatgtatgaattatgtttatatttgcaacataaatgtagtggcttgctgacatttgtcatgttcaaaattatatactttgaatataattttgaaagatgttaaatgccttcatggatttaaggatattttctcttgttcagcaatatttatattataaagtaggatgtttacaataaaatatgggtgctcaatcacaagctaaaagcagttaagtagtttaacatacgttaaatggggctgggcacggtgggtggctcatgcctgtaatccaagcactttgggaggccgaggcaggcagatcaggaatttgagaccagcctggccaacatagtgaaaccccatctctactaaaaatatgaaaaattagctgtgcacggtgacgcacgcctgtaatcccagcaacttgggaagctgaggcaggagaattgcttgaacctgggaggcggaggttgcaatgagtcgagatcaagccactgcactccagcctgggcaacagagcgagagtctgtctcaaaaaaaaaaaaaaagttaaatgaacacaccttttaacatctttggaaattaagcctcaaactaatgaaccccaggatcccaagcccatatccccatgtgtccccagccacagggacagtctgtccctctgaggctgcttttgaaagaaagtatcatgtctccagcccagaagcactgtgagtacttcaccactctgttgtcttagggaaagtgtaaagccatttttgaacgccttccatgatatctgttcattgctctgtcttaaacataaatgttctcttatttctacaatgagaaattattcaaatattttactagctttctacaatagcacaggggtataatagggtcactgtccagccctcagcaagttctagccttcagtttgctatcagctgacacacccatagttattacagcttgaccatgtgtaatccagaaatccaacattcaaaaggctccaaaatcctaaatgttctgagcgcttacatgacactcaaaggaaatactcactggagtatttcaaatttcagatttctggattagggatgctcaacctacgtataaaaaaaatccaatatatgaaatacttttggccccagccatttcagataagggatattcagcctgtactacttttgcatatagtttttttttaagtggtgcttttaaattttcagcagtggactgacaatggcacagtttacaaaatgccccacttttccttgtcctcaggcaagtgtgcacctgtctgtctggtgctccctgaggccttctctctccctctctctcgcccctcactcttcactgtctttccctccccctgtacgtgtccctttttccctctccattccttttccctattttttgtcatatatctctaccctttactggccatgaaactaaaatgtcactgtcattattattatttgctttatttattaaattgaatcctatggcacgtgctgttttttcccattaggtcaacaaccttcaacaatgaacaatgatgtatattcatgtcttcatacatttctttcacacatagtatacgtgatagatagatgataggcagtcagattggtggatgggtgagtggatggatggataatacaagtgatgatagatggaacatagatagatagatagataggattgattaaataagataagatgacgagcagcaggtgtccaatccacccctgcagtgagggcagatatttaagggtcttgtgggggtaacattgcaaactgcgaattgggagaattgctgaacatgtacattgcttattatcctctcactggtcataattaagaattctacaggcatgaaaggatgggctcgtgatggatactctggggtggcctgtaagtgccaagaagcagtttattgttgtgtgggagcagcatttgctgggaaaaggaggtatatgtcctgccagaagcccacggaatgaaagaggccactgcctttggtaggatgtccctgaggccatggcccaggttgaatttgttctttgccaatttatatggtggtgggttatactcaggtacagcagaaatcaggaaggggccaggcggcctctgggagactcgtggggctgtcatgtggaaggtgctttctcttccgcaggcttctctgtcataggaaggaaagccacttaacatttcctagtccattcattatcaattacatgaattatgtatgactttctgaggataattgtgttcttggatgccagaaaagttgctttctaaattacttatgaagtagaaccaggacaaacatggtctaaacaccaaaatacaactcccacttgcagacacaaacccaattcccatcacctcaaagttgctaacaaataattgatatgaattttcaacctaattcacataggcttttggcatggaggtgaaactgtacaacaaagtaggtagaagacagagtgagagaagacacttcctggaaaagtttattcatccactgctcatgaaacaaatatttactgaactcttagcatgtgtcaagggcaatgctaagtgcttgatatacatcaggaacaaaacaaaaagatatctgccctgggggagcttatagtatagggggtgatacagagaatgaatgaaacataataaatcaataaaccacagtgtgggctgggcgcagtggctcacgcctgaaataacagcactttgggaggccaaggcaggtggatcacctgaggtcaggagttaaagaccagcctcgccagcatggtgaaaccccatgtttctactaaaaatataaaaattaatcaggcgtggtggtgcacatctgcagtcctagcaactagggaggctgagacaggagaatcgcttgaacccgggaggcggaccttacaggtcattgtaggaactcttagtgaataaggtcccgttggaggattttgagtggaggaggggccctagctgacctacactgtgaaaacatcaccctggccactgtttcagaagatgccatagagaggcaaggagaccaggctgctgctataatctgggtgagaggtgcggatggttggaatcatggaagtagtgagattcaatttggatccagtttgaaagtagagccttgctgatgtgatggatgtggctcgtgatagcaagagaagcatcagggatgatgtcatggtttattctgagccattgggaagacaaacctgccttcccctgtggtctgaaggctgcaggtggagcaggtcccgggggtcaggagttcgagcctggccatgctgagtttaagatgtgcattagacacctgagtagggacctccttcaccatctgtaaggaactggtaatagcaataatcagtataataatcaagcataatcatggcaaaaatcactgcaatacatagctgtcatatttacagttttaactttttcaaatataaacttggttacctgtttccctgaaagtttagggacattttttttctggtaggaagactacttgccaagaaaatttcattttctttatatttagagtcaagttagcaaaaagcgataaaataaagagtatgctccttacgaacttcgcccgaaaatccttttgctggccacttattttgttgtttggccctaaaagactgatgacagggtaagaactgaaaccattctgtttacggagttcctcaagtcatggacagtcctgggaaatgtcaagtccttactggattacctgaactggttggaagggagctgtccaccagtgcttgctcctcatggtttcttgcttttgccttggtgatgtggttgaattccccactgccaccctacacccaggcgtacttcttgatttcgaaacacactcatcaccatactcttgagaccagtcttgttgcaggcagtggctcaccctctagtccacatccctgacctctgcactcatggtcctgaagcagagctctgggattctcttcatccaaacccccagtgacttcccatccatccacagattccaaaactggtcactgcatatttctatatcaggcgtaattctagctactatgaattaaaagcttcttatgaagctgagtctctgttaggagaaacaaaccaataagaaaatcagcacacagtgtgttgatggctgatgcgtgggtgggaaagaagaataaagcggggtagggactgccgggtggcagggagttgccattttctccggggctgcggaggatgtggcatctgagcagaagctggtagaagggaagggcacggcagtggcccaaggaggcaggctctcgagttcgagaggtacccgggctgtgtgtctggagaagagtgagccaggctgggtggggaggggaatcagggagccagggaatggggaggtcagatgacgagaggctttaggtttcacttggagatggaaagtactggattgtttttgttttgttttcataatatattcttttattgtgataaaatacatataacataaattttaccatcttagtcattattcagtgcaccttctgtggcatagaacattcacactgttgtgcaaccatcaccaagatccatctccagaactttctcatcttcccaaaatgaaactttgtgctcatgagacaactcccaactccttcctccccacagcccctggcaacgatgcttctactttctgtctctatgattttgacttatgtaagtggaatcatccagtattcgtccttctgtgacaggctcatgtcacttagcacaaagtcctcaagctttatcaatgttgtagcatgtgtcagaactgccttcctttgtaaggatgaatgatactccattgtgtgtctagactgcatttccattatccattcttctgcccatggacacttggattgtagcattgggttgcttggggttttttgttaccagtttttatatattatctttattattatttgtgtatatttaaagaggacaagtgcaatttgaatggaacatggagatattgtgtagtggtgaagtgcaggtgcagtttggatggaacacggagatactgtgtagtggtgaagtacaggtgcagtttggatggaacacggagatattgtgtagcggtgaaatacaggtgcagtttggatggaacacggagatactgtgtagtggtgaagtacaggtgcagtttggatggaacacggagatactgtgtagtggtgaagtacaggtgcagtttggatggaacacggagatactgtgtagtggtgaagtacaggtgcagtttgagtggaacatggagattctgtgtagtggtgaagtacaggtgcagtttggatggaacacggagatactatgtagtggtgaagtacaggtgcagtttggatggaacacggagatactgtgtagtggtgaagtacaggagcagtttgagtggaacacggagatactgtgtagtggtgaagtacaggtgaagtttggaacacggagatactgtgtagtggtgaagtacaggagcagtttgagtggaacacggagatactgtgtagtggtgaagtacaggtgcagtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggtgcggtttggatggaacacggagatactgtgtagtggtgaagtacaggagcggtttgagtgggacacggagatactgtgtagtggtgaagtacaggtgcagtttgagtggaacacggagatactgtgtagtggtgaagtacaggtgcagtttgagtggaacacggagatactgtgtagtggtgaagtacaggtgcagtttgggtggaacacggagatactgtgtagtggtgcagtacaggtgcagtttgggtggaacacggagatactgtgtagtggtgaagtacaggtgcagtttgagtggaacatggagatactgtgtagtggtgaagtacagatgcagtttgaatggaacatggagatactgtgtagtggtgaagtacaggtgcaatttgagtggaacatggagatactgtgtagtgaagtacaggtgcaatttgagtggaacatggagatattgtgtagtggtgaagtacaggtgcaatttgagtggaacatggagatactgtgtagtggtgaagtacaggtgcaatttgagtggaacatggagatactgtgtagtggtgaagtacaggtgcagtttgagtggaacatggagatattgtgtagtggtgaagtacaggtgcaatttgaatggaacatggagatactgtgtagtggtgaggtacacgtgcaatttggatggaacatggaggtattctgtactggtaaagtacaggtgcagcttgggtggaacatggagatattgtgtagtggtgaagtctgagtttttagtatatccatcacccaaataatgtacgttgtacccattaagtaatttttcatcatctaccccccaccaaccccctcacccttttgagtctcctgtgtccatcattctacagtctatgtcctgtatactgattatttagctcccacttgtaagtgagaacatgtggtgtttgtttttctgtttctgaattgttttccttaagatagtgacctccagttccctccatgtatctgcaaaagacatgatttcacttttttatggccaaaaagtattctattgcgtgtatatataccacatccagtcatccatgggtgagcactttggttgattccatatctttgcaattgtgaatatcactgtgatacacatatgagtgtaggtatctttttgacataatgatttcttttcctttggatatatacctagtagtgagattgcaggatagaatggtagttctatttttgattatttgaggaatctccatactgttttccatagaagttgtgctaatttacattcttaagaacaatgtataagtgtcccttttctctccatcctccccagcatgttatttttttgtctttttagtaatagccattctgactgctataagatgatatctcactgtggttttaatttgcatttatctgatgattagttatgttgagcattttttcatatgcttgttggccatttgtatgtcttctttttaaaagtgtctattcatgtcatttgcccactttttaatgggattatttgggggtttttgtagagttgagatttttataaattctggacatagtcccctgtcagatgcagagtttgcagatattttcactcattctgcaggttgtctgttcactctgctgattatttcttttgctgtgcagcagctttttagtttaattaagtcccatttgtctatttgtgttttcgttgcatttgctgttgaggtcttagtcatgaattctttgcctagaccaatgtccagaagagttttccctaggtttccttttagtatttttatagtttcaagtcttacaattaagcctgtaatccatcttcagtcgatttttgtatatagtgagagataggttacccctctcaggaacagtgagtattagaggaaaaagaattcgaaggaagaaagaaactgtgtgcccagcattgcggcccattaactgtccttccactttgatattcacatagacgtcctcctgttgagggaggctggtgaaagggaagcagggctgaagaataccaagggccttctcaatattgtgaagtactcttttttttttttttttaacttttatcttaagttcagggataatttgttacacagaaatgatttgttctcatcatttagctcacacttataggtgagaacatgtgttatttggtattctgttcttgtgttagtttgctaaaaataacctgcagctcgagctcctgacctcaagtgatccgcccgcctcggcctcccacagtgctggtattacaggcgtgagtcatcacaactggcctggccaacatggtgaaaccccgtctctactaaaaatacaaaaattagccgggcatggtggcgcacgcctgtagttccagctactggggaggctgaggcaggagaattgcttaaacctgggaggcagaggttgcagtgagtggagattgtgccaatacactccagcctgggtgatagagcgagactctgtctcaaaaaaaaaaaagaaagaaagaaaagaacaacctccagctctacatcctcgcaaaggacatggtctcattctttttacagctgcatagtaatcattggattgttttgagcccacctgtaatgcgatttgacttatggtttcaacgattcactggctgctgagtggaatggactgtggtgagaagggtgggaacagaccagataggaggctgtggccataactgcgtggactagatgatgatggcttcagtcaggaatttagcagtgcgagaatcagaatctggatagattctcaaggtacagccaagagtatttccttaaagatcacagggatgtctccaaggagtttggcctaagccgggccgtggggggaaaccgcaggtggagcaggtttggggaggaagattaagagttctgtttggcacttgctgagttgtagttgcctgttgaattcaagcagtgatgtcaggcaggcaattggatgtgcaagtccagaattcagaggagaggcctaagctggagatgtccatttggagtcactagtatattcatggtgtgcatgccacaaagtggagatcgccaaggcagtgagcttacatagagaggggggcccagcaccaaccctggatccctctaggattaggaggctgtgggaagagagggagccagcagtgaaccctgagcaaaagtggccatgatgtcttgaaggccaagacagtgtttcacggggagggtgtagtcagccctgtcaggtgctaatgatgagccaggtcagatgaggaccaaggtcccctgaaagcagcctgaatgtcattggtgaccctcatcacacaggagaaaccacacttccttgtccagcccagccttcttttttttttttttttaaagactttgtttgtccctactctaccctcacaggtcttcttacccattccaggaccatgataaatatttgctgagtgaatcggtgaatgacaggccttactttggttgagtcactctgcagggagtggaaggaatcgccactctccccagactctactctcctccaatcactcaccctgcactccaagctccaaagcaaaagagcgcctctgcccgttttactgctgtccctcttccctcacagtgtggcccaggaagctcagaaagtggctgtggctggagcagaaggccaggaagaggtactaagccaggctttggggctttcttatttgattatttggaattgtcaggaattccaagaatgttgggactatctctgtgcgaatttaaatctgaaacgcattagaaagttagataagtgttaccctcatgtaacatctgccatgctacccacctggataggataaatagtagagtttttaatgaagttattaaatgttcagagaggaattaatcatgttgagtgactaattccaactagaaactcttgtaatatatatagatccagatgtttctcttgggaaagaaaggagtcccattcccaggcatgtatccaaacggaatgaaaacatacatcaagactaaaacttgcacacgaatgtttatagcagcattactcaataatagccaaaaagtagaaacagcctaaatgttcatcaaatgacagatggatcaacaaatgtgtccgatccatgtaatggaatgccgttcggcaataacaaggactgaagtgctgacacatgccatatcagagatgacccttagaaacatcatgctaagtcagagaagccagtcacaaaaggccacatattgtgtgactccatttataggaaacgtccagcataggcaaatttgtagagacagaaaggagggaccaggcatggtggctcgtgcctataaatcccagcactttgggaggccaaggcgggcagatcacctgaggtcaggggttcaagaccagcctggccaacatggcaaaaccctgtctctacctaaaaaacaaaaattagctaggtgttgtggcatgcacctgcaattccagctgctaaggagtctgaggcaggagaatcacttaaacctgggaagcggaagttgcagtgagctgagatcgtgccactgccctccagcctgggcgacagagcgagactctatctcaaaaaaaaaaaggaagaaagcaggaggctgggggaggagaaatgaggagtgtctgctaatgggtcatgcagaattgcttttcttggtgataaaaatgttccaaaattgatggtggtgatgaaggcacaactctacaaatattctagagagaccattgaattctacacttaagattgttgaattgtatggtatataaattatatctcaataaagctgtgacatgaaaaaatagaaagaggctgaggtgggtggatcacttgaggccaggagttggataccagcctggccaacatggcaaaaccccgtctctactaaaactacaaaaattagcaaggcatggtggcatacatctgtaatcccagctacttgagaggctgagacatgaaactcactgggccccacaaggcaaaggttgcagtgaaccaagatcaagccactgcactccagtctgggcaacagagtaagattctgtctcaaaaaagacaaacagacaaaaaatagaaagagggaaaatgtaaaacttgaaatatttgagtccaagaactggaagaagtatttaagattatctagtgcaaaccacaagtatagaattacaaggtgtttgaatttctttggacaatattctttcccttttatgtcagttatttgttgacagacttacagctactgcagaattacagaggggctcagtggctgcctttgaaatctttccttttgtatgtatgtggaaattgtaatcccctcatacctttaagctagtgcaggtgttgagcttcctgtagccactgaggttccccagagctaggggctgcaccttctcagaacaaaagctcaatagaacaaaaacccttggctgggcagggcggctcacgcctataatgtcagcactttgggaggccgagacagatgcattgcttgagctcaggagttgtcagaaccaggctggacaacatagcgagaacctgtctctactaaaaatacaaaaaaatagccaggcgtggtggtgtgcgcctgtggtcccagctactcaggaggctgaggtgggaggatctcttgagcccaaaggggcggaggttgcagtaagccgagatcgcatcactgcgctccagcctgagtgacagagtgagatcctgtctcaaaaaaacccttgttggttcccaattaggacactatcagctaattttatggtgttgggtgcaagtacatctgcccattgataacacagagatcacaggcctgcacattgcctggcaggtgtgagctgttggtcccagtggggctgaatatggctggaccagtcccagctcgtcacctggaaaactcagcccggagcctgcagggaggggctgtccagtggtgggtcactagcatccccttaccccacagaggattgaggcttggtttacagcatgcagcccctcaggtacatttcagagctgcatctcacgttgtttatggcagtattctgctaccccttttcttccctgtggcatatttttcctagtcatctgttgcccttctgttccatccacgctggctataagtgcattggcctggacaagtcacattttcttgggccgttcctgaagggtctgaaggctgagagcaaatccaggaatctgtgggtgtggttgaagctcagttctgaatgcagtgcccttgggccgccccccacaccccgccgtgcttatgatctgagcacagcccctcctgcagctcagaacctgtgcccccttctctcttctgcaccaggacttaatgtggtgctctccacatgcaggtgttctgtagatgtttctagaatttcctttacttaccatgtgttgctcccctgccccaccattgaaatccgggccttgccacatttcccaaaagccacaacgccacattcctgggtgccttccttgcacccaaagccgccaaaggagcctcctggaccgtcccacggccccagcgcagccctccccacggctgatcgccacactcttgactgccccacggctgtgcttgtcctccctgacaccacatccccgcacccccactcgcttctcctttgtctttccagcctctttcaccatgtcctcctgccccttgcccctcacagactctcctcctttctgccccacccaccttcccctgtgagctgtgaactggtcagtcctggccttagcaccagcctctgcccaggaatactggattatcccttggaccaggcgaggaaactgttaaaactgttaactgtcagcagtcggagctaagaacccacccaccccagccgggattcgaaccccagcactcagataccaaaatctatgctcaaattgccataaacattatttcatgccttctcattagaaacggtgtgttttacactaaaatttgccaacagaaaaatatatttaattctatagtcactgtcagcaagaatatagcaagcccacctttacagatatttgctttaaatttaaccttgttcccccaaaaaaggaatctcctttctccattttgcttctcaacaggtaaaacagaaaggatacggagtgactaaaaagaccaagaataagagtaataccctaaaatgttacataattcaagcttgatgttccaaggagaatttttgtcagataacttagaggcttgtctggaagcaaagactaaagtcgtgattctttgaatcttttttccctgatgaaaatacctaattattttatttctgttcccacacaaataagtttctcttgtgagtctctttggcatgctgcaagtgcacagctttcttagagaattaggattttacagagagaatcttaggtctatgagtgggagtattgtgaaaagtcaaaagctaaaaaattaattttgtgggaaaaaaaatacttgaggaaaattctatgttccatcactggagactcgatttatttaattctgtactgtgggaactgattagtaaaagtttatactttccacagttgttttgtgagattataatttggaaataaaagatagcatccctagacaaatcttcaagtatctttgaaaaaatgagtaaaagcagtagaaatcctgcaactggccctggggggctatgaaggtcccttggcttcatcttcctgtacttgggaacctgcagggaattccgtttctgccccaggcctcaaggatcttgagagaattaatatgtgaaattagcaagcttgcaggacacaggaacaaatacagagatcaattgtatttctgggcactagaaatgaaaaaaatctgaaaaatgaaatttataaaacatgaacattaaaaatgacattcaggctgggcacggtggctcacgcctgtaatcccagcactttgggaggccaaggtgggtggatcacctgaggtcaggagttcaagaccagcccagccaacatggtgaaaccctgtctctactaaaaatacaaaaattagctgggtgtggtggcgcatgcctgtaatcccagctactcgggaggctgaggcaagagaatcacttgaacctgggaggcggaggtgtagtgagccgagctcgcaccactgcactccagcctgggtgacagagtgagactccgtctcaaaaaaaaaaattaagaaataaataacatccaaaaaagtttacttggaaaaaaatttaacagcagatgtaaaaaatctatatgctgaaaactacaaaacattattgagataatttaaataagatgtaagtaggccaggtgtggtggctcatacctgtaatcccagcactttgggaggccaaggcaggcagatcacgaggtcaggagttcgagaccagcctggccaacatgctgaaaccccatctctattaacgatacaacaaattagtcgagcatggtggcacacacctgtaatcccagctactcaggaggctgaggcaggagaattggtggaacccgggaggtggaggttgcagtgagccgagatcgctctattgcactccagctctggcgacagggtgagactccatcttaaaaaaaagaaaaaaagacataagtagagaaataacatgttcaattatgttcatggattacacaaccattaactgccattaaggtggcagttcttctcaagtgacccatagattcaatgtaatcccaatcaaaataccaacaggcttttttataaaaattgacaagctaatgctaaaatttatgcaaaaattcaaaagacttagaggcttgtctggaagcaaagactaaagtcgtgattcttcgaatcttttttccctgataaaaatacctaattattttattagaatagccgaaacaatgttagaaaaggagaacaaagttggagaagttagaactgcctaatttgaagacttactagaaagccacaataatcaagacagtatagtgctagcatgaggagagagctagagatcagtggaacagaattgagaattcagaaatagatccaccttcatatggtccattttaagagagatgtcaagcgcagtggcatgtgcctgtggtcccagctactcaggaggctgacatgggagggtggcttgagcccaggaggtcgaggctgcagtgagctgtgattgcaccactgcactccagcctggggaacagagcaagaccctgtgtctaaaacaaatttaaaaattaaaaaaaaattttaacagagagccagagatactaaggattcaaggagaaaggatagtcttttctctaaatgttcaggagaagctggatatccctatggaaaaaaagtgaatattgaccctttccttacaccatacccaaaaactaatttgcatggatcataggcctaaatgtaagagctgaaaactataaactttaagaaaaaaaatggaagaaatattttttactaagtggtaggcaaatatttctttgatagaacacaaaaaaaggataaactctaaaagaaaaaaatggattaattggacatcagaatttaaaactttgttcatcaggcacagaaaaacactgggccgggcacggtggctcatgcctggaatcccaacactttgggaggctgaggtgggtggatcatctgagaacaggagttcgagaccagtctgaccaacgtggtgaaaccccgtctctatcaaaaatacaaaaaatattagccaggcattgtggcaggtgcctgtaatcccagctacttgggaggctgaggcaggggaattgcttgaacctgggagacagatgttgcagtgaccagagatcattccattgcactccagcctgggcgacagagcaagactctgtctcagaaaagaaaagaaaaagaaaaaaaatgaaaaacaaaacaccatatggtctcatcatatatggagtcttaaaaagttgctcttggccaggtgccactagtggctcacacctgtaatcccagcactttggaaggccaaagcaggtggatcacttgaggtcaggagttggggaccagcctgaccaacatggcgaaacgctgtctatactaaaaatacaaaattaactgggcatggggtggcacatgcctgtaataccagctacttgggaggctaaggcaggagaatcacttgaattcgggaaacagaggtttcagtgaaccgagatcacgccactgcactccatcctgggcaataagagtgaaactccatctcaaaaaaaaaaaaaaaaagttgatttcatagaagtagagagtagaagtggttaccagaggttggagaggggaaagtagaggagagggaattggaagaagctgatcaatgggtacagagttacagttagacatgaagaataagtttgggcattctattacacagtagagtgactatagcaaataataatgtagtgtatatttcaagttagccagaagagcagacttggaatattatcaccccagagaaatgataaatatttaaagtgatagatatagtagttacccagatttgatcattatactatgtatatactcattgaagcaccacattataccccataaatatgaagttattatgtgtcaattatgtattagtccattctctatatatttctttatagtcactgctataaagaaatagctgagactgtgtaatttatgaagaaaagaggtttaattgactcatagttctgcaggctgtacaggaagcaaagcggcttctgcttctggggaggcctcaggaaacttagaatcatggcagaaggcaaaggagaagcagacatgtcttacctggccacagcaggagaaagagagaagcggggagatgctacacacttttaaacaagcagctctcacgagcactaagtcaccgagggggaagtctgcctccatgatccagtcacctcccaccaggccccacctccaacattggggattacaattcaacatgagatttaggctaagatacagttccaaaccatagcagattatatatttttaaatgttttaaataaaaaaataaactttgctcatcaaaaaatacttaagaaaaataaaatgcgaatttgggaaaaaatatttgcaaaacctatagctgataaaggattttaatccagattatttgttgtaatctcttacttttatttttaaaaaactcaataataagaaaacaattgttgagccaacagtttcataaaagatagccaacgtaattcatcattagggaaatgcaaattaaaaccacaacgagataccactacatgcccactagaataattaaaattaagaaaatgaagccaagcactggtgaggatgtgcaatactagaacccctaaacattgcccttgacaatgcaaaatggtgcaaccaccatggaaaacaatttgacaatttttaaaacttaaacgttcacttaccataaaaccctgcaattcccatcctgcccaaaagaaataaaaataaatgtccatactaagacttttacacaaatagtcctagtagcactattcacaatagtcaaaaactggaaataacccaagtgcccaccagctggtgaatggataaataaaacatgatatatccataaaatggaatagaactcagcaatttttttattttttatttatttaatttttttgagaccgagtcttgctctgtcacccaggctggagtgcagtagcacaatctcagctcactgcagcctctgcctcccgggttcaagcgattctcctgcctcagcctcctaagtagctgggactacaggcgcccgccaccacacccagctaatttttttgtatttttattagagacggggtttcaccattttagtcaggatggtctcgatctcctttcatgatccacctgcctcgggctcccaaagtgctgggattataggcatgagccaccgtacccggccactctaattttttttaaaaaggccagcctggccaacacggtgaaaccctgtctctactaaaaatacaaaaattagctggatctgatggtgggcacctgtaatcccagctactggggaggctgaggcaggagaatctcttgaacctgggaggcggaggttgcagtgagccaagatcgtgccactgcactccaacctgggcgacagagtgagacgctgtctcgaaagaaaaaaaaaaaaaaggaacaaactgttaatgtacgtaacaaaatggatgaatttcaaaaatgtgctaagtgaaagaagccagtcagaaaagactatggttttatttacatgaagtttctagaaaaggcaaaactatagagacagaaggaactgagtggtggttggggcctggggtggtggcacggcttggctccaaaggaggatgaggcacctttcgtgtgatagaggaattctaaaacctgggtgttggtggttgcatgactttataaatggatgaatttatggcatatatagtatactcaaaaagttgtttaaacatgacactaataataattgaagtaggctaagtcctaagggaaaaatagatgtgtggggtatattgcaaatttgtacttgtcctcactggtctcttatacccaaggaactgctattgagcagagaacagggaagctagccagcctcagagcatgcttttgaatccaggtttgaccacttcctagttaagtgaacttaagcagttttatgaagtattctcgatggttctcatgttgtcattgaattacctgcacagcaggattgtttgagacagatagaccttatacctaggctctcagaacattgcctgggcactcggtaaatgctaaggtaaacgttaacctcatgcagttgcttagttgttgaacttgttgcctgtggcgatcacgcttcttgctaggaaagatgctttcccctcaggaactgcaaaggcttttaattggcttgttttcgtgtagttttctttttgtttggttgtgttttttgagacagatttcttgctctgtcgcccaggctggagttctctgctcactgcaacctccacctcccaggttccagtgatctcctgtctcagcctcttgagtagctgggattgcaggtgcgtgccacaacaccgggctaatttttgtatttttagtagagacgggcttccaccatgttggccaggctggtctcgaactcctgggctctggtgatccgcccgcctcagcctcccaaagtgtcggtattacaggcgtgagccaccgcgcacaacccatttgtgaagttttttttaacgtggtttctgaaaactgttcctcaatagaattttagatatactggccaggcactatgcctcatgcctgtaatcccagcactttgggaggctgagacgggcggatcacttgaggccagcctggccaatgtggcaaaaccccatctgtactaaaaattaaaaaaaaaaaaaaaaattagccgggtgtggtggctcctgcctgtagtctcagctagtcaggaggctgaggcacgagaatggcttgaacccaggaggcagaggagaattttagataaactgtgtaacattaaatttcaggatggacagaaattatacataacaaaatcatagtgttataacactgtaaaataacaaaataacagatccagacttttaacttgatattcaccagatacctgaaaaaagaaacaacggatacctactagctcattctgttatccaagggataaaattgagtaggtgtattttaaatgtatcagacctaaactcgagttttaaaaaattcaatttaagtggttactgtcctgaaactgcctgtttctaaggctcatgtgattacagtggtggacattattcactaggttcccgtaaaaggttacattaaacaacttaaacgactgtgtgtgcttccacttggtccagaaaggatttagtgctgctcacaaagttacaatacaagttaataaagcaactgaaaatgtgggaggatgaaggcaggaacggggagagtgagttttgtggaaccgcagtgaagttactgctcagtgtgtctgaccgaggctttcacacgtgccgggacgcaccctgaaacttggtttctaagctttcacacagtgaactcagtgacgtaaatgaaagcagtaggaaagtcacaggatccacagattaaaaatgaataataaaatgtaaaaagcagttgcttgagaaatgcaagagtattcccgacactaaaattagaagtatttttcataagtcaccctagaaagaatattgtgcgaatgttgggaacattttcacactcaacccagggatgtggtccaccagcccacagggcagggctcagtgtggaccaccggccacccccagcatggcgagtgggagcagggcggcctgaccccacagccgccagacagacggcaccacactcacgtcaagtgatgggaaatagctctttgatttttatttcaaaatgaaaaatgtatcccaaatgtcagtgtaaccaaaaagtcatttgactgtgttatattagcacagaaattatgcaagaaatgttattcttacacttcttattgtcatctgtttgtttgcattttaaagtaattaagaagaatttaaaagcaaaaaactttcttttactcatctgagaatttcatgttatctttgagtatactttcaatataaaggtagtcgtagatagcagtatcagaaggaaaatgttgtcaatatgaaatagagtcattacatactttaattttttaaactttagtttaaatgttgatttaaaaatcaaaattgtacttcaaaattttaaagccttgcttttaaaaaataggtttaaacttggtatcattatttttattttatataactgaaatccctttgaatataaataacaaatattcacatatatcagttgctgaacataatgaagatcacatggcaattctcatattcagtaatacagaaaaaattaagaaatttaacttagtaattttctttttatgctttcagtttttatataccctcagatgtcaaaaacaggttacattttactgtgaattactcttttcagacacatacacacaaaaaaaaccctgaaattatttttatttagtattatttatcttagttgctaatgtagacatactaaaattattcttcatattaatttctccgttgttttttaaaatgtatgagatctatagggaaggaaaaaaactagagcctgtgaattaaaatgacaacagacaacaagagaaaagacatacaaattttattaatgtttttaattttatatgcacaggggcgtcacagaaaagaagtggaacttataaaaaaaaaagttagagttgggagcttatataccattttaacaaagggtgagaaattgtggaagaagtgactacaaatgaaaggggatttgggctcctagggttgatataattgtgagaaagggctattttagtaaggtttgcttatgcagactcatcttggtgacagctctctgtctctgcgataaaggtcactcttatcctagtacagggtgggggatatttatgctcttttttaggcagaaagggggagagcagacagctcttcttatatctgttgtttctcagttgccttcagctcaaaataatcaatatgccagtgtcacataattgggggtggcgtattttgatccctatcagaatcctagcctagctctgaacaaatgtttaattataccttaagaagcttcactggctggctaaatatttaatgacttcgcagaatccttttctatgtgtttatgtattttttagttgagataatggtattacggctattttttaaaagtctttatgttttagaaataatgttgaaataacatgatatgctgccttataaataaaatatttgtttcaaaataataaaagcagtgtaaaagtggatgagatatggatgaaaagaaattggccatgtgttgataaatgtggaacctgagtaatgggtacctggagattcattctctctatttttataagtttgaaacttttccatgtaaaacattgaagtaacaatcacaacagcagccctggaattcaccatgaagtttttagaaccctttaaaaagtaaatatgtggaatcatggggacttagctgtatgctagtccaacatgtttacccatgaggaagttgaagtgaagagagattgttgtcgcttttgtgtttcttcagtgttatctaaatcacctacttaaaaatcacagtgcctacaatccctgttttactcttgctatcaacacatgctccagaaattctgtatcttcattcttggttcaagcacttctctgtaattctctcaggcttcagctcaaacctagagtttcgtctcctataaagcagttccagtcctcaccagtgaatcttacccaacaacaggacattaagccttatcccatgaactgaatttacacttggcatctttgcaatgtatttgtgaatcgtggaaggaccacagaagttactgagctttacagacctgtgggaatcctcatgagtgtcttgtgttgctttaacaaggtacccttgtatgagatcccctgtactttatccagcgaagagtggtttttctaaaatagtggttttaaagaaacccagtagaaaactaaagaatagaagatgatttaatatatacataataaaaggtggtagtatgcatagtggttaaaaacacaggcagtggaataagatattctgctattacctgctgtgtgcccttgggtggattacttaactgatctgagcacaatgacaacagcgacctcttccagttttgtgagagttaaatgcgatcatgtgttaaagtactgtgcacagtaccctcgacaaaataagaactacgtaagaactattttaaatagggattatcatgtgagtaaggcccttactgtgacatgcaggaaattaacgcaaaatgagaaagggtattgcgaaggaagtgagaaaacagcagaagccgaagcctgaaggaatgagctgaggcctgggggcgctgaggatcccagccccgctgggcagggcctccaagctggggagctgcgggggtgcactgtttgcagaggcaggtggggcggtgatactgatatttctgcaggagggagccgggaggtccttgagcaggggccccagtaaatgcttcagagctagaatgtcctccctttccagctcaccaagggctgaagcacaagggcctcccgcctccctgcagcgcacatccgccctctggcgaggccaggccggcatccagtgtggcccggtggccagaacgcgcccaggccatggccgccgctccgtaggcctccgtttcacgggtgctaagtaagtcgaaaagcaagggcatctgataggagcctcagtttctcctccgcctgccaggaggtcttgtgcgtgcagagcggcggatgcgtgtggcaccgcaggcgcggggcgagggcggctccggagaggcccaggggcttagcgcgcctggctttccacagcccggcttcgggcctactcaagatggggtttctcgggcggggcgtggtgggcgcgcctgtggaatcacttgagcctggagttcgagaccagcctgggcaacattatgagacctcccttcccatcccccccaccgccaaatctcaaaacaaacaaacgaacaaacaaattagctggatattgtggtacacacctgtcgtccccgctgctcgggagcctgagggcagaggatcgcttgagcctaagagttggagaccagcttgggcaacatggtaaaaccccgtctctaggaaaatacaaaaattagccaggcgtggtggcacgcggctgtagtcccagctacttgggaggctgaggcaggaggattgcttgagctcgggaggtcaaggctgcaatgaacccagattgcaccactgcactccagcctgtgagaaggggcagagtgagaccacgtctcaaaaaattaaagttaaaaattaaaaagatcaatttctcaaccctctcactgacctctagccacatgcctgttagctctggggtcagcaccgtgtgggctcacttccatggctggttgggtagtagggtgaccacacgaccgtaccccacacggtttggagccctttctgccctgcagggcctggagcaatattgtttttctttttttttttctttttttttttttttttttgaccaaatttagattaattactccagtcttggtcgtttttaattccatttggaaacctattatagtgaataactcttatgtgaacttttataaaatctggtgtctgtggttcctgtgtttggataaatccagttaaatttgtttcaatgtagcatgagtctatagtttttatatatcatacgtctgtcgtgtccagagaaatcaggtgcccagagctggcaactggaagacccatcaccatgaggcagcgatgtcactctcttcatggccaacttgataacggcataatgcaggattgatctttacacgtgtgtgtgtgtgacgtgtgcgtgtgtatggacttggatggctcttcgaaggaaatctttttaaggaaacctacaaactcctttactgggttaatgggagcatcagaggaagattccagaaggaaacataaacctagggagacaagatgaaaactgagagctttagccacccccttcagggggagaatgtctctttttttgtcaactgggaaccatttgggtttgctgataatggtgacagacagatgcagatatagactatgccataacttacaagattgtctctgatactgtgaaatcagaattggattttctaaacgctaataaagcctacactctgggtacctcacttctactccccttccaaggatttatacctaattttgtattcatcatttagcctctgttttttcaaagagagccttccaaattgtgagcattaaacccctcacagtgcttggatccacctttgtatggaattgtgagtccttaagaaaaagtgccagtccttctttttttctccaaagaatctgtgttgattcctagaaatgtggcagctgataagcatgaaggaggagtcgctgggacaggccatggagcctgagaaccctgaagatcaaaggcaggcaaataactgctgtgggaagggcgtgaatttcccacaaagtgttttgtgaacatttacgtaacttcttttgtgttttgctactaatgtagtcatttgcttccatagccgttttgggtaatattaccaatatgaactcataagccgttcatttacttttgagatgaaaaatgttcttctttcagagcatcttgcttgccataataataggtgaagttgagcacagtgacctcatggctctggaatgaacgaaaaatcacaattcacacttaactcctagttttttttaaacgaaaaaagaaataaattatcatcctcaaagtgtaatttttaaagttccttttttatgtacctaatatgtctgggccaaatttaatccatctactcaaccatgtccattcctaatcataactcacatagaaaacgtaaaagacagaagctaagacaaaacttgtccttataatatgcatgtttttttcatggctgagatggggggggggttcaatgagttaagttactgaattctaaatactttagaattatgctgttaattgtatttatagctaacatatggtattaactttgatgtaatttgcattttgggggactttccagaatgataaaacgattttgggccaggcacggtggctcacgcttgtaatccaaacactttgggaggccgaggtgggaggatcacttgaggtcaggagttcgagaccagcctggtcaacatggtgaaaccccgtctctactaaagatacaaaaattagccaggcacagtggcatgtgcctgtaatcccagtttctcgggaggctgaggcaggagaatcgcttgaacctgggaggcggaggttgcggtgaaccaagatggcaccactgcactccagcctgggtgacagagcgagactgtgtctcaaaaaaaaaacaaaaaacttttgcaatttgacttgagtaatgataacatgcatgcatacattttttatcacactaaacatagagtcggtgcattttactatgcagacttccacaaagaaaatctgaatgctacttgcaaaaaacagtttttgcagtttctttttccttttaactttttaaaaggttattacacttttttttttttaatttgtaactctttcaaactttaggaattctttgaccatgtgaaaaaactttgggacgatgaaggcgtgaaggcatgctttgagagatccaacgaataccagctgattgactgtgcacaatagtaagttgtgtcctgtacaagttacagggccctttgaagaatatgattgcatgcatgattatgctgccttctcagtactgaagtttcttgagtgcaaggaatgaataattaaccttttatgacagaaatcaacttttaaaatgagacatgtttagtagatggaaaattgaaaatccagtaagaatcaatgtttctgagaaccagaaagtttcgttcattttgtcctgattgttttcagttctcttcattttttgacttttacatagagctatatgcatattggtaagttaaagtgaacttccatggttattgttatgttttgattttggagcaggggcctcaaattatattttaaaataatacgaaccttgtgatcaaggtcaggctcttaaagtaatgtgatcattctgtacacattagaagtataccagaaggctgggcccgatggctcacacctgtaattccagcactttgggaggccaaggcaggcggatcacctgaggtcaggagttcgagaccagcctggccaacatggtgaaaccccgtctctactaaaaatacaaaaattagctaggcgtggtggtgggtgcctgtaatcccagctactcgggaggctgaggcaggagaatcacttgaacctgggaggcggaggttgcagtgagctgagattgcgcccttgcactctggcctgggcgacagatgagactctgtctcaaaaaaaaaaaaaaaaaaaaggaaaaggacacttggtgccagggctggctcattgtttttccctgaaacacttacttttccattttgttttgctatgttagatatattctattgctattagaaaataatggtccttagaacaatgttaaattattaagaagttctagatatgtttgctgttttgataattaaaaattgtagttgactagttgttttatgtaaagctgtggatagcaagaaatgaaaaattttacattttaaatctctaaactctaaaaattcatatacatagaagagattccatagcataaaatctgggaattcatagtatttggaaccaatcagtggagctgcattgtgaatgttgatttccaggagggagtggttgtgttggcccaagctcctgcttactaggttctacttccactcaccatcccccatgggatgggcctgaagcacccagcgaagcccagagagccaggctccaaggggcccatggcaagggccagaggaggaggaggagcggggagggggaaggaagaggaggaggaggagcggggagggggaaggaagaggaggaggaggagcggggagcgggaaggaagaggaggaggaggagcggggagggggaaggaagaggaggaggaggaggaggaggagacactaagcatggctggggaaagaaagagtgctcataaagaagtgagggaggtcagtggggccagacacccccagggctgaagaccacagcaaggaacttgggttttctttcagctacagcaaccagccatcccagccataggctccaagcatgtggttgtcagggaaagtagattgcagaaggcagagaccctgtggggtgccttgggttgggctggggtagccatgcaggtgagtgctggaaactgaagggcagaactagctggtggacttaatgaggaaagggatccaggagggtacagggtgaaccacggtgccatttactgcaaaggggacatctaggagacttagggaagagatccagctctgatttggagaagttgagtttgagatgccaaatggagagccatgtggaggcatcgagcagtcagtctgttgtcagagcctggagctcggaagagaggcagatggccccagtgtaccggtcatcagctactgatggtatgcagagcctgggaatccagagcacacgaggagtgcccgcagagaggcaagggctctgggcctgagaaagttcagcttcagacaaggaagagcctcacagcacatctgggagagagggagtgcccagcagagggcacaggtcgtccgggccagatgctggagagacccggaaggaggagggctgatggtcctagtaagattgtacttaacctccttcttaccttgtccaaggacaggatgtcccatgctcagtccgcaggcatgtggtgagcgcgcttggcaaggcaaagtatagtaaagcatcgagaatggagcgcacacctgctgcaaacaccctcagaggaccgtagaaagggttagccacctccattatacactaaagaaagtatttactcatgaaaatttttaattaatgaaaaaagtatttaaaatgtcctggccgggcatgatggctcatgcctgtaatcccagcactttggtaggccgaggagggtggatcacctgaggtcaggagttcaagaccagcctggccaacatggtgaaaacctcatctctactaaaaatacaaaaattagccaggcatggtgctggacacctgtaatcccagctactcggaggttgaggcaggagaatcgctcgaacttgggaggcggaggtggcagtgagccgagattgtacacctgcactccagcctgagtgacagagcaaggctcgtctcaaaaaaatgaaaattaaaaaattaaaaatgtcctgtttcctgccatgcacgacaagtccttcaagattttctttaaatagctgggtgtggtggctcacacctgtaatcccagcactttgggaggccgagatgggcagatcacgaggtcaggagatcgagaccatcctggctaacacagtgaaaccccgtctctactaaaaatacaaaaaattagccgggcgtggtggccggcacctgtagtcccagctactctggaggctgaggcaggggaatggcgtgaacccgggaggcggagcttgcagtgagccgagatcgcgccactgcactccagcctgggcgacagagcgagactccgtctcaaggaaaaaaaaaaaaaaaaaagattttccttaaataatgtttgtaaattggccctgcccttgagtttacatttgtgaatacagttctgtggttgctctcatgttatttggttgctttcggtttggatgtgggagtttggaaaggctctcaggggaactccaatgcagtctgagcagccttgggaggcttgcaggtgctgaaaaggccttttatcttttcctcttactctcactcactctttctgcaatacttcaatcgttaagcagtcatttaaggcacaatagaagctgggcatggtggcacatgcctgtggtcccagctactcaggaggctgaggtgggaggatcccttgagcccaggagttggaggctgccgtgagctatcattgtgccacacactccagcctgggcgacagagtgagaccctgtctcaaaaataaaaagacacaatagaattatgttaaactcacgtgctttagaggtgggaagaatataccaggaaaatgctagcaactggaatttctaaagcagttcttaataatattaaaattcatacacctctgcatttgaagctgttgtggaatatgcgttctatcatcctttttcagtatttcagtatttattggacaaagactggccttggagaacaccacagttctgctcaatgccacattagaaatttttctgttgtaaaaaaaaaaaatggacaagttattcctgcatcaccgtagtaatagaattaataatttgatttaaaatagaatatgtgaaaataaatcatggaaaaagaaatggtgtgagcttggcaataggcagatcggaattcagattctggccccacccccgaccactgtgtgagactttgggcaaattacttttatgggaaaattaacaggatctattctcatatggttgtgggattaaataagtagatatccaaagtcctcaaaatagtgcctggcacttagtaagcactaggtaagtgttatctcttgctagaatgtgactgatacagaaactgacacctagagaggtgcctacgatcacagttacccaggaacagatgagaccacagccaaaatctgctatttcttttagatttattccttcaaattacagaaagccacagataaaaactgccttgtgagcgaggccccagggcctcaggccaagactcccttctaggcttgtcaggaataaaacacctttagccaaagcctctgtttcagctttctgaaccctgggcctgacagaattacagaaactggttcccccttgaggcctcactatcctaggctaattgtaacttctcccgtttagctcgtttcacccattttaatacatacaggaatcatgtggagcaataacctttgcatcctaataactatcttgtttttcctatagggtcagattctggaattgagggatgagggatttcaaaaacaactaacatttcaataaatctgttagaccaacatagagctgccaaattcttttactttgccaaataagatgttagaaaaaataaaagctgctcccatctcccaccaccgtcacttcataaaagaaaggacatttcaaaatcaggccagtaacaggacatgtctcagaccacagtctgagtgcagtcctcagaccacgagactcgtgtgtcttgttcagcctactaggttttgaattccccagggaatgtgtctcattcatatttaaatgcccagctcctaggtgggtgttcttgtataaagtcagcgctcagtcaacgggcgcagtagctcacgcctgtaatcccagcactttgggaggccgaggcgggtagatcacttgaggtcaggagttcgagaccaacctgacaaacatggtgaaaccccgtctctactaaaaatacaaaaatttgctgggcatggtggcacacacctgtaattccagctactcaggaagctgaggcaggagaattgcttgaacctgggaggcagaggtttcagtgagctgaaatccgtgtactccagcctgggcaagagagcaagactccatctcaaaaaataaataagtaaataaagtcagtgctcagtcggtgctatctgaaatatgtgaactgaccaaaaaagagccagtatttgatgtggtattaacggaaacagccaaccctcatctccttgacgggtccgtactcactcgcctttccccatgctgattcccactattcctgcgtgttttccattttctttacatctaaattgctcttggaaaagctctgacttaaatcttggtgtgacagtaagtcactgtgtcaccttgctcaacagaattgcatctttttttttttttttttttttgaggcggagtttcgctcttgttgcccaggctggagtgcaatggcacgatcttggctcaccacaacctccacctcccgggttcaagcgattctcctgcctcagcctcagaattgcatcttgaaccctagtgcttatgaagaaggtaataattctaagacctcttaaagtcatttattccatggcaggagtcatgggagtcttttgggcttctcagtgatgctgaaattgttagggcctcaggaggccaggctgccaattaagagaatccagtagttcctcaaaaggttcaacatcgaattacccagcaattccactcctaggtgtaaacccaagagaattaaaaacatacctccatacaaaaactggtagatggatgttcatagcaatatttgtcatcataactaaaaggtgaaaacaaccgaaatgttcatcaaatgaagaatgtatagacaaaacgtgatacatccacacaatggaatattactcggcaatgaaaaggaataaagtgctgacacatactacaacatggatgaaccttgaaaacatcacactaagtgaaagaaataagacacaaaaaagacacatattatatgattacatgtatatgaaatgaccagaataggtaaatctacagagacagaagtcaagtagtggtttctagggaatggggagagggggaacttaagagtgactgctaatggatgatggagggtttctctttgggataatgagcaagttctggaattggatagtggtgatagttgcacaattttgtgaatatactgaaaccatggaattgcatacatcaaaatggtgagttttgtcttatttgaaattataagttatgtaaattatatcttgatttaaaaaaaaaatgacaggagagggaagaatccagggcaggaaaggcaatgggagctctgtggtcaggcctggccatgggcacggtctaggggaggactcatgtgaagacagagatgctgccttgtccttcaggcctccttggtgagtcagcggtcctcttctgaagtacaggttaaggtcagaatatgtgtttataaggcctgcttcttcatacttgacccaccgaaagtacatgcccccacacactctctccaagcctgcaaatatgtgtgcggatagggacctccaaaactgtaaagcacccatgaaaaaagtctaaggctgtgaagatctgtgttgtgctgcttggcatgcaaggaatggaaacttaccaagtgacttcaagaaaaggggggttttctcctgaggatctggaagctggtattgagccaaaggccctatggggaaaagtattctgttcttgccttcaggcgtttcaactttgccatccctgtttactccactcgtaccttgtccactccgtaatctgcagcccaattccagagggagcaggggagatgggctttgctaaggccaggccaattcttacctagccttgtcccgtcaaacaggcccactggccagttgctggggctcacttgacccagtcggtggccatttgtgtgtccctgagcaggtgctgtggtgaggcagggccagccaggtggcactgagcagctgcaggactgatgtgattgcaggactgtgagaataattattatcactgagccttaaatactgtgttttctgtaaaagtgagtgtccttgaagcattgagcaagctgtgcaaattaccagagattatattttatattgaatttcccagttcctactgtgaaaatttaaaaacaacctctactgaattatcccccaaaatgcatttcctctaagtcctaatactaagtgattatgtaaccatggaaacggttattttgggagaaagaaaatcagagagaattggatctttagcatagctttataaaatcggactttgcttaatggaactttaaactccacagctcacagagtgtaggttcttatgctcagtacgacacatgataagctcctttagggtacatccatttcaataattggtttttgaattctctaaggagagacactatcccagaaaaaaagaaaagagcagcgaaaaaagtcaagtgatttttctgctgcagtcccagatctgttttctgtcatctttcaggagttttcttgaccccattggccctctttggcactgatttgatcctatggctgtcaagtcctggaatactcagctttcaggacaatctggaaattattagcacaaatgaaatttcaacttcaaagttaattttttgaaggtcattttaaaatgaaaggaattgacctggcgcggtggctcacacccgtaatcccagcactttgggaggccgaggtgggctaatcacctgaggtcaggagtttgagaccagcctggccaacgtggtgaaaccccatctctactaaaattgcagaaattagccgggcatggtggtgcacgcgcctttaatcccagctactcgggaggctgaggcaggagaattgcttgaacccgggaggcagaggtttcagtgagccgagaccacgccattgcactccagtctgggtgacaagagtgaaactccatctcaaaataaataaataaataaataaaaattttaaaaaatgagatgaaaggaactataattttaaattatgcatattactgtatatactgttgaacttattaaatatatagcagccacctagatgcatgtacatattcttcttacctctcattttgatctctatctctagccaaatttattttcagattttttttttttcgagtcattctgtcacccaggctggagtgcagtggcgcgatctccactcactgcaacctcagcctcccaggttcaagagattctcccacctcagcctccctagtagctggaattacaggggcccgccaccactcctggctaatttttgtatttttagtagagatgaggtttcacaatattggccaggctggtctcgaactcctgacccaagtgttctccccaccttggctcccaaagtgctgggattacaggcatgaaccactgtgccccgcctctcagatttgttttctagagctgcaaaattggaaatgttcctgtttatgagcactacaataacatttactttagctatgaaaaataaatgtagcagcagggactaggtaggtagaccagatgtctctgtgtgtggccatcagcccctgttacatgtttcttttattctcctctagaaacccttgccctgagacatccagaagaaaatttttagtgggattaccacaaatgtttttgtgtgtaaaacctgtttttaaaatatctcagaaaatctacttacttgggtggagaactgcagtggcttcccattccttatctaacgtaaccctcagacttcctatacagaaaggatgcctggaacaggatggccttcagggcctggcctctctgcccagcacatacatcacaggaatctccaatcatggcgtgaaaatcaggaccttcaaactataaaacaaaaatcacatttttgatgccttagagctggatttttcagtatttcttagaaatggaacgcttcgtcacacggaactgtatggaatgccactagcaaagagggagaagacaagtcccctggtgaggagccggagcccttgtattcgcttctgcctgctgctgtgtggcccctctgttgcctccaaatgtgaggctccccggggtgcggtttaaaaactagtgcccgttttcaaaattcataccagcagaaccaaatgcaatttatagccaatgccaaagcaaagtggtattttattaaaataaatatatgaaaccaaagtgaaaatttaataaagtaaaatttagtttacatattcacatttgtaacatttacttattataaacaaacctaaagatctctatggctatattgataagaacaaaattggaattcagtggtcttagatgacggttgagtttttcgctggactcaacatccagcttattcctgctagggaggggcgtgtgggaacatcctgacatatgcagatgagttgttgcaaatggtagcaaaaatggggtcttttgttaaggttgccttgtaattacaagaaacattttaagttaaatgatgtagaaatgtgaaaaggagaatcataagaaattttcttctggctgggcacagtggctcacgcctgtaattccagcactttgggaggccaaggtgggaggattgcttgagcccaggagttcaagaccagcctgggtctctagtgtctacaaaaaaaaaaatacccccaaaaattagctaagtggggtggtgtgtacctgtagtcccagctacttgggtggctgagacaggaggattgctggagccttggagtttgaggctacagtgagccaagactgtgccactgcattccagccaggacaacagagtgagactctgtctcaaaaaacaaagaaagaaattttgttccaagttgaattcttaacagtctctaacagctcttcacattctttaacacaaatgtgacatctgataaacgttatatttatagcgttgggttatgggttacgttgtcccctgtgagaacatggcagttgctctgtgaggagagcttgggccctgcaggtgagccccagctcaagtggccttgggcaaagcagggtgtgcatcctctgtgcctcttagggactcgcttctaccgtaagactattcaacaggtgctgcattgtctacaaaaatgaccagaacctccttccaaacaacacatttgaaagaagttgaaaatatgtgcataatatttttatgggaatttgagctacacagatagatgcatttttcaaaactcagcagatatacacttaagatttctgtttcattggacatgcattttacattgaaagaaaaagccatgaagaaatattgattcttagataacattctgtgagctgcagtatttcagagggagtgtactgctgtcttccctttttttgaaatacatcaaaaataagatgggttgaggaatggatacatggatagatctgtgattttttttttaagtacaataaaatgaagctgggcacagtggtgtttgcctgtaatttcagctactcaggagactgaggcaggaggttcactcgagcctaggagttcaaggccagcctaggcaatgtagcaagaacctgtctctaaaaagaaaaaaaaaagcctaaaagtacagtaaaacgaaaatgtcagaatctaaatagtaggtattcaagtgttcactacaaaattatttctaatttattttatgtttgaaatttttataataaaatgtagggggaataaagaaaaatatgtatatgtaaagctgtgatcagaagcttacagaaaaataaattacatagtagcacaggttaataaattgacagtaccctacaaattacagtgtgaaatgtaagacgtgtgtgtcaggttttatcacaatacttttgtttgctgaattctttgaaacacttccaaggaatccagggaactcagggaaaacaactaagtactgtactactattaataaaggtacagtttcagaattatttaagacgtgcgtcccaccaggcgaggtggctcacatctgtaatcccagcactttgggaggccaaggcaggagtactgcttgagtccaaaagtttgagaccagcctgggtaacatgttgagacctcgtctctacaaataatttttaaaaattagctgggcatagtgccacacacctgtagtcccagctactccagaggctgagttgggaggattactttagcccaggagtcaaggctgcagtgagtaatgatcatgccactgcactccagcttaagtgacagggtgaggccctgtcgcaaaaaataatactaataggccggctgtagtggcttatgcctgtaaaatcccagcactttgggaggccgaggtgggcggatcccctgaggttgggaggtcgcgaccagcctgaccaacatagagaaaccctgtctctattaaaaatacaaaattagctgggcatggtggcgcatgtctgtaatcccagctactcaggaggctgagacaggagaatcacttggacctgggaggcggaggttgtggtgagccgagatcacaccattgcactccagcctgggcaacaagagcaaaactcttatctcaaaaaataataataataaataataataataaagaggtgtgtgctctgtattgcttagtacccaagtgtagctgtaagatagctcatatttattgaaacttgccctgtggaggcacattttatgcacattagatgaactaacatagtaatcctcacactaacccaatgagttcattatctttattttgcagatgaggatataaaggcacataagattacataagactacacaataccaactattacttgattgatccaggatttcaaattttaaacctaaagattatgagagactactttagattaaaagttcaccaagcattctgtgagcatcagatgcatgctagacactgccagccactgaatgacaaagatgaatgaggcatggacctatgcatttgaaggagattgcctcagggacatcctttttctcagattctgaaggaactgtcatcaacttcacatctccatccacttcatatcttgaacctagttttccaatgaaagccaggatagctttttcttgagatggagtctcgctctgtcacccaggctggagtgcagtggcacagttttggctcactgaaacctccacctcctgggttcaagctattctcctgcctcagcctgctgagtaactgggattacaggcacatgccaccacgcccagttaatttttgtatttttagacatgccaccacgcccagttaatttttgtatttttagtagtgctggcgtttcaccatgttgggcaggctggtctcgaactcctggcccacctcggcctcccaaagtgctgggattacaggcgtgaaccaccacactcagcccaggatagcttttgatgtacatatagaggtccttatgattcaagaaagtgaaaaaacaagctcatagaagggggaaaatgtttataaatcatgcatctgataaaggacttgtatctagaatcataaagaactcttacaaatcaataataacataagtaaaccgatttttaaatcagcaaaggatctaaatagacatctcccaagtaagatagatgaatggctaatcagccacgaaaagatgctaaacatctttagctgttaggaaaatgcaaagcaaaaccacagtgacattccacttcataaccctagggtggctgtgcctaaaaagtcagataaaacaagtgttgctgaggatgtgaagaaattgggatcctcatatactgctggtgggaatgtaaaatagtccagccactttggaaaacaaactggtagttctaaaaaacgttaaacacagttgccatatggcccaccaattccactcccaagtgtatgtccaagagaattgaaaatatacatccatgcaaaaacttctaggcaaatactcatagcagcattattcataataatcaaaatgtgtaaacaggctgggcatggtggctcacacctgtaatcccagcactttggaggccaaggtgagtggatcacttgagctcaggagtttgagaccagccttggcaacatggtgaaatcccttcttacgaaaaattatccaggcatggtgatgcgcactggtagtcccagctacttggggggccgaggcgggaggattgcttgagcccaggaggtcgaggcttcagtgagccaagattgcatcactgcactccagcctaggtgacaaaatgagacccagtctcaaaaaaaagcgttaacgacccaaatgcccatcagccgatgagtggataaacaaaatgtgacgcatccacacgatagaatgttattcagttacaaaaagaaatgaagtcctgatgcatgctacaacatggatgatccttgaaaacattatgctaagtcaaagccaccagacacaaaagaccacatatggtatcatttcatttatatgaaatgtccagaataggtgtatctacagagacagagtagacgaatgttgcatggagccaggtggacttgggggatgaggaatcgctttaatgtgtacagagtttcctcttggagtgatgaaaatgttctaaaagtgattgatggttgcacaactttgaatatacaacaaaaaagtcattgaattgtgcatcttaatcaagactccctgttgcaactggcttgtgagagctttacttggacacttcagtaaaattaatttggagactcctgatcattcgagtctgcctaatttcagggccttctgatttaataccaagtgggtaagatgcattatgtttcatccatgtgccacgcatggtgacagtgtggggatgcttctgagaggatgcggtatgagatccatagcatttcagattgttcatgtacttactttgtctttttaaaaatcagtttaagagagaaaggcggaagaaatgaaatagaaaagcaaaaaatactgattcaaaatacagtacccatactgaaaatatgtcttaaaaatctcttctaaaagactatctttgtggaaattggcaggtaatttctaagttgtttccataggatttggagttgttcttctccatgcagtgcgtgacacagtaacatataaatatgagatgttatagcatatatcactacttcattcctttcaatagttgaaccacatgaatgaacaactaaatctaaccaaattttgtaaactgttcattatgatgggctaactgacatgaaattattgtgtaccactgtgcctagaacaatgcctggcatataataggtgtttaataaacttgttacatgaatagatttttgtgttatcactgttctttttctgctctctctaccattcatagcccagtattcctatgtatacaaaattaatttttggctgggtacagtggctcacgcctgtaatctcaacactttgggaggccaaggcaggaggatcacttgagcctaggagtttgagagcagcctgggcatcttggcgagacctcatctctactaaaagtaaaaaaaaaaaaaaattagctgggccatggtggtgtgcacctgtggtcccagctccttgatatggtgaaagcgggaggattgcttgagtctagagtttgaggctgcagtaagccatgatcaagtcactgcactccagcctgggtggcagagcaagaccttgcctcaaaaaaaaaaaaaaaaattttttttctttaaagctcagatagacgtaagcataatatctcttcatagcagcattattcacaattgggcaaaaagtggaaacaacccaagtgtcatcagctgacaaatggataaacaaaatgtggtgtatccctacaatggaatatgattccaccgcaaaaaggaatgaggagctgaactgtgctacagcacagcctcctttagagcacaagcgttctagctcagagaaaagcgaaagctgtaatcgtgcattgtgtgagcacttccttcgtcccagtgaacaataataatgtgttcacaatatcaaggaatgtaatgaaagcttttcaaatagaacttaaggtataaggaagctgctctttgataatttcatatcaaagaaagcattcaaaaaaggaagaaagtaaaacctttcttagggtagtgcatgcattagcggttagaattaaatatctttatttcaccaaactcccttgatcacataataactaggatccatgatatcaacaaaagctacctttgaactatggcattattctattaactgacttaaggaactctccacacaaatagaatttccatttacttacaattttattcatgtgacaacaattaagtgcatgggatgctcgtatttctcattctattcgatatttgtgaaaatgcagtgctctgcattatttctgtgaagaaagaatgaaagattccttatccagctactgaggcaggtagtcattgctaagaaagaaactgctttgccatgactttctgccaggatacatgcagtgactaaatatatcttctaagatagacaagacttaacagatcgttttgccaaaaaaggaaattttttattaacctcaattgaacatgtttaatgacatcccagatctcttttaaaataggagactcagcagaggtgaacactgatgcaaaggtcagtttctcattccagctcttcctttgaaacatagtgtgacctgagcaaatctgtgtgtttggtatctggggcaccccctctgagaaagctctttatctaggagtcctcagacttccctttgttaccctttgttgaccacgtgcttctcatagttagcgttcttccctggaatgatctacaggagacctggtggacgtattcgggaatgctgtgacagattcagtcaatgtacacagtcatagctgcagagaagaggaagcacccggaggcctccagtgagcaagagccatgttgaatgcactttgtccttctttctttgatttagttcacgtaaatgacattgaggaatttgctttttcttttctttttttttttttttttgagacacagtctcactctgtcgcccagactggagtcagtgctacgatcacagctcactgcggcctggacctcccaggctcaagtgacctcccacctcagccacctgaatagccaagatcacagatgtgcaccacgatgcccagctaattttttgtatttttagtagagacgaggtttcgccgtgttgcccaggctggtcttgaactcctgggctcaagcaacctgcccacctcggcctcccaaagtgttaggattacaggcatgaaccaccgcacccagccttcatcagctttcaattctcttttaacccctaaactaatcttaaaatacactgtctcctacaaatatgttaagatttactatgaagtattgtttttcagatggcagtcacagctacaatttattatatttagggcaaggttaatcatgattatttgaagacagctttgtggttcccggaaaagatatacctgctagggccgggcacggtggcctataatcccagcactttcggaggccaagttgggcagatcacctggggtcaggagttcaagaccagcctggccaaaatggtgaaaccctgcctttactaaaaatacaaaaattagtcgggcatggtgacgcatgctgtaatcccagctactctggaggctaaggcaggagaatcgcttgaacctgggaggcagaggttgcagtgagccaatatcctgccactgcacttcagcctgggcaacagagtaagggtctgtctcaaaaaaaaaaaaaaaaaagaaagaaagaaagaaatacctgctagaattcctcattcatcagatttatggaggaagctctgtgagcacctctgcttcccaactgcggggatccttccatgagaagaaattctggggaagataaaaacctctgctttatggaatgttacaaatgcatcaggtttgcatagggaattacttacctatcccccatactgttcattctctagcctgaaactcagtaaatgtttgtggaatatacgtatgaactggaagattgtagcccaactccatcaaataacgaaagaaaacatatgttatattttagttcagattttttttttttttgagacggagtttcgctgttgttgcccaggctgaagtgcaatggtgcaatctcgactcacctcaaccttcgcctcccgggttcaagcaattctcctgcctcagcctcccgagtagctgggattacaggcatgtgctatcatgcccagttaattttgtatatgttttttttttagtagagacagggtttctccatgttggtcaggctggtctcgaattcccgacctcaggtgatacacctgccttggcctcccaaattgctgggattacaggcatgagccaccgcgctcggcctagttcagatatcttatatctaatgacttggtgtagcggcccccagcctttttggcaccagggactggtttcgtggaaaactatttttccatggaccgtggtggggtggatggtttcaagatgaatccagtgcattacgtttactgtgctcttctattacgattacgttgtaatatataatgaaataattatataactcaccataatgtagaatcagtgggacccctgagcttgttttcctgcaactagatggtcccatctgggggtgatgggagacagtgacagatcatcaggcattcgattctcataaggagcatgcaacctagatccctcacatgcaaagtttacaatagggttcgtgctcctatgagaatctaatgccatcgctgatatgacaggaggtggagctcaagtggtaatgcgattgatggggaacagctgtaaatacagatgacgttttgcttgctggctggcactcacctcctgctgtgtggcctggttcacacagcatgagaatgtggcctagggtttggagacccctgacttagttcatactcattgtaaagttagcacttcttttaaaaacattcatctttggccttctttcaactgttgactatttgattgttgactgtttcactgttgagtgactttagggtgttgactgtttcagaagaaactgaatgctattatggtacattataaaggtacaaaagtgataagtattctgctttgttagaggatttttcacattctaaagagatgttaaggccaggcacagtggctcatgcctgtaatctcaacatgttgggaggccaaggtgaaaagatcccttgagcccaggagtttgaggctacattggactatgattgtgccactgtactgcagctgaacaacaaagcaagaccctgtcgctaaaaaaaaaaagaaaaaaggggctgggtgtggtggctcacgcctgtaatcctagcactttgggaggccaaggtgggtggatcacctgaggtcaggaattcaagaccagcctgaccaatatggtttcacctactaaaaatagaaaatatgtaaaattttttactactaaaaatacaaaaattagcggagagtggtggtgcacacctgtaatcccagctactcaggaggctgaggcaggataatcacttgaagcagggaggtggaggttgcagtgagctgagatcgcaccactgcactccagcctgggcgacagagcaagactctgtctcaaaaataaataaatagataagaagaaaagaaaaaaaggaaagaaataaagagatgttaaaaataaattatttctagtaggactcaccaagttaagttatatttgatttctaataccattttgcttcctaacgaaagacagtagcttaaactaacaaaatcgttatgtcaccaggcacagtggctcacacctgtaatcccagcactttgggaggccgaggtgggcagatcacctgaggtcgggagtttgagaccagcctgactaacatggataaaccccatctctactaaaaatacaaaattagcagggcatggtggtgcatgcctgtaatcccagctacttgggaggctgaggcaggagaatcgcttgaacctgggaggcagaggtttcagtgagccgagattgtgccattgcactccagcctgggcaacgggagtgaaactccatctcaaaaaaaaacaaaaaactttatgttttggccgaaaagtttaattacagttattccttgacgaaaagaaatggccaagattagccttccatggacactaatggctaatgactccttactttacatgaagtgtgtgggttagactagaattgaagggattttaaattgaaaggttctcagggccgggcacagtggctcacgcctgtaatcccagcactttgagaggccgaggcaggcagatcacctgaggtcaggagtttgaggccagcctggccaacacagtgaaaccctgtctctactaaaaatgcaaaaattagctgggcgtggtggcgggcgcctgtagtcccagctactcaggaggctgaggcaggagaatcgcttgaacccgggaagcagaggttgcagtgagccgagatcacaccactgcactccaacctgggtgacaagagcgaaactctatctcaaaataaataaataaataggttctcaggatacatggttcctggaaccagtgctctccattctgtggccatggccgttttaaatcacttgtgccttaaacttaatctaattgagacatgatgctaagtaaaagctgggtctaaggcagatcaatcacagcatcaattaagggtacgagtgagtcagctgcacacacatgtccccgggaacagcagtgaagaagaaaaatgctttttccctccaccaccatctaccaatactcaatgcagactagccctgggaagagctcctgacttctcacaatctgttcccatcatgaacagaaggtgctccccgaggaggacgagagaagattttaatgtgcaacaaaattaactggccaatttgtacattcgccatttttattttttatttatttattttttgatttattttattttattttatttgagatggagtttcactcttgttgcccaggctggagtgcaatgatgtgatcttggctcactgcaacctccgcctcctgggttcaagcggttctcctgcctcagcctcccaagtagctgggactacaggcatgtgccaccacacccggctgatttttgtatttttagtaaagacagggtttcaccatgttagccaggctggtctcaatctcctgaccttgtgatccgcccacctcagcctcccaaagtgctgggattacagatgtgagccaccacgcccagcctcgctatttttaacttatagattgtaagcttaatgttcaggttaaacataaaaattacgtaagaacaactggcttccatattccttcagaggtgggatttgtaccgtattacgccagacgcaatcctgtccttctatcttcaggtttgtaatagtaaaatcattttccatccagaagcatgttttcagaataaactatgtttcccagtagtcaaccagacagctgtgagatatggcaaaatgctgtgtgcaggagagtgcacttttcttgaaaataaaaaaaaaagtagcataataaagactgctaacaaataataagaacacagattggaaagaggaatatatcgtgtgtttcatagacctaacaatatttcacgtgtttggttccctgccatattgcttgagtttgtacgttagtttgtaggaacctgaccaaaggagtagattttcctcctacaaaatgctctccaactagaggagtcagagagaaatgggcccagttgaggcagaggtagcttgcgccactccgggtaacagagtgacaccctgaaggaaagaaataaaggaaggaagggagggagggagagagggaaggaaggaagagaaaagagagagagagagagagaaagagagagggagggagagggggagagagacagagagagaaggggcccagtcagccttctcagttttgctgtgtcagctctatatggtcttcacaatatttgcaaatatccaatacaaactgccaggaaaacgccacttaaataatacatttttttctcttagtaagtcttgtttcagggattttttaagttaagcggttttgagtggccctctctggtgatctgacctgatgagggattattacccactgttagatcataggccttattttttccagctttataaatgaggaaattgaggccagaaaagagaggcatcccgtaccagccttagagggacaggtgtgttgagatttcattttcagcttctcaaagtggtaagttgttcagtgtaaagaaggaggaggagaatcactgtgttgctaaatgagtgaatgccaacttcgactaacatggaagttaggtttctcataacagtattaactcaccacatcaccaagaggaataataaaccgtgcatttaaactgcagatggaggaatgcagaattctgtttacatgctgctgttctatttctttctctttcttatttttatttttaaatcgacatcatttttttatatataaatatataaatacatatataaatatataaatataaatatatatataaatatacacacacacacacacacacacacacacagactcactctgtcagccaggctggaatacagtggcatgattatagctcattgtagtctcgaactcctggacacagagatcctcccacctcagcatcccaagtagctgggactacaggcacatgctaccatgcccagctaatttttattttttattttttgtagagaccgggtcttgccatcttgtccaggctgttgttaacatattttgagggacagtgactttggttttgttttgttgttgttgtttttgagacaggatcttgttctgtcaccccgaatggagtgcagtggggccatcatagcccactgcaacctcaaactcctgggccgcctcatcctcccaaagtgttgtgattacaggcatcggccatcacacccagccctggacagtgactttgaaatttcccaaggcttgaggagtctcagcgtttttaattctagctgtgttcccaaataaaaaggtgatgcagaggaaaatatatatatacatacatttaatacatgtgtaataaattaatattatatatgtatgtaatgtataatatatatatacatatatgtaatgtattcctcaaaatggtcaccaagtgaatatgtatccaatgccatttcctttggctacattcatatattcctacattcaacagaaatttattgggtatctgttttccagaggttgaatagtaccaggtataggtagtcaaccagaggtcaccatgccacacctaataggaaagcctgctgctcagcaaacacaaatatatgcaaaaaatgcaaactgtgataagtaggaggagcccttagtttgagatataacatttcaaaatcaccagaaacatactgatgagaatccagaaatagtttaactcacaagagaaagttatttttcacatcttttattttcttttttttttttttgagtgacttcagacttagcaaatgattcttttccagtgaagtaaaactgaatgtcctgcttaaatattttcattttaaccacttgtattatttcaaagtaaggaaagaaatcagccagatgtgccaaatgatagcatatttttcttccccatttcatgataaaaataattaactggtttgacccgcagctttctagaaatgtctgcactttaatcaaaaatttctttttttttcactaagaaaatttggctatgggacttgcaaggtttcaatgttaacgtggtagttgctaaacaattccagtcattataaactgtatgttctgttttcacatctgtattgggtccaaaacattttcacgtacaattagcatggttgtaggaccaaaacctcctttaataataatcccaaaataatcttccttttgctagatacacaaaaagagaaagcataccaagctgaaacagaccctgaaggttgtcaacctcaatggaatataaagaatggaaaaaacttagagccagggtagatgcagattcagataccacggtggtcagagataattcttccaaatccaactcgaatgacaaggtagatggaggtgccagtagtaaatgggaatgagcagatttgggggaaaggactgagttcagtcactttgaggagttgccaatcagtcttgggccacagagcgcccagcaccaaggagacagctgggcatatgggtttggagctcagaagcaaatctctccgtggaaatgtgaacatgagagcagttagctatggatcctattgaacaccgtggaaatggctggggtgttgataagagaaagaagagggcagggctggggaactgtcagggagaggaggagaacagccttctcttcagaattctagtcagccctggtctctctgggaagatggcttctggaactccacctataccttctctccagcacagaaagtagaggtgtcatcatccaaggaatgtagcttcaggtgatggcaacttcttttagcaaccatactttttctttttttttcttttctttttttttttttgagaaggagtctctctctgtcacccaggctgtagtgcactggcgtgatctcagctcactgcaacctccacctttccagttcaagcgattctcctgtctcagccttccaagtggctgggattacaggcatgcaccaccacacccagctaatttttgtatttttagtagagacgggctttccccatgttggccaggctggtcttgaacccctggcctcaagcgatccacccacctcagccccccaaagtgctaggattacattcctgagccaccatgcccagtcaacaaccacactttataaagttcatattaactttgcagtcaatgcaactctgtcttctcaaaaaaacagctggaaagtcaacttctttctttaggctacatgatgatcattattttattcttattaattcatgttcattggattccccattgccttattactggtcccaatacatccatggaatccggtttctaacacttacctaatgtgtgggttcaggcaagtttcttagctctctgtgcttccttttcattacctgtgaactggagacatttatagtatctacttcatagggtttgcgaagctgaaatgagttaatttatgtaaagtgcccagaacaatgtctggtgcataagtactttattaatgttagttgtcattatcatcatcatctatcggagtctctcatgattatattccaaaccatctcacctagtcagaatttgtcattaagaaaactatgggctgggcacggtggcccacgcctataatcccagcactttggaatgccaaggtgggcagatcaactgaggtcaggagttcgaggccagcctgaccaacatggcgaaaccccagctgtactgaaaatacaaaaattagccggtagtgctggcgtgtgcctgtaatctcagctactcgggagactgaggcaggagaatcgcgtgaacccagaagggggaggctgcagtgagccaagatcacactactgcactccagcctgagtgacaaagcaagatttcatctcaaaaaaaaaaaaaaaaaaaagaaagaaaagaaaaaagaaaactatggagcatgacatggaagctggataagaaacttgtttgcaaactccaagaaaaaccaggacctaaaagaagggaagagtggaatggagggtgagcagagtagggggcctaagctctctatatccactttgaacagatttcttccccttttttttggttgtttatattccatttctagaaaagtttcatttggaaaaagggttctactgctttgcggggcaaaaaggaaccatagctaagctgctaactagggtaacatcctatcccctctgaaatacaattaaattcagcaacgccatctggattcagtggctgctgtgtgcagagcgcctgctgggctccatggagaggggaaggaatcagtgtctgcatccaagtagttcacactgtaacaggtgacctatacacacagaccccttgccaggcaatgtgccctgtagtatggccacacagaggaggagtcctggggaggctccatggtgaaggtggcttttgaaggactggcagggtgtcaataggcagagatgcatggacactaaaatccaggcaacaggaacagcatagccacaggaccgggcgtcagtcacattctccccgctggccgaatgacgcaaataatgctcaaattccatgctgcggtttgctttgactggcggaggtggaggggcgcgaaagacgagccagcatgctgcacagcaggtttagcctccatgcccggggtttggagcaagagagtgaaaacatcagagctatgtcttcagaaggtcttgtgttgtgaaactgactggcaagctatcacaggaagctaagcgaggtggcaggctgctagattaaaatgatctctaaacagaattcgctctcagagttcattttcagcctacctcattagggcagatttggtcttctcacattgttattgctgtatcggaggaatctttctaaagagaaaagtgacagcacacattgctgccatttgaaagccagcatttttaaagggcgtaatacatagtgacagttttgatgtatgcccgtgggtgtgggtcctgatatctcaacatactctgcctgcataatttaaaatgacagtttagcctgtcaaacttaaatgtgagtgaagttccttggatatttataaaacatcaaaaagacagaatggtgtcagcaaaagatgggtctttttattactaaaccacgggacgatttttggataattgctggcaactcacctagaagctgaacttccagacaaagtataaatttggtggtgccacagttttgaagattattttcactcaatgcaataaaaggatattacagaactctagcacaaattgtagtcaaagatggagacgcgtcaatgacccgtttgtttttaatgtgttttaatgtttttgtttttaatgtatccatggtttccagttcccctgagaaagtggaaattacccatgctaataaagcaagcgttagtgcgaagacctagctgatgtttggctggtttttagcccgctagccagtcaccctttataaagcctgccaagtttggagcgtgggtaattttacacgcgggtctccacagatcctgtctacttcggtagatttattactaggaaagtgcgtgggggcactaatttttgtgaattgactaatgcgttagaattttaaaaaccgggtagaacgcagcgcaggaagcgagcgttccccgccgcagcgccggagcgtccagccagaatccccctgcatgcgcagcccctggcgtcttacgtcccacaggccccaccccggcgccttccgtcccgcaggctccgcctttggcgctgggctctgacgtcaccacctgcgccgctcacagtagaaacaggaagtgggaccaaaacaaaggagcggcggccgggagcggacttaccttaccttctctgccttcggcgcgcttctcagccgggccgccgacccaaaggagccgtccgactatgtctaacatggagaaacacctgttcaacctgaagttcgcggccaaagaactgagtaggagtgccaaaaaatgcgataaggaggaaaaggccgaaaaggccaaaattaaaaaggccattcagaagggcaacatggaagttgcgaggatacacgccgaaaatgccatccgccagaagaaccaggcggtgaatttcttgagaatgagtgcgcgagtcgatgcagtggctgccagggtccagacggcggtgacgatgggcaaggtgaccaagtcgatggctggtgtggttaagtcgatggatgcgacattgaagaccatgaatctggagaagatttctgctttgatggacaaattcgagcaccagtttgagactctggacgtccagacgcagcaaatggaagacacgatgagcagcacgacgacgctcaccactccccagaaccaagtggatatgctgctccaggaaatggcagatgaggcgggcctcgacctcaacatggagctgccgcagggccagaccggctccgtgggcacgagcgtggcttcggcggagcaggatgaactgtctcagagactggcccgccttcgggatcaagtgtgacggcagaacccgctctgaggtttcctggccatagccaccctttgaaatgctctctgtgtgttagagagatactataccctagaaactctgaacacgccagaatgctgaaatgcccttctacctttgggtttacagccccctccacataaattaagaaattcagtatttctgcactcttagctggattctaaagttctgtatagctcgtaatgatggtatttttatagcagccttttaacagaactagttaatttcgtgtatatgaatctttctcgaagatctggtcaaaactgtattcagtttcctgcccagaatgatcagattgaaggtggttggtttttattattatttagtgtgattgatagtatctagaatggcaggtggtgcataaaagttaaagagaggggaaagattacttagtttggttatacagttataaacaccatgcagtgtattcggtggactgtgctatttctgtttatcctttgggttttggtttttgttttttttttttttgccttcacagtgagactgcaaatgattgttctcataacgtatattattaataaatgtggtcctataatttatactgaaattaccttaggatatttttgcataatactctcttactgcttacattctataaatttttcacgtgataattgtctttgcgtaactgggaaaaatgccgaataacttcctttattatctggaaaaattaaatttgttcatttatattttctacttactaaattgagtttttaaaaagacttagtgtgacatttgacagtgtctttcaaacgaacttctctaacaagtttatagttattttcctgtttcaacactattagaagtcttataaattatgctaattagcatggcagtcatgttacacactcttaacattgccaaagaactgttgatttcgtttgagaaaaccctaggactgtgtgtgtgtaggttttgttttgattttaacaaccaaaaatagaaataaaattagaactgcgttttaagttctaatttgcatttattaatttgtccaaaagcaagaactcttggaaatccttgaaaatataagctggaatgttttacttagccatgcaagtcatttatgtatacatccagccagctggaaatctgagaagtaaagaggtaggactggaaggaaggagaaagcttgagtctttaaggctagagcccagctgtgctgcctgccatcttctcaggaatggcagtgcgtattttctggctgaaaagtaaagcatgtatccaccgctttctcatagcctcgaaacatggagaaaagcaacttgcttttgccttggcaagcatgctaacctaagttaattcaagttttttttaacttaccctttccttcactggaagatttttccataagagaattccattgtttcagaaaataattataggggcccttccaagttctttgaaagattcataaccaactattcactattataacatgtttcccagtgtaaatgagtaaggaaaaaaaaagtgtaacaggtgcgtgcagatgaggagtgaccctcatatttaagttattttatatttgactggacattgttcagaagtgtgctttaagggacacttgttagttgtctgcccagcatctctcaagaatatccctcctgtcctccacatggttgtgcagggccatgtgtgaagacagcatgagtcttaacccctcttttattttatttttgagacagagtctcgctctgttgcccaggctggagtgctgtggcgcgatctctgctcactgcaacctccacctcccgggttcaagtgattctcctgcctcagcctgccgagtagctggaattacaggtgtgcaccaccatgcccagctagtttttttgtatttttagtagagacagggtttcactatgttggccaggctggtcttgaactcctgacctcaggtgatccgcccacctcagcctcccaaagtgctgggattacaggcatgagccactgcacctggccttaacccctctttagattggaaaaaataattacaactttaaaaatagcttagtgttgaaccctttggtaaactaaagacccttttataatgcacatattcccaacaaaattaatatattttgtgagattaaacaatgcttgtatatgcttgaactttcttaaaatatgtccatgtcatactattatgaatgtacatttttatgagtcataaatattattttcaaaagcactacaggcccatgaattacttcctcacttttgcagttgattactgaaatgtaaatcacaagaatttgtcaattaaatcattttaaactgcatgttattggatgtgagtgtgcatcctgttttaaaaaacacttaagaaaaaagaattgcggggcacagtggctcacgcctgtaatcccagcactttgggaggctgaggtgggcagatcacctgaggtcaggagtttgagactagcctgaccaacatggagaaaccccgcctctactaaaaatatgaaattagctgggcgtggtgctgcacacctgtaatctcagctactcaggaggctgaggcaggagaattgcttgaacctgggaggcggaggttgaggtgagccaagattgcgccattgcactctagcctgcgcaacaaggatgaaactcagtctcaaaaaaaggaaaaaaaaaaattctgaggtagatttgggtcagaaagcatgatatttttccaaattcaccctcagtcttagcacttaaatttttgtttggttagtatggctttttcttgcatatttctaggatccctggcttatttttgttgttgctgttttgagacggagtctcactgtcgcccaggctgaagtgccaccacgatctcggctcactgctgcctccacctcccaggttctagcgatcctcctgcctcagccccctgagtagctgggactacagatgcgcaccaacacacctggctaatttttgtttttgttgttgttgttgttgttttagtagagtcggggtttcaccatgttggccaggcttgtctcggactcctgaccctcaagtgatccgcctgcctcggcctcccaaagtgctgggattacaggcgtgagccaccgcgcccggtcccctggcttcttcactgtaattgtttcttcataactacaagattttttttcacattgaaacttccctacaccaaagatggatataatttacaaaagtatccaactgaaacacatcagggaactagttaactagttaatcttccatttaactactagttatatttcttcctttcatttttccgccttcaagtgcacaatttctaacatggatttaggattgaaagaaaaaaggtagactggattaattcacaaaatactttcctctacttttagtttatttcagttcaggaacccagatgatactgtgtgtgctgctggggaattttcttactaaagctgtttcttgctgaaggaatgccaatgtatctagatgccttttaaatacaggcagaccatgaggtttggacactgtgtgcccgccctgctgtctttgatttggaaaagactgctgggccaggtgcagtggctcacgcctgtaatcccaacactttgggaggccgaggcaggcagatcatctgaggttggaagttcgagaccagcctgaccaacatgaagaaaccccgtctctactaaaaatacaaaattagccgggcatggtggcacatgcctgtaatcccagctacttgggaggctgaggcaggagaattgcttaaacccgggaggtggaggttgcggtgagccgagatcgcgccattgcactccagcctgggcaacaagagcgaaactccatctcaaaaaaaaaaaagtgctgaaaataagcaccagtctgataagagaaccttattaactgagcatagtatcagcatcagctggccaagcacactataaagcagaagggagggacttcctctgcaaaaacacaacatcctcttggcaattctgtagccaaagtccaaagctcaagatctttatagcaaaaagctgcaagccagtggacactaaggaaactgatgagcagacccctgtttttgtttttgtgttgaatcaactgactggaattagcaaaatgctgtctggtttacctaggtttgtttttctacattagattgcaaattccatagggagcccgggccaggcaatgtctgtcatagtgtcagtctctgggcctggtgtggtgcctcccacagtgggctccttgcactgcatagcaaacgtgtgcctggcctgggtggtcactcctgatagtgcctcgagctttctgatgacagcagacaggcaggtaaagcaggcaccctaagcgcacagagtcccaagtatggacactcaagagctgtgtcctcttccttcacagcatttgtccacaatgaaatgtttgcctctgttcttcagggtttgtacatcctctccctgctgagaaatgccaggacaaacaattcagcagtgtttactcaaaatgacaatacaggtggggcgtggtggctcacacctgtaatcccagcactttgggaggccgaggtgggcagatcacctgaggtcaggagttcgagaccagcctggccaacagggtgaaaccccgtctctactaaaaatgcaaaaaaataattagccaggcatgatggtgcacacctgtaatcccagctacttcgggaggctgagacaggagactcccttgaacccaggagggggaagttgctgtgagccatgattgtgccactgcactccagcctgggtgacagagtgagactgtctcaaaaaaataaaaataaatagttttttaaatttaaaaacgttttaaatgacaatccagggaaagatttagtccaaaacatgcatgccatgtggggcttctttctagagatgagaacatgcttctgcccttaaatgatattcagtacagataggtagataaaacatacaaagaaaaagaaaaatggacaaggcgtacccagatgttaaaaaaaaaaatcaagaacacttaagatcaggactgtttacacagtttatgttattttatacctcaacctttaaaaaggattaaatcacagtataaggcagcatgccatgctcaccaaattgtgaaacagtaagtgccggaggaaagggatattgtgctgtgtcatggcagttagaagtggcttcccagagccagcagggtttaatctcaggtttcaaagggggaaaaaaagcaggaatgacacaggatctgagaggccaaaaactacagggagttttgactggagtggagggttcacgtgatgttaaaagagactgaaggggagactggacagactgcaaagggctggaggccacattaaaagtgtggactttaccccatgggaaggaggaagcgcaggagtcatgagaaatgatggttcaggcagagattagagcataaagcagggaggtggggaaagccaagaggaacacgctcttggatgacgagtcaccatgaatcactgatcacctgggacatggttcagctgcacatgctgacgcagtgggtctgccagtgacgccgatatgctgcgagtctgggaaccccttttaaggagcagggaatcttaactctggctgtgcattacaactgcctggtactttgtcaccacgtgagtaatgtttttctccaccagtacgaactcctgggctccagtgatcctgctgcctcagcctcctgagtatctgggatccatctatatttttatgtcttgtagataaatatcatgagtcaaatttcattaacattgggaaaagttttttacttgccttgacagatttccaattgctatatgttttcacacacaaaaaattttaaatacatggaaattcagttggtactttttttctcagatccagtagtttgattacaaaggcatttaacagaatagttttcatcttttcatgactctccttgttcctcttcatgatcaattccatggccataagaattctttgacgccaacaacagatgggatataaacagtgcattgttacttttgctaatatcaaacagaagggggatctaaatgaaaatgaaaacatttctgcaaacatgagtgtcctcagctgtcacaggaaataatgaaaaaaaaatcagagtaaatttctttttaactacttccttatagtgtctacaatgcaagtgattttttacaatgctaatttggtatagttttgcacctctgcatctaatagttggagtgaatagtctgcaagggatttttagatctgcttaattcttctggtcagcatgcagtggcatatttaaggaggcaatgttataaccctttaaaatgtaatcatatgcaaaattctatgtatgatttgggagacccctttaaaattctctctatgtatgtaatcacagaagccccaatttgaaaaagtactcctgcaacatgcttggaaatatgccagttgaaacaaggatcaaggcagaaggctggcagaaatctaccactctattatttattccttagaaagtctttcaaagaattgggggcatattcacattttggggtgaatattcacattttattttcattgcacaaacttgaaatgcatgaactcggtaccattcagcatcagcaacaaatcagaagcagctcttggaaatgttcaatgagacctcccacgtctcacttagatctctgagaatcagagctggaaggactctgaatatccattttggaggttggcgacaggagaagacccaccagcctcatcactgtcagcatctcaaagtcactggtctttcatagtgcacaggaagatgcactcacacgtcctctcatcacatctctctagcactccagaggcattttttggttaaccatacttcattctttctacttagactagttattatcatctgaatacattcaccacctacctccaacctatgtgcatcccattttaaataggttacttcctaaactctaactatagccacaggcttcatatttaattttcagttccttccagctcttttcatcagagcgaattcatttctgtatcctttgctctggaccctgtcaaaagttgcgaccatttctgattacaactgttgtagcctgtgagggaagcagccttctcacccagaaacccatctgactgtccagccccagttcagagctcccgtcccaccccggcagaaacttctcctaccttctcatggattccaaggtctttttgctcctttgttgctggatggtcaggaactcagtgtgtccttccaccacgctcttcaccccaccacattccaggccagcaaccaccatgccaaaactgcattgtactttccaaaccaggcctcaggcaatggcagggaattcctgcctccaggctcccaagtctctccagctcccagagggtcccttccctttgttccttcccctctggagccagctccacacccttggccaactcaccaaccttctggtgcgcagtagcacctcccagagcttgcagcacctattccaaagacaccactggctgatcagggtccctttggcctctcaatagccctgtcacctgggccatacctctgcagagccaggagcagttgacattcttttttttttttttgagacggagtctcactctatcgcccaggctggagtgcagtggtgtgatctcggctcactacaacctctgcctcccgggttcaagcgattctcctgcctcagcctcccaagtagctgggattacaggcatgcgtgccaccacacctggctaatttttgtagttttcgtagagacagggtttcacaatgttggctaggttggtctcaaactcctgacctcaggcgatccacccacctccacctcccaaagtgctgggattaaaggcgtgcaccactgcacctggctaacattctttaatgactgcacaccagacaatgcagtcacagacaccactcccatagcctgtttcccttggcttccagggaaatgactcattcatgacagttgaggtcacagttgcccccactgtttcctatcgctatgaaaggccatcccaaacaccagcagatccactcctgcccctttgtgtattctgcttctgggttacttgccctgggggtgccaaacccaagttcatgggtgaaggctgccaaaatgtcatcccatggccatgcctcagttttggccactagggactgtctctagctttccaaaaggaaggagttaagatgtccaaggaaattatgtagtcttactgtctttggccaagtggttcctaaaatggaccccttttgacctctgtagggaaggaaaaagaacttccctctttcccattaggttctgtagctgagtgaactaacaaagacggattaacaggaggaaagcatacacattttatttaatatttttacatgcacacgggaactttcataagaaaaatgaagacccaaagaagctgttaggaccgagagctaatatacccttttaacaaaagatgataaatttatggagaagtgacacaggagaaaggttcaagtttctaggggcagtcattgtggggcagtgactaatgaaagacaagggttattttggtgggtttgtacagatcagtttcagggtggactccgaacccctggtgataagaacattctcctcttcctggtacagggcaggcacgtttcttagggaagatttcatgacctgctttttgggacagagcgggaggtcagccagccagccagccttgcagctgaggcttctcaagtgccttcagctacaattagtcaacatgctgaagggctcattgtggggtggcgttttgtgttctgaaccgtttcgtctccctcttgcccacactgaggttcacaggcgcctgcagaggagctggtgtgggaggatggggagattgggaggcaacatcgcctcctctgcatgaaatgctcatgggcacatgtctgctgcctctacctaccaaaggacagaaccagccaactggcatggcaggcagggagccagcgcagcctccaggccgtccatcctctctcctcagtaccagggcctcccgtcaacgccagcaccaacagagagcctgggcccccccgacccctccctcctgctggctctccttcttccttctagggtccctgctgcccctctgtctccagaattgtcccctgcttgccatttaacccattcccagtgcttgttggtccccgagggacccagcctctcagccctcaatggtcacctgtcccagccgcggaaggagaaggggacagaggacactggttcattccaccatatttactggggccaggcctgcactaggtgctggggactcccaggtggacaagacagagacctgccctgagggcctaacatgttagtggagaagataaataacaacagatcaaccaagagtcagtgggaaacgtgcagcctggatagatgccttggtaaagccaggctggcacagagagggcgggggaggcctgtgcaggggccgtggtcactcaggagagggagctcggcggcctcccagctccctctcggaagggtcatcacccaagagcggcgcacagccttccttggctcccatcctgccttgtgtgggacacagtgggcgccaggcagatctgacaccaacaggcgtcgccaggtttgccagcacacacactcaaatatgcacactcacgttctcgctctcgcacactttccgcacatactctcacactcacccttacacttttacacatttactcttgcacaccacatactcgctctccacactcagtcgctcttacacatattcacgcagtcatacacacacacacacacacacacacacaacatctggatttgattaggaaactaaagggacatctgtcaccttccatgttttgtttacattgcaacacattcttgtactcgcttagccttggacgggaggctccatgctctctcccagtttctgagtagctcccacccccagcgctgtggcagtggagaagagaggggagagaaggcaacattaaaaaaaagaaaaaaaaagaatgcagttctccctccctgggtcaagaatgttgcattatctagacagatacaaattcaggaaacaaagtaagaactcacttcagcttcttggcctgcccccatccttgctgtacttgggtgatgtcccattaatttctccccaaccccaggggaaagtgggcagagaacaggcctcaacccttgctggctttctttgcagcttcctggaaagaatcgacagcgtcagcttggttgactacacacccacagaccaggtatgtggaattagggtcccccaccacacaccagaaactttgagattcatttccaatatgatttaatgattatttcagaatgaattaaggaaaaatccttaagatacctactttttgcaaattgtttgtactgccctgtgtgtgtgcgtggattggtcatcaaaccttgtgccttgctcccctgactctagcacttagtgattatcaaaattgccagcatgcaaatgagtttatctgcagataaggaatatgccgttccttttcctcctggagctgtcctcaggaaagcgaagtctttacttcctggcttcataaactatgcccattctgtttgtggccctgggcctggggggtcccatatcacagtgaactttccaggtgtgacccagagcatcccctttgggttgagtcaaaaaaaaagcactccaccattttttttttttttttgaaatggagtcttgctctgtcgcccagactggagtgctatgttgcaatctctgctcactgcaacctctgcctcccaagttcaagcgattctcctgcctcagccttccaagtagctgggattacaggtgcttgccacctcgcccagctaatttttgtatttttagtggagacagggttttgccatgttggccagactggtctcaaactcctgacctcaagtgatacacctgccttggcctcccaaaatgctgggattacaggcgtgagccaccgtgcccagcccactccaccaattcttaaatagcagagagggaatctttgtgccccttcgaccctcgggcagctgcagagacttggagagcccccggattgctcaattcaggagacttcaggtgcttctagagccagtgaaccccaaggaagttagagggatgtctctgttcaaaccacactctccaaacttcccccagagtctgtattccctgcaaggccttggccctgggcccccaaggtgggaggtctgaggcctgagcttctctctctcccatgaagtttatcctctctaaggctgatcctgagagtttgaaattatctgacaaagcaatggaggcttcctcgtcttgagtcagtccctcaactaacttacctgtcctaaacagttctcgttatactaaaatgggaggaccacctcccatgttccagaattattacagaatggctgcttccttaaaatcacatggatttgggggtcaggaaaaaaaaaaactctgacatctgccaaatgtaccagggatgccagctttgtgaggctgcgtggctttctcgtcccctcggggtaccacatcttttatcttggaggaaacagaaatgtgcagggaagagagaataaatgaaatcaactagcatccagttcataaggctgaagacttgtgagtgcagagttacacttcctttggtttctcacccaagccatcgtgagtttcacccaagccaacagagcaccagcagggatgtccctctggaatgctgatgccctgactgtggttgtcaggtatatgttagtaagattcttgcagacatgattttaagtcttaaaacaaaagcacagttatcaggtctcttggggccaaaccaacacattaactgtggctttctctggaggataaaacagagaacattcgtctgagttctttttttttttttttttttgagacggagtctcactcgtcacccaggctggaatgcaatggcgcgatctcagctccctgcaacttctgcctcttgggttcaagcgattctcctgcctcagcttcccaagtagctgggattacagctgcctgtgaccacacctggctaatttggtttttttgtatttttagtagagatagggtttcaccgtgttggccaggctggtctcaactcctgacctcaggtgatctgcccacctcagcctcccaaagtgctgggattacaggcgtgagtcaccatgctcggccaatgttggttcttaattgttgtttcctttacagtggaacaaattatttgaaaaatattgatgaggtatagttatacccgggctttaccttgaagaagaacagtaatgtaactcagcttgtttccctcttcttgttcccaggacctcctcagatgcagagttctgacatctgggatttttgagacacgattccaagtggacaaagtaaacttccagtgagtatgttgttaagagctgcatggcccagggccacatgatgtcccagagccgaagggctgtgaggtttaagggggcttcattcctgaatgtgcatggcagccctttcaggtaagagcagctggggtgaaatttgaactctagcaagagttgaagtgttgattttactgcctactttaaagtagaaatgaggacacctgaaaggaaggcagtgtttgcatctgagttgttgatttgattggcccttttttttagatcaggagactcatgaaattttgatctttcagatgcacagtcacaggttgagaactgaaatgagcagtttatgtgtgtctccagcccatctcttgggttgcccaactgcaaactcagccaaatgagcttactttcttcattttgctccctcatgataagacattaattctccctccacaaagctcttttccatcctcatagctatcaccctagtcctgctccatcacttcccccgcccctagactctctcggcacagccctggcttgtctcctgtcccctggcttacccatttcagtcgcccttctcagtgccacgaaatgaatcttcctacggcacggcctggatcgtgctgttcctctgctgaagaaggttcggggctcactactgcttgtgcaccgtatgacccaagccctctgcctatgaccagccccgtccccaggcctgctttgtcccctgccacctgcctcaccaggctgggctgttgtcaggacccttccagttctcccagcttttcctgccgtagcgcttctgttcatcttgtcctctgagcctcagatgccacctgtgctaagcaaaccccacaaatctatcaaagcttccttcaggccaggcatagtgattcacgcctgtaatctcagcactttgggaggcaaaggcagtaggattgcttgagaccgggtgttcaagaccagcctgggcaacatagcaagaccctgtctctaccaaaaaaaaaaaaaaaaaattagccaagcttgatggcatacacctgtggtcctagctactcaggaggctgaggtgggaggatctcttaagcccaggaattcaaggctgcagtgggccatgattaggccatttcactccagcctgggtgacagagtaagaccctgtctctaaaaaaaaaaaaaaaaaaaaagcatccaagcatccctcaaatctcagcccatggtgaccccttagaattcccattgcatatctactatgcagccattgatggtatactgccctttaccttgcccagtcaccctcagcatgtctgactcgactctaagggacctcaagggtgaggtcccttaaagcaagagtttcatgatctccagtgtgttctgagactcctgcagtgcccagcgccctcctcagtgcacaggtgacagacacctacttccagatttcccaagaacccttcacatggccactgtcaggctggataaaagccagctacatctctctgctgggaggcgcacagtgcagtatggagttggcgtgacttgtggttttgcccacatcataaccaagtccctggcatagtggaattactaacaatgaagctgtatctcacatctgtgattttgtttgaaaggattctgctcatcagctcatccattttcatcaagtatctcagcgtgccccacgtgtgctaatgtaaagcgtacggattaaattctaataggatcagcaacatacccgcccaggacaaaaaaatctagcccgtgtggtcctaaggcacatgcattatgtttagggaggggagaaatctcagaggcagggaagcctgcgaaggttggaaacatccagcagaatctgggtaggtagaaactctcccagggagaaacgccttatgcgctgagcaggggctgtgagaagaggggcctgggccggcctgatgaagtgcagggtgcagctgagaaactctgagagaactggctgaggtcgccttggctgagcgagaccgggaagcagctcgggctgggcagagcagcctgcactgaatccaggcgtgatcaagacccagtgcaggtcaccaggcagaggtggctagagataagatgcatgtagtgtagacagatcagcaaggagcctttctcaattctccatgggagggaagacacagtgtcctgagagctggaacaaatcacaggaaggatctgccctgggcagtgacttagcctgtactcagagaggcctgagtgtctgagcaggtaacctctagcattactgagctagtaaccactggatttattaaggacctactgtgggacctcagacactgccggctgctgtctgcttgatctgtcacccctgtagccccactgtggggtgactgaggagggactgcggctgagagaggtggagccacttgcagtccaggatcacacagggagtgatggtgcaggccagtgacccgagcttgactcaagacccatgtgtgaacgctggaacctgctgatagcaaaagaggaaagcaagcacgtttgccattgtcccctgcttcccccaaataattgtgttcctcttgctcttccacagcatgtttgatgttggtggccagagggatgagaggagaaaatggatccagtgctttaacggtgatttttttatgctctctcaagaaaataggagtgaattctaacactcagcactgctgtgcttaactattcttgaattagataatctctaactaatatgtaaagtatagcatttatagattatgatgctccttccttagatactaatctcattaaaaaaagcatttaagggcctgacacggtggctcacgcccgtaatcccagcaccttgggaggccaaggagggcggataacctgaagtcaggagttcgagaccagcctggccaacatgtcgaaactggggctctgttaaaaatacaaaaattaggccgggcgtggtggctcatacctgtaatcccagcactttgggaggccgaggcgggcagatcacgaggtcaggagatcgagaccatcctggctaacgaggtgaaaccccgtctgtactaaaaatacaaaaaattagccgggtgcggtggtgggtgcctgtagtcccagctactcaggaggctgaggcaggagaatggcgtgaacctgggaggcagagcttgcagtgagctgagatcgcaccactgcagcagtccggcctgggcaaaagagcgagactctgtcaaaaaaaaataataataataaaaaaattagccgggtgtggtggctcatgcctgtaatcccagcactttgggaggctggggtgggtggatcacctgaggtcaggagttcaagatcagcctgaccaacatagtgaaaccccgtctctactaaaaatgcaaaaattagccgggtgttatggcgcacacctttaatcccagctactcgggaggctgaggcaggagaatcacctgaacccgggaggcagaggttgcagtgagccaagattgcgccattgcactccagcctgggcaacaagagtgaaactctgtctcggaagaaaatacaaaaatacaaaaattagccaggtgtggtgacacatgcctataatcctagctactagggaggctgaggcaggagaatcacttgaacctgggaggcggaagttgcagtgagccaagatcatgccattgcactccagcctgggagacaagagtgaaactccatctcaaaaaaaaaaaagcacttaagcatcccaaatttacatgtgtcttttggggtgggctcttcatcaagtgcgttactgaagcaaccagtggtgcgcggcgcacctgcaggctgttctgtgactgaatagtcctatcactgaatgaatgtttttgtggaactgagtagctgctgggtgtgtactttcttgtaactccacagtgaggatgtctaactgaggtgctttcctttttctccccaccaagatgtcacagctatcatttacgtcgcagcctgcagtagctacaacatggtgattcgagaagataacaacaccaacaggctgagagagtccctggatctttttgaaagcatctggaacaacaggtgacaaaaatagcaaattcagtcttaccattggattgcaaattttcttttgttaaaaatacgctcaggccaggcgttgtggctcacacctgtaatctcaacactgggaggccgaggcaggtgtgtcacttgagctcagcagttggagactagcctgggcaacatggagaaaccctgtctctacaaaaaatacaaaaattagccaggtgtggtggtgcacacctgcccagtgactcaggagcttgaggtagggggatcacttgagcccaggaggtaagggttgcaatgagctgagatcacactactgcactcctgcctgggcaacagagcaagaccttgtctcaaaacacacacacatacctacacccacacccacacacccacacactctctttactgataaatccagaaccgtacgaagtatctcttttagttcatctattgtatagatatacttaaaatgtggaatatttttttttattattttttttttattttgagatagagtctcgctgtgtcacccaggctggagtgcagtggcgccatctcggcccactgcaagctccacctcccgggttcatgccattctccttcctcagcctcctgagtagctgggactacaggcgccctccaccacacccagctaatattttgtatttttagtagagaccgggtttcaccatgttagccaggatggtcttgatctcctgacctcgtgatccacccacctcggcctcccaaagtgctgggattacaggcgtgagccaccgcacccggccaatgtggagtattctttgatgaagttctgatagttctttgtcaagaaaatttaagtctctgttttgaaaggctaagattatattcggccttccatatcttcaggttccacatctgcggattcaaccaaccacagattgaaaatattcaaaaataaatttaagatagcagtacaacaacaaaaaataatacaagattacagtggcatacgcctataatcccagcactttgggaggctgagacgggggaattgcttgagctcaggaattggagaccagtctgggcaacatgttgaaaccctgtctctacaaaaaatacaaaaattaggcatggtggggcgcgcttgtagtcccagctacttgggaagctgaggcaggaggatcacttgagcccaggaggcggaggttacagtaagctgcgattgcatcactgcacgtcagtctgggcgacagagggagactgtgtctcaaaaaacgtaattaattaaaataaaaaattgagcaaataaaaaaattgactaaaaataatacaaataaacaatacagtataacaactacttacctagcttttacatcgtgttaggtattttaggtaattggagatgatttaagcatacgggaggatgtgcatggtttatatgcaaatactacaccatttcatataagggagcctcctcaaattttggttttcacagggagtcctagaccaatcccccacgggtatcaagggatgactctatatctagggttcaaaataggttttgtttggtttttaaattttaggaagtaccagtttaggggccgggaacagtggctcatgcctgtaatcccagcactttgggaggccaaggcaggaagatcacctgagatcaggagttcgagaccaacctggccaatatggcgaaaccccgtctctactaaaactacaaaaattagccaggagtggtggtgggcgcctgtaatcccagctactcgggaggctgaggcaggagaatcgcttgaacccgggaggtggaggttgcagcgagctgagatcgcaccactgaactccagcctgggcaacagagtgagactctgtctcaaaaagtaaaaaaaaaaatttttttaattttgaaaataaataaagtaccagtttaggacatccactaataactagatgatctctaagatcccttacagctcacaatcaccacataatcatgtttgaaactactagcattgcagattggcagaagtgattatttcagaaaggaatatttagcgcgcacacacacaatacatacatatatagttagaaatcaaagtttcttctgaaatattttggaagaaatattaccaaggaggagaggaaacataagtttaccttataaaagttttggattatcagaaaagtgtttttgtatgaaatgttcaacaaccatcctcaaatttgtggtaataagactgttaataggaaagaaacagggtcaggaaataccaggtatacagaaggatactatagagttattatgaatttatttataaacactatatgtgtaagtgggaaaatgaccacaatcttcttataggaaaaagctgattttaaaacactatgatctcgtttttaagtatgtgtgaggacagaaaaaaacttgggcatgaaaacataaaatttctgtagtaattgtttctgggtataattacatttgatttttttctgtatgtttttctataagatttgtttttacaaaggttatatattttattactagaagaagcaggtaagctatttccatttgacgggaaagtggattgtgtgtgtgggtttttctttctttttttttttttttgagacagagtctcgctctgtcccccaggctggagtgcagtggtgcaatctcagctcaccacaacctccacctccccagttcaagcgattctcctgcctcagcctcccgagtagctgggactgcaggcacgtgccaccacgcccagctactttttgtatttttagtagagacagggtttcacagtgttggtcaggctggtctcgaactcctgacctcgtgatctgcccacctctgccccacaaagtgctgggattacaggcgtgagccaccacacgtggccaaattgtgttattaattgatagtaagattcctgtagactaatcagttagcttgattcctttgaagtgatggaggcggaagaagaaccaagccagctgcatgttaagctctgtgttattagctaatgagtcatatattactttgttgttgttaacactttcacttctaatgtgagttttccgaccttttattggtaaattacaccacagaaattcaagtgaactcattacataagtaaatcttagctttggttccaataaatctatatcccccatgggactgaattagaaagtgcttaactacagattgagtatcccttatccgaaatgcttgggaccaaaagtgtttaagatttcttgttgttgttgttttttggaatagttgcattatacttactagtccagcatccctaatatgaaaatgcaaagtgtcaaatgctccgacaagcatttccttcgagcatcatgtcagtgctgaaaaagttttggattttgaagcattttggattttgtgtttttggattagggatgctcaacctgtacctatatttgtttatcattctttacagatggaatgaggatactggtgtactgaactttcttgaatcctatacatttttaggaagacgatggtattcttttaatttagcaacttctatgttagaggcactttcatgtactagtgtatgtgaaatttgtatgtttatttttccttttttaggtggttacggaccatttctatcatcttgttcttgaacaaacaagatatgctggcagaaaaagtcttggcagggaaatcaaaaattgaagactatttcccagaatatgcaaattatactgttcctgaagacggtaagatttcaaaacacattcttatgattgaggaatagaattgttttattaatagtcctgtaactctaattcacatacctctgatgaatcaaagaaattcactttatttaaatcaattttctttctactgcccatatcctaaagtattagagtgttacaaggtcctatttgtaatcggatcccatttgtaaatgtttccgagtttgactttccattgaaaccgtgcagcagaagaaagagccattttgggatgtgactgtgtcatgctggtatgagctctccctctagtgcctttggctgggtgtgacatgacaggtgccttgttagtccttgatacagaccttctcacttcaccctcagatgaccctgcatggtagtttctattattttttcccgtggaaagagagtctgagagaggagagatagcgtgttcaaagccacacgggtagagtcatgcaggcaaggcagtccttgcaccctttgtccccaagcccatgctggcagcagccgcgcttatctgctcctgtctctagccttgctgtggctgcagccttggggatttccagcagggccagcagccatcccaggccacctgagactgaggggtttccggggcacgggaccgtcagtgctaaagctgggaaggtagaccaggaagagttggtcactccgtttccagtcgcttacacttcagtttccagtgaacaccaggtctaccagaggctcataggagtgatttaaggtgaaataacaccgcttcttacatcttgaattccaaactagaaaacgcagaataaaagacactttcctggaaaatatagttaagatttggaaaatttattttttatcctcaatgaagagggaaaagaaaacttgcatttgtcattaaacttttttgctccttatgttcaatgttcttttctccctcatgggagaggcattatataagtatattcatagtaaaatcctgacccttgtggggccttccaacaaaatcctactgctatcacctgtgtcataaagacccgaaaaaaattcctactgctacatatttccttataggaataaataatagacattggaaatgggcctgagcaccagctctgcctgtggtctcaccagacccagctgctttcatgagccaccagcagtctccgcacccgccagagggtcactcagcgtgaggtcaaggtccaggctcttcctgagaccaaatcaagtggaaatctcattgcagttcatcactgccttgctctacccgcagcctgatgatgtgctttccaggactgaggccgggtgccgcttgcccatggcacatcatcagagcatggcttctgctgcgctttctgtggctggcattgccagtttcccagcaagctgggtctttaattctcccgctaaccgcctcttgccacctcctgtcactcagctcaggcagtggctcggcggccggggggtccttccaacagggtctgcctccccaggcccttccctctttccctcctcatggctgtggtccaggccctcactcctctcgtctcagcagctgccacagcttcctgcctgacctcctgtagctggtcactcacctttccagaacattctgtgaactaccaaagtcacccttctgagacacaaccttacctgcttaggagcaccaaggagaagcaccaccactggctgacagccaaggccacctgcccagccgcgggtgctgaagggcttccgtccaggggctgaggggaccctggcttgctgcctcggtgccaggcccagtgactgctcttcacccagcagcatgcgtcatctccatctgtgccctgcctctcccaagagactcacccatccctgagcatctgcagcacctgctggaagcctgggaccaccatcaactccaacgtcaactctcacttagcaattaaaaggaactaacagttggtccatgtgacggcatgggttaaactcacagtaattgtgctgacagaaagaatcaaagcaaaaactacacaccatgtgaatgcatttgtgtaaatgtctaaaaagtaaattagctgggcgtggtggtgtgcgcctgtagtcccagctactcgggaggctgaggcaggagaatcacttgaacccaggaggcggaggttgcagtgagccaagatcgtgccactgcactccagcctgggcgacagaacaaggctccgtctcaaaaaaacaaaaaaaaagcactccagcctgggtgacggagtgagacttagtctcaagaaaaaaaaaaagaactcctcagatggtacacggacactctgggcattccagagtatgtcagtgacccccacccccgccaagataaatgacatgggcgctccccagccactccagaacacagcccaggcagagacccacagcggttgctgcacccttccctccctcactggatgctgcacccacccaccccccacagcaggtgctgcgccctcccctgcccatcactgggtgctgcaccctcccttctatgaatctgtgagtaagtgtcctcacagcctcacacaccctgcaacagagaaggcagagagtggtaagaacactcagctgtgtcgcctagatctcagatttcgtggggttacagaaaataatgataggaccacatgaatgtggactcttccgggggagaatgcctcagaaaggcctagagctagagggtcggccatagtgatctgggtgctccgcatgcctgagtggggagcaggaacagggaaggggagtctcagaagaggaggctgatggaagctgagaggcagtccatggaggccctgccagtgctgcccccagggaggccagggcccagctcctgtgcctggaggctccgagctttcctctcccaacagctctgcagggagggcagctctggggctcaggcaggtcagtaggatttctcccccaccccagcctgtcttgcttgcgctgctgtaacaaaataccttaggctggtgataggatttagatctgtgtccctaaccaaatcttttgtggaattgtaatccccagtgtttgaggtggggcctggtgggaggtgactggatcttggggatggatcatttttgaatggtttagcaccatcctcttgacactgttttcaagatagtgaatgggttctgcaacagcaggtcatttaacagggtgtagcacctcccccatctctctctcgctcctgccctggccacgtgagatgtctcactccctgtgcatcttctgccatgattggaagcttcctgaggcctccccagaagcctagcagataccagcatcgtgcttcccgtacagcctgcagaaccatgagccaattaaacctctgttctttataaattacccagtctcaggtatgtctttatagcaatgcaagaatggactaacacagctggataatttatgaacaacagaaatgtattagtcacagtactggaggctgaaatgtcaaagattaagacaccggccgattcagtgtctggtaagggtttctctgcttcatagatggcactgtctcatttcctcctcacatggtggagagggtgaggggtctctctcaggcctcttacaaggatataatcccatccatgaaggcggagccctcgtgacctcatcacctctcaaaggctgcccctcttgatattgttgcattggagattagcagtcaacatatgaatttggaggagacaaaaacattgagaccatagcacccccagagaaaagttttatcagagcaataactattaaaatgatgtaggagaagagggcaatgaaaattacatgtttggctgggcgcagtgactcacgcttgtaatcccagcactttgggaggccaaggtggccctgaagtcaggagtctgagaccagactggccaacatggtgaaaccctgtctctacaaaaattagccagttgtgatggcgggtgcccgtaattccagctagtagggaggctgaggtgggagaatcacttgaaccggggaggtggaggttgcagtgagccgagatcatgccactgcactccagcctgggcaagagagtgagactccgtctcttaaaaaaaaaagtacatgtttgtttttgccttgctgtacatgtattttaatgctgggaatatacagcagtctaacgttgaaattccttctgtgttttcgtagagttgtatctttgtttcatgttgcttaaataaagttccatttgtcatttctacagcaacaccagatgcaggagaagatcccaaagttacaagagccaagttctttatccgggacctgtttttggtaagcaattttgttaacctttgtttttctacctcccttcttaatcttttgtttcttacaatatgcaaattactccttgatgatctcatttaatcttccttaacattacgagcgatgacaaagggtatgttactttaatttcacagctgagcacaccgaggttcagagaggttagatttctcacccaaggtcacacagcttccaaccagcagagcctgggtgagaacacagcgttccagggaatggcacttggatgggctagacttcattcacttgttgtattttccaaaaaggacagcgtccttcagcagagtctaagcacccatactcttcctccatccaaaagcactaacaggctgatggtttatgtaaaaatggatgtgctcaaattcagatatttacttttttttcttcagctttttgagattatttcaaacttacatatgaggcagaagatttgtacaaataactcccatatactcaggccgggtgcggtggctcatgcctgtcatcccagcactttgggaggctgaggcagatggatcacctgaggtcaggagttcgagactagcctggccaacatggtgaaaccccatctctactaaaaatacaaaaaattagctgggtgtggtggcatgcgcctgtaatcccagctactctggaggctgaggcaggagaattgctgaaacccaggaggcggaggttgcggtgagccgagaccacaccactgcactccagcctggccgacagagcgagactctgtctcaaacaaaacaaacaaaactcccatatagtctttacccaaattgaccaattggcaaaaatttgccacatatgctttatctgtctatatgattttctttgaaataacctacagcgtatagctagctgtgagtttttactcgcttcaaactattacatgccaaggcattcccaagggcgatgttcccggtagtactccaagtgcacttaacacgtgtggacaagatcttagtccaggtagcagtagggctgatcctcggctgtggggcctttccacggggaccggcactccagcctggcctttcaccacccttttcccggcacacccctgtgggatgactcttgaatcctacagtgagttagaaagtaacaatgaggagaaatcaaggccccagggtgagttcagaggacaagagttcccccagaagtgcatggccgtaagtgcatggcaatacccagagagcttctgcagctgaataaatctgggacatgctatgttaaatacagcaagagagcctttacttaagccaattaatgtgcattgcaaatctcttcaataagggaatttagtttgtaatagctcaagggatcccttttttaaggagcattttgtaagaccatagcacataaagacacaagaaaatgctgttctaatgagaactaaatgttaattaagcatgcatatgttgggcatttaaatgaagattagaaacatccaggccggatgcagttgctcatggatgtaatcccaacactttgggaggccaaggcaggtggatcacctgaggccagaagttcgagatcagctagacaacatatcgagacctcatctctacaaaaaattttaaaacgtacctgggtatggtggcacaggcgtgtagtcccagctactcaggaggctgaggtgggaggatcccttgagcctaggaatttgaggtggcagtgagctatgattgtgccactgcaccccaacctgggtgacagagcaacactccatctcaaaataaaaacaaaaacaaaactatttgaagcaacgacctttgtaatgactccattcccttaattttaaagctctagacagggacaaatagtcacactattacagaagaaagccttttttagtttgtttatttgggacattgcttcacactgtcacccaggctgcctggagtgcagtggcaagatcgtagctcactgcagcctccaactcctgggctcaagcaaccctcctgcctcagcctcctgagtagctgggactacaggcacacaccaccgcacccggctaatttttaaattttatagagatacagccttgctatgttggccaggctggtctcgaactcctcgcctcaagtgatcctcctgcctccacctcccaaagtgctgggattacaggtgtgagccactgcacccagccaaaagcctatttttcatccattcactacacacttactgtgtatgtgccaaatacctcacctggtgctttgggattaaaaagatatataactggttggaggagtggggagggatagcattaggagatacacctaatgtaaatgacgagttaatgggtgcagcacaccaacatggcacatgtatacatatgtaacaaacctgcacgttgtgcacatgtatcctagaacttacagtataataaaatatatatatattaaaaatatatatatataacctactcattttagtgtccataagttttttttttaatgctcaagagaagcctgactattgctaaaataggtctatccccgtgaggagtttcatacctcatgacatctttaggataccctttctgtggtccaagcagctctctaaaggctgtttcccaggatcgaaaaaggagtggaggggatgaggaggaggcccagggattctcgtggcaccaaggaagaaggtgagcttccacaggacctctgtctaagtctgcccgggctgccataacaaagtgccgcagaggctgagtgtcttaaacagcagaaatgtatttctcacagctctggaggctggaagtctgaaatcaaggtgcggggtcttccctcgcacatggctgcatcctcctggtttcctcttctctctaggacaccagtcatattgcattagggcccaccctaatgacctcattttaccgtaattgcctctgtaaaggccttatctccaaatatagtcacatctgaggtgccagtcgtcagggattccacacaacttcagggaacacaatttggccctaacagcctgtccccacacccactgcacttagcttcatagtgcacattagcacatcactgaggtggtccccacaaaccatgattcgtaatgtctaaccctgtttctcaggtacttgataacagatcacttttctcccagaaggcagcaaacgttcccccgataaccaggggacaccctgcttagcagatgctaaactgcccctgtgtggaggccgccaccgggccgctgcaccgtccagtacagaaccactgggcacatatggacatttaattaaaactagctggccgggcgcggtggctcacgcctgtaatcccagcactttgggaggccgaggcaggcggatcatgaggtcaggagttcaagaccagcctgaccaacatggtgaaaccccgtctctactaaaaatacaaaaagtagccaggcgtggtggcgcacgcctgtaatcccagctactcaggaggctgaggcaggagaattgcttgaacccgggaggcagaggttgcagtgagccgagatggcaccactgcactccagcctgggggacacagcgagactccatctcaaacaaacaaaaaaacaaacaagctaattaggtggggcacagtggctcatgcctataatcccagcactttgggaggccgaggtgggcggatcacttgaggtcaggagtttgagaccagcctggccaacatggtgacaccccgtctctactaaaaatacaaaaattagccaggcgtgggcacggtgatggacgcctgtaatctcagccacttgagaggctgaggcaggagaatcccttgaacctgggaggtggaggttgcagtgagccaagactgcaccactgcactccagcctgggcaacagagctagactcagtctcaaaaatatataaataaacaaacaaaataaaattagctaattagaaatcagccccttggtggtatcagctagatagcgactatcatattggcagggcagatagtggacattcccattgtcacagaaaactctgttcagaccttcctttttggaagctcctcccttgacttgcctgccggggctttctctgacctatccgtgctgcttcagcctcctgggggcaatgataagggtgaggttatctgggtcctcggccgatgcttgcattgagaccattcctgcctctaagtgctcccatgcaaaacaagcaggccactgtcaccaaagcctccagcacctgctcagcgtggcctagtccttccccagagtacatgctggggccgcgcagggctagtgcacacgctctctcttgcagaggatcagcacggccaccggtgacggcaaacattactgctacccgcacttcacctgcgccgtggacacagagaacatccgcagggtgttcaacgactgccgcgacatcatccagcggatgcacctcaagcagtatgagctcttgtgaggatgctgccgccaccctgcgacggagcggcgccccggactgcctgactgccagccccatgccatggtaggaggcagagtctctagttccatctcgctgccgtctgtcccgttctgtgtcgaccaccaagcctctggctacctctgtcccctcaggtttggttgtgtagcttctgttgtcattgaatacggcctcccgcagcatcccacccccaaaccaccgactctcattgccgacactgcagcagaatctctccgggtgggagccccattattcattctccctttattgattcatcgaggagaacttggtagatggggagaaaacacagttggtttttttttccacgttatcaaccgtgactgcaagagcgttcgtgcagtgccctgagccacggccgtctctgattctccctttatgaagctgcaggctgacgagagatggtcccttcccattggccttagcccaagacttggagtcgaccccaagcgacagagtgaccagaaacccttttacagtcacattcagagtcgctgctggcctcaggcatttgaattagagctactttgagcctcttaggcagaaaacctaccacattcactactgcaaaatgtgtcctgtctaaaaatgattctctaaactttccctatacttaggcatagtcttctttcttagattctctttgttgttgtccctattgctggtttattacactgtacagaccacaaaatgtaatattcttttgtataactactaaagaaaaatccttgtagatctttgtgccttcaccatggctatctatacctgtacatgaaatgtgtttgtattgtgctgaagagcttaatgtcaacattacctgctgcttactctgaaaaaaggaatgaatggtagctgtagaatttaggatattttatcaggttggcactttataaaatactccctgatttaaaaaattgtaagttatacacgttaatcatccacattctatcgacaatgtaccaacatcacaagctgttgcaaccacctgctgttacttctctgagctgtaaaaacctgaactcaattcaggggtacaaattgcaatctaatcttttcagggaaccagggatttttttctctctctctagacaatatgtttcctcattagtctgctaatgaaacacttcttcaagttccccaagtgggaacaggtccatcattcccttagtcaaaactttggacacaggctacgtcatacaagtaagcaaacagtaagagaaaaacaaaatgtggccaggcgcggtggctcacgcctgtaatcccagcactttgggaggccgaggcaggcggatcacgaggccaggagatcaagaccatcctggctaacatggcgaaaccctgtctctaccaaaaatacaaaaattagccaggcgtggtggcgggcacctgtaatctcagctactcgagaggctgaggcaggagaatctcttgaacctgggaggtggagattgcagtgagccgaggtcgtgccatcgcactccagcctggacatcaaagtgagactcaggccaaaaaaaaaaaaaaaaaaaaccttgacgtgtcaatgtttgtgtctggcctaggagaatgaggatgacagcttcacttgccttttgaagaagaaacattacaaaaccttaatctgaagtataaagtcaaaagatacggctctttctcacacttgcaagacttacaaatacagcctcaaacattatgacacaccaacaatatctagaaagtaagaactggtgtagcaatgatgcttcatattctagctgtagccacagagttgagggtagtttttgtaggtctaaaataataatctataaagatgtccaaagttaaattttcaacaatacaaatctagagaagtgacagcctacattacttcattattactcttcttttagtctttagtctttaatattttaaagtttactctataaatcagcattttgtaatcctttataaactcatccagatttaaatgctactttttcatgaagaaaggataactttatagacagtcagtgcaacacacacattttatctcatcaccgtcttactgcctccccatccactgtctcataaagccctcagctgaactaagataaataatacaatggaaattattttcagttccccttgcactgtcaaagtaaaacaagaaaactgaaaagctgcacccccagcaagaaagggaagtatgctgttgtatgcatcattactcaacaattaccctctaactaaacatcctgtttaagagtttaattcaaacaacagccagactgttaagaaaaaaaacaaaaagaataacttttatctggcttacaattattaaagcatttattttcaggtaccaaaagccatatcccattccactttttaagtttcttttgatcactgacaggcattaacagatgtagcaacgtggtctcctatagagaaaattacacttatctaaaaatctgattccattaattgatcaagtataaaaatctacgaaaacaatatgttctgcacatcacatctgtacttttttttttttaaatatattttttgagacggagtctcactctgttgcccaggctggagtgcagtggcatgatcttggctcactgcaacctccgcctcccgggctcaagggattctcctgcctcagcctcagctggtattataggcacttgctaccatgcttggctaatttttgtatttctagcggagacgaggtttcaccatgttggccaggctggtcttgaactcctgacctcaagtgatccacccgcctcagcctcccaaagtgctgggattacaggtgtgagccactgtgcccggccacatctgtacttttaagggtacagctttacagtacataggaatttgagaaccacttcacaggaagagggaaacagcccaatatttatttatgtatacacataatcccaagtgtgtgctggggccaccaggcccttcctgggggaacaaggactgtcgtgcatgtgagtgacgacattaatagcatttacatactgtacagatgcaacctttgatgatacatatatttgataaaaatgagaaaacagatttgttgtagagtacctgtccacttttatagcatgagaacagtacaatcaactatttattttgcagttactcatttcagtgattgagaatttctgtgctgtgcagagagacggcctgtaattggtctcatcatccacttgattctaacatgatctctg

SEQ ID NO: 13 - DNA, 21 bp, Artificial sequence
  Primer
cctcacaagagctcatactgc

SEQ ID NO: 14 - DNA, 25 bp, Artificial sequence
  Primer
caccatgggtctgtgctacagtctg

SEQ ID NO: 15 - DNA, 21 bp, Artificial sequence
  Primer
tcacaagagctcatactgctt

SEQ ID NO: 16 - DNA, 22 bp, Artificial sequence
  Primer
gaacaacaaaaaccgatacgtc

SEQ ID NO: 17 - DNA, 25 bp, Artificial sequence
  Primer
gttcggtttaaagtagataagtcga

SEQ ID NO: 18 - DNA, 25 bp, Artificial sequence
  Primer
taccaaacaacaaaaaccaatacat

SEQ ID NO: 19 - DNA, 25 bp, Artificial sequence
  Primer
gtttggtttaaagtagataagttga

SEQ ID NO: 20 - DNA, 26 bp, Artificial sequence
  Primer
taaagtagataagtcgaaggagaagc

SEQ ID NO: 21 - DNA, 29 bp, Artificial sequence
  Primer
tttaaagtagataagttgaaggagaagtg

SEQ ID NO: 22 - DNA, 20 bp, Artificial sequence
  Primer
taagagagttaggcggtcgc

SEQ ID NO: 23 - DNA, 24 bp, Artificial sequence
  Primer
cctaatctaaaatcccgatacgaa

SEQ ID NO: 24 - DNA, 24 bp, Artificial sequence
  Primer
gtgtaagagagttaggtggttgtg

SEQ ID NO: 25 - DNA, 25 bp, Artificial sequence
  Primer
tccctaatctaaaatcccaatacaa

SEQ ID NO: 26 - DNA, 22 bp, Artificial sequence
  Primer
ttcgttcgttaggagtagggac

SEQ ID NO: 27 - DNA, 21 bp, Artificial sequence
  Primer
cgactaaaacgcttacacgct

SEQ ID NO: 28 - DNA, 25 bp, Artificial sequence
  Primer
tttttgtttgttaggagtagggatg

SEQ ID NO: 29 - DNA, 23 bp, Artificial sequence
  Primer
accaactaaaacacttacacact

SEQ ID NO: 30 - DNA, 250 bp, Artificial sequence
  Probe
gcggccgcaagggacacggcccggaccctgctccctcggggcggcgaagggagcccggcatgcgctcggcccaaagcagacaagccgaaggagaagcggcagcgcaccgagcagctgagtgccgaggagcgcgaggcggccaaggagcgcgaggcggtcaaggaggcgaggaaagtgagccggggcatcgaccgcatgctgcgcgaccagaagcgcgacctgcagcagacgcaccggctcctgctgctcg

SEQ ID NO: 31 - DNA, 405 bp, Artificial sequence
  Probe
gataacaacaccaacaggctgagagagtccctggatctttttgaaagcatctggaacaacaggtggttacggaccatttctatcatcttgttcttgaacaaacaagatatgctggcagaaaaagtcttggcagggaaatcaaaaattgaagactatttcccagaatatgcaaattatactgttcctgaagacgcaacaccagatgcaggagaagatcccaaagttacaagagccaagttctttatccgggacctgtttttgaggatcagcacggccaccggtgacggcaaacattactgctacccgcacttcacctgcgccgtggacacagagaacatccgcagggtgttcaacgactgccgcgacatcatccagcggatgcacctcaagcagtatgagctcttg

SEQ ID NO: 32 - DNA, 145 bp, Artificial sequence
  Probe
atggggtgtttgggcggcaacagcaagacgacggaagaccagggcgtcgatgaaaaagaacgacgcgaggccaacaaaaagatcgagaagcagttgcagaaagagcgcctggcttacaaggctacccaccgcctgctgctcctgg

SEQ ID NO: 33 - DNA, 22 bp, Artificial sequence
  Primer
caggatcctcatctgtttgacg

SEQ ID NO: 34 - DNA, 30 bp, Artificial sequence
  Primer
ggtaccaccatggggtgtttgggcggcacc

SEQ ID NO: 35 - DNA, 20 bp, Artificial sequence
  Primer
caaggaggcgaggaaagtga

SEQ ID NO: 36 - DNA, 21 bp, Artificial sequence
  Primer
aaagagcgcctggcttacaag

SEQ ID NO: 37 - DNA, 21 bp, Artificial sequence
  Primer
gtttgacgatggtgcttttcc

SEQ ID NO: 38 - DNA, 15 bp, Artificial sequence
  Primer
gacgcaccggctcct

SEQ ID NO: 39 - DNA, 22 bp, Artificial sequence
  Primer
gatggtgcttttcccagactca

SEQ ID NO: 40 - DNA, 15 bp, Artificial sequence
  Primer
accagcccccaggag

SEQ ID NO: 41 - DNA, 15 bp, Artificial sequence
  Primer
ccagccccgagcagc

SEQ ID NO: 42 - protein, 73 aa, Homo sapiens
MGCLGGNSKTTEDQGVDEKERREANKKIEKQLQKERLAYKATHRLLLLGAGESGKSTIVKQMRILHVNGFNPE

SEQ ID NO: 43 - protein, 71 aa, Homo sapiens
MGCLGNSKTEDQRNEEKAQREANKKIEKQLQKDKQVYRATHRLLLLGAGESGKSTIVKQMRILHVNGFNGE

SEQ ID NO: 44 - protein, 150 aa, Homo sapiens
MGLCYSLRPLLFGGPGDDPCAASEPPVEDAQPAPAPALAPVRAAARDTARTLLPRGGEGSPACARPKADKPKEKRQRTEQLSAEEREAAKEREAVKEARKVSRGIDRMLRDQKRDLQQTHRLLLLGAGESGKSTIVKQMRILHVNGFNPE

SEQ ID NO: 45 - protein, 173 aa, Homo sapiens
TPRPTRASAWRGKSESSRGRRVYYDEGVASSDDDSSGDESDDGTSGCLRWFQHRRNRRRRKPQRNLLRNFLVQAFGGCFGRSESPQPKASRSLKVKKVPLAEKRRQMRKEALEKRAQKRAEKKRSKLIDKQLQDEKMGYMCTHRLLLLGAGESGKSTIVKQMRILHVNGFNGE